(19) United States
(12) Patent Application Publication
(10) Pub. No.: US 2022/0387397 A1
(43) Pub. Date: Dec. 8, 2022

(54) COMPOUNDS AND COMPOSITIONS FOR TREATING CONDITIONS ASSOCIATED WITH NLRP ACTIVITY

(71) Applicant: Novartis AG, Basel (CH)

(72) Inventors: William ROUSH, Boston, MA (US); Shankar VENKATRAMAN, Lansdale, PA (US); Shomir GHOSH, Brookline, MA (US); Dong-Ming SHEN, Edison, NJ (US); Jason KATZ, Newton, MA (US); Hans Martin SEIDEL, Concord, MA (US)

(21) Appl. No.: 17/287,837

(22) PCT Filed: Oct. 23, 2019
(86) PCT No.: PCT/US2019/057676

§ 371 Date: Apr. 22, 2021

Related U.S. Application Data
(60) Provisional application No. 62/749,713, filed on Oct. 24, 2018.
(60) Provisional application No. 62/749,725, filed on Oct. 24, 2018.
(60) Provisional application No. 62/749,730, filed on Oct. 24, 2018.
(60) Provisional application No. 62/822,405, filed on Mar. 22, 2019.
(60) Provisional application No. 62/822,418, filed on Mar. 22, 2019.
(60) Provisional application No. 62/822,465, filed on Mar. 22, 2019.

(51) Int. Cl. A61K 31/426; A61P 29/00; A61P 1/00; A61K 45/06; A61K 39/395; A61K 31/341; C07D 307/64; C07D 277/36; C07C 311/51; A61K 31/18

(52) U.S. Cl. CPC A61K 31/426; A61P 29/00; A61P 1/00; A61K 45/06; A61K 31/18; A61K 31/341; C07D 307/64; C07D 277/36; C07C 311/51; A61K 39/3955

(57) ABSTRACT

In one aspect, compounds of Formula A, or a pharmaceutically acceptable salt thereof, are featured (Formula A) or a pharmaceutically acceptable salt thereof, wherein the variables shown in Formula A can be as defined anywhere herein.

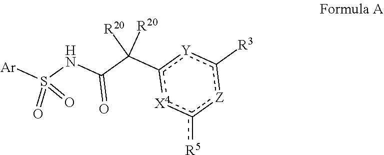

### TECHNICAL FIELD

This disclosure features chemical entities useful, e.g., for treating a condition, disease or disorder in which a decrease or increase in NLRP1/3 activity (e.g., an increase, e.g., a condition, disease or disorder associated with NLRP1/3 signaling) contributes to the pathology and/or symptoms and/or progression of the condition, disease or disorder in a subject (e.g., a human); as well as other methods of using and making the same. The present disclosure relates to, in part, methods and compositions for treating anti-TNFα resistance in a subject with an NLRP3 antagonist. The present disclosure also relates, in part, to methods, combinations and compositions for treating TFNα related diseases and anti-TNFα resistance in a subject that include administration of an NLRP3 antagonist, an NLRP3 antagonist and an anti-TNFα agent, or a composition encompassing an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

The NLRP1 inflammasome is a component of the inflammatory process and its aberrant activation is pathogenic in inherited disorders such as generalized vitiligo associated with autoimmune disease (autoimmune thyroid disease, latent autoimmune diabetes in adults, rheumatoid arthritis, psoriasis, pernicious anemia, systemic lupus erythematosus, and Addison's disease).

NLRP1 and NLRP3 can form a complex and they have been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NRLP1/3, wherein the compounds inhibit NLRP1 or NLRP3 or both NLRP3 and NLRP1.

Several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents. In light of the this, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353:2462-76). Anti-TNFα therapies, however, do not show complete efficacy, however, other cytokines such as IL-1β, IL-6, IL-12, IL-18, IL-21, and IL-23 have been shown to drive inflammatory disease pathology in IBD (Neurath M F Nat Rev Immunol 2014; 14; 329-42). IL-1β and IL-18 are produced by the NLRP3 inflammasome in response to pathogenic danger signals, and have been shown to play a role in IBD. Anti-IL-1β therapy is efficacious in patients with IBD driven by genetic mutations in CARD8 or IL-10R (Mao L et al, J Clin Invest 2018; 238:1793-1806, Shouval D S et al, Gastroenterology 2016; 151:1100-1104), IL-18 genetic polymorphisms have been linked to UC (Kanai T et al, Curr Drug Targets 2013; 14:1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1β, either spontaneously or when stimulated by LPS, and this IL-1β production can be blocked by the ex vivo addition of a NLRP3 antagonist. Based on strong clinical and preclinical evidence showing that inflammasome-driven IL-1β and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1β, IL-6, and IL-18, by genetic factors that pre-dispose IBD patients to having NLRP3 inflammasome activation such as mutations in genes including ATG16L1, CARD8, IL-10R, or PTPN2 (Saitoh T et al, Nature 2008; 456:264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin S et al, Autoimmun Rev 2014; 13:24-30, Steenholdt C et al Gut 2014; 63:919-27). Anti-TNF reduces inflammation in IBD by causing pathogenic T cell apoptosis in the intestine, therefore eliminating the T cell mediated inflammatory response (Van den Brande et al Gut 2007:56:509-17). There is increased NLRP3 expression and increased production of IL-1β in the gut of TNF-non-responsive CD patients (Leal R F et al Gut 2015; 64:233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0:1-15). IL-1β signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1β, which can be inhibited by the addition of an NLRP3 antagonist. NLRP3 inflammasome antagonists—in part by blocking IL-1P secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1β and TNF-α has been shown to increase the risk of opportunistic infections (Genovese M C et al, Arthritis Rheum 2004; 50:1412), therefore, only blocking the NLRP3 inflammasome at the site of inflammation would reduce the infection risk inherent in neutralizing both IL-1□ and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

The present invention also provides alternative therapies for the treatment of inflammatory or autoimmune diseases, including IBD, that solves the above problems associated with anti-TNFα agents.

### SUMMARY

This disclosure features chemical entities (e.g., a compound that modulates (e.g., antagonizes) NLRP1 or NLRP3 or both NLRP1 and NLRP3, or a pharmaceutically acceptable salt, and/or hydrate, and/or cocrystal, and/or drug combination of the compound) that are useful, e.g., for treating a condition, disease or disorder in which a decrease or increase in NLRP1 or NLRP3 or both NLRP1 and NLRP3 activity, also referred to herein “NLRP1/3” activity (e.g., an increase, e.g., a condition, disease or disorder associated with NLRP1/3 signaling).

In some embodiments, provided herein is a compound of Formula A

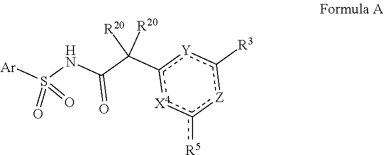

or a pharmaceutically acceptable salt thereof, wherein the variables in Formula A can be as defined anywhere herein.

In some embodiments, provided herein is a compound of Formula I

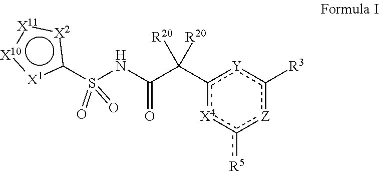

or a pharmaceutically acceptable salt thereof, wherein the variables in Formula I can be as defined anywhere herein.

In some embodiments, provided herein is a compound of Formula IIa

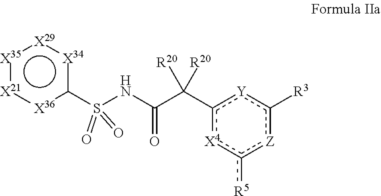

or a pharmaceutically acceptable salt thereof, wherein the variables in Formula IIa can be as defined anywhere herein.

In some embodiments, provided herein is a compound of Formula II

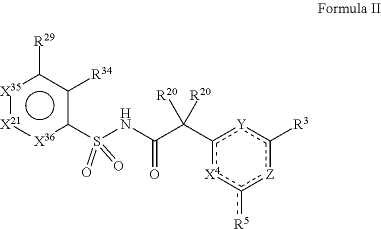

or a pharmaceutically acceptable salt thereof, wherein the variables in Formula II can be as defined anywhere herein.

This disclosure also features compositions as well as other methods of using and making the same.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an compound of Formula I or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount a compound for Formula I or a pharmaceutically acceptable salt, solvate, or co-crystal thereof, wherein the NLRP3 antagonist is a gut-targeted NLRP3 antagonist.

Provided herein are methods of treating a subject in need thereof, that include: (a) identifying a subject having resistance to an anti-TNFα agent; and (b) administering a treatment comprising a therapeutically effective amount of a compound for Formula I, or a pharmaceutically acceptable salt, solvate, or co-crystal thereof to the identified subject.

Provided herein are methods of treating a subject in need thereof, that include: administering a treatment comprising a therapeutically effective amount of a compound for Formula I or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of a compound for Formula I or a pharmaceutically acceptable salt, solvate, or co-crystal thereof.

Provided herein are methods of selecting a treatment for a subject in need thereof, that include selecting a treatment comprising a therapeutically effective amount of a compound for Formula I or a pharmaceutically acceptable salt, solvate, or co-crystal thereof for a subject identified as having resistance to an anti-TNFα agent.

In some embodiments of any of the methods described herein, the treatment further includes a therapeutically effective amount of an anti-TNFα agent, in addition to the NLRP3 antagonist.

An “antagonist” of NLRP1/3 includes compounds that inhibit the ability of NLRP1/3 to induce the production of IL-10 and/or IL-18 by directly binding to NLRP1/3, or by inactivating, destabilizing, altering distribution, of NLRP1/3 or otherwise.

In one aspect, pharmaceutical compositions are featured that include a “chemical entity described herein” which term refers to e.g., a compound described generically or specifically herein or a pharmaceutically acceptable salt thereof or compositions containing the same, and one or more pharmaceutically acceptable excipients.

In one aspect, methods for modulating (e.g., agonizing, partially agonizing, antagonizing) NLRP1 or NLRP3 or both NLRP1 and NLRP3 activity are featured that include contacting NLRP1 or NLRP3 or both NLRP1 and NLRP3 with a “chemical entity described herein” (. Methods include in vitro methods, e.g., contacting a sample that includes one or more cells comprising NLRP1 or NLRP3 or both NLRP1 and NLRP3 (also referred to herein as “NLRP1/3”), as well as in vivo methods.

In a further aspect, methods of treatment of a disease in which NLRP1/3 signaling contributes to the pathology and/or symptoms and/or progression of the disease are featured that include administering to a subject in need of such treatment an effective amount of a “chemical entity described herein”.

In a further aspect, methods of treatment are featured that include administering to a subject a “chemical entity described herein”, wherein the chemical entity is administered in an amount effective to treat a disease in which NLRP1/3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP1/3” is meant to include, without limitation, nucleic acids, polynucleotides, oligonucleotides, sense and antisense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP1/3, or a pharmaceutically acceptable salt and/or hydrate and/or cocrystal thereof;) being administered which will relieve to some extent one or more of the symptoms of the disease or condition being treated. The result includes reduction and/or alleviation of the signs, symptoms, or causes of a disease, or any other desired alteration of a biological system. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids. The term “pharmaceutically acceptable salt” may also refer to pharmaceutically acceptable addition salts prepared by reacting a compound having an acidic group with a base to form a saltor by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine, or formed by reacting with dicyclohexylamine, N-methyl-D-glucamine or tris(hydroxymethyl)methylamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with “excipients”, such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, where “disorder” where used herein is always to be understood as meaning “disorder, disease, or condition” or one or more of the symptoms associated with the disorder; or to slowing the progression, spread or worsening of a disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 12 carbons, such as 3 to 8 carbons, such as 3 to 7 carbons, which may be optionally substituted. Examples of carbocyclic rings include five-membered, six-membered, and seven-membered carbocyclic rings. Carbocyclic rings include monocyclic or bicyclic rings, and when a carbocyclic ring is a bicyclic ring, the bicyclic ring can be fused bicyclic, bridged bicyclic, or spirocyclic.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclic rings include five-membered, six-membered, and seven-membered heterocyclic rings. When a heterocyclic ring is a bicyclic ring, the bicyclic ring can be fused bicyclic, bridged bicyclic, or spirocyclic.

The term “cycloalkyl” as used herein includes an aromatic or nonaromatic cyclic hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl. Cycloalkyl rings include monocyclic or bicyclic rings, and when a carbocyclic ring is a bicyclic ring, the bicyclic ring can be fused bicyclic, bridged bicyclic, or spirocyclic.

The term “heterocycloalkyl” refers to an aromatic or nonaromatic 5-8 membered monocyclic, 8-12 membered bicyclic, or 11-14 membered tricyclic ring system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like. When a heterocycloalkyl ring is a bicyclic ring, the bicyclic ring can be fused bicyclic, bridged bicyclic, or spirocyclic.

The term “hydroxy” refers to an OH group.

The term “amino” refers to an NH₂ group.

The term “oxo” refers to O. By way of example, substitution of a CH₂ a group with oxo gives a C═O group.

As used herein, a curved line connecting two atoms indicates a chain of length as specified by the recited number or number range. For example, a chain connecting an atom “Atom 1” to an atomo “Atom 2” may be depicted as

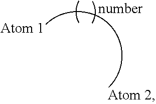

where the number outside the parenthetical indicates the number or range of numbers in the chain.

As used herein, the terms “patient” or “subject” refer to a mammalian organism, preferably a human being, who is diseased with the condition (i.e. disease or disorder) of interest and who would benefit from the treatment.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition.

As used herein, the term “therapeutically effective amount” refers to an amount of the compound of the invention, e.g. tropifexor (as herein defined, e.g. in free form or as a stereoisomer, an enantiomer, a pharmaceutically acceptable salt, solvate, prodrug, ester thereof and/or an amino acid conjugate thereof), or cenicriviroc (in free form or as a pharmaceutically acceptable salt, solvate, prodrug, and/or ester thereof, e.g. in free form or as a pharmaceutically acceptable salt thereof), which is sufficient to achieve the stated effect. Accordingly, a therapeutically effective amount used for the treatment or prevention of a liver disease or disorder as hereinabove defined is an amount sufficient for the treatment or prevention of such a disease or disorder.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The details of one or more embodiments of the invention are set forth in the accompanying drawings and the description below. Other features and advantages of the invention will be apparent from the description and drawings, and from the claims.

### DESCRIPTION OF THE DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

### DETAILED DESCRIPTION

In some embodiments (EAA), provided herein is a compound of Formula AA

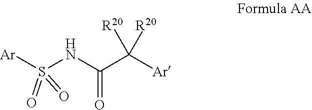

or a pharmaceutically acceptable salt thereof, wherein:
Ar is a heteroaryl group

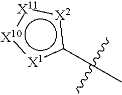

or an aryl or heteroaryl group

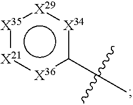

### Ar′ is

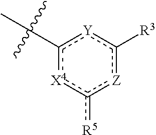

or a 5-membered heteroaryl optionally substituted with one or more R3.
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

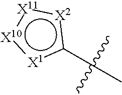

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

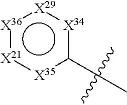

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is N or CR²;

### Z is N or CR⁸;

R⁸ is selected from H, CN, halo, CO₂C₁-C₆ alkyl, CO₂C₃-C₈ cycloalkyl, SO₂NR¹¹R¹², CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy.
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

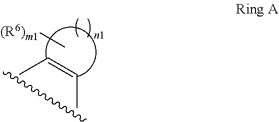

and ring B is

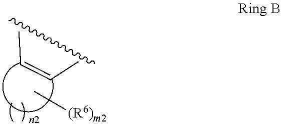

wherein
ring A is a carbocyclic ring or a heterocyclic ring containing 1 or 2 heteroatoms independently selected from O, N, and S;
n1 is from 2 to 5;
ml is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, hydroxy, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₁-C₆ alkyl, NH₂, NHC₁-C₆ alkyl, N(C₁-C₆ alkyl)₂, NO₂, COC₁-C₆ alkyl, SF5, and S(O₂)C₁-C₆ alkyl; wherein the C₁-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl;
   each occurrence of Z¹ is independently selected from O, NR¹⁷C(O), 5-to-10-membered heteroarylene, and 3-10 membered heterocycloalkyl;
   each occurrence of Z² is C₁-C₆ alkylene;
   Z³ is selected from NHCO₂R¹⁵ and 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH,
   o is selected from 0 and 1;
   p is selected from 0, 1, 2, 3, 4, 5, 6, 7, or 8;
   each of R⁷ and R⁸ at each occurrence is independently selected from hydrogen and C₁-C₆ alkyl; with the proviso (P1) that the compound is not a compound selected from the group consisting of:

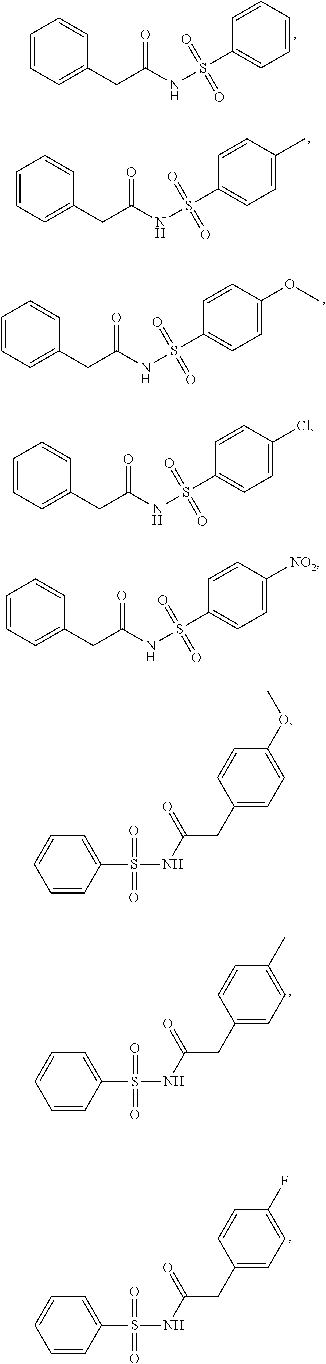

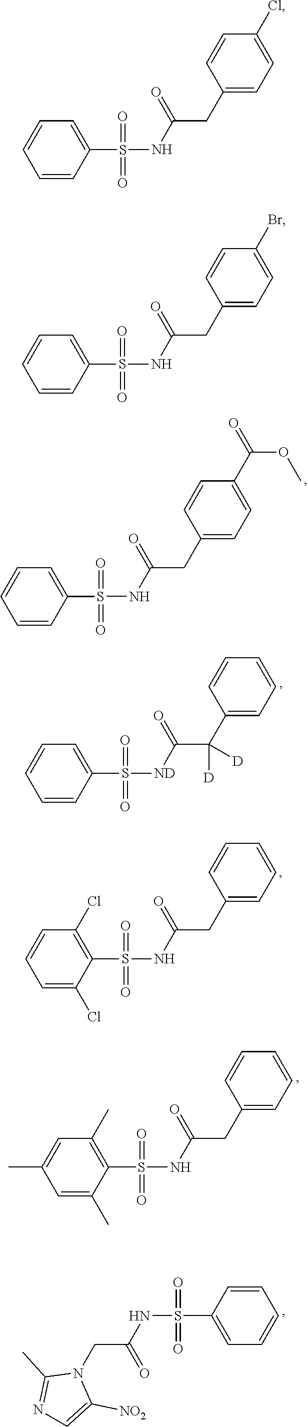

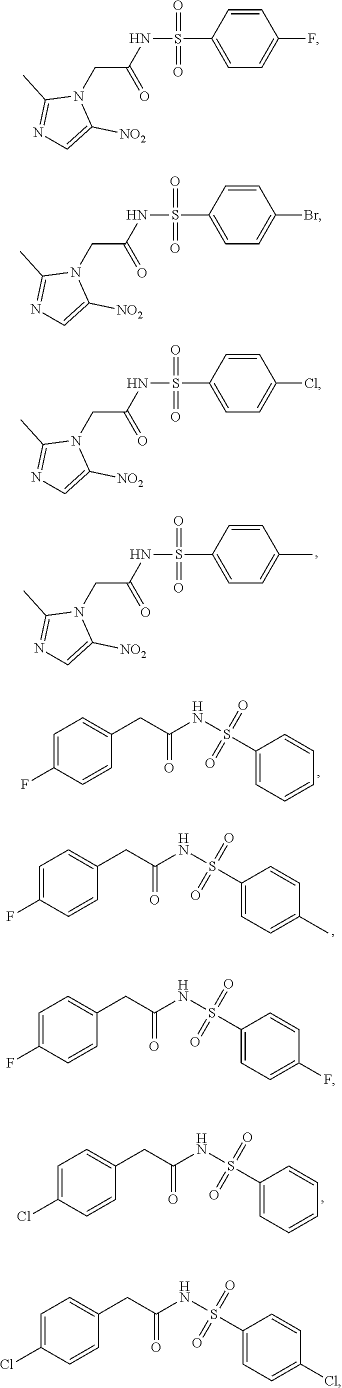

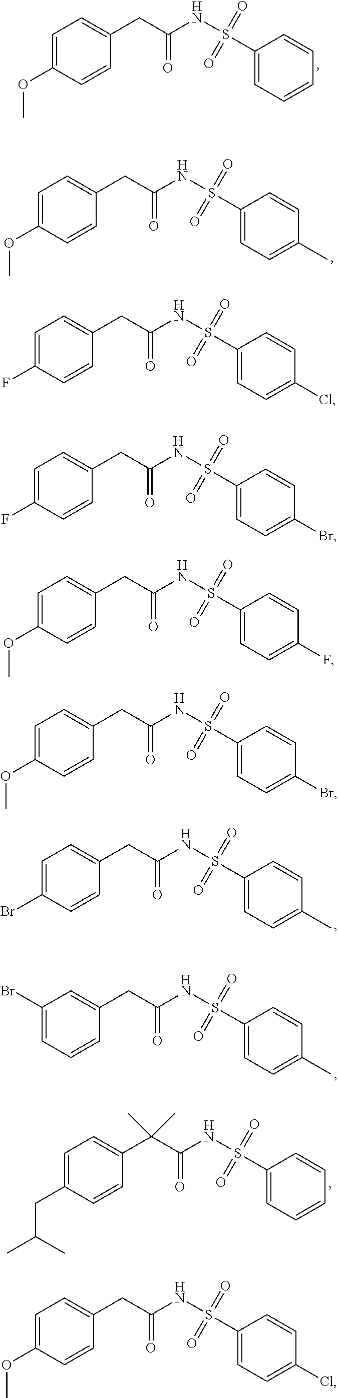

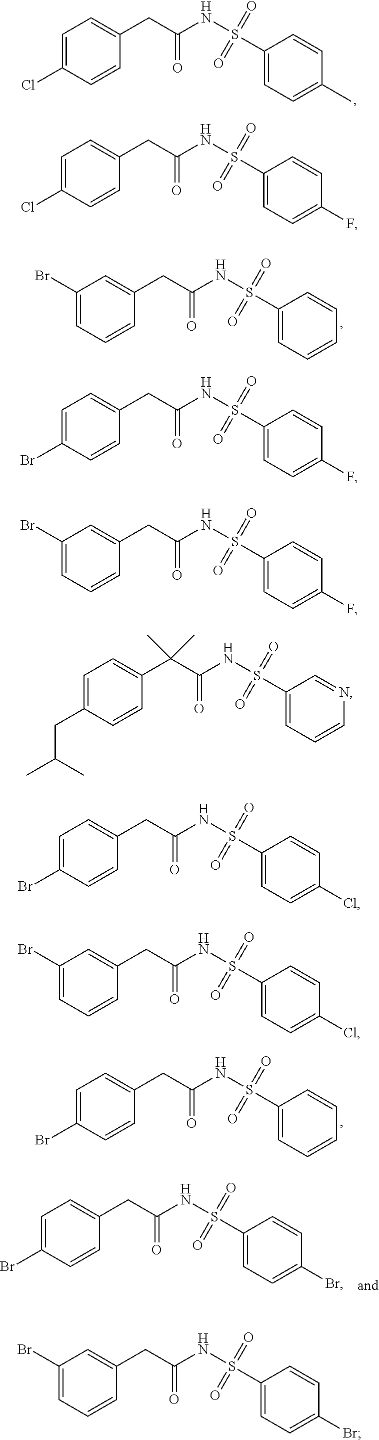

and
with the proviso (P2) that the compound is not a compound selected from the group consisting of the compounds disclosed in Table 1A and Table 1B.
In some embodiments (EAB), provided herein is a compound of Formula AA as shown for embodiments (EAA)
   or a pharmaceutically acceptable salt thereof, wherein:
   Ar is a heteroaryl group

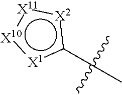

or an aryl or heteroaryl group

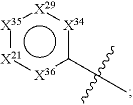

### Ar′ is

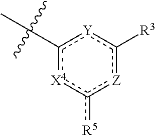

or a 5-membered heteroaryl optionally substituted with one or more R3.
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

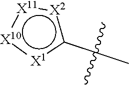

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

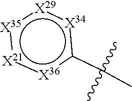

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is N or CR²;

### Z is N or CR⁸;

R⁸ is selected from H, CN, halo, CO₂C₁-C₆ alkyl, CO₂C₃-C₈ cycloalkyl, SO₂NR¹¹R¹², CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy.
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, hydroxy, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₁-C₆ alkyl, NH₂, NHC₁-C₆ alkyl, N(C₁-C₆ alkyl)₂, NO₂, COC₁-C₆ alkyl, SF₅, and S(O₂)C₁-C₆ alkyl;
wherein the C₁-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   wherein when X³⁵ is CR³⁵, X²¹ is CH, X³⁶ is CH, X²⁹ is CH, and X³⁴ is CH, then R³⁵ is selected from hydroxy, C₂-C₆ alkyl, C₁-C₆ haloalkyl, CN, I, CO₂C₂-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₂-C₆ alkyl, NH₂, NHC₁-C₆ alkyl, N(C₁-C₆ alkyl)₂, COC₁-C₆ alkyl, SF₅, and S(O₂)C₁-C₆ alkyl;
   wherein the C₂-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
   wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   wherein when one of X²¹ and X²⁹ is N and the other of X²¹ and X²⁹ is CH, then one of X³⁴, X³⁵, and X³⁶ is other than CH;
   wherein when R
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl;
   each occurrence of Z¹ is independently selected from O, NR¹⁷C(O), 5-to-10-membered heteroarylene, and 3-10 membered heterocycloalkyl;
   each occurrence of Z² is C₁-C₆ alkylene;
   Z³ is selected from NHC₀₂R¹⁵ and 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH,
   o is selected from 0 and 1;
   p is selected from 0, 1, 2, 3, 4, 5, 6, 7, or 8;
   each of R⁷ and R⁸ at each occurrence is independently selected from hydrogen and C₁-C₆ alkyl; and
   with the proviso that the compound is not a compound selected from the group consisting of the compounds disclosed in Table 1A and Table 1B.
In some embodiments (EAC), provided herein is a compound of Formula AA as shown for embodiments (EAA),
   or a pharmaceutically acceptable salt thereof, wherein the compound of Formula AA is a compound of Formula AA-1:

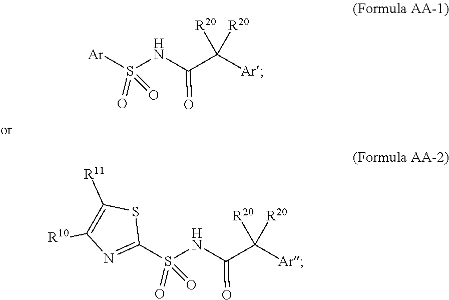

wherein
Ar is a heteroaryl group

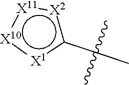

or an aryl or heteroaryl group

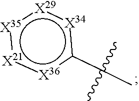

### Ar′ is

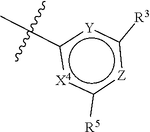

or a 5-membered heteroaryl optionally substituted with one or more R³;

### Ar″ is

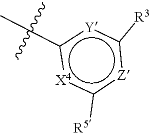

or a 5-membered heteroaryl optionally substituted with one or more R³;
X¹ is S, CR⁴¹, or NH;
X¹⁰ is CR¹⁰,

### X¹¹ is N;

X² is O or CR⁴²;
X³⁵ is CR³⁵;
X²¹ is N or CR²¹;
X³⁶ is CR³⁶;
X²⁹ is N or CR²⁹;
X³⁴ is CR³⁴;
X⁴ is CR⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is CR²;

### Y′ is CR^{2′};

### Z is CR⁸;

### Z′ is CR^{8′};

R⁸ is selected from CN, F, 3-to-10-membered heterocycloalkyl optionally substituted with haloalkyl, and NR¹⁷SO₂R¹⁵;

wherein when R⁸ is F, one of R³⁴, R²⁹, R³⁵, R²¹ and R³⁶ is —(C₁-C₆ alkylene)ₒ-(Z¹-Z²)ₚ-Z³;

R^{8′} is selected from H, 3-to-10-membered heterocycloalkyl optionally substituted with haloalkyl, and NR¹⁷SO₂R¹⁵.
R² is hydrogen or C₁-C₆ alkyl;
R^{2′} is hydrogen;
R³ is hydrogen or CO₂R¹⁵.
R⁴ is hydrogen or C₁-C₆ alkyl;
R⁵ is hydrogen or halo;
R^{5′} is hydrogen;
provided that at least one of R², R³, R⁴, and R⁵ is not hydrogen;
each of R¹⁰, R¹¹, R⁴¹ and R⁴² is independently selected from H and C₁-C₆ alkyl, wherein the C₁-C₆ alkyl, is optionally substituted with one or more substituents each independently selected from hydroxy and —(C₁-C₆ alkoxylene)-5-to-10-membered heterocycloalkyl;

wherein the —(C₁-C₆ alkoxylene)-5-to-10-membered heterocycloalkyl is optionally substituted with one or more hydroxy and/or —(C₁-C₆ alkyl)-OH;

each of R³⁴, R²⁹, R³⁵, R²¹ and R³⁶ is independently selected from H, hydroxy, SO₂NR¹¹R¹², C₂-C₆ alkyl optionally substituted with one or more hydroxy, halo, CONR¹¹R¹², and —(C₁-C₆ alkylene)ₒ-(Z¹-Z²)ₚ-Z³, OC₁-C₆ alkyl;
or R²⁹ and R³⁵, taken together with the atoms connecting them form one monocyclic 5- to 12-membered heterocyclic ring containing 1-3 heteroatoms independently selected from O, N, NH, NR¹³ and S, wherein the heterocyclic ring is optionally substituted with one or more oxo;
wherein at least one of R³⁴, R²⁹, R³⁵, R²¹ and R³⁶ is selected from hydroxy, SO₂NR¹¹R¹², C₂-C₆ alkyl optionally substituted with one or more hydroxy, halo, CONR¹¹R¹², and —(C₁-C₆ alkylene)ₒ-(Z¹-Z²)ₚ-Z³, and OC₁-C₆ alkyl;
wherein when one of X³⁵, X²¹, X³⁶, X²⁹, and X³⁴ is C—C(CH₃)₂OH, then at least two of the remaining X³⁵, X²¹, X³⁶, X²⁹, and X³⁴ are N;
R¹³ is C₁-C₆ alkyl;
each of R¹¹ and R¹² at each occurrence is independently selected from hydrogen and C₁-C₆ alkyl; R¹⁵ is C₁-C₆ alkyl;
each occurrence of Z¹ is independently selected from O, NR¹⁷C(O), 5-to-10-membered heteroarylene, and 3-10 membered heterocycloalkyl;
each occurrence of Z² is C₁-C₆ alkylene;
Z³ is selected from NHCO₂R¹⁵ and 5-to-10 membered monocyclic or bicyclic heterocycloalkyl containing 1-3 heteroatoms selected from O, N, NH, and S, wherein the heterocycloalkyl is optionally substituted with one or more oxo, hydroxy, or —(C₁-C₆ alkylene)-OH,
o is selected from 0 and 1;
p is selected from 0, 1, 2, 3, 4, 5, 6, 7, or 8; and
R¹⁷ at each occurrence is independently selected from hydrogen and C₁-C₆ alkyl; and with the proviso that the compound is not a compound selected from the group consisting of the compounds disclosed in Table 1A and Table 1B.

In some embodiments (EAD), provided herein is a compound of Formula A

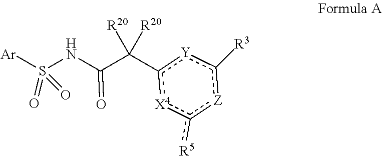

or a pharmaceutically acceptable salt thereof, wherein:
Ar is a heteroaryl group

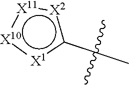

or an aryl or heteroaryl group

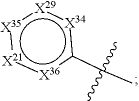

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

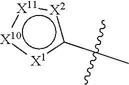

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

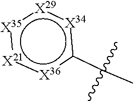

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl; R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B, wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R8, S(O₂)C₁-C₆ alkyl, S(O₂)NR¹⁷R8, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl; and each of R¹⁷ and R⁸ at each occurrence is independently selected from hydrogen and C₁-C₆ alkyl.

In some embodiments (EAE), provided herein is a compound of Formula A

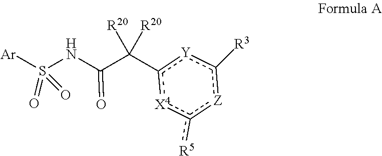

or a pharmaceutically acceptable salt thereof, wherein:
Ar is a heteroaryl group

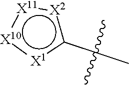

or an aryl or heteroaryl group

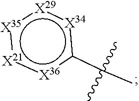

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

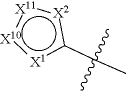

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

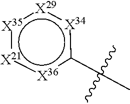

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;
   R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.
In some embodiments (EAF), provided herein is a compound of Formula A as shown for embodiments (EAE),
   or a pharmaceutically acceptable salt thereof, wherein:
   Ar is a heteroaryl group

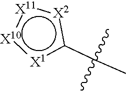

or an aryl or heteroaryl group

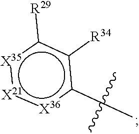

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;
X³⁵ is N or CR³⁵;
X²¹ is N or CR²¹;
X³⁶ is N or CR³⁶;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;
   R¹³ is C₁-C₆ alkyl;
   each of R¹¹ and R¹² at each occurrence is independently selected from hydrogen, C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl;
   each of R¹⁷ and R⁸ at each occurrence is independently selected from hydrogen and C₁-C₆ alkyl.

In some embodiments (EAG), provided herein is a compound of Formula I

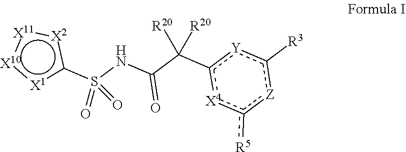

or a pharmaceutically acceptable salt thereof, wherein:
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

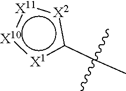

comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl; R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, C₁-C₆ alkyl, oxo, C₁-C₆ alkoxy, NR¹¹R¹², ═NR¹³, COOC₁-C₆ alkyl, C₆-C₁₀ aryl, and CONR¹¹R¹²;
or any two of R⁴¹, R⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.

In some embodiments (EAH), provided herein is a compound of Formula A

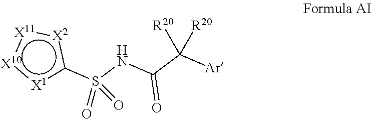

or a pharmaceutically acceptable salt thereof, wherein:

### Ar′ is

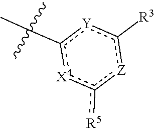

or a 5-to-10-membered heteroaryl optionally substituted with one or more R3
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

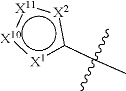

comprises at least one of CR⁴¹, CR¹⁰, CR¹, and CR⁴²; X⁴ is CR⁴, N or NR²⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl;
each of R¹⁷ and R¹⁸ at each occurrence is independently selected from hydrogen and C₁-C₆ alkyl; and
with the proviso that the compound is not a compound selected from the group consisting of the compounds disclosed in Table 1A and Table 1B.
In some embodiments (EAI), provided herein is a compound of Formula I as shown for Embodiments (EAG),
   or a pharmaceutically acceptable salt thereof, wherein:
   X¹ is O, S, N, CR⁴¹ or NR⁴¹;
   X¹⁰ is O, S, N, CR¹⁰ or NR¹⁰;
   X¹¹ is O, S, N, CR¹ or NR¹;
   X² is O, S, N, CR⁴² or NR⁴²;
   X⁴ is CR⁴, N or NR²⁴;
   each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, C₁-C₆ alkyl, oxo, C₁-C₆ alkoxy, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl;
each of R¹⁷ and R¹⁸ at each occurrence is independently selected from hydrogen and C₁-C₆ alkyl.

In some embodiments (EAJ), provided herein is a compound of Formula AII

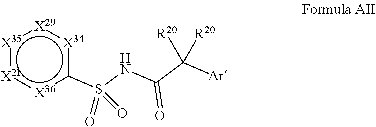

or a pharmaceutically acceptable salt thereof, wherein:

### Ar′ is

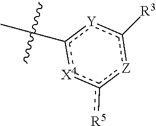

or a 5-membered heteroaryl optionally substituted with one or more R³;
X³⁵ is N or CR³⁵;
X²¹ is N or CR²¹;
X³⁶ is N or CR³⁶;
X²⁹ is N or CR²⁹;
X³⁴ is N or CR³⁴;

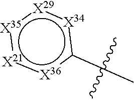

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy.
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

each of R³⁴, R²⁹, R³⁵, R²¹ and R³⁶ is independently selected from H, hydroxy, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₁-C₆ alkyl, NH₂, NHC₁-C₆ alkyl, N(C₁-C₆ alkyl)₂, NO₂, COC₁-C₆ alkyl, SF₅, and S(O₂)C₁-C₆ alkyl; wherein the C₁-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl;
   each occurrence of Z¹ is independently selected from O, NR¹⁷C(O), 5-to-10-membered heteroarylene, and 3-10 membered heterocycloalkyl;
   each occurrence of Z² is C₁-C₆ alkylene;
   Z³ is selected from NHCO₂R¹⁵ and 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH,
   o is selected from 0 and 1;
   p is selected from 0, 1, 2, 3, 4, 5, 6, 7, or 8;
   each of R¹⁷ and R¹⁸ at each occurrence is independently selected from hydrogen and C₁-C₆ alkyl;
   with the proviso (P1) defined for Embodiments (EAA); and
   with the proviso that the compound is not a compound selected from the group consisting of the compounds disclosed in Table 1A and Table 1B.
In some embodiments, (EAK) provided herein is a compound of Formula AII as shown for embodiments (EAJ), or a pharmaceutically acceptable salt thereof, wherein:

### Ar′ is

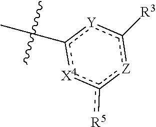

or a 5-membered heteroaryl optionally substituted with one or more R³;
X³⁵ is N or CR³⁵;
X²¹ is N or CR²¹;
X³⁶ is N or CR³⁶;
X²⁹ is N or CR²⁹;
X³⁴ is N or CR³⁴;

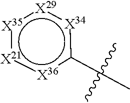

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen, C₁-C₆ alkyl optionally substituted with NR¹⁷CO₂R¹⁵, and NR¹⁷CO₂R¹⁵;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy.
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

each of R³⁴, R²⁹, R³⁵, R²¹ and R³⁶ is independently selected from H, hydroxy, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₁-C₆ alkyl, NH₂, NHC₁-C₆ alkyl, N(C₁-C₆ alkyl)₂, NO₂, COC₁-C₆ alkyl, SF₅, and S(O₂)C₁-C₆ alkyl; wherein the C₁-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   wherein when X³⁵ is CR³⁵, X²¹ is CH, X³⁶ is CH, X²⁹ is CH, and X³⁴ is CH, then R³⁵ is selected from hydroxy, C₂-C₆ alkyl, C₁-C₆ haloalkyl, CN, I, CO₂C₂-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₂-C₆ alkyl, NH₂, NHC₁-C₆ alkyl, N(C₁-C₆ alkyl)₂, COC₁-C₆ alkyl, SF₅, and S(O₂)C₁-C₆ alkyl;
   wherein the C₂-C₆ alkyl, C₃-C₇ cycloalkyl, and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl, wherein the C₆-C₁₀ aryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   wherein when one of X² and X²⁹ is N and the other of X² and X²⁹ is CH, then one of X³⁴, X³⁵, and X³⁶ is other than CH;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl;
   each occurrence of Z¹ is independently selected from O, NR¹⁷C(O), 5-to-10-membered heteroarylene, and 3-10 membered heterocycloalkyl;
   each occurrence of Z² is C₁-C₆ alkylene;
   Z³ is selected from NHCO₂R¹⁵ and 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH,
   o is selected from 0 and 1;
   p is selected from 0, 1, 2, 3, 4, 5, 6, 7, or 8;
   each of R¹⁷ and R⁸ at each occurrence is independently selected from hydrogen and C₁-C₆ alkyl;
   with the proviso P1 as defined under Embodiments (EAA); and
   with the proviso that the compound is not a compound selected from the group consisting of the compounds disclosed in Table 1A and Table 1B.

In some embodiments (EAL), provided herein is a compound of Formula IIa

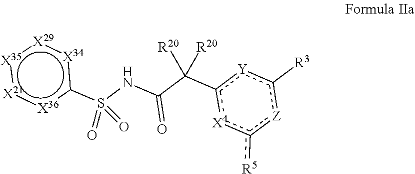

or a pharmaceutically acceptable salt thereof, wherein:
X³⁵ is N or CR³⁵;
X²¹ is N or CR²¹;
X³⁶ is N or CR³⁶;
X³⁴ is N or CR³⁴;
X²⁹ is N or CR²⁹;

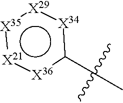

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA); each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.
   In some embodiments (EAM), provided herein is a compound of Formula II

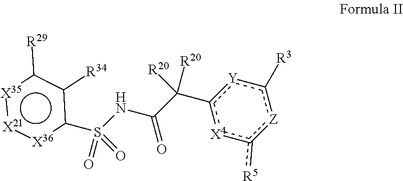

or a pharmaceutically acceptable salt thereof, wherein:
X³⁵ is N or CR³⁵;
X²¹ is N or CR²¹;
X³⁶ is N or CR³⁶;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;
   R¹³ is C₁-C₆ alkyl;
   each of R¹¹ and R¹² at each occurrence is independently selected from hydrogen, C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl;
   each of R¹⁷ and R⁸ at each occurrence is independently selected from hydrogen and C₁-C₆ alkyl.

In some embodiments (EAN), provided herein is a compound of Formula A

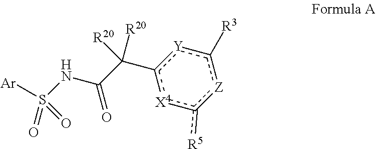

or a pharmaceutically acceptable salt thereof, wherein:
Ar is a heteroaryl group

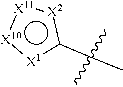

or an aryl or heteroaryl group

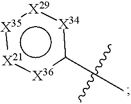

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

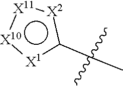

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

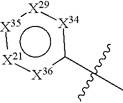

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, and m2 are as defined in embodiments (EAA) and wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
or any two of R⁴¹, R⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
   R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.
In some embodiments(EAO), provided herein is a compound of Formula A as shown for embodiments (EAN),
   or a pharmaceutically acceptable salt thereof, wherein:
   Ar is a heteroaryl group

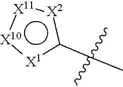

or an aryl or heteroaryl group

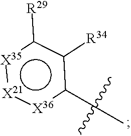

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;
X³⁵ is N or CR³⁵;
X²¹ is N or CR²¹;
X³⁶ is N or CR³⁶;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;
   R¹³ is C₁-C₆ alkyl;
   each of R¹¹ and R¹² at each occurrence is independently selected from hydrogen, C₁-C₆ alkyl, CO₂R¹⁵ and CONR¹⁷R¹⁸;
   R¹⁵ is C₁-C₆ alkyl;
   each of R¹⁷ and R⁸ at each occurrence is independently selected from hydrogen and C₁-C₆ alkyl.
In some embodiments (EAP), provided herein is a compound of Formula I as shown for embodiments (EAI),
   or a pharmaceutically acceptable salt thereof, wherein:
   X¹ is O, S, N, CR⁴¹ or NR⁴¹;
   X¹⁰ is O, S, N, CR¹⁰ or NR¹⁰;
   X¹¹ is O, S, N, CR¹ or NR¹;
   X² is O, S, N, CR⁴² or NR⁴²;

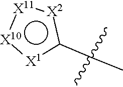

comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R18;
R¹⁵ is C₁-C₆ alkyl; and
each of R¹⁷ and R¹⁸ at each occurrence is independently selected from hydrogen and C₁-C₆ alkyl.
In some embodiments EAQ), provided herein is a compound of Formula I as shown for Embodiments (EAG)

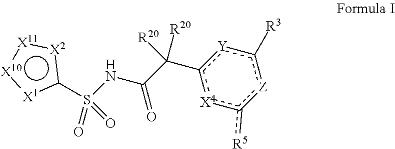

or a pharmaceutically acceptable salt thereof, wherein:
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R8;
R¹⁵ is C₁-C₆ alkyl;
each of R¹⁷ and R⁸ at each occurrence is independently selected from hydrogen and C₁-C₆ alkyl.
In some embodiments (EAR), provided herein is a compound of Formula IIa as shown for embodiments (EAL),
   or a pharmaceutically acceptable salt thereof, wherein:
   X³⁵ is N or CR³⁵;
   X²¹ is N or CR²¹;
   X³⁶ is N or CR³⁶;
   X²⁹ is N or CR²⁹;
   X³⁴ is N or CR³⁴;

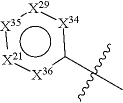

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA);
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
   R¹³ is C₁-C₆ alkyl;
   each of R¹¹ and R¹² at each occurrence is independently selected from hydrogen, C₁-C₆ alkyl, CO₂R¹⁵ and CONR¹⁷R18;
   R¹⁵ is C₁-C₆ alkyl; and
   each of R¹⁷ and R¹⁸ at each occurrence is independently selected from hydrogen and C₁-C₆ alkyl.
In some embodiments (EAS), provided herein is a compound of Formula II as shown for embodiments (EAM)

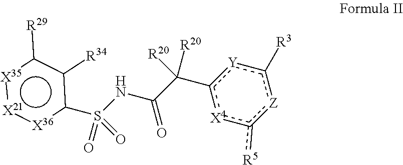

or a pharmaceutically acceptable salt thereof, wherein:
X³⁵ is N or CR³⁵;
X²¹ is N or CR²¹;
X³⁶ is N or CR³⁶;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA); each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;
   R¹³ is C₁-C₆ alkyl;
   each of R¹¹ and R¹² at each occurrence is independently selected from hydrogen, C₁-C₆ alkyl, CO₂R¹⁵ and CONR¹⁷R18;
   R¹⁵ is C₁-C₆ alkyl;
   each of R¹⁷ and R¹⁸ at each occurrence is independently selected from hydrogen and C₁-C₆ alkyl.
   In some embodiments (EAT), provided herein is a compound of Formula A as shown above for Embodiments (EAF),

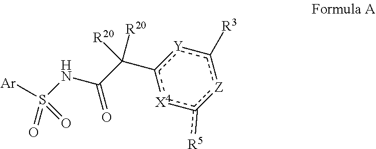

or a pharmaceutically acceptable salt thereof, wherein:
Ar is a heteroaryl group

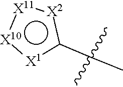

or an aryl or heteroaryl group

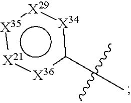

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

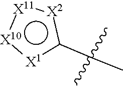

comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴;

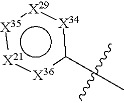

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA); two of R⁴¹, R¹⁰, R¹, and R⁴² are present on adjacent atoms, and taken together with the atoms connecting them form at least one monocyclic or bicyclic 3-to-12-membered nonaromatic carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
two of R³⁴, R²⁹, R³⁵, R²¹ and R³⁶ on adjacent atoms, taken together with the atoms connecting them form at least one monocyclic or bicyclic 3-to-12-membered non-aromatic carbocyclic ring or at least one monocyclic or bicyclic 5- to 12-membered (e.g., non-aromatic) heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R³⁴, R²⁹, R³⁵, R²¹ and R³⁶ that are not taken together with the atoms connecting them to form at least one ring, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and each of R¹⁷ and R⁸ at each occurrence is independently selected from hydrogen and C₁-C₆ alkyl.

In some embodiments (EAX), when R¹ and R¹⁰ are taken together with the atoms connecting them to form a 3-to-8-membered carbocyclic or heterocyclic ring containing 1 or 2 heteroatoms independently selected from O, N, and S;

then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³ COOC₁-C₆ alkyl, C₆-C₁₀ aryl, and CONR¹¹R¹²; and
wherein R¹² is selected from C₁-C₆ haloalkyl, (C═NR¹⁵)NR¹⁷R8, S(O₂)C₁-C₆ alkyl, S(O₂)NR¹⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;

when two of R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³ COOC₁-C₆ alkyl, C₆-C₁₀ aryl, and CONR¹¹R¹²; and
wherein R¹² is selected from C₁-C₆ haloalkyl, (C═NR¹⁵)NR¹⁷R8, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; and

when two of R³⁴, R²⁹, R³⁵, R²¹ and R³⁶ that are on adjacent ring carbon atoms taken together with the adjacent ring carbons form a 3-5 membered or 7-12 membered aromatic carbocyclic ring (e.g., 9-12 membered), a 3-4 membered or 9-12 membered non-aromatic carbocyclic ring, a 7-12-membered aromatic heterocyclic ring, or a 9-12-membered nonaromatic heterocyclic ring, then the carbocyclic or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;

wherein each of R¹¹ and R¹² at each occurrence is independently selected from hydrogen, C₁-C₆ alkyl, C₁-C₆ haloalkyl, (C═NR^{1S})NR¹⁷R8, S(O₂)C₁-C₆ alkyl, S(O₂)NR¹⁷R8, COR¹, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

In some embodiments, when R¹ and R¹⁰ are taken together with the atoms connecting them to form a 3-to-8-membered carbocyclic or heterocyclic ring containing 1 or 2 heteroatoms independently selected from O, N, and S;

then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;

wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and

wherein R¹² is selected from C₁-C₆ haloalkyl, (C═NR¹⁵)NR¹⁷R¹⁸, S(O₂)C₁-C₆ alkyl, S(O₂)NR¹⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;

when two adjacent X²⁹, X³⁴, X²¹, and X³⁶ are other than N, and two of R³⁴, R²⁹, R³⁵, R² and R³⁶ that are on adjacent ring carbon atoms taken together with the atoms connecting them form a 6-membered aromatic ring, a five-to-eight-membered carbocyclic non-aromatic ring, a five- or six-membered heteroaromatic ring or a five-to-eight-membered heterocyclic non-aromatic ring, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;

wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR¹⁷R¹⁸, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; and

when two adjacent X²⁹, X³⁴, X²¹, and X³⁶ are other than N, and two of R³⁴, R²⁹, R³⁵, R²¹ and R³⁶ that are on adjacent ring carbon atoms taken together with the adjacent ring carbons form a 3-5 membered or 7-12 membered aromatic carbocyclic ring (e.g., 9-12 membered), a 3-4 membered or 9-12 membered non-aromatic carbocyclic ring, a 7-12-membered aromatic heterocyclic ring, or a 9-12-membered nonaromatic heterocyclic ring, then the carbocyclic or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;

wherein each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

In some embodiments (EAY), provided herein is a compound of Formula A as shown for embodiments (EAF),
or a pharmaceutically acceptable salt thereof, wherein:
Ar is a heteroaryl group

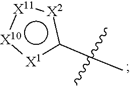

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;

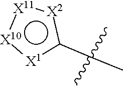

comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA; two of R⁴¹, R¹⁰, R¹, and R⁴² are present on adjacent atoms, and taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹ ‘R’, ═NR¹³ COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R8, S(O₂)C₁-C₆ alkyl, S(O₂)NR¹⁷R8, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and each of R¹⁷ and R⁸ at each occurrence is independently selected from hydrogen and C₁-C₆ alkyl.
In some embodiments (EAZ), provided herein is a compound of Formula A

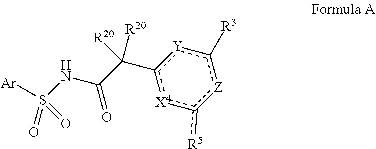

or a pharmaceutically acceptable salt thereof, wherein:
Ar is a heteroaryl group

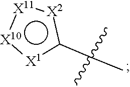

X¹ is O, S, N, CR⁴¹ or NR⁴¹ (e.g., X¹ is S, N, CR⁴¹ or NR⁴¹
X¹⁰ is O, S, N, CR¹⁰ or NR¹⁰;
X¹¹ is O, S, N, CR¹ or NR¹;
X² is O, S, N, CR⁴² or NR⁴² (e.g., X² is S, N, CR⁴¹ or NR⁴¹

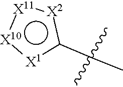

comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;

### Y is N or CR²;

### Z is N or CR⁸;

R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA); two of R⁴¹, R¹⁰, R¹, and R⁴² are present on adjacent atoms, and taken together with the atoms connecting them form at least one monocyclic or bicyclic 3-to-12-membered nonaromatic carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.
In some embodiments of the one or more formulae herein, when R¹ and R¹⁰ are taken together with the atoms connecting them to form a 3-to-8-membered carbocyclic or heterocyclic ring containing 1 or 2 heteroatoms independently selected from O, N, and S, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR⁷R8, S(O₂)C₁-C₆ alkyl, S(O₂)NR⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl.
   In some embodiments (EBA), provided herein is a compound of Formula A

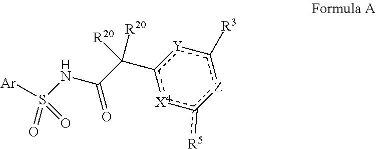

or a pharmaceutically acceptable salt thereof, wherein:
Ar is a heteroaryl group

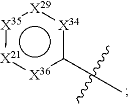

X³⁵ is N or CR³⁵;
X²¹ is N or CR²¹;
X³⁶ is N or CR³⁶;
X²⁹ is N or CR²⁹;
X³⁴ is N or CR³⁴;

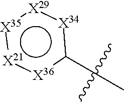

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR⁸;

each of R¹, R³, R⁴, R²⁴ is as defined under embodiment (EAZ), provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA) two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R³⁴, R²⁹, R³⁵, R²¹ and R³⁶ that are not taken together with the atoms connecting them to form at least one ring, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and each of R¹⁷ and R¹⁸ at each occurrence is independently selected from hydrogen and C₁-C₆ alkyl.
In some embodiments (EBB), provided herein is a compound of Formula A

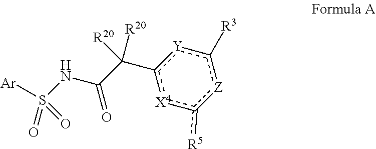

or a pharmaceutically acceptable salt thereof, wherein:
Ar is a heteroaryl group

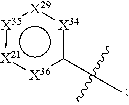

X³⁵ is N or CR³⁵;
X²¹ is N or CR²¹;
X³⁶ is N or CR³⁶;
X²⁹ is N or CR²⁹;
X³⁴ is N or CR³⁴;

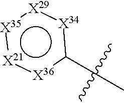

comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;

### Y is N or CR²;

### Z is N or CR;

each of R¹, R³, R⁴, R²⁴ is as defined under embodiment (EAZ),
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein rings A and B as well as n1, m1, n2, m2 and R⁶ are as defined in embodiments (EAA) two of R³⁴, R²⁹, R³⁵, R²¹ and R³⁶ on adjacent atoms, taken together with the atoms connecting them form at least one monocyclic or bicyclic 3-to-12-membered non-aromatic carbocyclic ring or at least one monocyclic or bicyclic 5- to 12-membered (e.g., non-aromatic) heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R³⁴, R²⁹, R³⁵, R²¹ and R³⁶ that are not taken together with the atoms connecting them to form at least one ring, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹¹ is C₁-C₆ alkyl, C₁-C₆ haloalkyl, C₆-C₁₀ aryl, or 5- to 10-membered heteroaryl; and each of R¹⁷ and R⁸ at each occurrence is independently selected from hydrogen and C₁-C₆ alkyl.

In some embodiments (EBC), when two adjacent X²⁹, X³⁴, X²¹, and X³⁶ are other than N, and two of R³⁴, R²⁹, R³⁵, R²¹ and R³⁶ that are on adjacent ring carbon atoms taken together with the atoms connecting them form a 6-membered aromatic ring, a five-to-eight-membered carbocyclic non-aromatic ring, a five- or six-membered heteroaromatic ring or a five-to-eight-membered heterocyclic non-aromatic ring, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR¹⁷R¹⁸, S(O₂)C₁-C₆ alkyl, S(O₂)NR¹⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl.

In some embodiments (EBD), when two adjacent X²⁹, X³⁴, X²¹, and X³⁶ are other than N, and two of R³⁴, R²⁹, R³⁵, R²¹ and R³⁶ that are on adjacent ring carbon atoms taken together with the adjacent ring carbons form a 3-5 membered or 7-12 membered aromatic carbocyclic ring (e.g., 9-12 membered), a 3-4 membered or 9-12 membered non-aromatic carbocyclic ring, a 7-12-membered aromatic heterocyclic ring, or a 9-12-membered nonaromatic heterocyclic ring, then the carbocyclic or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;

wherein each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

In some embodiments (EBE) of the one or more formulae herein, when two of R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR¹⁷R¹⁸, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; and
   when two of R³⁴, R²⁹, R³⁵, R²¹ and R³⁶ that are on adjacent ring carbon atoms taken together with the adjacent ring carbons form a 3-5 membered or 7-12 membered aromatic carbocyclic ring (e.g., 9-12 membered), a 3-4 membered or 9-12 membered non-aromatic carbocyclic ring, a 7-12-membered aromatic heterocyclic ring, or a 9-12-membered nonaromatic heterocyclic ring, then the carbocyclic or heterocyclic ring is substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;

wherein each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

In some embodiments the variables shown in the formulae herein are as follows:

### Formula AA

In some embodiments, the compound of Formula AA is a compound of Formula AA-1 as shown above under embodiments (EAC), or a compound of the formula AA-2 as shown above under embodiments (EAC).

### The Group Ar′

In some embodiments of one or more formulae herein, Ar′ is triazolyl (e.g., 1-triazolyl); or is pyrazolyl (e.g., 1-pyrazolyl); or is pyrrolyl (e.g., 1-pyrrolyl); or is imidazolyl (e.g., 1-imidazolyl); or is furanyl; or is thiophenyl.

### The Group Ar″

In some embodiments of one or more formulae herein, Ar″ is unsubstituted phenyl.
In some embodiments of one or more formulae herein, Ar″ is

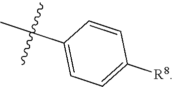

In certain embodiments of the foregoing, R⁸ is NR¹⁷SO₂R¹⁵ (e.g., NHSO₂CH₃).
In some embodiments of one or more formulae herein, Ar″ is

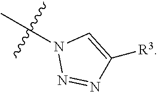

In certain embodiments of the foregoing, R⁸ is CO₂R¹⁵ (e.g., CO₂CH₃).
The Groups X¹, X¹⁰, X¹¹ and X²

In some embodiments of one or more formulae herein,

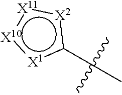

comprises at least one of CR⁴¹, CR¹⁰, CR¹, and CR⁴².

In some embodiments of one or more formulae herein, X¹ is O; or is S; or is N; or is CR⁴¹; or is NR⁴¹.

In some embodiments of one or more formulae herein, X¹⁰ is O; or is S; or is N; or is CR¹⁰; or is NR¹⁰.

In some embodiments of one or more formulae herein, X¹¹ is O; or is S; or is N or is CR¹; or i is NR¹.

In some embodiments of one or more formulae herein, X² is O; or is S; or is N; or is CR⁴²; or is NR⁴²;

The Groups X³⁵, X²¹, X³⁶, X²⁹, and X³⁴

In some embodiments of one or more formulae herein,

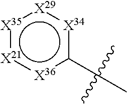

comprises at least two of CR³⁵, CR²¹, CR³⁶, CR²⁹, and CR³⁴.

In some embodiments of one or more formulae herein, X³⁵ is N; or is CR³⁵.

In some embodiments of one or more formulae herein, X²¹ is N; or is CR²¹.

In some embodiments of one or more formulae herein, X³⁶ is N; or is CR³⁶.

In some embodiments of one or more formulae herein, X²⁹ is N; or is

In some embodiments of one or more formulae herein, X²⁹ is CR²⁹.

In some embodiments of one or more formulae herein, X³⁴ is N; or is CR³⁴.

In some embodiments of one or more of formulae herein, X³⁴ is CR³⁴; and X²⁹ is CR²⁹.

In some embodiments of one or more formulae herein, one or two of X²⁹ and X³⁴ is each independently N; and one or two of X²¹ and X³⁶ is each independently N.

In some embodiments, two adjacent X²⁹, X³⁴, X²¹, and X³⁶ are other than N (i.e.,

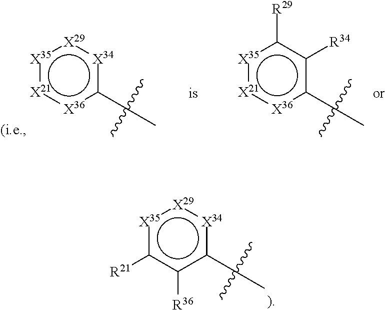

### The Group X⁴

In some embodiments of one or more formulae herein, X⁴ is CR⁴; or is N; or is NR²⁴;

### The Group R²⁰

In some embodiments, each R²⁰ is independently selected from (i) hydrogen, C₁-C₆ alkyl optionally substituted with NR¹⁷CO₂R¹⁵, and NR¹⁷CO₂R¹⁵; or (ii) each R²⁰ is hydrogen; or (iii) from hydrogen and C₁-C₆ alkyl optionally substituted with NR¹⁷CO₂R¹⁵; or (iv) from hydrogen and NR¹⁷CO₂R¹⁵; or (v) R²⁰ is C₁-C₆ alkyl; or (vi) one R²⁰ is hydrogen and the other R²⁰ is C₁-C₆ alkyl; or (vii) one R²⁰ is hydrogen, the other R²⁰ is C₁-C₆ alkyl, and the carbon bonded to each R²⁰ has (S) stereochemistry; or (viii) one R²⁰ is hydrogen, the other R²⁰ is C₁-C₆ alkyl, and the carbon bonded to each R²⁰ has (R) stereochemistry.

### The Group Y

In some embodiments of one or more formulae herein, Y is CR²; or is N.

### The Group Y′

In some embodiments of one or more formulae herein, Y is CR^{2′}.

The Groups R², R^{2′}, R⁴, R³, R⁵, R^{5′}, and R²⁴

In some embodiments of one or more formulae herein, R² is hydrogen; or is C₁-C₆ alkoxy; or is methoxy; or is halo; or is chloro; or is fluoro; or is C₁-C₆ haloalkyl; or is CF₃; or is C₃-C₇ cycloalkyl; or is cyclopropyl; or is C₁-C₆ alkyl optionally substituted with hydroxy; or is isopropyl; or is methyl; or is hydrogen.

In some embodiments of one or more formulae herein, R³ is (ii) hydrogen, C₁-C₆ alkoxy, halo, C₁-C₆ haloalkyl, CN, C₁-C₆ haloalkoxy, C₃-C₇ cycloalkyl, CO₂R¹⁵, or C₁-C₆ alkyl optionally substituted with hydroxy; or is hydrogen, halo, C₁-C₆ haloalkyl, CN, C₃-C₇ cycloalkyl, CO₂R¹⁵, or C₁-C₆ alkyl optionally substituted with hydroxy; or is hydrogen, halo, CN, CO₂R¹⁵, or C₁-C₆ alkyl optionally substituted with hydroxy; or is hydrogen; or is C₁-C₆ alkoxy; or is methoxy; or is C₁-C₆ haloalkoxy; or is CN; or halo; or is chloro; or is fluoro; or is C₁-C₆ haloalkyl; or is CF₃; or is C₃-C₇ cycloalkyl; or is cyclopropyl; or is C₁-C₆ alkyl optionally substituted with hydroxy; or is isopropyl; or is methyl; or is CO₂R¹⁵; or is CO₂Me. In some embodiments of one or more formulae herein, R⁴ is hydrogen; or is C₁-C₆ alkoxy; or is methoxy; or is halo; or is chloro; or is fluoro; or is C₁-C₆ haloalkyl; or is CF₃; or is C₃-C₇ cycloalkyl; or is cyclopropyl; or is C₁-C₆ alkyl optionally substituted with hydroxy; or is isopropyl; or is methyl.

In some embodiments of one or more formulae herein, R⁵ is hydrogen; or is C₁-C₆ alkoxy; or is methoxy; or is C₁-C₆ haloalkoxy; or is CN; or is halo; or is chloro; or is fluoro; or is C₁-C₆ haloalkyl; or is CF₃; or is C₃-C₇ cycloalkyl; or is cyclopropyl; or is C₁-C₆ alkyl optionally substituted with hydroxy; or is hydrogen.

In some embodiments of one or more formulae herein, each of R² and R⁴ is hydrogen or each of R² and R⁴ is C₁-C₆ alkyl optionally substituted with hydroxy.

In some embodiments of one or more formulae herein, R⁵ is isopropyl or is methyl.

In some embodiments of one or more formulae herein, each of R² and R⁴ is isopropyl; or each of R² and R⁴ is t-butyl; or, each of R² and R⁴ is methyl; or each of R² and R⁴ is hydroxymethyl.

In some embodiments of one or more formulae herein, each of R³ and R⁵ is hydrogen; or each of R³ and R⁵ is C₁-C₆ alkyl optionally substituted with hydroxy; or each of R³ and R⁵ is isopropyl; or each of R³ and R⁵ is t-butyl; or each of R³ and R⁵ is methyl; or In some embodiments of one or more formulae herein, each of R³ and R⁵ is hydroxymethyl.

In some embodiments of one or more formulae herein, each of R³ and R⁵ is hydrogen and each of R² and R⁴ is C₁-C₆ alkyl optionally substituted with hydroxy; or each of R³ and R⁵ is hydrogen and each of R² and R⁴ is isopropyl.

In some embodiments of one or more formulae herein, each of R³ and R⁵ is hydrogen and each of R² and R⁴ is t-butyl; or each of R³ and R⁵ is hydrogen and each of R² and R⁴ is methyl; or each of R³ and R⁵ is hydrogen and each of R² and R⁴ is hydroxymethyl; or each of R² and R⁴ is hydrogen and each of R³ and R⁵ is C₁-C₆ alkyl optionally substituted with hydroxy.; or each of R² and R⁴ is hydrogen and each of R³ and R⁵ is isopropyl; or each of R² and R⁴ is hydrogen and each of R³ and R⁵ is t-butyl; or each of R² and R⁴ is hydrogen and each of R³ and R⁵ is methyl; or each of R² and R⁴ is hydrogen and each of R³ and R⁵ is hydroxymethyl.

In some embodiments of one or more formulae herein, R² and R³ taken together with the carbons connecting them form ring A.

In some embodiments of one or more formulae herein, R⁴ and R⁵ taken together with the carbons connecting them form ring B.

In some embodiments of one or more formulae herein, R² and R³ taken together with the carbons connecting them form ring A and R⁴ and R⁵ taken together with the carbons connecting them form ring B.

In some embodiments of one or more formulae herein, at least one of R², R³, R⁴ and R⁵ is not hydrogen.
In some embodiments of one or more formulae herein, R² and R⁴ are not both hydroxymethyl.
In some embodiments of one or more formulae herein, at least one of R², R³, R⁴ and R⁵ is not hydrogen and R² and R⁴ are not both hydroxymethyl.

In some embodiments of one or more formulae herein, R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxyl; or R²⁴ is C₁-C₆ alkyl and R⁵ is ═O; or R²⁴ is C₃-C₈ cycloalkyl and R⁵ is ═O.

### Rings A and B

In some embodiments of one or more formulae herein, ring A is a carbocyclic ring or ring A is a heterocyclic ring containing 1 or 2 heteroatoms independently selected from O, N, and S. In some embodiments of one or more formulae herein, ring B is a carbocyclic ring; or ring B is a heterocyclic ring containing 1 or 2 heteroatoms independently selected from O, N, and S.

In some embodiments, ring A is a carbocyclic ring and n1 is 3; or is a carbocyclic ring and n1 is 4; or is a heterocyclic ring containing 1 or 2 heteroatoms independently selected from O, N, and S and n1 is 3; or is a heterocyclic ring containing 1 or 2 heteroatoms independently selected from O, N, and S and n1 is 4.

In some embodiments, ring B is a carbocyclic ring and n2 is 3; or is a carbocyclic ring and n2 is 4; or is a heterocyclic ring containing 1 or 2 heteroatoms independently selected from O, N, and S and n2 is 3; or is a heterocyclic ring containing 1 or 2 heteroatoms independently selected from O, N, and S and n2 is 4.

### I

In some embodiments, ring A is Ring A

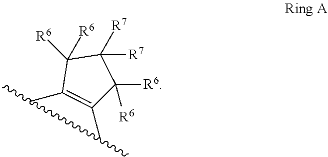

In some embodiments, ring B is

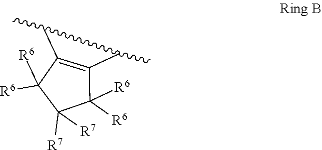

and is the same as ring A.
In some embodiments, ring A is a heterocyclic ring of the formula

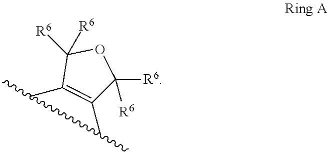

In some embodiments, ring A is a heterocyclic ring of the formula

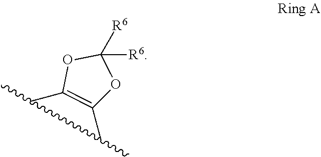

The Groups R⁶ and R⁷ and the Variables n1, n2, m1 and m2 in Ring A and Ring B
In some embodiments of one or more formulae herein, R⁶ is H; or R⁶ is F; or R⁶ is C₁-C₆ alkyl; or
R⁶ is C₁-C₆ alkoxy; or R⁶ is methoxy; or R⁶ is NR¹¹R¹²; or R⁶ is oxo; or R⁶ is —NR¹³.
In some embodiments of one or more formulae herein, n1 is 2; or n1 is 3; or n1 is 4; or n1 is 5.
In some embodiments of one or more formulae herein, n2 is 2; or n2 is 3; or n2 is 4; or
In some embodiments of one or more formulae herein, n2 is 5.
In some embodiments of one or more formulae herein, ml is 1; or ml is 2; or ml is 3; or ml is 4.
In some embodiments of one or more formulae herein, m2 is 1; or m2 is 2; or m2 is 3; or m2 is 4.

In some embodiments of one or more formulae herein, two R⁶ taken together with the atom or atoms connecting them form a 3-to-8-membered carbocyclic or saturated heterocyclic ring containing 1 or 2 heteroatoms independently selected from O, N, and S.
In some embodiments of one or more formulae herein, each R⁶ in each ring is H, or is F; or is C₁-C₆ alkyl.
In some embodiments of one or more formulae herein, each R⁷ in each ring is H; or is C₁-C₆ alkyl.
In some embodiments of one or more formulae herein, each R⁶ in each ring is H and each R⁷ in each ring is H; or each R⁶ in each ring is H and each R⁷ in each ring is C₁-C₆ alkyl; or each R⁶ in each ring is C₁-C₆ alkyl and each R⁷ in each ring is H; or each R⁶ in each ring is C₁-C₆ alkyl and each R⁷ in each ring is C₁-C₆ alkyl.

### The Group Z

In some embodiments of one or more formulae herein, Z is N and X⁴ is CR⁴-; or Z is N and X⁴ is NR²⁴; or Z is CR⁸.

### The Group Z′

In some embodiments of one or more formulae herein, Z′ is CR^{8′}.

### The Group R⁸

In some embodiments of one or more formulae herein, R⁸ (i) is selected from H, CN, halo, CO₂C₁-C₆ alkyl, CO₂C₃-C₈ cycloalkyl, SO₂NR¹¹R¹², CONR¹¹R¹², 3-to-10-membered heterocycloalkyl optionally substituted with haloalkyl, C₁-C₆ alkyl optionally substituted with hydroxy, C₁-C₆ alkoxy, C₁-C₆ haloalkoxy, NR¹⁷SO₂R¹⁵, and C₁-C₆ haloalkyl; or (ii) is selected from H, CN, halo, CO₂C₁-C₆ alkyl, CONR¹¹R¹², 3-to-10-membered heterocycloalkyl optionally substituted with haloalkyl, C₁-C₆ alkoxy, NR¹⁷SO₂R¹⁵, and C₁-C₆ haloalkyl; or (iii) is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl; or (iv) is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl; or (v) is selected from H, CN, Cl, F, CO₂C₁-C₆ alkyl, CO₂C₃-C₈ cycloalkyl, CONR¹¹R¹², C₁-C₆ alkyl optionally substituted with hydroxy, C₁-C₆ haloalkoxy, and C₁-C₆ haloalkyl; or (vi) is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl; or (vii) is selected from H, CN, Cl, F, CO₂C₁-C₆ alkyl and CONH₂; or (viii) is H; or (ix) is CN; or (x) is halo; or (xi) is C₁; or (xii) is F; or (xiii) is CO₂C₁-C₆ alkyl; or (xiv) is CO₂C₃-C₈ cycloalkyl; or (xv) is CONH₂; or (xvi) is CONR¹¹R¹²; or (xvii) is C₁-C₆ alkyl optionally substituted with hydroxy; or (xviii) is C₁-C₆ alkyl (e.g., isopropyl); or (xix) is 3-to-10-membered heterocycloalkyl (e.g., diazirine (e.g., 3H-diazirine)) optionally substituted with haloalkyl (e.g., trifluoromethyl); or (xx) is NR¹⁷SO₂R¹⁵ (e.g., NHSO₂CH₃); or (xxi) is C₁-C₆ alkyl substituted with hydroxy (e.g., hydroxyethyl (e.g., 1-hydroxyeth-1-yl)); or (xxii) is C₁-C₆ alkoxy; or (xxiii) is C₁-C₆ haloalkoxy; or (xxiv) is OCF₃; or (xxv) is C₁-C₆ haloalkyl; or (xxvi) is CF₃; or (xxvii) is CHF₂.

### The Group R^{8′}

In some embodiments of one or more formulae herein, R^{8′} (i) is selected from H, 3-to-10-membered heterocycloalkyl optionally substituted with haloalkyl, and NR¹⁷SO₂R¹⁵; or (ii) is selected from H and NR¹⁷SO₂R¹⁵; or (iii) is selected from H; or (iv) is selected from 3-to-10-membered heterocycloalkyl optionally substituted with haloalkyl; or (v) is selected from NR¹⁷SO₂R¹⁵.
The Groups R1, R¹⁰, R⁴¹ and R⁴²

In some embodiments of one or more formulae herein, each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, —(C₁-C₆ alkoxylene)-5-to-10-membered heterocycloalkyl, and CONR¹¹R¹²;

wherein the —(C₁-C₆ alkoxylene)-5-to-10-membered heterocycloalkyl is optionally substituted with one or more hydroxy or —(C₁-C₆ alkyl)-OH;

In some embodiments of one or more formulae herein, each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹².

In some embodiments of one or more formulae herein, each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹².

In some embodiments of one or more formulae herein, each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹².

In some embodiments of one or more formulae herein, R¹ is H, or is C₁-C₆ alkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₃-C₇ cycloalkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₁-C₆ alkyl optionally substituted with one or more substituents each independently selected from hydroxy, amino and oxo; or is C₁-C₆ alkyl substituted with hydroxy; or is 2-hydroxy-2-propyl; or is C₁-C₆ alkyl optionally substituted with C₆-C₁₀ aryl; or is methyl; or is isopropyl; or

is benzyl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₆-C₁₀ aryl; or is phenyl; or is C₃-C₇ cycloalkyl optionally substituted with one or more substituents each independently selected from hydroxy, amino and oxo; or is C₃-C₇ cycloalkyl; or is C₃-C₇ cycloalkyl substituted with hydroxy; or is 1-hydroxy-1-cyclopropyl; or is 1-hydroxy-1-cyclobutyl; or is 1-hydroxy-1-cyclopentyl; or is 3- to 7-membered heterocycloalkyl optionally substituted with one or more substituents each independently selected from hydroxy, amino and oxo; or is 3- to 7-membered heterocycloalkyl; or 3- to 7-membered heterocycloalkyl substituted with hydroxy; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, oxo, C₁-C₆ alkoxy, NR¹¹R¹², ═NR¹³, COOC₁-C₆ alkyl, and CONR¹¹R¹². In some embodiments of one or more formulae herein, R¹ is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, amino and oxo; or is pyridyl optionally substituted with one or more substituents each independently selected from hydroxy, amino and oxo. In some embodiments, R¹ is pyrimidinyl optionally substituted with one or more substituents each independently selected from hydroxy, amino and oxo; or is pyrrolyl optionally substituted with one or more substituents each independently selected from hydroxy, amino and oxo; or is pyrazolyl optionally substituted with one or more substituents each independently selected from hydroxy, amino and oxo; or; is imidazolyl optionally substituted with one or more substituents each independently selected from hydroxy, amino and oxo; or is oxazolyl optionally substituted with one or more substituents each independently selected from hydroxy, amino and oxo; or is thiazolyl optionally substituted with one or more substituents each independently selected from hydroxy, amino and oxo; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH.

In some embodiments of one or more formulae herein, R¹⁰ is selected from H, C₁-C₆ alkyl, C₃-C₇ cycloalkyl and 3- to 7-membered heterocycloalkyl, wherein R¹⁰ is optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is H; or is C₁-C₆ alkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₃-C₇ cycloalkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₁-C₆ alkyl optionally substituted with one or more substituents each independently selected from hydroxy, amino and oxo; or is C₁-C₆ alkyl substituted with hydroxy; or is 2-hydroxy-2-propyl; or is C₁-C₆ alkyl optionally substituted with C₆-C₁₀ aryl; or is methyl; or is isopropyl; or is benzyl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₆-C₁₀ aryl; or is phenyl; or is C₃-C₇ cycloalkyl optionally substituted with one or more substituents each independently selected from hydroxy, amino and oxo; or is C₃-C₇ cycloalkyl; or is C₃-C₇ cycloalkyl substituted with hydroxy; or 1-hydroxy-1-cyclopropyl; or is 1-hydroxy-1-cyclobutyl; or is 1-hydroxy-1-cyclopentyl; or is 3- to 7-membered heterocycloalkyl optionally substituted with one or more substituents each independently selected from hydroxy, amino and oxo; or is 3- to 7-membered heterocycloalkyl; or is 3- to 7-membered heterocycloalkyl substituted with hydroxy; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, oxo, C₁-C₆ alkoxy, NR¹¹R¹², ═NR¹³, COOC₁-C₆ alkyl, and CONR¹¹R¹². In some embodiments of one or more formulae herein, R¹⁰ is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, amino and oxo; or is pyridyl optionally substituted with one or more substituents each independently selected from hydroxy, amino and oxo; or is pyrimidinyl optionally substituted with one or more substituents each independently selected from hydroxy, amino and oxo; or is pyrrolyl optionally substituted with one or more substituents each independently selected from hydroxy, amino and oxo; or is pyrazolyl optionally substituted with one or more substituents each independently selected from hydroxy, amino and oxo; or is imidazolyl optionally substituted with one or more substituents each independently selected from hydroxy, amino and oxo; or is oxazolyl optionally substituted with one or more substituents each independently selected from hydroxy, amino and oxo; or is thiazolyl optionally substituted with one or more substituents each independently selected from hydroxy, amino and oxo; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH.

In some embodiments of one or more formulae herein, R⁴¹ is H; or is C₁-C₆ alkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₃-C₇ cycloalkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₁-C₆ alkyl optionally substituted with one or more substituents each independently selected from hydroxy, amino and oxo; or is C₁-C₆ alkyl substituted with hydroxy; or is 2-hydroxy-2-propyl; or is C₁-C₆ alkyl optionally substituted with C₆-C₁₀ aryl; or is methyl; or is isopropyl; or is benzyl; or is C₆-C₁₀ aryl; or is phenyl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₃-C₇ cycloalkyl optionally substituted with one or more substituents each independently selected from hydroxy, amino and oxo; or is C₃-C₇ cycloalkyl; or is C₃-C₇ cycloalkyl substituted with hydroxy; or is 1-hydroxy-1-cyclopropyl; or is 1-hydroxy-1-cyclobutyl; or is 1-hydroxy-1-cyclopentyl; or is 3- to 7-membered heterocycloalkyl optionally substituted with one or more substituents each independently selected from hydroxy, amino and oxo; or is 3- to 7-membered heterocycloalkyl; or is 3- to 7-membered heterocycloalkyl substituted with hydroxy; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, amino and oxo; or is pyridyl optionally substituted with one or more substituents each independently selected from hydroxy, amino and oxo; or is pyrimidinyl optionally substituted with one or more substituents each independently selected from hydroxy, amino and oxo; or is pyrrolyl optionally substituted with one or more substituents each independently selected from hydroxy, amino and oxo; or is pyrazolyl optionally substituted with one or more substituents each independently selected from hydroxy, amino and oxo; or is imidazolyl optionally substituted with one or more substituents each independently selected from hydroxy, amino and oxo; or is oxazolyl optionally substituted with one or more substituents each independently selected from hydroxy, amino and oxo; or is thiazolyl optionally substituted with one or more substituents each independently selected from hydroxy, amino and oxo; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH.

In some embodiments of one or more formulae herein, R⁴² is selected from H, C₁-C₆ alkyl, C₃-C₇ cycloalkyl and 3- to 7-membered heterocycloalkyl, wherein R⁴² is optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is H; or is C₁-C₆ alkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₃-C₇ cycloalkyl optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is C₁-C₆ alkyl optionally substituted with one or more substituents each independently selected from hydroxy, amino and oxo; or is C₁-C₆ alkyl substituted with hydroxy; or is 2-hydroxy-2-propyl; or is C₁-C₆ alkyl optionally substituted with C₆-C₁₀ aryl; or is methyl; or is isopropyl; or is benzyl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₆-C₁₀ aryl; or is phenyl; or is C₃-C₇ cycloalkyl optionally substituted with one or more substituents each independently selected from hydroxy, amino and oxo; or is C₃-C₇ cycloalkyl; or is C₃-C₇ cycloalkyl substituted with hydroxy; or is 1-hydroxy-1-cyclopropyl; or is 1-hydroxy-1-cyclobutyl; or is 1-hydroxy-1-cyclopentyl; or is 3- to 7-membered heterocycloalkyl optionally substituted with one or more substituents each independently selected from hydroxy, amino and oxo; or is 3- to 7-membered heterocycloalkyl; or is 3- to 7-membered heterocycloalkyl substituted with hydroxy; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, amino and oxo; or is pyridyl optionally substituted with one or more substituents each independently selected from hydroxy, amino and oxo; or is pyrimidinyl optionally substituted with one or more substituents each independently selected from hydroxy, amino and oxo; or is pyrrolyl optionally substituted with one or more substituents each independently selected from hydroxy, amino and oxo; or is pyrazolyl optionally substituted with one or more substituents each independently selected from hydroxy, amino and oxo; or is imidazolyl optionally substituted with one or more substituents each independently selected from hydroxy, amino and oxo; or is oxazolyl optionally substituted with one or more substituents each independently selected from hydroxy, amino and oxo; or is thiazolyl optionally substituted with one or more substituents each independently selected from hydroxy, amino and oxo; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH; or is —(C₁-C₆ alkoxylene)-5-to-10-membered heterocycloalkyl, wherein the —(C₁-C₆ alkoxylene)-5-to-10-membered heterocycloalkyl is optionally substituted with one or more hydroxy or —(C₁-C₆ alkyl)-OH.

In some embodiments of one or more formulae herein, one of R¹ and R¹⁰ is C₁-C₆ alkyl optionally substituted with one or more substituents each independently selected from hydroxy, amino and oxo, and the other of R¹ and R¹⁰ is C₃-C₇ cycloalkyl optionally substituted with one or more substituents each independently selected from hydroxy, amino and oxo; or one of R¹ and R¹⁰ is 2-hydroxy-2-propyl and the other of R¹ and R¹⁰ is 1-hydroxy-1-cyclobutyl; or one of R¹ and R¹⁰ is 2-hydroxy-2-propyl and the other of R¹ and R¹⁰ is 1-hydroxy-1-cyclopentyl. In some embodiments of one or more formulae herein, R¹ is optionally substituted with one or more substituents each independently selected from hydroxy, amino and oxo, and the hydroxy, amino or oxo substituent is at the carbon of R¹ directly bonded to the five-membered heteroaryl ring of the formulae herein.
In some embodiments of one or more formulae herein, R¹⁰ is optionally substituted with one or more substituents each independently selected from hydroxy, amino and oxo, and the hydroxy, amino or oxo substituent is at the carbon of R¹¹ directly bonded to the five-membered heteroaryl ring of the formulae herein.
In some embodiments of one or more formulae herein, wherein R⁴¹ and R¹⁰, taken together with the atoms connecting them form a monocyclic or bicyclic 3-to-12-membered carbocyclic ring or a monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R¹⁰ and R¹, taken together with the atoms connecting them form a monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R¹ and R⁴², taken together with the atoms connecting them form a monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R1.
In some embodiments of one or more formulae herein, R¹ and R¹⁰ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³, and S; or a five-membered heterocyclic ring containing 1 or 2 heteroatoms independently selected from O, N, NH, NR¹³, and S; or a six-membered heterocyclic ring containing 1 or 2 heteroatoms independently selected from O, N, NH, NR¹³ and S (e.g., a six-membered heterocyclic ring containing 2 heteroatoms wherein one heteroatom is N; and the other heteroatom is selected from O, NH, and NMe (e.g., the other heteroatom is O)); or a seven-membered heterocyclic ring containing 1 or 2 heteroatoms independently selected from O, N, NH, NR¹³, and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².

In certain embodiments of the foregoing, each of R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²; and

each of R⁴¹ and R⁴² when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²; or each of R⁴¹ and R⁴² is H; or one of R⁴¹ and R⁴² is H; and the other one of R⁴¹ and R⁴² is other than H; or, one of R⁴¹ and R⁴² is H; and the other one of R⁴¹ and R⁴² is C₁-C₆ alkyl which is optionally substituted as described elsewhere herein.
In some embodiments of one or more formulae herein, R¹ and R⁴² taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³, and S; or a five-membered heterocyclic ring containing 1 or 2 heteroatoms independently selected from O, N, NH, NR¹³ and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from O, NH, and NMe, e.g., the other heteroatom is O); or a seven-membered heterocyclic ring containing 1 or 2 heteroatoms independently selected from O, N, NH, NR¹³ and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².

In certain embodiments of foregoing, each of R⁴ and R¹⁰ when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²; and

each of R⁴¹ and R¹⁰ when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R1.

In certain embodiments, each of R⁴¹ and R¹⁰ is H; or one of R⁴¹ and R¹⁰ is H; and the other one of R⁴¹ and R¹⁰ is other than H; or one of R⁴¹ and R¹⁰ is H; and the other one of R⁴¹ and R¹⁰ is C₁-C₆ alkyl which is optionally substituted as described elsewhere herein.
In some embodiments of one or more formulae herein, R⁴¹ and R¹⁰ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³, and S; or a five-membered heterocyclic ring containing 1 or 2 heteroatoms independently selected from O, N, NH, NR¹³ and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from O, NH, and NMe (e.g., the other heteroatom is O)); or a seven-membered heterocyclic ring containing 1 or 2 heteroatoms independently selected from O, N, NH, NR¹³ and S; or an eight-membered heterocyclic ring containing 1 or 2 heteroatoms independently selected from O, N, NH, NR¹³, and S; or a nine-membered heterocyclic ring (e.g., spirocyclic nine membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².

In certain embodiments of foregoing, each of R¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²; and

each of R¹ and R⁴² when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R1, ═NR¹³ COOC₁-C₆ alkyl, C₆-C₁₀ aryl, and CONR¹¹R¹².
In certain embodiments, each of R¹ and R⁴² is H; or one of R¹ and R⁴² is H; and the other one of R¹ and R⁴² is other than H; or one of R¹ and R⁴² is H; and the other one of R¹ and R⁴² is C₁-C₆ alkyl which is optionally substituted as described elsewhere herein.
   The Groups R¹¹ and R¹²

In some embodiments of one or more formulae herein, each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;
In some embodiments of one or more formulae herein, R¹¹ is hydrogen; or is C₁-C₆ alkyl optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or is CO₂R¹⁵; or is CONR¹⁷R¹⁸; or is C₁-C₆ haloalkyl; or is (C═NR¹⁵)NR¹⁷R¹⁸; or is S(O₂)C₁-C₆ alkyl; or is S(O₂)NR¹⁷R¹⁸; or is COR¹.
In some embodiments of one or more formulae herein, R¹² is hydrogen; or is C₁-C₆ alkyl optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or is CO₂R¹⁵; or is CONR¹⁷R¹⁸; or is C₁-C₆ haloalkyl; or is (C═NR¹⁵)NR¹⁷R¹⁸; or is S(O₂)C₁-C₆ alkyl; or is S(O₂)NR¹⁷R¹⁸; or is COR¹.
In some embodiments of one or more formulae herein, the group NR¹¹R¹² is amino; or is methylamino; or is dimethylamino; or R¹¹ and R¹² taken together with the nitrogen they are attached to in the NR¹¹R¹² group form a 3- to 7-membered ring optionally containing one or more heteroatoms in addition to the nitrogen they are attached to.

In some embodiments of one or more formulae herein, R¹² is selected from C₁-C₆ haloalkyl, (C═NR¹⁵)NR¹⁷R¹⁸, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or is selected from C₁-C₆ haloalkyl, (C═NR¹⁵)NR¹⁷R¹⁸, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl.

In some embodiments of one or more formulae herein, each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

The Groups R¹³, R¹⁵, R¹⁷ and R¹⁸

In some embodiments of one or more formulae herein, R¹³ is C₁-C₆ alkyl.

In some embodiments of one or more formulae herein, R¹¹ is C₁-C₆ alkyl.

In some embodiments of one or more formulae herein, R⁷ is hydrogen; or is C₁-C₆ alkyl.

In some embodiments of one or more formulae herein, R⁸ is hydrogen; or is C₁-C₆ alkyl.

The Groups R³⁴, R²⁹, R³⁵, R²¹ and R³⁶
In some embodiments, each of R³⁴, R²⁹, R³⁵, R²¹ and R³⁶ is independently selected from H, hydroxy, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, —(C₁-C₆ alkylene)ₒ-(Z¹-Z²)ₚ-Z³, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
In some embodiments, each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

In some embodiments of one or more formulae herein, each of R³⁴, R²⁹, R³⁵, R² and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl,

wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂.

In some embodiments of one or more formulae herein, each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³ COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂.

In some embodiments of one or more formulae herein, R³⁴ is H; or is CN; or is C₁-C₆ alkyl; or is CH₃; or is halo; or is C₁; or is F; or is hydroxy; or is —(C₁-C₆ alkylene)ₒ-(Z¹-Z²)ₚ-Z³.

In some embodiments of one or more formulae herein, R²⁹ is H; or is CN; or is C₁; or is F; or is C₁-C₆ alkyl; or is CH₃; or is C₁-C₆ alkyl substituted with hydroxy; or is 2-hydroxy-2-propyl; or is 1-hydroxy-1-cyclopropyl; or is C₁-C₆ alkyl substituted with oxo; or is C₁-C₆ alkyl substituted with C₁-C₆ alkoxy; or is C₁-C₆ alkyl substituted with NR¹¹R¹²; or is C₁-C₆ alkyl substituted with COOC₁-C₆ alkyl; or is C₁-C₆ alkyl substituted with CONR¹¹R¹²; or is C₁-C₆ alkyl substituted with C₃-C₇ cycloalkyl; or is C₁-C₆ alkyl substituted with 3- to 7-membered heterocycloalkyl; or is C₁-C₆ alkyl substituted with C₆-C₁₀ aryl; or is C₁-C₆ alkyl substituted with 5- to 10-membered heteroaryl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₁-C₆ alkyl substituted with NHCOC₆-C₁₀ aryl; or is C₁-C₆ alkyl substituted with NHCO(5- to 10-membered heteroaryl); or is C₁-C₆ alkyl substituted with NHCO(3- to 7-membered heterocycloalkyl); or is C₁-C₆ alkyl substituted with NHCO(3- to 7-membered heterocycloalkyl) optionally substituted with oxo; or is C₁-C₆ alkyl substituted with NHCOC₂-C₆ alkynyl; or is C₁-C₆ haloalkyl; or is halo; or is C₃-C₇ cycloalkyl; or is C₃-C₇ cycloalkyl substituted with hydroxy; or is C₃-C₇ cycloalkyl substituted with C₁-C₆ alkoxy; or is C₃-C₇ cycloalkyl substituted with NR¹¹R¹²; or is C₃-C₇ cycloalkyl substituted with COOC₁-C₆ alkyl; or is C₃-C₇ cycloalkyl substituted with CONR¹¹R¹²; or is C₃-C₇ cycloalkyl substituted with C₁-C₆ alkyl; or is 3- to 7-membered heterocycloalkyl; or is 3- to 7-membered nonaromatic monocyclic heterocycloalkyl; or is 1,3-dioxolan-2-yl; or is 3- to 7-membered nonaromatic monocyclic heterocycloalkyl substituted with hydroxy; or is 3- to 7-membered nonaromatic monocyclic heterocycloalkyl substituted with oxo; or is 3- to 7-membered nonaromatic monocyclic heterocycloalkyl substituted with C₁-C₆ alkoxy; or is 3- to 7-membered heterocycloalkyl substituted with C₁-C₆ alkyl; or is 3- to 7-membered nonaromatic monocyclic heterocycloalkyl substituted with C₁-C₆ alkyl; or is 2-methyl-1,3-dioxolan-2-yl; or is 3- to 7-membered heterocycloalkyl substituted with hydroxy; or is 3- to 7-membered heterocycloalkyl substituted with C₁-C₆ alkoxy; or is 3- to 7-membered heterocycloalkyl substituted with NR¹¹R¹²; or is 3- to 7-membered heterocycloalkyl substituted with COOC₁-C₆ alkyl; or is 3- to 7-membered heterocycloalkyl substituted with CONR¹¹R¹²; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, C₁-C₆ alkoxy, NR¹¹R¹², COOC₁-C₆ alkyl, and CONR¹¹R¹²; or is pyridyl optionally substituted with one or more substituents each independently selected from hydroxy, C₁-C₆ alkoxy, NR¹¹R¹², COOC₁-C₆ alkyl, and CONR¹¹R¹²; or is pyrimidinyl optionally substituted with one or more substituents each independently selected from hydroxy, C₁-C₆ alkoxy, NR¹¹R¹², COOC₁-C₆ alkyl, and CONR¹¹R¹²; or is pyrrolyl optionally substituted with one or more substituents each independently selected from hydroxy, C₁-C₆ alkoxy, NR¹¹R¹², COOC₁-C₆ alkyl, and CONR¹¹R¹²; or is pyrazolyl optionally substituted with one or more substituents each independently selected from hydroxy, C₁-C₆ alkoxy, NR¹¹R¹², COOC₁-C₆ alkyl, and CONR¹¹R¹²; or is imidazolyl optionally substituted with one or more substituents each independently selected from hydroxy, C₁-C₆ alkoxy, NR¹¹R¹², COOC₁-C₆ alkyl, and CONR¹¹R¹²; or is oxazolyl optionally substituted with one or more substituents each independently selected from hydroxy, C₁-C₆ alkoxy, NR¹¹R¹², COOC₁-C₆ alkyl, and CONR¹¹R¹²; or is thiazolyl optionally substituted with one or more substituents each independently selected from hydroxy, C₁-C₆ alkoxy, NR¹¹R¹², COOC₁-C₆ alkyl, and CONR¹¹R¹²; or is S(O₂)C₁-C₆ alkyl; or is S(O₂)CH₃; or is hydroxy; or is —(C₁-C₆ alkylene)ₒ-(Z¹-Z²)ₚ-Z³.

In some embodiments of one or more formulae herein, R³⁵ is H; or is CN; or is Cl; or is F; or is C₁-C₆ alkyl; or is CH₃; or is C₁-C₆ alkyl substituted with hydroxy; or is 2-hydroxy-2-propyl; or is 1-hydroxy-1-cyclopropyl; or is C₁-C₆ alkyl substituted with oxo; or is C₁-C₆ alkyl substituted with C₁-C₆ alkoxy; or is C₁-C₆ alkyl substituted with NR¹¹R¹²; or is C₁-C₆ alkyl substituted with COOC₁-C₆ alkyl; or is C₁-C₆ alkyl substituted with CONR¹¹R¹²; or is C₁-C₆ alkyl substituted with C₃-C₇ cycloalkyl; or is C₁-C₆ alkyl substituted with 3- to 7-membered heterocycloalkyl; or is C₁-C₆ alkyl substituted with C₆-C₁₀ aryl; or is C₁-C₆ alkyl substituted with 5- to 10-membered heteroaryl; or is C₁-C₆ alkyl substituted with NR¹¹R¹²; or is C₁-C₆ alkyl substituted with N12; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or

In some embodiments of one or more formulae herein, R³⁵ is S(O₂)CH₃; or is C₁-C₆ alkyl substituted with NHCOC₆-C₁₀ aryl; or is C₁-C₆ alkyl substituted with NHCO(5- to 10-membered heteroaryl); or is C₁-C₆ alkyl substituted with NHCO(3- to 7-membered heterocycloalkyl); or is C₁-C₆ alkyl substituted with NHCO(3- to 7-membered heterocycloalkyl) optionally substituted with oxo; or is C₁-C₆ alkyl substituted with NHCOC₂-C₆ alkynyl; or is C₁-C₆ haloalkyl; or is halo; or is C₃-C₇ cycloalkyl; or is C₃-C₇ cycloalkyl substituted with hydroxy; or is C₃-C₇ cycloalkyl substituted with C₁-C₆ alkoxy; or is C₃-C₇ cycloalkyl substituted with NR¹¹R¹²; or is C₃-C₇ cycloalkyl substituted with COOC₁-C₆ alkyl; or is C₃-C₇ cycloalkyl substituted with CONR¹¹R¹²; or is C₃-C₇ cycloalkyl substituted with C₁-C₆ alkyl; or is 3- to 7-membered heterocycloalkyl; or is 3- to 7-membered nonaromatic monocyclic heterocycloalkyl; or is 1,3-dioxolan-2-yl; or is 3- to 7-membered nonaromatic monocyclic heterocycloalkyl substituted with hydroxy; or is 3- to 7-membered nonaromatic monocyclic heterocycloalkyl substituted with oxo; or is 3- to 7-membered nonaromatic monocyclic heterocycloalkyl substituted with C₁-C₆ alkoxy; or is 3- to 7-membered heterocycloalkyl substituted with C₁-C₆ alkyl; or is 3- to 7-membered nonaromatic monocyclic heterocycloalkyl substituted with C₁-C₆ alkyl; or is 2-methyl-1,3-dioxolan-2-yl; or is 3- to 7-membered heterocycloalkyl substituted with hydroxy; or is 3- to 7-membered heterocycloalkyl substituted with C₁-C₆ alkoxy; or is 3- to 7-membered heterocycloalkyl substituted with NR¹¹R¹²; or is 3- to 7-membered heterocycloalkyl substituted with COOC₁-C₆ alkyl; or is 3- to 7-membered heterocycloalkyl substituted with CONR¹¹R¹²; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, C₁-C₆ alkoxy, NR¹¹R¹², COOC₁-C₆ alkyl, and CONR¹¹R¹²; or is pyridyl optionally substituted with one or more substituents each independently selected from hydroxy, C₁-C₆ alkoxy, NR¹¹R¹², COOC₁-C₆ alkyl, and CONR¹¹R¹²; or is pyrimidinyl optionally substituted with one or more substituents each independently selected from hydroxy, C₁-C₆ alkoxy, NR¹¹R¹², COOC₁-C₆ alkyl, and CONR¹¹R¹²; or is pyrrolyl optionally substituted with one or more substituents each independently selected from hydroxy, C₁-C₆ alkoxy, NR¹¹R¹², COOC₁-C₆ alkyl, and CONR¹¹R¹²; or is pyrazolyl optionally substituted with one or more substituents each independently selected from hydroxy, C₁-C₆ alkoxy, NR¹¹R¹², COOC₁-C₆ alkyl, and CONR¹¹R¹²; or is imidazolyl optionally substituted with one or more substituents each independently selected from hydroxy, C₁-C₆ alkoxy, NR¹¹R¹², COOC₁-C₆ alkyl, and CONR¹¹R¹²; or is oxazolyl optionally substituted with one or more substituents each independently selected from hydroxy, C₁-C₆ alkoxy, NR¹¹R¹², COOC₁-C₆ alkyl, and CONR¹¹R¹²; or is thiazolyl optionally substituted with one or more substituents each independently selected from hydroxy, C₁-C₆ alkoxy, NR¹¹R¹², COOC₁-C₆ alkyl, and CONR¹¹R¹²; or is S(O₂)C₁-C₆ alkyl; or is S(O₂)CH₃.

In some embodiments of one or more formulae herein, R²¹ is hydroxy; or is —(C₁-C₆ alkylene)ₒ-(Z¹-Z²)ₚ-Z³; or is H; or is CN; or is C₁; or is F; or is C₁-C₆ alkyl; or is CH₃; or is C₁-C₆ alkyl substituted with hydroxy; or is 2-hydroxy-2-propyl; or is 1-hydroxy-1-cyclopropyl; or is C₁-C₆ alkyl substituted with oxo; or is C₁-C₆ alkyl substituted with C₁-C₆ alkoxy; or is C₁-C₆ alkyl substituted with NR¹¹R¹²; or is C₁-C₆ alkyl substituted with COOC₁-C₆ alkyl; or is C₁-C₆ alkyl substituted with CONR¹¹R¹²; or is C₁-C₆ alkyl substituted with C₃-C₇ cycloalkyl; or is C₁-C₆ alkyl substituted with 3- to 7-membered heterocycloalkyl; or is C₁-C₆ alkyl substituted with C₆-C₁₀ aryl; or is C₁-C₆ alkyl substituted with 5- to 10-membered heteroaryl; or is C₁-C₆ alkyl substituted with NR¹¹R¹²; or is C₁-C₆ alkyl substituted with NH₂; or is C₁-C₆ alkyl substituted with NH(C₁-C₆ alkyl); or is C₁-C₆ alkyl substituted with N(C₁-C₆ alkyl)₂; or is dimethylaminomethyl; or is C₁-C₆ alkyl substituted with NR¹¹R¹², wherein R¹¹ and R¹² taken together with the nitrogen they are attached to form a 3- to 7-membered ring optionally containing one or more heteroatoms in addition to the nitrogen they are attached to; or is S(O₂)C₁-C₆ alkyl; or is S(O₂)CH₃; or is C₁-C₆ alkyl substituted with NHCOC₆-C₁₀ aryl; or is C₁-C₆ alkyl substituted with NHCO(5- to 10-membered heteroaryl); or is C₁-C₆ alkyl substituted with NHCO(3- to 7-membered heterocycloalkyl); or is C₁-C₆ alkyl substituted with NHCO(3- to 7-membered heterocycloalkyl) optionally substituted with oxo; or is C₁-C₆ alkyl substituted with NHCOC₂-C₆ alkynyl; or is C₁-C₆ haloalkyl; or is halo; or is C₃-C₇ cycloalkyl; or is C₃-C₇ cycloalkyl substituted with hydroxy; or is C₃-C₇ cycloalkyl substituted with C₁-C₆ alkoxy; or is C₃-C₇ cycloalkyl substituted with NR¹¹R¹²; or is C₃-C₇ cycloalkyl substituted with COOC₁-C₆ alkyl; or is C₃-C₇ cycloalkyl substituted with CONR¹¹R¹²; or is C₃-C₇ cycloalkyl substituted substituted with C₁-C₆ alkyl.

In some embodiments of one or more formulae herein, R²⁹ is 3- to 7-membered heterocycloalkyl.

In some embodiments of one or more formulae herein, R²¹ is 3- to 7-membered nonaromatic monocyclic heterocycloalkyl; or is 1,3-dioxolan-2-yl; or is 3- to 7-membered nonaromatic monocyclic heterocycloalkyl substituted with hydroxy; or s 3- to 7-membered nonaromatic monocyclic heterocycloalkyl substituted with oxo; or is 3- to 7-membered nonaromatic monocyclic heterocycloalkyl substituted with C₁-C₆ alkoxy; or is 3- to 7-membered heterocycloalkyl substituted with C₁-C₆ alkyl; or is 3- to 7-membered nonaromatic monocyclic heterocycloalkyl substituted with C₁-C₆ alkyl; or is 2-methyl-1,3-dioxolan-2-yl; or is 3- to 7-membered heterocycloalkyl substituted with hydroxy; or is 3- to 7-membered heterocycloalkyl substituted with C₁-C₆ alkoxy; or is 3- to 7-membered heterocycloalkyl substituted with NR¹¹R¹²; or is 3- to 7-membered heterocycloalkyl substituted with COOC₁-C₆ alkyl; or is 3- to 7-membered heterocycloalkyl substituted with CONR¹¹R¹²; or is 5- to 7-membered aromatic monocyclic radical having 1-3 heteroatoms selected from O, N, or S, wherein 0, 1, 2 or 3 atoms of each ring are optionally substituted with one or more substituents each independently selected from hydroxy, C₁-C₆ alkoxy, NR¹¹R¹², COOC₁-C₆ alkyl, and CONR¹¹R¹²; or is pyridyl optionally substituted with one or more substituents each independently selected from hydroxy, C₁-C₆ alkoxy, NR¹¹R¹², COOC₁-C₆ alkyl, and CONR¹¹R¹²; or is pyrimidinyl optionally substituted with one or more substituents each independently selected from hydroxy, C₁-C₆ alkoxy, NR¹¹R¹², COOC₁-C₆ alkyl, and CONR¹¹R¹²; or is pyrrolyl optionally substituted with one or more substituents each independently selected from hydroxy, C₁-C₆ alkoxy, NR¹¹R¹², COOC₁-C₆ alkyl, and CONR¹¹R¹²; or is pyrazolyl optionally substituted with one or more substituents each independently selected from hydroxy, C₁-C₆ alkoxy, NR¹¹R¹², COOC₁-C₆ alkyl, and CONR¹¹R¹²; or is imidazolyl optionally substituted with one or more substituents each independently selected from hydroxy, C₁-C₆ alkoxy, NR¹¹R¹², COOC₁-C₆ alkyl, and CONR¹¹R¹²; or is oxazolyl optionally substituted with one or more substituents each independently selected from hydroxy, C₁-C₆ alkoxy, NR¹¹R¹², COOC₁-C₆ alkyl, and CONR¹¹R¹²; or is thiazolyl optionally substituted with one or more substituents each independently selected from hydroxy, C₁-C₆ alkoxy, NR¹¹R¹², COOC₁-C₆ alkyl, and CONR¹¹R¹²; or is S(O₂)C₁-C₆ alkyl; or is S(O₂)CH₃.

In some embodiments of one or more formulae herein, R³⁶ is H; or is CN; or is C₁-C₆ alkyl; or is CH₃; or is halo; or is C₁; or is F; or is hydroxy; or is —(C₁-C₆ alkylene)ₒ-(Z¹-Z²)ₚ-Z³.
In some embodiments of one or more formulae herein, wherein R³⁴ and R²⁹, taken together with the atoms connecting them form a monocyclic or bicyclic 3-to-12-membered carbocyclic ring or a monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R²⁹ and R³⁵, taken together with the atoms connecting them form a monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R³⁵ and R²¹, taken together with the atoms connecting them form a monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R²¹ and R²⁶, taken together with the atoms connecting them form a monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².
In some embodiments of one or more formulae herein, R³⁴ and R²⁹ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³ and S; or a five-membered heterocyclic ring containing 1 or 2 heteroatoms independently selected from O, N, NH, NR¹³, and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from 0, NH, and NMe (e.g., the other heteroatom is O)); or a seven-membered heterocyclic ring containing 1 or 2 heteroatoms independently selected from O, N, NH NR¹³ and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².
In certain embodiments of the foregoing, each of R²¹, R³⁵, and R³⁶ is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²; or each of R²¹, R³⁵, and R³⁶ is H; or one of R²¹, R³⁵, and R³⁶ is H; or two of R²¹, R³⁵, and R³⁶ are H.
In some embodiments of one or more formulae herein, R²⁹ and R³⁵ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³ and S; or a five-membered heterocyclic ring containing 1 or 2 heteroatoms independently selected from O, N, NH, NR¹³, and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from 0, NH, and NMe (e.g., the other heteroatom is O)); or a seven-membered heterocyclic ring containing 1 or 2 heteroatoms independently selected from O, N, NH NR¹³ and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².
In certain embodiments of the foregoing, each of R²¹, R³⁴, and R³⁶ is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²; or each of R², R³⁴, and R³⁶ is H; or one of R²¹, R³⁴, and R³⁶ is H; or two of R²¹, R³⁴, and R³⁶ are H.
In some embodiments of one or more formulae herein, R³⁵ and R²¹ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH NR¹³ and S; or a five-membered heterocyclic ring containing 1 or 2 heteroatoms independently selected from O, N, NH, NR¹³, and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from 0, NH, and NMe (e.g., the other heteroatom is O)); or a seven-membered heterocyclic ring containing 1 or 2 heteroatoms independently selected from O, N, NH, NR¹³ and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl.
In some embodiments of one or more formulae herein, R³⁵ and R²¹ taken together with the atoms connecting them form a carbocyclic ring substituted with CONR¹¹R¹².
In certain embodiments of the foregoing, each of R²⁹, R³⁴, and R³⁶ is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²; or each of R²⁹, R³⁴, and R³⁶ is H; or one of R²⁹, R³⁴, and R³⁶ is H; or two of R²⁹, R³⁴, and R³⁶ are H.
In some embodiments of one or more formulae herein, R²¹ and R³⁶ taken together with the atoms connecting them form a three-membered carbocyclic ring; or a four-membered carbocyclic ring; or a five-membered carbocyclic ring; or a six-membered carbocyclic ring; or a seven-membered carbocyclic ring; or an eight-membered carbocyclic ring; or a three-membered heterocyclic ring containing 1 or 2 heteroatoms independently selected from O, N, NH, NR¹³, and S; or a four-membered heterocyclic ring containing 1 or 2 heteroatoms independently selected from O, N, NH, NR¹³, and S; or a five-membered heterocyclic ring containing 1 or 2 heteroatoms independently selected from O, N, NH, NR¹³, and S; or a six-membered heterocyclic ring containing 1 or 2 heteroatoms independently selected from O, N, NH, NR¹³, and S (e.g., a six-membered heterocyclic ring containing 2 heteroatoms wherein one heteroatom is N; and the other heteroatom is selected from 0, NH, and NMe (e.g., the other heteroatom is O)); or a seven-membered heterocyclic ring containing 1 or 2 heteroatoms independently selected from O, N, NH, NR¹³ and S; or an eight-membered heterocyclic ring containing 1 or 2 heteroatoms independently selected from O, N, NH, NR¹³, and S; or a nine-membered heterocyclic ring (e.g., spirocyclic nine-membered heterocyclic ring) containing from 1-3 heteroatoms independently selected from O, N, NH, NR¹³, and S; or a carbocyclic ring substituted with hydroxy; or a carbocyclic ring substituted with oxo; or a carbocyclic ring substituted with C₁-C₆ alkoxy (e.g., the ring is substituted with one C₁-C₆ alkoxy which is unsubstituted; or which is substituted as described elsewhere herein); or a carbocyclic ring substituted with C₁-C₆ alkyl (e.g., the ring is substituted with one C₁-C₆ alkyl which is unsubstituted; or which is substituted as described elsewhere herein); or a carbocyclic ring substituted with NR¹¹R¹²; or a carbocyclic ring substituted with ═NR¹³; or a carbocyclic ring substituted with COOC₁-C₆ alkyl; or a carbocyclic ring substituted with CONR¹¹R¹².
In certain embodiments of the foregoing, each of R²⁹, R³⁴, and R³⁵ is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹².
In certain embodiments, each of R²⁹, R³⁴, and R³⁵ is H; or one of R²⁹, R³⁴, and R³⁵ is H; or two of R²⁹, R³⁴, and R³⁵ are H.
The Groups Z¹, Z², and Z³
In some embodiments of one or more formulae herein, each occurrence of Z¹ is independently selected from O, NR¹⁷C(O), 5-to-10-membered heteroarylene, and 3-10 membered heterocycloalkyl.
In some embodiments of one or more formulae herein, each occurrence of Z¹ is O; or is NR¹⁷C(O; or is 5-to-10-membered heteroarylene (e.g., triazolyl); or is 3-10 membered heterocycloalkyl (e.g., diazirine (e.g., 3H-diazirine)).
In some embodiments of one or more formulae herein, each occurrence of Z² is C₁-C₆ alkylene; or is methylene; or is ethylene; or is propylene; or is butylene.
In some embodiments of one or more formulae herein, Z³ is selected from NHCO₂R¹⁵ (e.g., NHCO₂tBu) and 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH. In some embodiments of one or more formulae herein, Z³ is NHCO₂R¹⁵; or Z³ is 5-to-10 membered monocyclic or bicyclic heterocycloalkyl containing 1-3 heteroatoms selected from O, N, and S, wherein the heterocycloalkyl is optionally substituted with one or more oxo, hydroxy, or —(C₁-C₆ alkylene)-OH; or Z³ is (3aR,6aS)-tetrahydro-1H-thieno[3,4-d]imidazol-2(3H)-onyl.

### The Groups o and p

In some embodiments of one or more formulae herein, o is 0 or 1. In some embodiments of one or more formulae herein, o is O.
In some embodiments of one or more formulae herein, o is 1.
In some embodiments of one or more formulae herein, p is selected from 0, 1, 2, 3, 4, 5, 6, 7, or 8; or is selected from 4, 5, 6, or 7; or is selected from 6 or 7; or is 6; or is 7.

### The Moieties

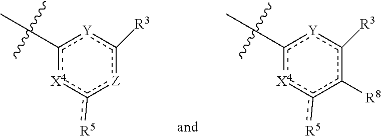

In some embodiments of one or more formulae herein, the moiety

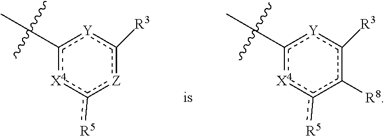

In some embodiments of one or more formulae herein,

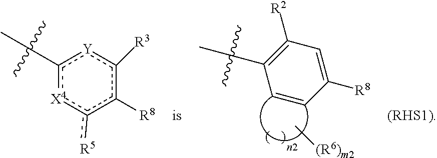

In some embodiments of one or more formulae herein, RHS1 is

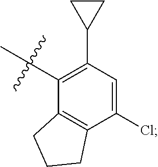 [SMILES: *c1c(C2CC2)cc(Cl)c2c1CCC2]

or is

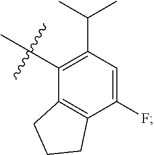 [SMILES: *c1c(C(C)C)cc(F)c2c1CCC2]

or is

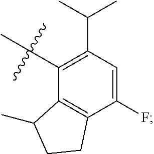 [SMILES: *c1c(C(C)C)cc(F)c2c1C(C)CC2]

or is

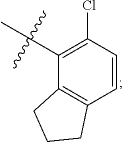 [SMILES: *c1c(Cl)ccc2c1CCC2]

or is

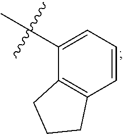 [SMILES: *c1cccc2c1CCC2]

or is

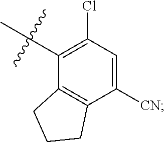 [SMILES: *c1c(Cl)cc(C#N)c2c1CCC2]

or is

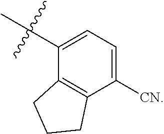 [SMILES: *c1ccc(C#N)c2c1CCC2]

In some embodiments of one or more formulae herein,

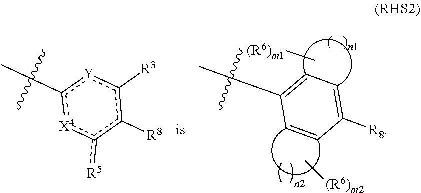

In some embodiments of one or more formulae herein, RHS2 is

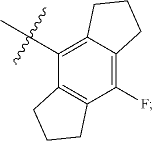 [SMILES: *c1c2c(c(F)c3c1CCC3)CCC2]

or is

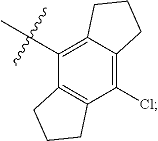 [SMILES: *c1c2c(c(Cl)c3c1CCC3)CCC2]

or is

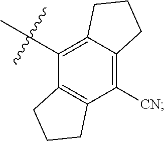 [SMILES: *c1c2c(c(C#N)c3c1CCC3)CCC2]

or is

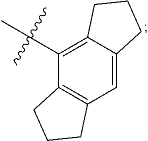 [SMILES: *c1c2c(cc3c1CCC3)CCC2]

or is

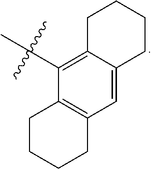 [SMILES: *c1c2c(cc3c1CCCC3)CCCC2]

In some embodiments of one or more formulae herein,

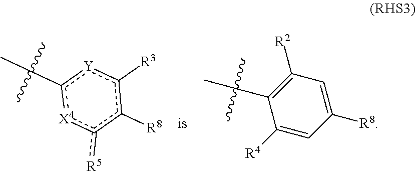

In some embodiments of one or more formulae herein, RHS3 is

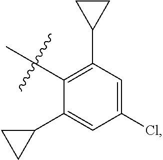 [SMILES: *c1c(C2CC2)cc(Cl)cc1C1CC1]

or is

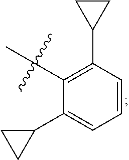 [SMILES: *c1c(C2CC2)cccc1C1CC1]

or is

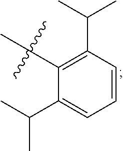 [SMILES: *c1c(C(C)C)cccc1C(C)C]

or is

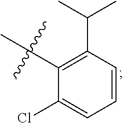 [SMILES: *c1c(Cl)cccc1C(C)C]

or is

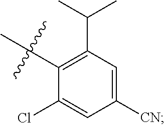 [SMILES: *c1c(Cl)cc(C#N)cc1C(C)C]

or is

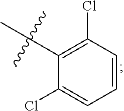 [SMILES: *c1c(Cl)cccc1Cl]

or is

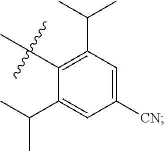 [SMILES: *c1c(C(C)C)cc(C#N)cc1C(C)C]

or is

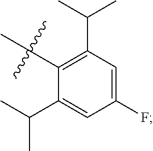 [SMILES: *c1c(C(C)C)cc(F)cc1C(C)C]

or is

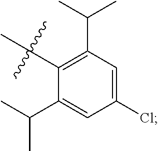 [SMILES: *c1c(C(C)C)cc(Cl)cc1C(C)C]

or is

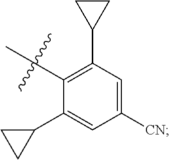 [SMILES: *c1c(C2CC2)cc(C#N)cc1C1CC1]

or is

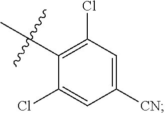 [SMILES: *c1c(Cl)cc(C#N)cc1Cl]

or is

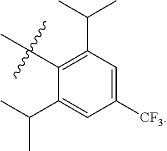 [SMILES: *c1c(C(C)C)cc(C(F)(F)F)cc1C(C)C]

In some embodiments of one or more formulae herein,

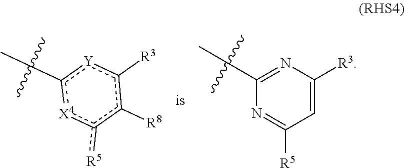

In some embodiments of one or more formulae herein, RHS4 is

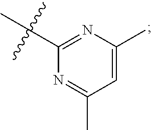 [SMILES: *c1nc(C)cc(C)n1]

or is

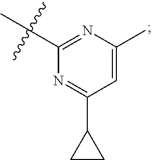 [SMILES: *c1nc(C)cc(C2CC2)n1]

or is

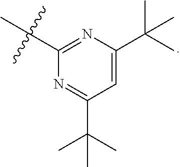 [SMILES: *c1nc(C(C)(C)C)cc(C(C)(C)C)n1]

In some embodiments of one or more formulae herein,

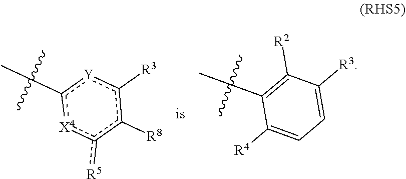

In some embodiments of one or more formulae herein, RHS5 is

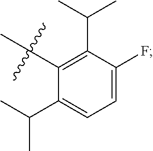 [SMILES: *c1c(C(C)C)ccc(F)c1C(C)C]

or is

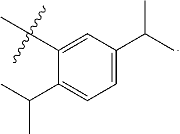 [SMILES: *c1cc(C(C)C)ccc1C(C)C]

In some embodiments of one ore more formulae herein,

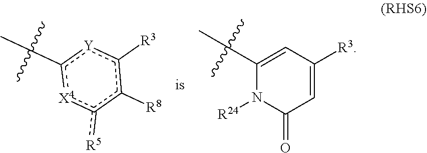

In some embodiments of one or more formulae herein, RHS6 is

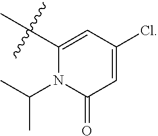 [SMILES: *c1cc(Cl)cc(=O)n1C(C)C]

In some embodiments of one or more formulae herein,

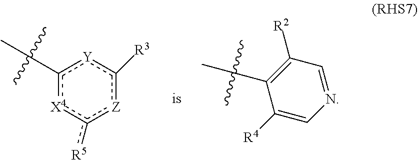

In some embodiments of one or more formulae herein, RHS7 is

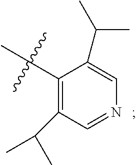 [SMILES: *c1c(C(C)C)cncc1C(C)C]

or is

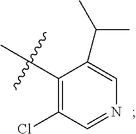 [SMILES: *c1c(Cl)cncc1C(C)C]

or is

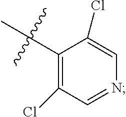 [SMILES: *c1c(Cl)cncc1Cl]

or is

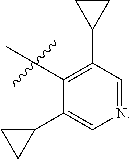 [SMILES: *c1c(C2CC2)cncc1C1CC1]

In some embodiments of one or more formulae herein,

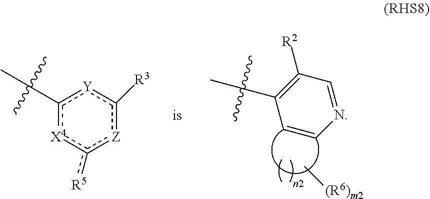

In some embodiments of one or more formulae herein, RHS8 is

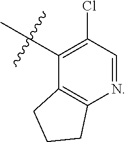 [SMILES: *c1c(Cl)cnc2c1CCC2]

In some embodiments of one or more formulae herein,

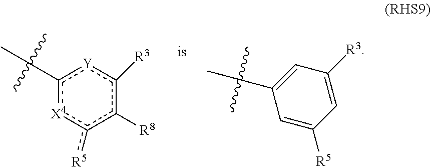

In some embodiments of one or more formulae herein, RHS9 is

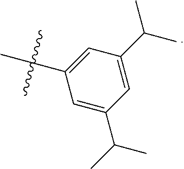 [SMILES: *c1cc(C(C)C)cc(C(C)C)c1]

In some embodiments of one or more formulae herein,

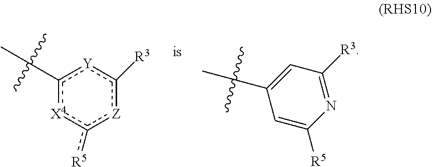

In some embodiments of one or more formulae herein, RHS10 is

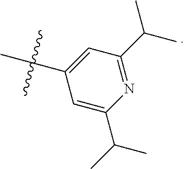 [SMILES: *c1cc(C(C)C)nc(C(C)C)c1]

In some embodiments of one or more formulae herein,

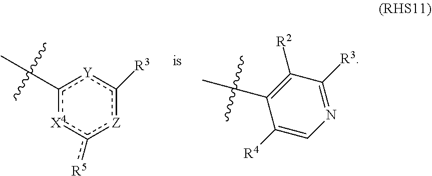

In some embodiments of one or more formulae herein, RHS11 is

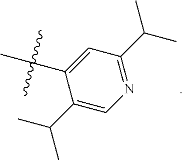 [SMILES: *c1cc(C(C)C)ncc1C(C)C]

In some embodiments of one or more formulae herein,

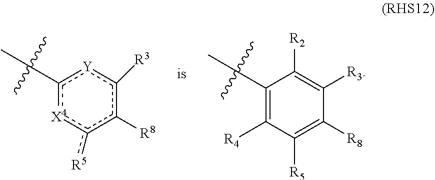

### The Moiety

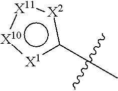

In some embodiments of one or more formulae herein,

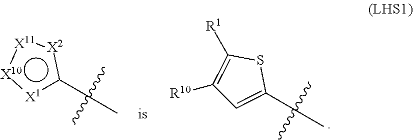

In some embodiments of one or more formulae herein, LHS1 is

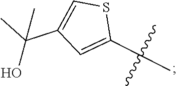 [SMILES: *c1cc(C(C)(C)O)cs1]

or is

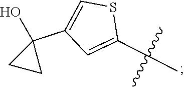 [SMILES: *c1cc(C2(O)CC2)cs1]

or is

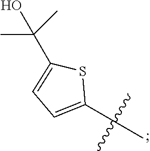 [SMILES: *c1ccc(C(C)(C)O)s1]

or is

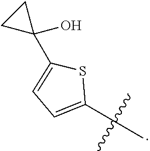 [SMILES: *c1ccc(C2(O)CC2)s1]

In some embodiments of one or more formulae herein,

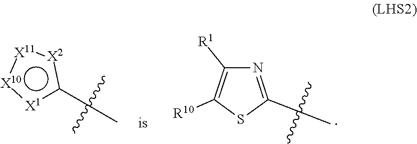

In some embodiments of one or more formulae herein, LHS2 is

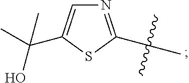 [SMILES: *c1ncc(C(C)(C)O)s1]

or is

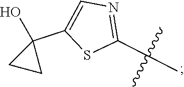 [SMILES: *c1ncc(C2(O)CC2)s1]

or is

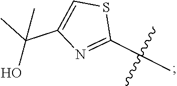 [SMILES: *c1nc(C(C)(C)O)cs1]

or is

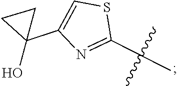 [SMILES: *c1nc(C2(O)CC2)cs1]

or is

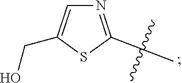 [SMILES: *c1ncc(CO)s1]

or is

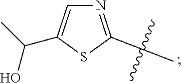 [SMILES: *c1ncc(C(C)O)s1]

or is

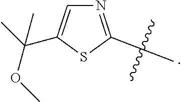 [SMILES: *c1ncc(C(C)(C)OC)s1]

In some embodiments of one or more formulae herein,

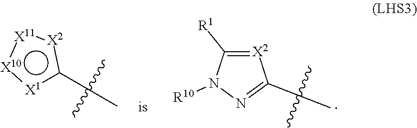

In some embodiments of one or more formulae herein, LHS3 is

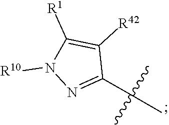

or is

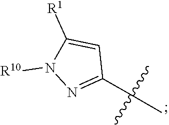

or is

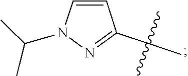 [SMILES: *c1ccn(C(C)C)n1]

or is

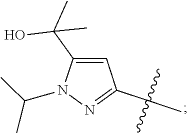 [SMILES: *c1cc(C(C)(C)O)n(C(C)C)n1]

or is

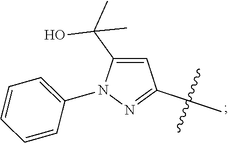 [SMILES: *c1cc(C(C)(C)O)n(-c2ccccc2)n1]

or is

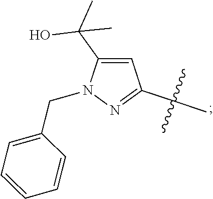 [SMILES: *c1cc(C(C)(C)O)n(Cc2ccccc2)n1]

or is

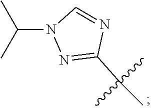 [SMILES: *c1ncn(C(C)C)n1]

or is

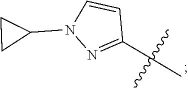 [SMILES: *c1ccn(C2CC2)n1]

or is

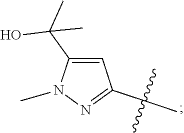 [SMILES: *c1cc(C(C)(C)O)n(C)n1]

or is

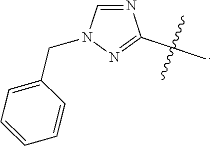 [SMILES: *c1ncn(Cc2ccccc2)n1]

In some embodiments of one or more formulae herein, i

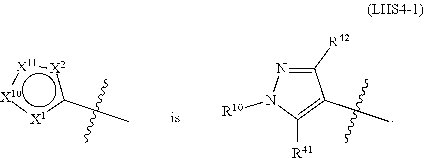

In some embodiments of one or more formulae herein,

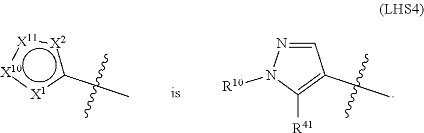

In some embodiments of one or more formulae herein, LHS4 is

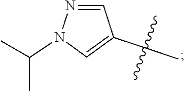 [SMILES: *c1cnn(C(C)C)c1]

or is

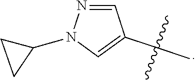 [SMILES: *c1cnn(C2CC2)c1]

In some embodiments of one or more formulae herein,

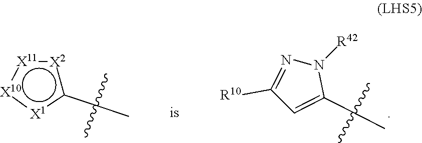

In some embodiments of one or more formulae herein,

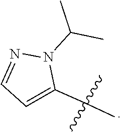 [SMILES: *c1ccnn1C(C)C]

In some embodiments of one or more formulae herein,

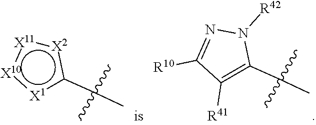

In some embodiments of one or more formulae herein,

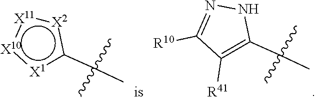

In some embodiments of one or more formulae herein,

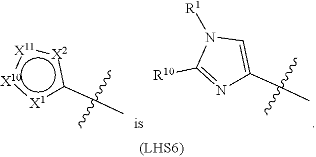

In some embodiments of one or more formulae herein,

In some embodiments of one or more formulae herein, LHS6 is

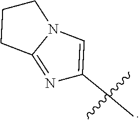 [SMILES: *c1cn2c(n1)CCC2]

In some embodiments of one or more formulae herein,

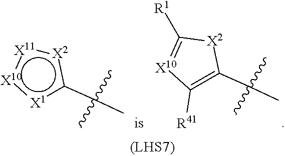

In some embodiments of LHS7, X¹⁰ is N; and X² is O; or X¹⁰ is N; and X² is S.
In some embodiments of one or more formulae herein, LHS7 is

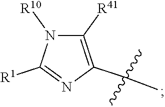

or is

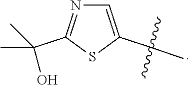 [SMILES: *c1cnc(C(C)(C)O)s1]

In some embodiments of LHS7, X¹⁰ is CR¹⁰; and X² is O; or X¹⁰ is CR¹⁰; and X² is S; or X¹⁰ is CH; and X² is O; or X¹⁰ is CH; and X² is S.

In some embodiments of one or more formulae herein is

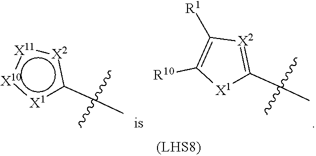

In some embodiments of LHS8, X¹ is O; and X² is N; or X¹ is S; and X² is N; or X is O; and X² is CR⁴²; or X¹ is S; and X² is CR⁴²; or X¹ is O; and X² is CH; or X¹ is S; and X² is CH; or X¹ is O; and X² is CCH₃; or X¹ is S; and X² is CCH₃.

In some embodiments of one or more formulae herein, is

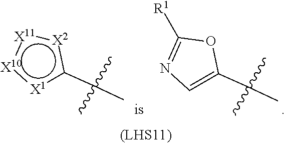

In some embodiments of one or more formulae herein, LHS11 is

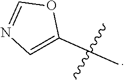 [SMILES: *c1cnco1]

In some embodiments of one or more formulae herein, is

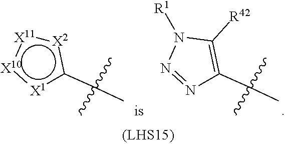

In some embodiments of one or more formulae herein, LHS15 is

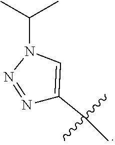 [SMILES: *c1cn(C(C)C)nn1]

In some embodiments of one or more formulae herein,

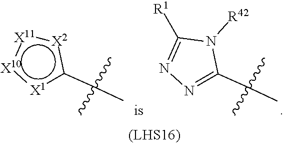

In some embodiments of one or more formulae herein, LHS16 is

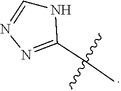 [SMILES: *c1nnc[nH]1]

In some embodiments of one or more formulae herein is

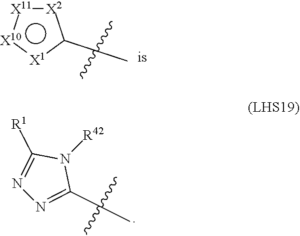

In some embodiments of one or more formulae herein, the moiety

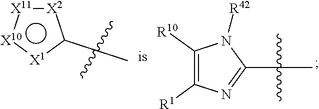

or is

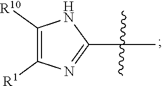

or is

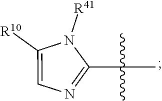

or is

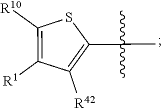

or is

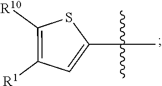

or is

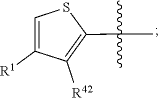

or is

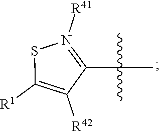

or is

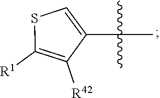

or is

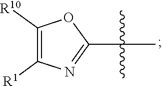

or is

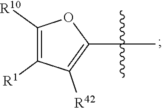

or is

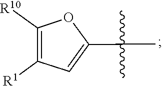

or is

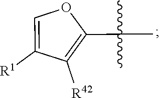

or is

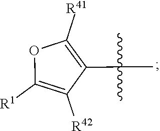

or is

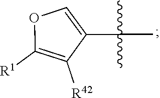

or is

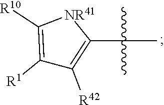

or is

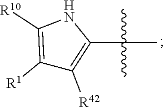

or is

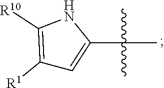

or is

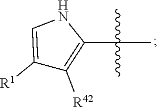

or is

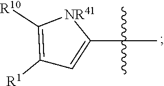

or is

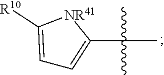

or is

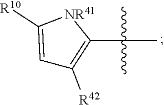

or is

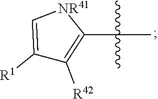

or is

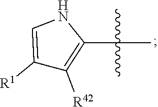

or is

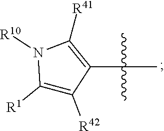

or is

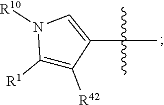

or is

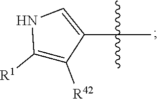

or is

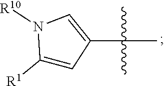

or is

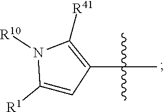

or is

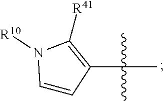

or is

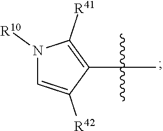

or is

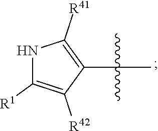

or is

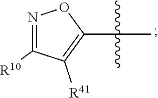

or is

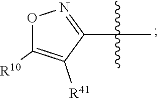

or is

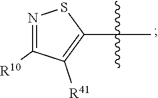

or is

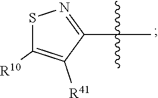

or is

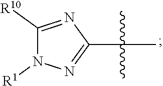

or is

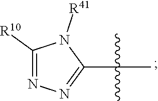

or is

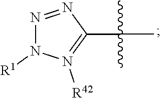

or is selected from the group consisting of:

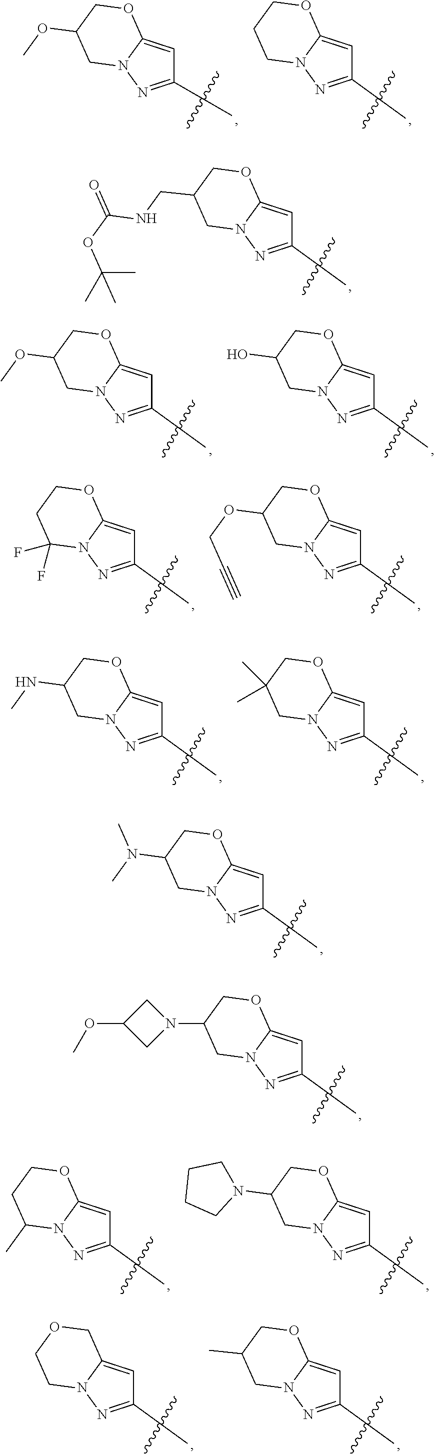

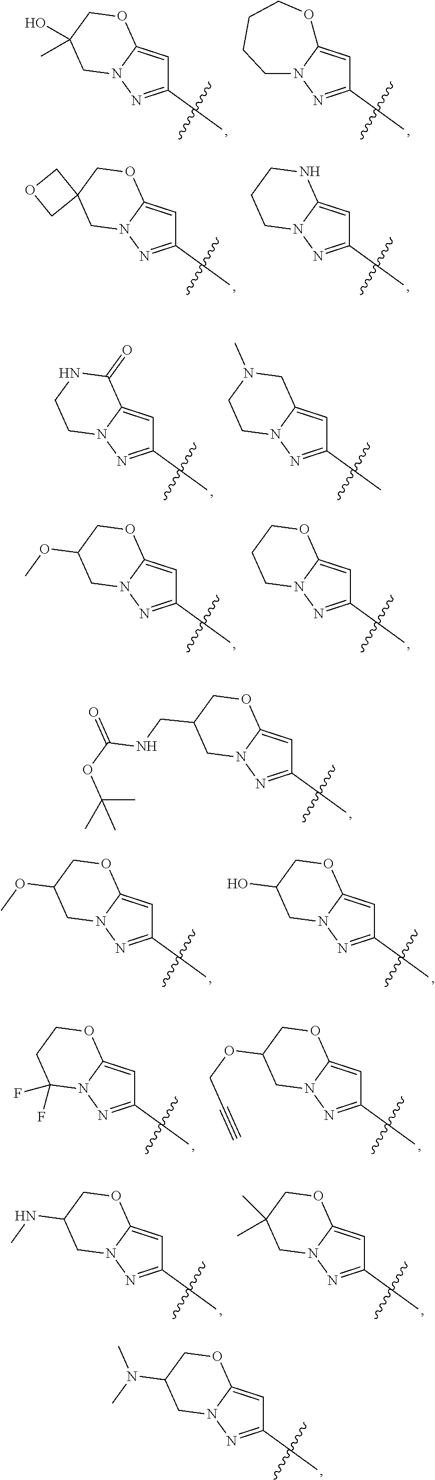

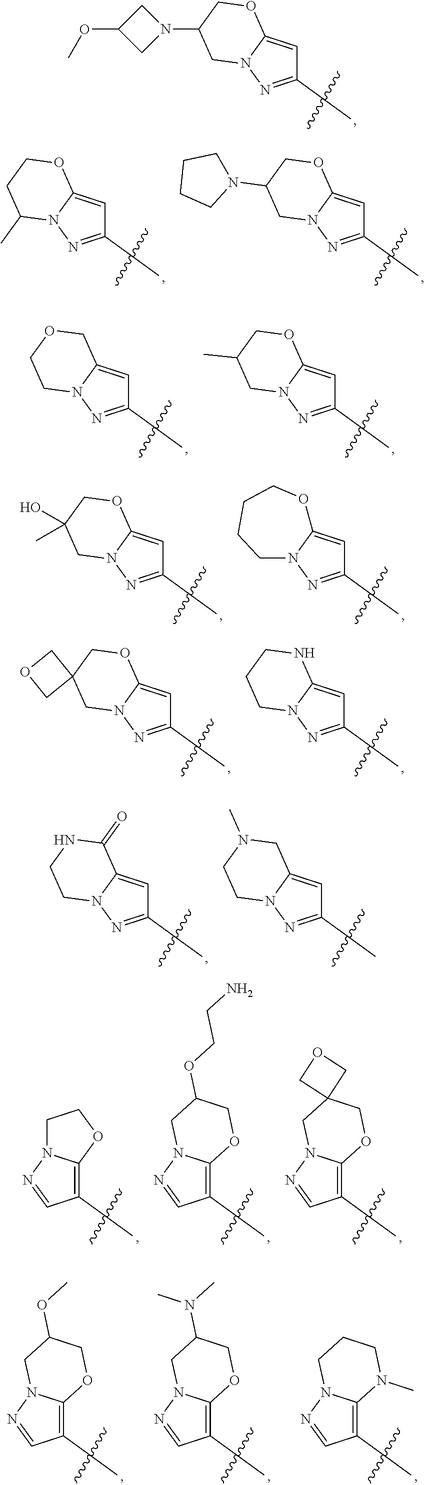

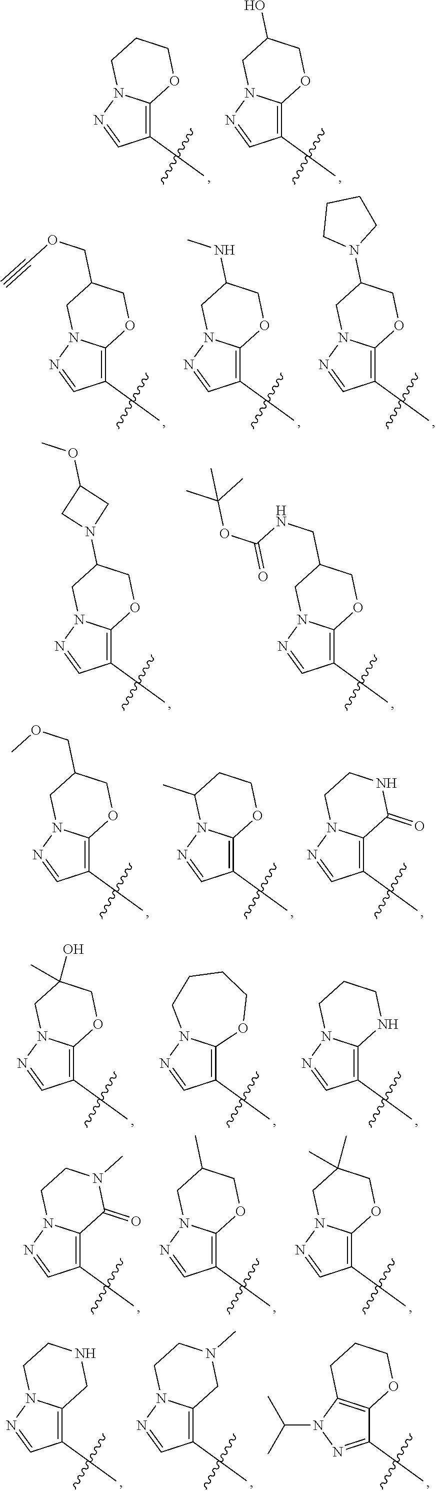

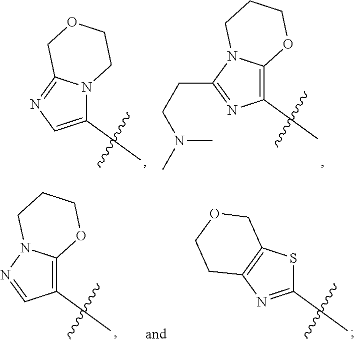 [SMILES: *c1cnc2n1CCOC2 ; *c1nc(CCN(C)C)n2c1OCCC2 ; *c1cnn2c1OCCC2 ; *c1nc2c(s1)COCC2]

or is selected from the group consisting of:

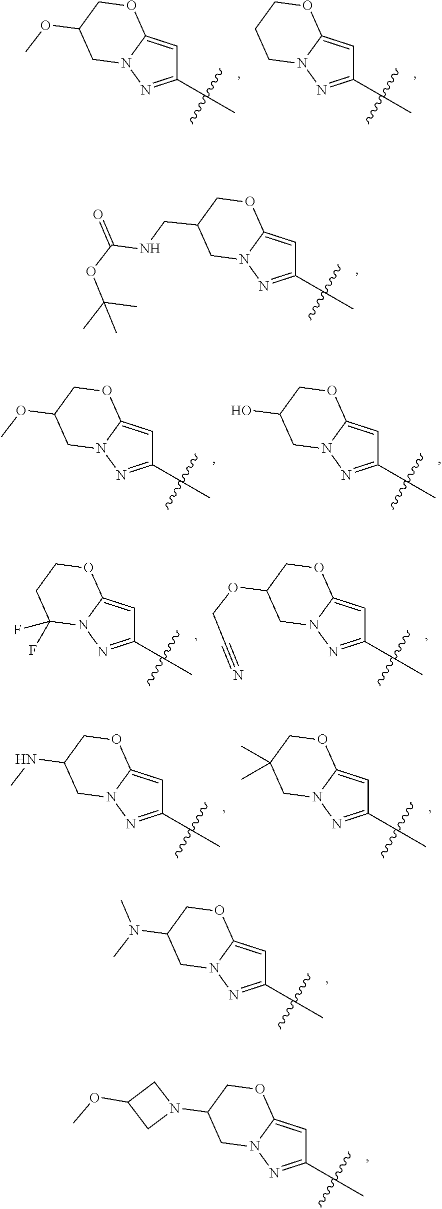

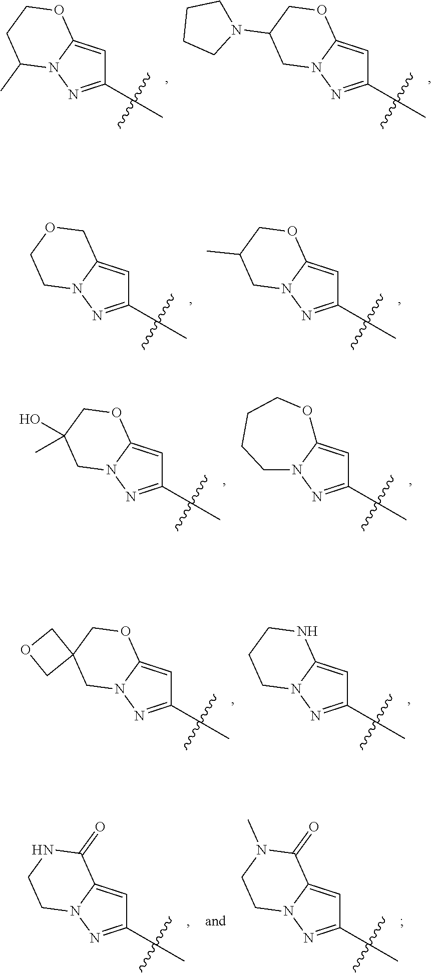

or is selected from the group consisting of:

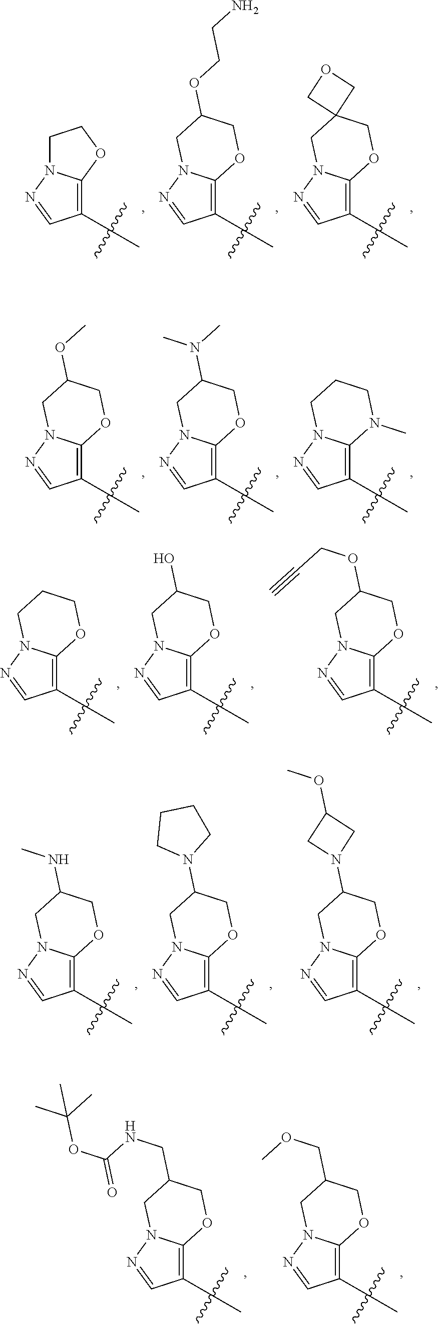

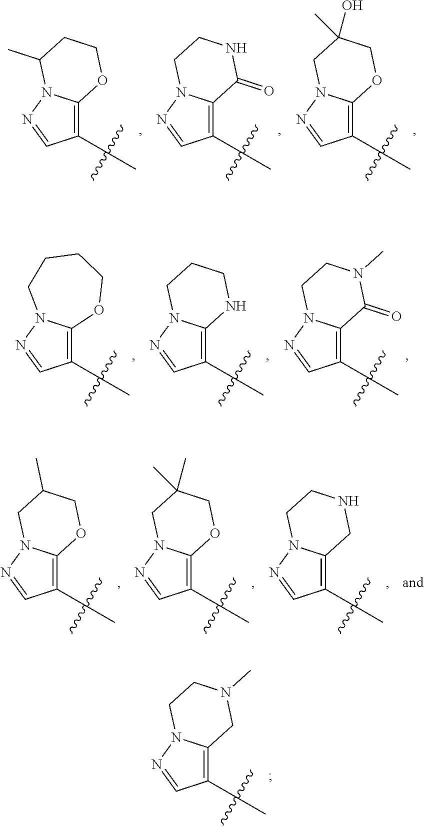

or is selected from the group consisting of:

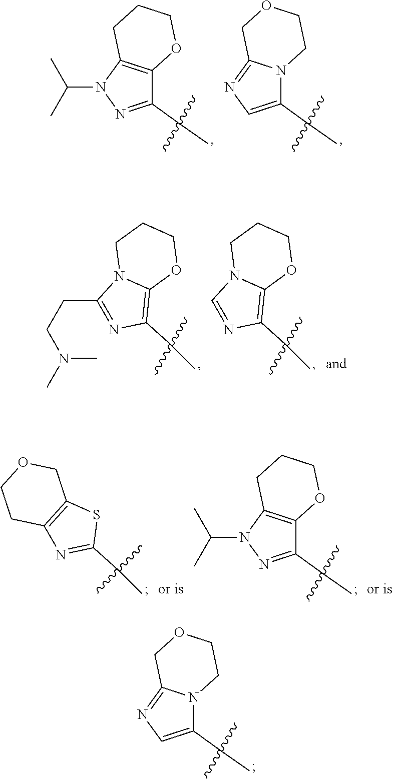

or is selected from the group consisting of:

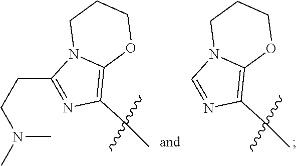 [SMILES: *c1nc(CCN(C)C)n2c1OCCC2 ; *c1ncn2c1OCCC2]

or is

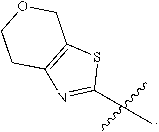 [SMILES: *c1nc2c(s1)COCC2]

### The Moiety

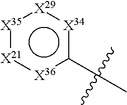

In some embodiments of one or more formulae herein,

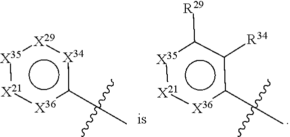

In some embodiments of one or more formulae herein,

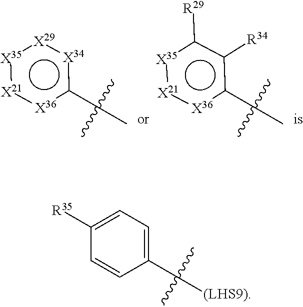

In some embodiments of one or more formulae herein, LHS9 is

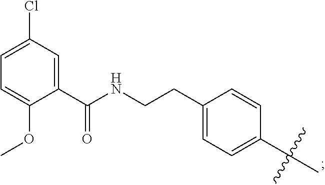 [SMILES: *c1ccc(CCNC(=O)c2cc(Cl)ccc2OC)cc1]

or is

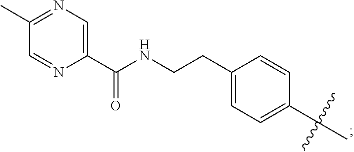 [SMILES: *c1ccc(CCNC(=O)c2cnc(C)cn2)cc1]

or is

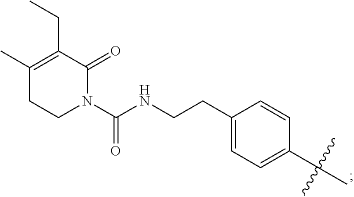 [SMILES: *c1ccc(CCNC(=O)N2CCC(C)=C(CC)C2=O)cc1]

or is

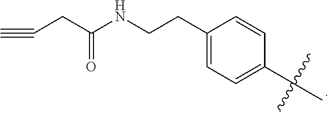 [SMILES: *c1ccc(CCNC(=O)CC#C)cc1]

In some embodiments of one or more formulae herein,

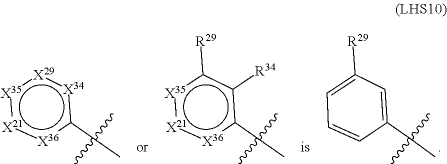

In some embodiments of one or more formulae herein, LHS10 is

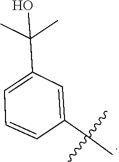 [SMILES: *c1cccc(C(C)(C)O)c1]

In some embodiments of one or more formulae herein,

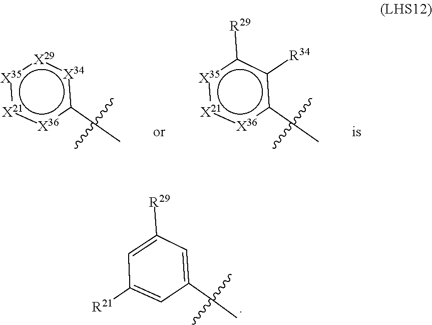

In some embodiments of one or more formulae herein, LHS12 is

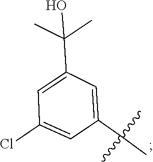 [SMILES: *c1cc(Cl)cc(C(C)(C)O)c1]

or is

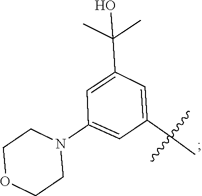 [SMILES: *c1cc(N2CCOCC2)cc(C(C)(C)O)c1]

or is

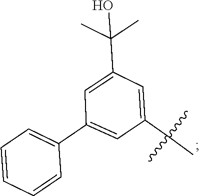 [SMILES: *c1cc(-c2ccccc2)cc(C(C)(C)O)c1]

or is

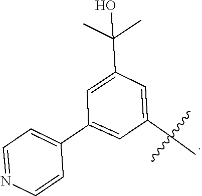 [SMILES: *c1cc(-c2ccncc2)cc(C(C)(C)O)c1]

In some embodiments of one or more formulae herein,

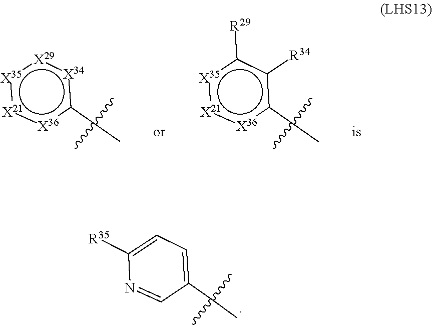

In some embodiments of one or more formulae herein, LHS13 is

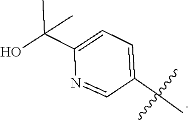 [SMILES: *c1ccc(C(C)(C)O)nc1]

In some embodiments of one or more formulae herein,

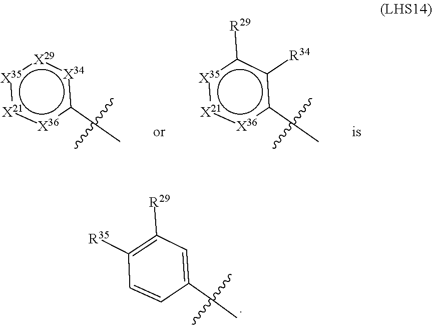

In some embodiments of one or more formulae herein, LHS14 is

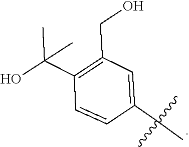 [SMILES: *c1ccc(C(C)(C)O)c(CO)c1]

In some embodiments of one or more formulae herein,

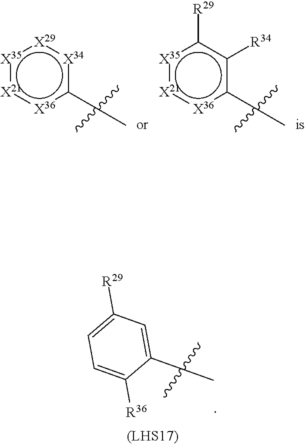

In some embodiments of one or more formulae herein, LHS17 is

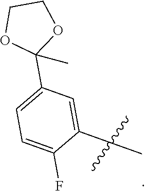 [SMILES: *c1cc(C2(C)OCCO2)ccc1F]

In some embodiments of one or more formulae herein,

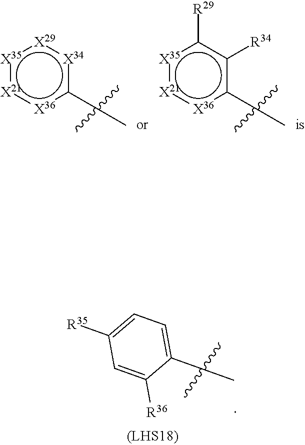

In some embodiments of one or more formulae herein,

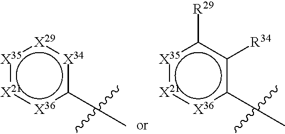

is

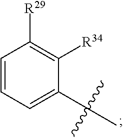

or is

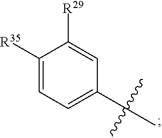

or is

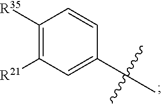

or is

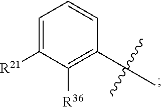

or is

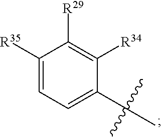

or is

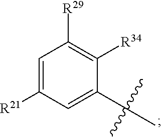

or is

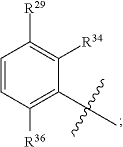

or is

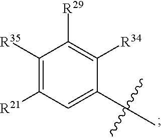

or is

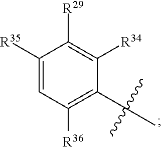

or is

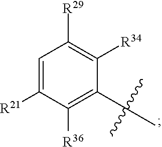

or is

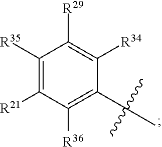

or is

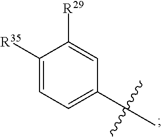

or is

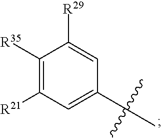

or is

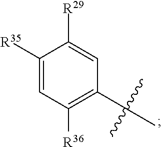

or is

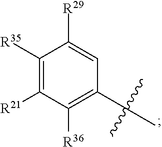

or is

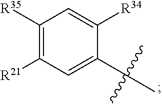

or is

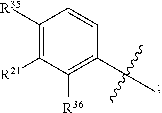

or is

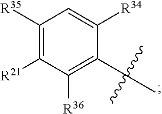

or is

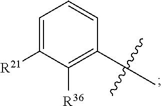

or is

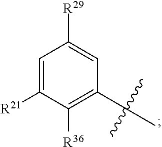

or is

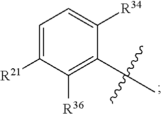

or is

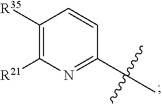

or is

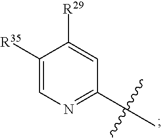

or is

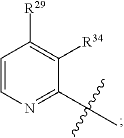

or is

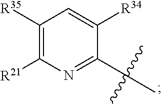

or is

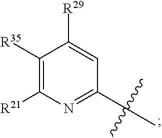

or is

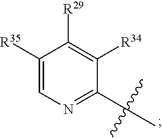

or is

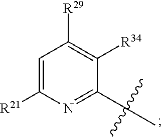

or is

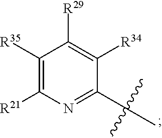

or is

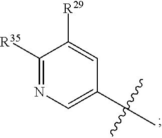

or is

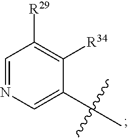

or is

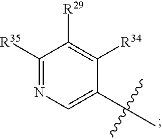

or is

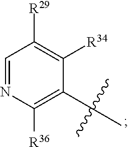

or is

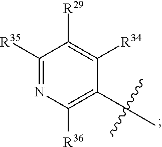

or is

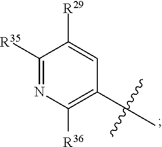

or is

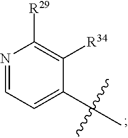

or is

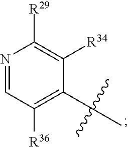

or is

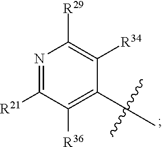

or is

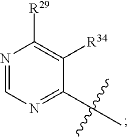

or is

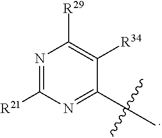

In some embodiments of one or more formulae herein,

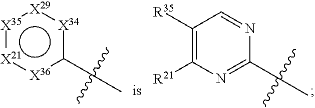

or is

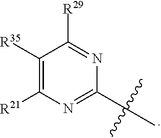

In some embodiments of one or more formulae herein,

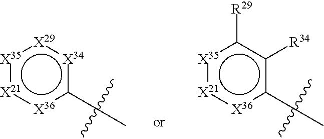

is

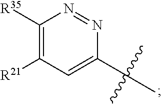

or is

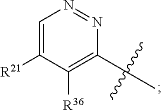

or is

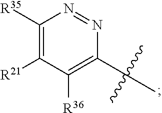

or is

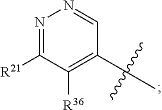

or is

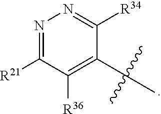

In some embodiments of one or more formulae herein,

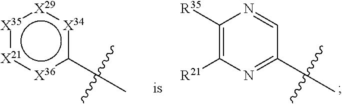

or is

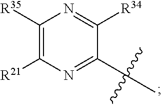

or is

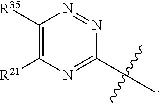

In some embodiments of one or more formulae herein,

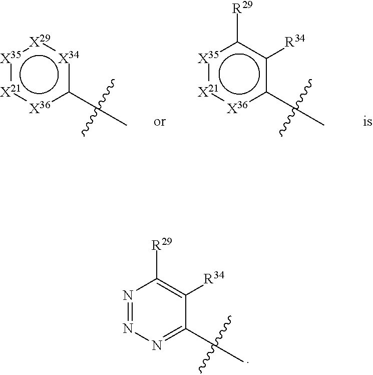

### Non-Limiting Combinations

In some embodiments of one or more formulae herein Ar is LHS1,

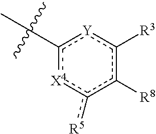

is RHS1, each R²⁰ is hydrogen; or Ar is LHS1,

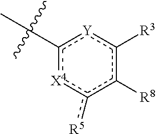

is RHS2, each R²⁰ is hydrogen; or Ar is LHS1,

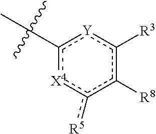

is RHS3, each R²⁰ is hydrogen; or Ar is LHS1,

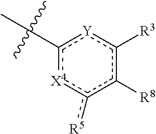

is RHS4, each R²⁰ is hydrogen; or Ar is LHS1,

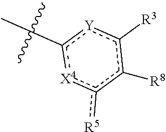

is RHS5, each R²⁰ is hydrogen; or Ar is LHS1,

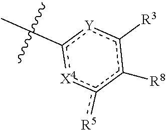

is RHS6, each R²⁰ is hydrogen; or Ar is LHS1,

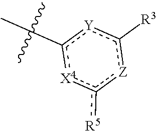

is RHS7, each R²⁰ is hydrogen; or Ar is LHS1,

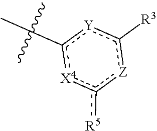

is RHS8, each R²⁰ is hydrogen; or Ar is LHS2,

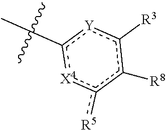

is RHS1, each R²⁰ is hydrogen; or Ar is LHS2,

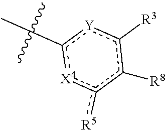

is RHS2, each R²⁰ is hydrogen; or Ar is LHS2,

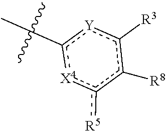

is RHS3, each R²⁰ is hydrogen; or Ar is LHS2,

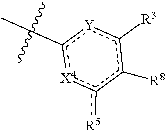

is RHS4, each R²⁰ is hydrogen; or Ar is LHS2,

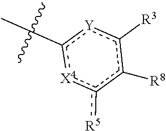

is RHS5, each R²⁰ is hydrogen; or Ar is LHS2,

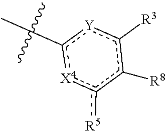

is RHS6, each R²⁰ is hydrogen; or Ar is LHS2,

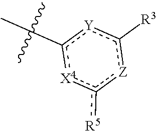

is RHS7, each R²⁰ is hydrogen; or Ar is LHS2,

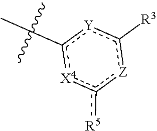

is RHS8, each R²⁰ is hydrogen; or Ar is LHS3,

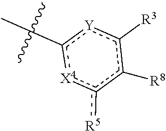

is RHS1, each R²⁰ is hydrogen; or Ar is LHS3,

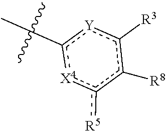

is RHS2, each R²⁰ is hydrogen; or Ar is LHS3,

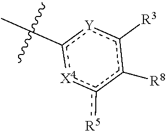

is RHS3, each R²⁰ is hydrogen; or Ar is LHS3,

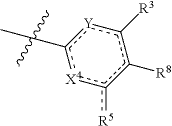

is RHS4, each R²⁰ is hydrogen; or Ar is LHS3,

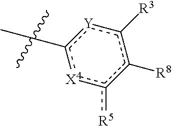

is RHS5, each R²⁰ is hydrogen; or Ar is LHS3,

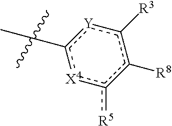

is RHS6, each R²⁰ is hydrogen; or Ar is LHS3,

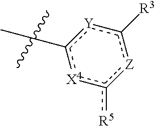

is RHS7, each R²⁰ is hydrogen; or Ar is LHS3,

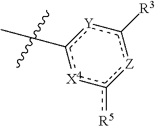

is RHS8, each R²⁰ is hydrogen; or Ar is LHS4,

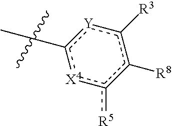

is RHS1, each R²⁰ is hydrogen; or Ar is LHS4,

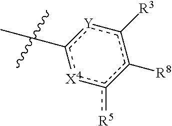

is RHS2, each R²⁰ is hydrogen; or Ar is LHS4,

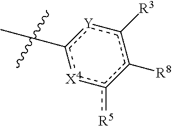

is RHS3, each R²⁰ is hydrogen; or Ar is LHS4,

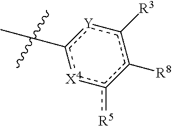

is RHS4, each R²⁰ is hydrogen; or Ar is LHS4,

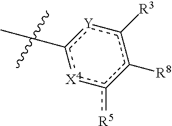

is RHS5, each R²⁰ is hydrogen; or Ar is LHS4,

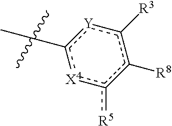

is RHS6, each R²⁰ is hydrogen; or Ar is LHS4,

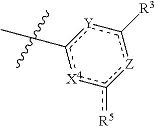

is RHS7, each R²⁰ is hydrogen; or Ar is LHS4,

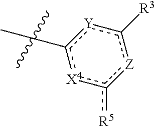

is RHS8, each R²⁰ is hydrogen; or Ar is LHS5,

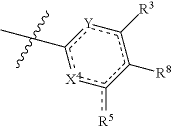

is RHS1, each R²⁰ is hydrogen; or Ar is LHS5,

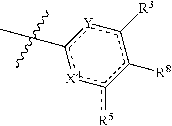

is RHS2, each R²⁰ is hydrogen; or Ar is LHS5,

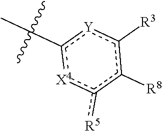

is RHS3, each R²⁰ is hydrogen; or Ar is LHS5,

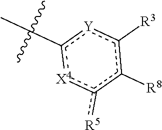

is RHS4, each R²⁰ is hydrogen; or Ar is LHS5,

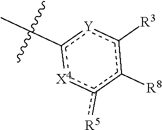

is RHS5, each R²⁰ is hydrogen; or Ar is LHS5,

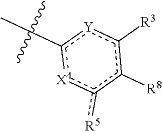

is RHS6, each R²⁰ is hydrogen; or Ar is LHS5,

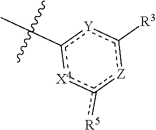

is RHS7, each R²⁰ is hydrogen; or Ar is LHS5,

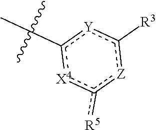

is RHS8, each R²⁰ is hydrogen; or Ar is LHS6,

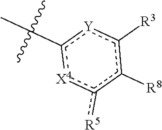

is RHS1, each R²⁰ is hydrogen; or Ar is LHS6,

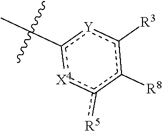

is RHS2, each R²⁰ is hydrogen; or Ar is LHS6,

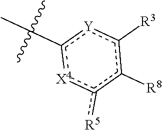

is RHS3, each R²⁰ is hydrogen; or Ar is LHS6,

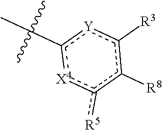

is RHS4, each R²⁰ is hydrogen; or Ar is LHS6,

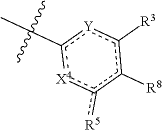

is RHS5, each R²⁰ is hydrogen; or Ar is LHS6,

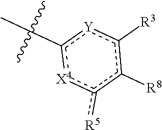

is RHS6, each R²⁰ is hydrogen; or Ar is LHS6,

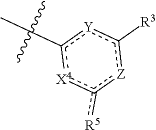

is RHS7, each R²⁰ is hydrogen; or Ar is LHS6,

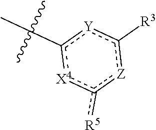

is RHS8, each R²⁰ is hydrogen; or Ar is LHS7,

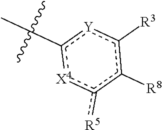

is RHS1, each R²⁰ is hydrogen; or Ar is LHS7,

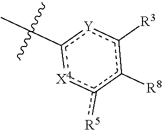

is RHS2, each R²⁰ is hydrogen; or Ar is LHS7,

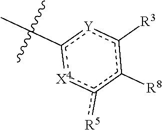

is RHS3, each R²⁰ is hydrogen; or Ar is LHS7,

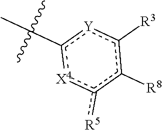

is RHS4, each R²⁰ is hydrogen; or Ar is LHS7,

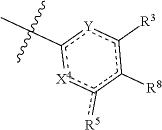

is RHS5, each R²⁰ is hydrogen; or Ar is LHS7,

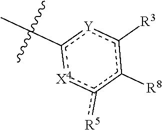

is RHS6, each R²⁰ is hydrogen; or Ar is LHS7,

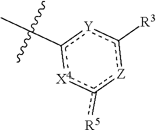

is RHS7, each R²⁰ is hydrogen; or Ar is LHS7,

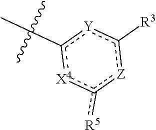

is RHS8, each R²⁰ is hydrogen; or Ar is LHS8,

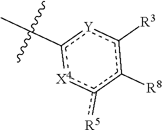

is RHS1, each R²⁰ is hydrogen; or Ar is LHS8,

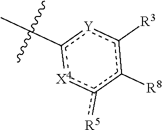

is RHS2, each R²⁰ is hydrogen; or Ar is LHS8,

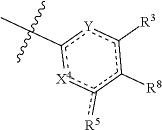

is RHS3, each R²⁰ is hydrogen; or Ar is LHS8,

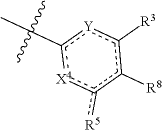

is RHS4, each R²⁰ is hydrogen; or Ar is LHS8,

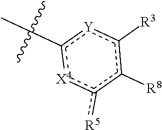

is RHS5, each R²⁰ is hydrogen; or Ar is LHS8,

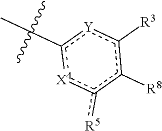

is RHS6, each R²⁰ is hydrogen; or Ar is LHS8,

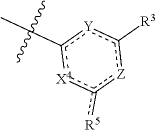

is RHS7, each R²⁰ is hydrogen; or Ar is LHS8,

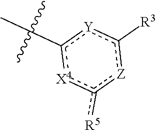

is RHS3, each R²⁰ is hydrogen; or Ar is LHS9,

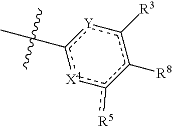

is RHS4, each R²⁰ is hydrogen; or Ar is LHS9,

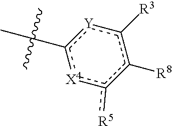

is RHS2, each R²⁰ is hydrogen; or Ar is LHS9,

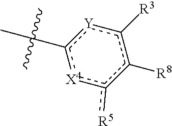

is RHS3, each R²⁰ is hydrogen; or Ar is LHS9,

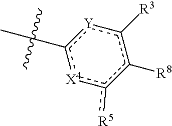

is RHS4, each R²⁰ is hydrogen; or Ar is LHS9,

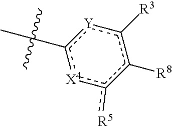

is RHS5, each R²⁰ is hydrogen; or Ar is LHS9,

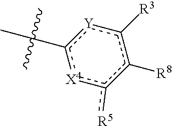

is RHS6, each R²⁰ is hydrogen; or Ar is LHS9,

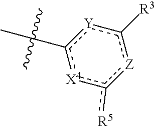

is RHS7, each R²⁰ is hydrogen; or Ar is LHS9,

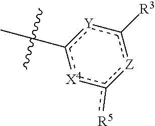

is RHS8, each R²⁰ is hydrogen; or Ar is LHS10,

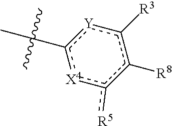

is RHS1, each R²⁰ is hydrogen; or Ar is LHS10,

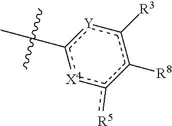

is RHS2, each R²⁰ is hydrogen; or Ar is LHS10,

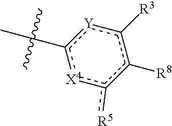

is RHS3, each R²⁰ is hydrogen; or Ar is LHS10,

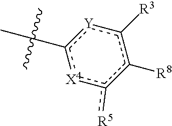

is RHS4, each R²⁰ is hydrogen; or Ar is LHS10,

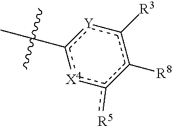

is RHS5, each R²⁰ is hydrogen; or Ar is LHS10,

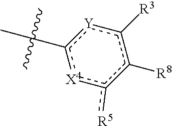

is RHS6, each R²⁰ is hydrogen; or Ar is LHS10,

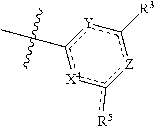

is RHS7, each R²⁰ is hydrogen; or Ar is LHS10,

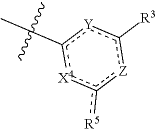

is RHS8, each R²⁰ is hydrogen; or Ar is LHS11,

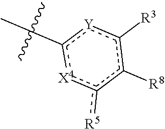

is RHS1, each R²⁰ is hydrogen; or Ar is LHS11,

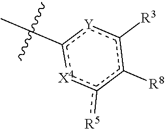

is RHS2, each R²⁰ is hydrogen; or Ar is LHS11,

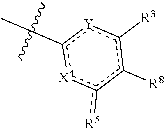

is RHS3, each R²⁰ is hydrogen; or Ar is LHS11,

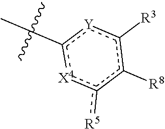

is RHS4, each R²⁰ is hydrogen; or Ar is LHS11,

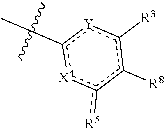

is RHS5, each R²⁰ is hydrogen; or Ar is LHS11,

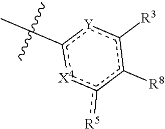

is RHS6, each R²⁰ is hydrogen; or Ar is LHS11,

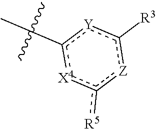

is RHS7, each R²⁰ is hydrogen; or Ar is LHS11,

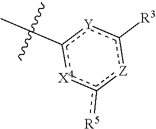

is RHS1, each R²⁰ is hydrogen; or Ar is LHS11,

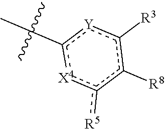

is RHS1, each R²⁰ is hydrogen; or Ar is LHS12,

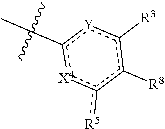

is RHS2, each R²⁰ is hydrogen; or Ar is LHS12,

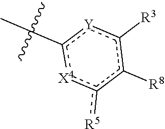

is RHS3, each R²⁰ is hydrogen; or Ar is LHS12,

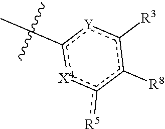

is RHS4, each R²⁰ is hydrogen; or Ar is LHS12,

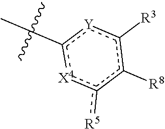

is RHS5, each R²⁰ is hydrogen; or Ar is LHS12,

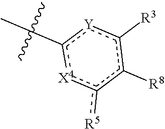

is RHS6, each R²⁰ is hydrogen; or Ar is LHS12,

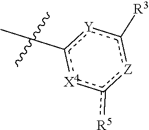

is RHS7, each R²⁰ is hydrogen; or Ar is LHS12,

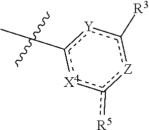

is RHS8, each R²⁰ is hydrogen; or Ar is LHS13,

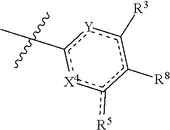

is RHS1, each R²⁰ is hydrogen; or Ar is LHS13,

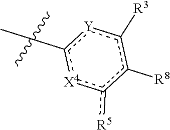

is RHS2, each R²⁰ is hydrogen; or Ar is LHS13,

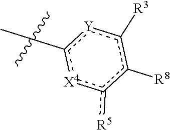

is RHS3, each R²⁰ is hydrogen; or Ar is LHS13,

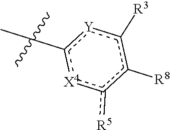

is RHS4, each R²⁰ is hydrogen; or Ar is LHS13,

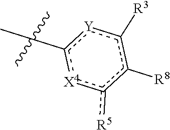

is RHS5, each R²⁰ is hydrogen; or Ar is LHS13,

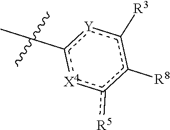

is RHS6, each R²⁰ is hydrogen; or Ar is LHS13,

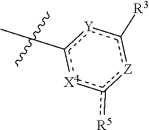

is RHS7, each R²⁰ is hydrogen; or Ar is LHS13,

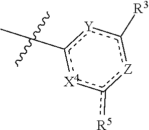

is RHS8, each R²⁰ is hydrogen; or Ar is LHS14,

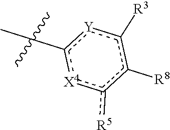

is RHS1, each R²⁰ is hydrogen; or Ar is LHS14,

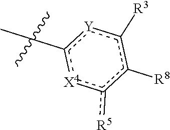

is RHS2, each R²⁰ is hydrogen; or Ar is LHS14,

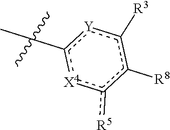

is RHS3, each R²⁰ is hydrogen; or Ar is LHS14,

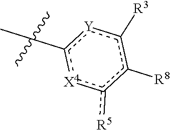

is RHS4, each R²⁰ is hydrogen; or Ar is LHS14,

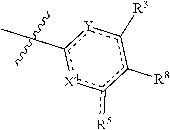

is RHS5, each R²⁰ is hydrogen; or Ar is LHS14,

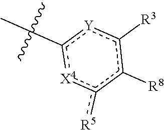

is RHS6, each R²⁰ is hydrogen; or Ar is LHS14,

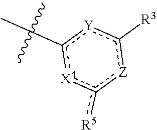

is RHS7, each R²⁰ is hydrogen; or Ar is LHS14,

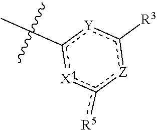

is RHS8, each R²⁰ is hydrogen; or Ar is LHS17,

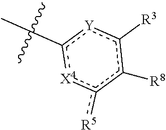

is RHS1, each R²⁰ is hydrogen; or Ar is LHS17,

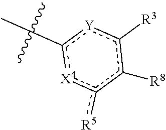

is RHS2, each R²⁰ is hydrogen; or Ar is LHS17,

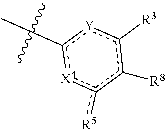

is RHS3, each R²⁰ is hydrogen; or Ar is LHS17,

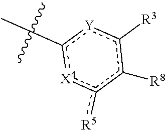

is RHS4, each R²⁰ is hydrogen; or Ar is LHS17,

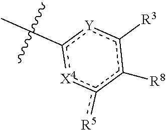

is RHS5, each R²⁰ is hydrogen; or Ar is LHS17,

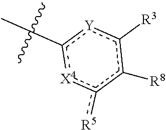

is RHS6, each R²⁰ is hydrogen; or Ar is LHS17,

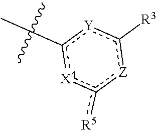

is RHS7, each R²⁰ is hydrogen; or Ar is LHS17,

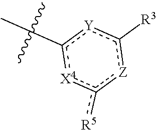

is RHS8, each R²⁰ is hydrogen; or Ar is LHS18,

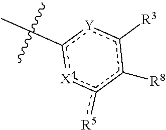

is RHS1, each R²⁰ is hydrogen; or Ar is LHS18,

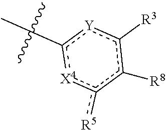

is RHS2, each R²⁰ is hydrogen; or Ar is LHS18,

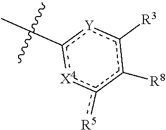

is RHS3, each R²⁰ is hydrogen; or Ar is LHS18,

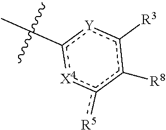

is RHS4, each R²⁰ is hydrogen; or Ar is LHS18,

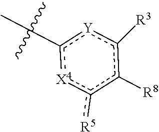

is RHS5, each R²⁰ is hydrogen; or Ar is LHS18,

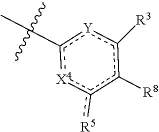

is RHS6, each R²⁰ is hydrogen; or Ar is LHS18,

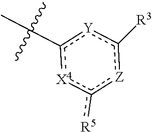

is RHS7, each R²⁰ is hydrogen; or Ar is LHS18,

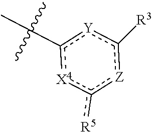

is RHS8, each R²⁰ is hydrogen; or Ar is LHS1,

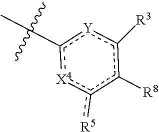

is RHS9, each R²⁰ is hydrogen; or Ar is LHS1,

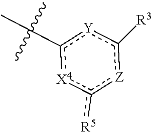

is RHS10, each R²⁰ is hydrogen; or Ar is LHS1,

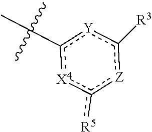

is RHS11, each R²⁰ is hydrogen; or Ar is LHS1,

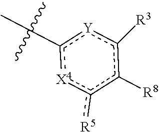

is RHS12, each R²⁰ is hydrogen; or Ar is LHS2,

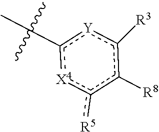

is RHS9, each R²⁰ is hydrogen; or Ar is LHS2,

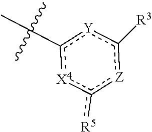

is RHS10, each R²⁰ is hydrogen; or Ar is LHS2,

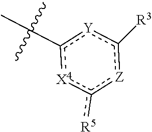

is RHS11, each R²⁰ is hydrogen; or Ar is LHS2,

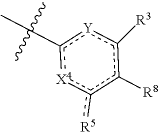

is RHS12, each R²⁰ is hydrogen; or Ar is LHS3,

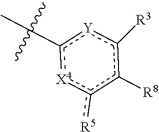

is RHS9, each R²⁰ is hydrogen; or Ar is LHS3,

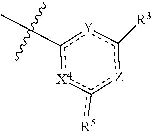

is RHS10, each R²⁰ is hydrogen; or Ar is LHS3,

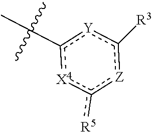

is RHS11, each R²⁰ is hydrogen; or Ar is LHS3,

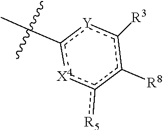

is RHS12, each R²⁰ is hydrogen; or Ar is LHS4,

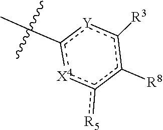

is RHS9, each R²⁰ is hydrogen; or Ar is LHS4,

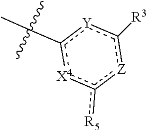

is RHS10, each R²⁰ is hydrogen; or Ar is LHS4,

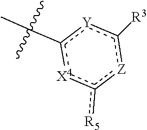

is RHS11, each R²⁰ is hydrogen; or Ar is LHS4,

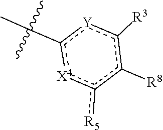

is RHS12, each R²⁰ is hydrogen; or Ar is LHS5,

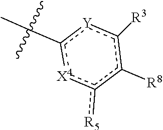

is RHS9, each R²⁰ is hydrogen; or Ar is LHS5,

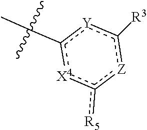

is RHS10, each R²⁰ is hydrogen; or Ar is LHS5,

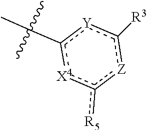

is RHS11, each R²⁰ is hydrogen; or Ar is LHS5,

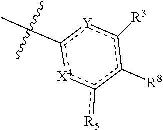

is RHS12, each R²⁰ is hydrogen; or Ar is LHS6,

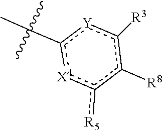

is RHS9, each R²⁰ is hydrogen; or Ar is LHS6,

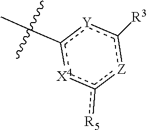

is RHS10, each R²⁰ is hydrogen; or Ar is LHS6,

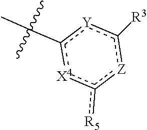

is RHS11, each R²⁰ is hydrogen; or Ar is LHS6,

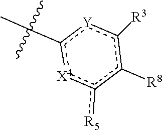

is RHS12, each R²⁰ is hydrogen; or Ar is LHS7,

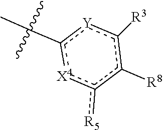

is RHS9, each R²⁰ is hydrogen; or Ar is LHS7,

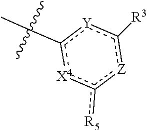

is RHS10, each R²⁰ is hydrogen; or Ar is LHS7,

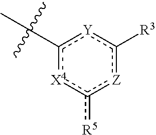

is RHS11, each R²⁰ is hydrogen; or Ar is LHS7,

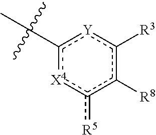

is RHS12, each R²⁰ is hydrogen; or Ar is LHS8,

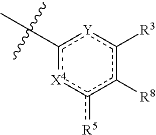

is RHS9, each R²⁰ is hydrogen; or Ar is LHS8,

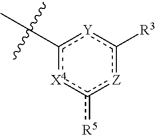

is RHS10, each R²⁰ is hydrogen; or Ar is LHS8,

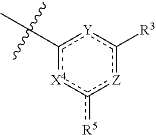

is RHS11, each R²⁰ is hydrogen; or Ar is LHS8,

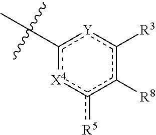

is RHS12, each R²⁰ is hydrogen; or Ar is LHS9,

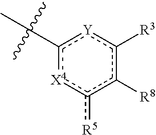

is RHS9, each R²⁰ is hydrogen; or Ar is LHS9,

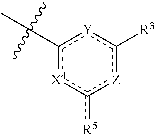

is RHS10, each R²⁰ is hydrogen; or Ar is LHS9,

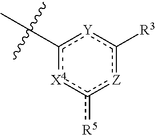

is RHS11, each R²⁰ is hydrogen; or Ar is LHS9,

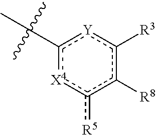

is RHS12, each R²⁰ is hydrogen; or Ar is LHS10,

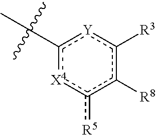

is RHS9, each R²⁰ is hydrogen; or Ar is LHS10,

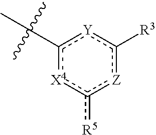

is RHS10, each R²⁰ is hydrogen; or Ar is LHS10,

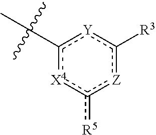

is RHS11, each R²⁰ is hydrogen; or Ar is LHS10,

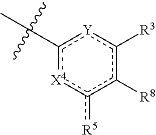

is RHS12, each R²⁰ is hydrogen; or Ar is LHS11,

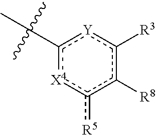

is RHS9, each R²⁰ is hydrogen; or Ar is LHS11,

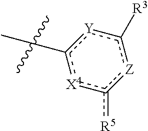

is RHS10, each R²⁰ is hydrogen; or Ar is LHS11,

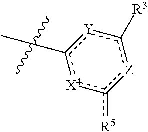

is RHS11, each R²⁰ is hydrogen; or Ar is LHS11,

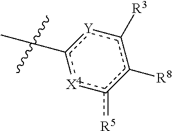

is RHS12, each R²⁰ is hydrogen; or Ar is LHS12,

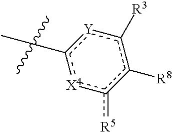

is RHS9, each R²⁰ is hydrogen; or Ar is LHS12,

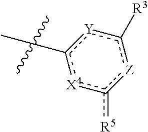

is RHS10, each R²⁰ is hydrogen; or Ar is LHS12,

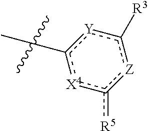

is RHS11, each R²⁰ is hydrogen; or Ar is LHS12,

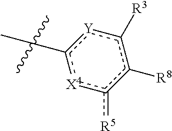

is RHS12, each R²⁰ is hydrogen; or Ar is LHS13,

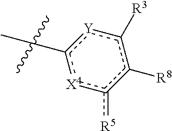

is RHS9, each R²⁰ is hydrogen; or is LHS13,

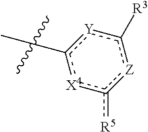

is RHS10, each R²⁰ is hydrogen; or Ar is LHS13,

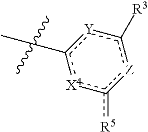

is RHS11, each R²⁰ is hydrogen; or Ar is LHS13,

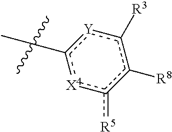

is RHS12, each R²⁰ is hydrogen; or Ar is LHS14,

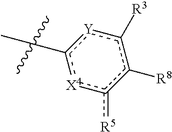

is RHS9, each R²⁰ is hydrogen; or Ar is LHS14,

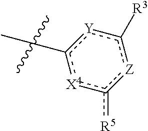

is RHS10, each R²⁰ is hydrogen; or Ar is LHS14,

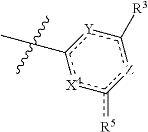

is RHS11, each R²⁰ is hydrogen; or Ar is LHS14,

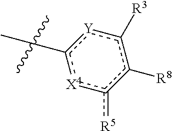

is RHS12, each R²⁰ is hydrogen; or Ar is LHS17,

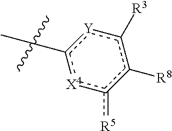

is RHS9, each R²⁰ is hydrogen; or Ar is LHS17,

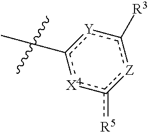

is RHS10, each R²⁰ is hydrogen; or Ar is LHS17,

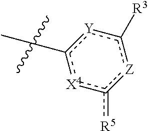

is RHS11, each R²⁰ is hydrogen; or Ar is LHS17,

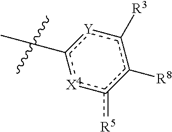

is RHS12, each R²⁰ is hydrogen; or Ar is LHS18,

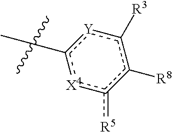

is RHS9, each R²⁰ is hydrogen; or Ar is LHS18,

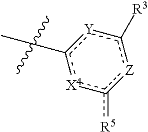

is RHS10, each R²⁰ is hydrogen; or Ar is LHS18,

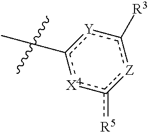

is RHS11, each R²⁰ is hydrogen; or Ar is LHS18,

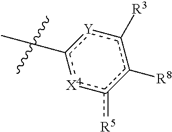

is RHS12, each R²⁰ is hydrogen; or Ar is LHS19,

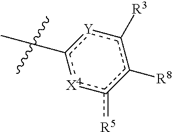

is RHS1, each R²⁰ is hydrogen; or Ar is LHS19,

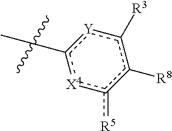

is RHS2, each R²⁰ is hydrogen; or Ar is LHS19,

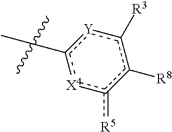

is RHS3, each R²⁰ is hydrogen; or Ar is LHS19,

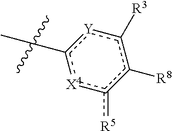

is RHS4, each R²⁰ is hydrogen; or Ar is LHS19,

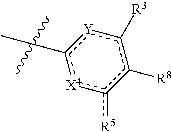

is RHS5, each R²⁰ is hydrogen; or Ar is LHS19,

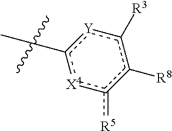

is RHS6, each R²⁰ is hydrogen; or is LHS19,

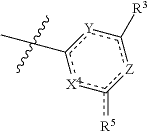

is RHS7, each R²⁰ is hydrogen; or Ar is LHS19,

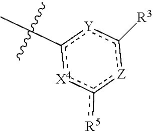

is RHS8, each R²⁰ is hydrogen; or Ar is LHS19,

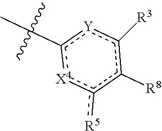

is RHS9, each R²⁰ is hydrogen; or is LHS19,

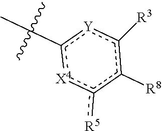

is RHS10, each R²⁰ is hydrogen; or Ar is LHS19,

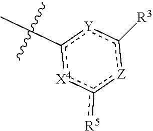

is RHS11, each R²⁰ is hydrogen; or Ar is LHS19,

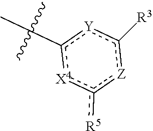

is RHS12, each R²⁰ is hydrogen.

In some embodiments, the moiety

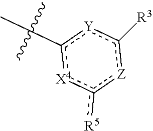

is selected from the group consisting of:

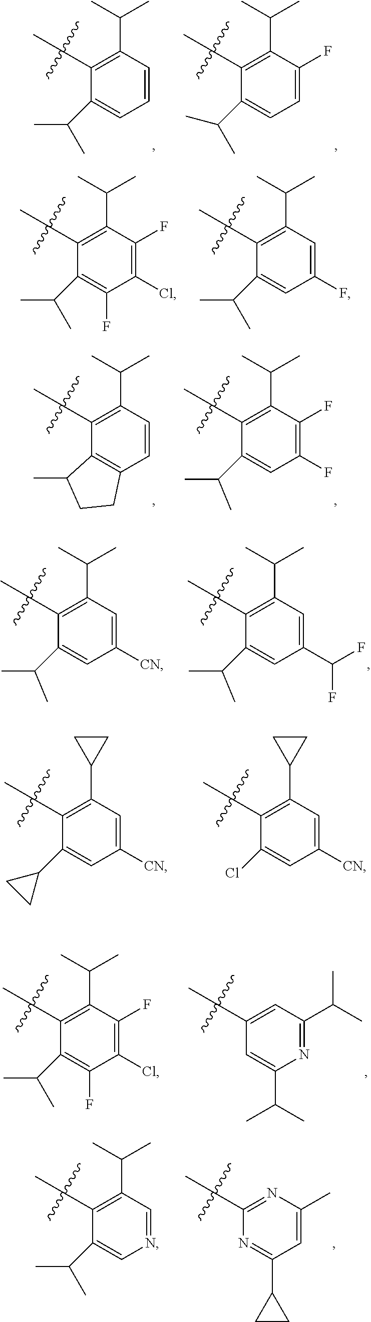

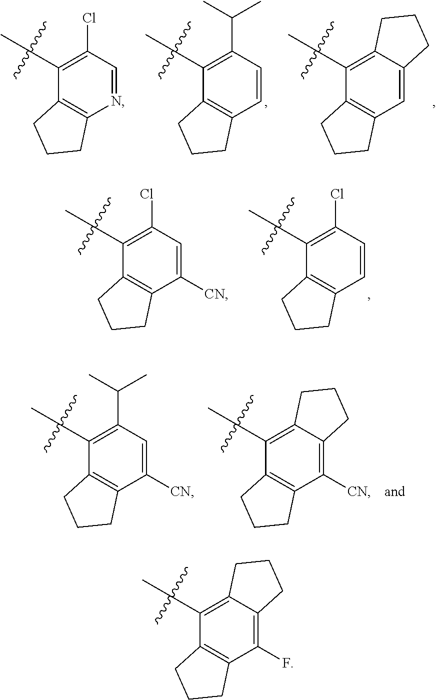

In some embodiments of the compound of Formula A,
Ar is a heteroaryl group

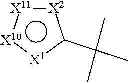

or an aryl or heteroaryl group

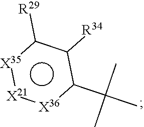

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;
X³⁵ is N or CR³⁵;
X²¹ is N or CR²¹;
X³⁶ is N or CR³⁶;
X⁴ is CR⁴, N or NR²⁴;
each R²⁰ is hydrogen;

### Y is CR²;

### Z is N or CR⁸;

R⁸ is selected from H, CN, Cl, F, C₁-C₆ alkyl, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy; or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

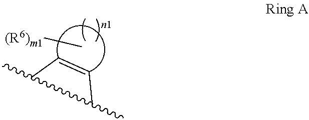

and ring B is

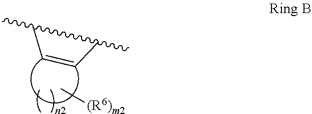

wherein
each R⁶ in each ring is H;
each of R¹, R¹⁰, R⁴¹ and R⁴² when bonded to carbon is independently selected from H, C₁-C₆ alkyl, C₁-C₆ haloalkyl, halo, C₆-C₁₀ aryl, C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, and NR¹¹R¹²;
and each of R¹, R¹⁰, R⁴¹ and R⁴² when bonded to nitrogen is independently selected from H, C₁-C₆ alkyl, C₁-C₆ haloalkyl, C₆-C₁₀ aryl, C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, and NR¹¹R¹²;
or R¹ and R¹⁰ taken together with the atoms connecting them form a 3-to-8-membered carbocyclic ring;
each of R³⁴, R²⁹, R³⁵, R²¹ and R³⁶ is independently selected from H, C₁-C₆ alkyl, C₁-C₆ haloalkyl, halo, C₃-C₇ cycloalkyl, 3- to 7-membered heterocycloalkyl, C₆-C₁₀ aryl, and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, and NR¹¹R¹²,
or two groups selected from R³⁴, R²⁹, R³⁵, R²¹ and R³⁶ that are on adjacent ring carbon atoms taken together with the adjacent ring carbons form a 6-membered aromatic ring;
each of R¹¹ and R¹² at each occurrence is hydrogen.
In some embodiments of the compound of Formula A or Formula I,
Ar is a heteroaryl group

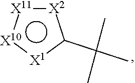

X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²;
each of R¹, R¹⁰, R⁴¹ and R⁴² when bonded to carbon is independently selected from H, C₁-C₆ alkyl, C₁-C₆ haloalkyl, halo, C₆-C₁₀ aryl, C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, and NR¹¹R¹²;
and each of R¹, R⁰, R⁴¹ and R⁴² when bonded to nitrogen is independently selected from H, C₁-C₆ alkyl, C₁-C₆ haloalkyl, C₆-C₁₀ aryl, C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, and NR¹¹R¹²;
or R¹ and R¹⁰ taken together with the atoms connecting them form a 3-to-8-membered carbocyclic ring.

In some embodiments of the compound of Formula A or Formula II,

Ar is an aryl or heteroaryl group

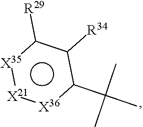

X³⁵ is N or CR³⁵;
X²¹ is N or CR²¹;
X³⁶ is N or CR³⁶;
each of R³⁴, R²⁹, R³⁵, R²¹ and R³⁶ is independently selected from H, C₁-C₆ alkyl, C₁-C₆ haloalkyl, halo, C₃-C₇ cycloalkyl, 3- to 7-membered heterocycloalkyl, C₆-C₁₀ aryl, and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, and NR¹¹R¹²,
or two groups selected from R³⁴, R²⁹, R³⁵, R²¹ and R³⁶ that are on adjacent ring carbon atoms taken together with the adjacent ring carbons form a 6-membered aromatic ring.
In some embodiments of the compound of Formula A or I,
Ar is a heteroaryl group

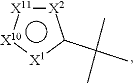

wherein

### X¹ is O, S, N or CH;

X¹⁰ is N, CR¹⁰ or NR¹⁰;
X¹¹ is N, CR¹ or NR¹;

### X² is O, S, N or CH;

each of R¹ and R¹⁰ when bonded to carbon is independently selected from H, C₁-C₆ alkyl, C₆-C₁₀ aryl, S(O₂)C₁-C₆ alkyl and C₃-C₇ cycloalkyl, wherein the C₁-C₆ alkyl and C₃-C₇ cycloalkyl is optionally substituted with one or more substituents each independently selected from hydroxy, oxo, C₁-C₆ alkoxy, and NR¹¹R¹²;
and each of R¹, R¹⁰ when bonded to nitrogen is independently selected from H, C₁-C₆ alkyl, C₆-C₁₀ aryl, and C₃-C₇ cycloalkyl, wherein the C₁-C₆ alkyl and C₃-C₇ cycloalkyl is optionally substituted with one or more substituents each independently selected from hydroxy and C₁-C₆ alkoxy;
R⁸ is selected from H, CN, Cl, F, C₁-C₆ alkyl, C₁-C₆ alkoxy, C₁-C₆ haloalkoxy, and C₁-C₆ haloalkyl;
R² is hydrogen, C₁-C₆ haloalkyl, C₃-C₇ cycloalkyl or C₁-C₆ alkyl;
R³ is hydrogen or halo;
R⁴ is hydrogen, C₁-C₆ haloalkyl, C₃-C₇ cycloalkyl or C₁-C₆ alkyl;
R⁵ is hydrogen or halo.
In some embodiments, the compound of formula I is a compound of formula Ia

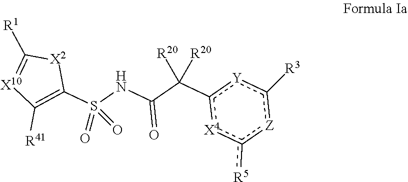

wherein
X¹⁰ is N or CR¹⁰; and X² is O, S, or NR⁴² or
X¹⁰ is N; and X² is O; or
X¹⁰ is N; and X² is S; or
X¹⁰ is CR¹⁰; and X² is O; or
X¹⁰ is CR¹⁰; and X² is S; or
X¹⁰ is CH; and X² is O; or
X¹⁰ is CH; and X² is S.
In some embodiments, the compound of formula I is a compound of formula Ib

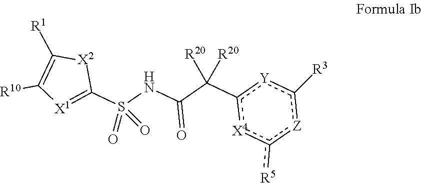

wherein
X¹ is O, S, or NR⁴¹; and
X² is N or CR⁴².
In some embodiments of the compound of formula Ib
X¹ is O; and X² is N; or
X¹ is S; and X² is N; or
X¹ is O; and X² is CR⁴²; or
X¹ is S; and X² is CR⁴²; or
X¹ is O; and X² is CH; or
X¹ is S; and X² is CH; or
X¹ is S; and X² is CCH₃.
In some embodiments of the compound of formula A, I, Ia or Ib, R¹ is C₁-C₆ alkyl optionally substituted with hydroxy; or R¹⁰ is C₁-C₆ alkyl optionally substituted with hydroxy; or R¹ is 2-hydroxy-2-propyl; or R¹⁰ is 2-hydroxy-2-propyl; or R¹ is C₃-C₇ cycloalkyl optionally substituted with hydroxy; or R¹⁰ is C₃-C₇ cycloalkyl optionally substituted with hydroxy; or R¹ is 1-hydroxy-1-cyclopropyl; or R¹⁰ is 1-hydroxy-1-cyclopropyl; or R⁴¹ is C₁-C₆ alkyl optionally substituted with hydroxy; or R⁴² is C₁-C₆ alkyl optionally substituted with hydroxy; or R⁴¹ is 2-hydroxy-2-propyl; or R⁴² is 2-hydroxy-2-propyl; or R⁴¹ is C₃-C₇ cycloalkyl optionally substituted with hydroxy; or R⁴² is C₃-C₇ cycloalkyl optionally substituted with hydroxy; or R⁴¹ is 1-hydroxy-1-cyclopropyl; or R⁴² is 1-hydroxy-1-cyclopropyl; or R¹ is C₁-C₆ alkyl optionally substituted with NR¹¹R¹², wherein each of R¹¹ and R¹² is independently selected from hydrogen and C₁-C₆ alkyl; or R¹ is aminomethyl; or R¹ is methylaminomethyl; or R¹ is dimethylaminomethyl; or R¹ is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R¹ is S(O₂)C₁-C₆ alkyl; or R¹ is S(O₂)CH₃; or
R¹⁰ is C₁-C₆ alkyl optionally substituted with NR¹¹R¹², wherein each of R¹¹ and R¹² is independently selected from hydrogen and C₁-C₆ alkyl; or R¹⁰ is aminomethyl; or R¹⁰ is methylaminomethyl; or R¹⁰ is dimethylaminomethyl; or R¹⁰ is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R¹⁰ is S(O₂)C₁-C₆ alkyl; or R¹⁰ is S(O₂)CH₃; or R⁴¹ is C₁-C₆ alkyl optionally substituted with NR¹¹R¹², wherein each of R¹¹ and R¹² is independently selected from hydrogen and C₁-C₆ alkyl; or R⁴¹ is aminomethyl; or R⁴¹ is methylaminomethyl; or R⁴¹ is dimethylaminomethyl; R⁴¹ is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R⁴¹ is S(O₂)C₁-C₆ alkyl, or R⁴¹ is S(O₂)CH₃; or R⁴² is C₁-C₆ alkyl optionally substituted with NR¹¹R¹², wherein each of R¹¹ and R¹² is independently selected from hydrogen and C₁-C₆ alkyl; or R⁴² is aminomethyl; or R⁴² is methylaminomethyl; or R⁴² is dimethylaminomethyl; or R⁴² is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R⁴² is S(O₂)C₁-C₆ alkyl; or R⁴² is S(O₂)CH₃.
In some embodiments of the compound of Formula A or II,
Ar is an aryl or heteroaryl group

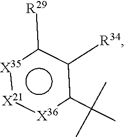

X³⁵ is CR³⁵;
X²¹ is N or CR²¹;
X³⁶ is CR³⁶;
each of R³⁴, R²⁹, R³⁵, R²¹ and R³⁶ is independently selected from H, C₁-C₆ alkyl, halo, C₃-C₇ cycloalkyl, 3- to 7-membered nonaromatic monocyclic heterocycloalkyl, C₆-C₁₀ aryl, and S(O₂)C₁-C₆ alkyl;
wherein the C₁-C₆ alkyl, 3- to 7-membered nonaromatic monocyclic heterocycloalkyl, and C₃-C₇ cycloalkyl is optionally substituted with one or more substituents each independently selected from hydroxyl, C₁-C₆ alkyl, oxo, NR¹¹R¹², and 3- to 7-membered heterocycloalkyl, R⁸ is selected from H, CN, Cl, F, C₁-C₆ alkyl, C₁-C₆ alkoxy, C₁-C₆ haloalkoxy, and C₁-C₆ haloalkyl;
R² is hydrogen, C₁-C₆ haloalkyl, C₃-C₇ cycloalkyl or C₁-C₆ alkyl;
R³ is hydrogen or halo;
R⁴ is hydrogen, C₁-C₆ haloalkyl, C₃-C₇ cycloalkyl or C₁-C₆ alkyl;
R⁵ is hydrogen or halo.
In some embodiments of the compound of formula A or II, R³⁵ is 2-hydroxy-2-propyl; or R²¹ is 2-hydroxy-2-propyl; or R²⁹ is 2-hydroxy-2-propyl; or R³⁵ is 1-hydroxy-1-cyclopropyl.

In some embodiments of the compound of formula A or II, R²¹ is 1-hydroxy-1-cyclopropyl; or

R²⁹ is 1-hydroxy-1-cyclopropyl; or R³⁵ is C₁-C₆ alkyl optionally substituted with NR¹¹R¹², wherein each of R¹¹ and R¹² is independently selected from hydrogen and C₁-C₆ alkyl; or R³⁵ is aminomethyl; or R³⁵ is methylaminomethyl; or R³⁵ is dimethylaminomethyl; or R³⁵ is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R³⁵ is S(O₂)C₁-C₆ alkyl;
or R³⁵ is S(O₂)CH₃; or R²¹ is C₁-C₆ alkyl optionally substituted with NR¹¹R¹², wherein each of R¹¹ and R¹² is independently selected from hydrogen and C₁-C₆ alkyl; or R²¹ is aminomethyl; or
R²¹ is methylaminomethyl; or R²¹ is dimethylaminomethyl; or R²¹ is C₁-C₆ alkyl optionally substituted with NR¹¹R1, wherein R¹¹ and R¹² taken together with the nitrogen they are attached to form a 3- to 7-membered ring optionally containing one or more heteroatoms in addition to the nitrogen they are attached to; or R²¹ is S(O₂)C₁-C₆ alkyl; or R²¹ is S(O₂)CH₃; or R²⁹ is C₁-C₆ alkyl optionally substituted with NR¹¹R¹², wherein each of R¹¹ and R¹² is independently selected from hydrogen and C₁-C₆ alkyl; or R²⁹ is aminomethyl; or R²⁹ is methylaminomethyl; or R²⁹ is dimethylaminomethyl; or R²⁹ is C₁-C₆ alkyl optionally substituted with NR¹¹R¹², wherein R¹¹ and R¹² taken together with the nitrogen they are attached to form a 3- to 7-membered ring optionally containing one or more heteroatoms in addition to the nitrogen they are attached to; or R²⁹ is S(O₂)C₁-C₆ alkyl; or R²⁹ is S(O₂)CH₃; or R³⁵ is 5-membered nonaromatic monocyclic heterocycloalkyl optionally substituted with C₁-C₆ alkyl; or R³⁵ is 6-membered nonaromatic monocyclic heterocycloalkyl optionally substituted with C₁-C₆ alkyl; or R³⁵ is 7-membered nonaromatic monocyclic heterocycloalkyl optionally substituted with C₁-C₆ alkyl; or R²⁹ is 5-membered nonaromatic monocyclic heterocycloalkyl optionally substituted with C₁-C₆ alkyl; or
R²⁹ is 6-membered nonaromatic monocyclic heterocycloalkyl optionally substituted with C₁-C₆ alkyl; or R²⁹ is 7-membered nonaromatic monocyclic heterocycloalkyl optionally substituted with C₁-C₆ alkyl; or R²¹ is 5-membered nonaromatic monocyclic heterocycloalkyl optionally substituted with C₁-C₆ alkyl; or R²¹ is 6-membered nonaromatic monocyclic heterocycloalkyl optionally substituted with C₁-C₆ alkyl; or R²¹ is 7-membered nonaromatic monocyclic heterocycloalkyl optionally substituted with C₁-C₆ alkyl; or R³⁵ is 1,3-dioxolan-2-yl; or R²¹ is 1,3-dioxolan-2-yl; or R²⁹ is 1,3-dioxolan-2-yl; or R³⁵ is 2-methyl-1,3-dioxolan-2-yl; or R²¹ is 2-methyl-1,3-dioxolan-2-yl; or R²⁹ is 2-methyl-1,3-dioxolan-2-yl; or R³⁵ is S(O₂)C₁-C₆ alkyl; or
R²¹ is S(O₂)C₁-C₆ alkyl; or R²⁹ is S(O₂)C₁-C₆ alkyl; or R³⁵ is S(O₂)CH₃; or R²¹ is S(O₂)CH₃; or
R²⁹ is S(O₂)CH₃; or R²⁹ is C₁-C₆ alkyl; or R³⁵ is C₁-C₆ alkyl; or R²¹ is C₁-C₆ alkyl; or R³⁴ is C₁-C₆ alkyl; or R³⁶ is C₁-C₆ alkyl; or R²⁹ is CH₃; or R³⁵ is CH₃; or R²¹ is CH₃; or R³⁴ is CH₃; or R³⁶ is CH₃; or R²⁹ is halo; or R³⁵ is halo; or R²¹ is halo; or R³⁴ is halo; or R³⁶ is halo.
In some embodiments, provided herein is a compound of Formula III

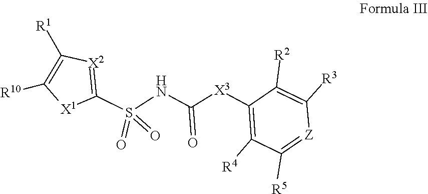

or a pharmaceutically acceptable salt thereof, wherein:

### X¹ is O, S, or NH;

X² is N or CR⁹;
X³ is CH₂;
or X³ and R² taken together with the atoms connecting them form a four-to-seven-membered carbocyclic ring optionally substituted with one or more R16;
or X³ and R⁴ taken together with the atoms connecting them form a four-to-seven-membered carbocyclic ring optionally substituted with one or more R¹⁶;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, or C₁-C₆ alkyl optionally substituted with hydroxy;
R³ is hydrogen, C₁-C₆ alkoxy, halo, C₁-C₆ haloalkyl, or C₁-C₆ alkyl optionally substituted with hydroxy;
R⁴ is hydrogen, C₁-C₆ alkoxy, halo, C₁-C₆ haloalkyl, or C₁-C₆ alkyl optionally substituted with hydroxy;
R⁵ is hydrogen, C₁-C₆ alkoxy, halo, C₁-C₆ haloalkyl, or C₁-C₆ alkyl optionally substituted with hydroxy;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

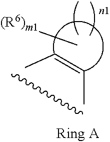

and ring B is

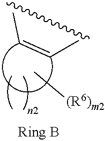

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹¹, oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
R¹ is selected from H, C₁-C₆ alkyl, C₃-C₆ cycloalkyl and C₃-C₆ heterocycloalkyl;
wherein R¹ is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹⁰ is selected from H, C₁-C₆ alkyl, C₃-C₆ cycloalkyl and C₃-C₆ heterocycloalkyl;
wherein R¹⁰ is optionally substituted with one or more substituents each independently selected from hydroxy, oxo, C₁-C₆ alkoxy, NR¹¹R¹², ═NR¹³, COOC₁-C₆ alkyl, and CONR¹¹R¹²; or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R18;
R¹⁵ is C₁-C₆ alkyl;
each of R¹⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³.

In some embodiments, provided herein is a compound of Formula III:

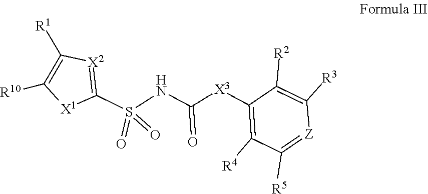

or a pharmaceutically acceptable salt thereof, wherein:

### X¹ is O, S, or NH;

X² is N or CR⁹;
X³ is CH₂;

### Z is N or CR⁸;

R⁸ is selected from H, CN, Cl, F, CO₂C₁-C₆ alkyl and CONH₂;
R⁹ is selected from H and C₁-C₆ alkyl;
R² is hydrogen or C₁-C₆ alkyl optionally substituted with hydroxy;
R³ is hydrogen or C₁-C₆ alkyl optionally substituted with hydroxy;
R⁴ is hydrogen or C₁-C₆ alkyl optionally substituted with hydroxy and is the same as R²;
R⁵ is hydrogen or C₁-C₆ alkyl optionally substituted with hydroxy and is the same as R³;
provided that at least one of R², R³, R⁴ and R⁵ is not hydrogen, and that R² and R⁴ are not both hydroxymethyl;
or R² and R³ taken together with the carbons connecting them form a five-membered ring A and R⁴ and R⁵ taken together with the carbons connecting them form a five-membered ring B,
wherein ring A is

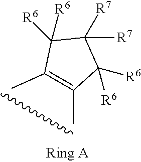

and ring B is

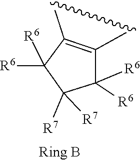

wherein each R⁶ in each ring is the same and is H or C₁-C₆ alkyl, and each R⁷ in each ring is the same and is H or C₁-C₆ alkyl;
R¹ is selected from H, C₁-C₆ alkyl and C₃-C₆ cycloalkyl, wherein R¹ is optionally substituted with one or more substituents each independently selected from hydroxy, amino and oxo;
R¹⁰ is selected from H, C₁-C₆ alkyl and C₃-C₆ cycloalkyl, wherein R¹⁰ is optionally substituted with one or more substituents each independently selected from hydroxy, amino and oxo;
or R¹ and R¹⁰ taken together with the atoms connecting them form a five-membered, a six-membered, or a seven-membered carbocyclic or heterocyclic ring.

In some embodiments, provided herein is a compound of Formula III:

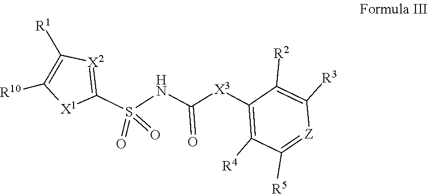

or a pharmaceutically acceptable salt thereof, wherein:

### X¹ is O, S, or NH;

X² is N or CR⁹;
X³ is CH₂;

### Z is N or CR⁸;

R⁸ is selected from H, CN, Cl, F, CO₂C₁-C₆ alkyl and CONH₂;
R⁹ is selected from H and C₁-C₆ alkyl;
R² is hydrogen or C₁-C₆ alkyl optionally substituted with hydroxy;
R³ is hydrogen or C₁-C₆ alkyl optionally substituted with hydroxy;
R⁴ is hydrogen or C₁-C₆ alkyl optionally substituted with hydroxy and is the same as R²;
R⁵ is hydrogen or C₁-C₆ alkyl optionally substituted with hydroxy and is the same as R³;
or R² and R³ taken together with the carbons connecting them form a five-membered ring A and R⁴ and R⁵ taken together with the carbons connecting them form a five-membered ring B,
wherein ring A is

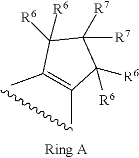

and ring B is

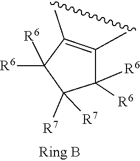

wherein each R⁶ in each ring is the same and is H or C₁-C₆ alkyl, and each R⁷ in each ring is the same and is H or C₁-C₆ alkyl;
R¹ is selected from H, C₁-C₆ alkyl and C₃-C₆ cycloalkyl, wherein R¹ is optionally substituted with one or more substituents each independently selected from hydroxy, amino and oxo;
R¹⁰ is selected from H, C₁-C₆ alkyl and C₃-C₆ cycloalkyl, wherein R¹⁰ is optionally substituted with one or more substituents each independently selected from hydroxy, amino and oxo;
or R¹ and R¹⁰ taken together with the atoms connecting them form a five-membered, a six-membered, or a seven-membered carbocyclic or heterocyclic ring. =.

In some embodiments, the compound of Formula III is a compound of Formula IIIa

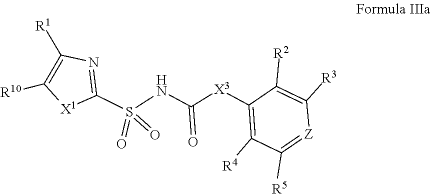

or a pharmaceutically acceptable salt thereof, wherein:

### X¹ is O, S, or NH;

X³ is CH₂;
or X³ and R² taken together with the atoms connecting them form a four-to-seven-membered carbocyclic ring optionally substituted with one or more R¹⁶;
or X³ and R⁴ taken together with the atoms connecting them form a four-to-seven-membered carbocyclic ring optionally substituted with one or more R¹⁶;

### Z is N or CR⁸;

R⁸ is selected from H, CN, Cl, F, CO₂C₁-C₆ alkyl and CONH₂;
R² is hydrogen, C₁-C₆ alkoxy, halo, C₁-C₆ haloalkyl, or C₁-C₆ alkyl optionally substituted with hydroxy;
R³ is hydrogen, C₁-C₆ alkoxy, halo, C₁-C₆ haloalkyl, or C₁-C₆ alkyl optionally substituted with hydroxy;
R⁴ is hydrogen, C₁-C₆ alkoxy, halo, C₁-C₆ haloalkyl, or C₁-C₆ alkyl optionally substituted with hydroxy;
R⁵ is hydrogen, C₁-C₆ alkoxy, halo, C₁-C₆ haloalkyl, or C₁-C₆ alkyl optionally substituted with hydroxy;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

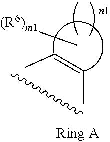

and ring B is

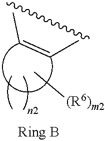

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
R¹ is selected from H, C₁-C₆ alkyl and C₃-C₆ cycloalkyl, wherein R¹ is optionally substituted with hydroxy, amino or oxo;
R¹⁰ is selected from H, C₁-C₆ alkyl and C₃-C₆ cycloalkyl, wherein R¹⁰ is optionally substituted with hydroxy, amino or oxo;
wherein R¹⁰ is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R18;
R¹⁵ is C₁-C₆ alkyl;
each of R¹⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³.

In some embodiments, the compound of Formula III is a compound of Formula IIIa

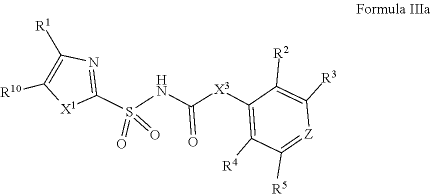

or a pharmaceutically acceptable salt thereof,

wherein

### X¹ is O, S, or NH;

X³ is CH₂;
or X³ and R² taken together with the atoms connecting them form a four-to-seven-membered carbocyclic ring optionally substituted with one or more R¹⁶;
or X³ and R⁴ taken together with the atoms connecting them form a four-to-seven-membered carbocyclic ring optionally substituted with one or more R¹⁶;

### Z is N or CR⁸;

R⁸ is selected from H, CN, Cl, F, CO₂C₁-C₆ alkyl and CONH₂;
R² is hydrogen or C₁-C₆ alkyl optionally substituted with hydroxy;
R³ is hydrogen or C₁-C₆ alkyl optionally substituted with hydroxy;
R⁴ is hydrogen or C₁-C₆ alkyl optionally substituted with hydroxy and is the same as R²;
R⁵ is hydrogen or C₁-C₆ alkyl optionally substituted with hydroxy and is the same as R³;
provided that at least one of R², R³, R⁴ and R⁵ is not hydrogen, and that R² and R⁴ are not both hydroxymethyl;
or R² and R³ taken together with the carbons connecting them form a five-membered ring A and R⁴ and R⁵ taken together with the carbons connecting them form a five-membered ring B,
wherein ring A is

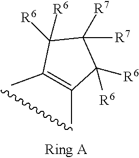

and ring B is

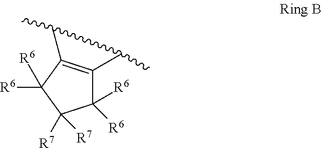

wherein each R⁶ in each ring is the same and is H or C₁-C₆ alkyl, and each R⁷ in each ring is the same and is H or C₁-C₆ alkyl;
R¹ is selected from H, C₁-C₆ alkyl and C₃-C₆ cycloalkyl, wherein R¹ is optionally substituted with one or more substituents each independently selected from hydroxy, amino and oxo;
R¹⁰ is selected from H, C₁-C₆ alkyl and C₃-C₆ cycloalkyl, wherein R¹⁰ is optionally substituted with one or more substituents each independently selected from hydroxy, amino and oxo;
or R¹ and R¹⁰ taken together with the atoms connecting them form a five-membered, a six-membered, or a seven-membered carbocyclic or heterocyclic ring.

In some embodiments, the compound of Formula IIIa is a compound of Formula IIIa-i:

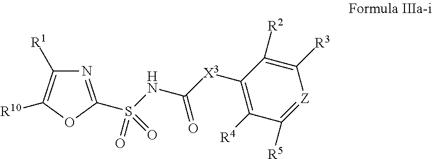

or a pharmaceutically acceptable salt thereof,

wherein:
X³ is NB, 0 or CH₂;

### Z is N or CR⁸;

R⁸ is selected from H, CN, Cl, F, CO₂C₁-C₆ alkyl and CONH₂;
R² is C₁-C₆ alkoxy, halo, C₁-C₆ haloalkyl, or C₁-C₆ alkyl optionally substituted with hydroxy;
R³ is hydrogen;
R⁴ is C₁-C₆ alkoxy, halo, C₁-C₆ haloalkyl, or C₁-C₆ alkyl optionally substituted with hydroxy;
R⁵ is hydrogen;
R¹ is selected from H, C₁-C₆ alkyl and C₃-C₆ cycloalkyl, wherein R¹ is optionally substituted with hydroxy, amino or oxo;
R¹⁰ is selected from H, Ci-C alkyl and C₃-C₆ cycloalkyl, wherein R¹⁰ is optionally substituted with hydroxy, amino or oxo;
or R¹ and R¹⁰ taken together with the atoms connecting them form a five-membered, a six-membered, or a seven-membered carbocyclic or heterocyclic ring.

In some embodiments, the compound of Formula IIIa is a compound of Formula IIIa-i:

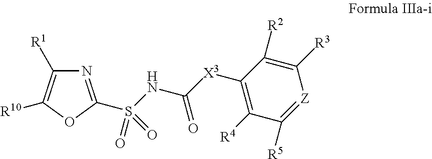

or a pharmaceutically acceptable salt thereof,

wherein:
X³ is NH, 0 or CH₂;

### Z is N or CR⁸;

R⁸ is selected from H, CN, Cl, F, CO₂C₁-C₆ alkyl and CONH₂;
R³ is C₁-C₆ alkoxy, halo, C₁-C₆ haloalkyl, or C₁-C₆ alkyl optionally substituted with hydroxy;
R² is hydrogen;
R⁵ is C₁-C₆ alkoxy, halo, C₁-C₆ haloalkyl, or C₁-C₆ alkyl optionally substituted with hydroxy;
R⁴ is hydrogen;
R¹ is selected from H, C₁-C₆ alkyl and C₃-C₆ cycloalkyl, wherein R¹ is optionally substituted with hydroxy, amino or oxo;
R¹⁰ is selected from H, C₁-C₆ alkyl and C₃-C₆ cycloalkyl, wherein R¹⁰ is optionally substituted with hydroxy, amino or oxo;
or R¹ and R¹⁰ taken together with the atoms connecting them form a five-membered, a six-membered, or a seven-membered carbocyclic or heterocyclic ring.
In some embodiments, the compound of Formula IIIa is a compound of Formula IIIa-i:

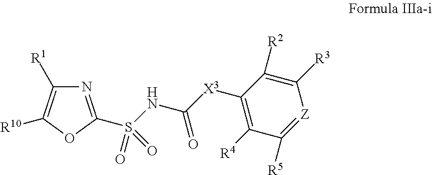

or a pharmaceutically acceptable salt thereof,

wherein:
X³ is NH, 0 or CH₂;

### Z is N or CR⁸;

R⁸ is selected from H, CN, Cl, F, CO₂C₁-C₆ alkyl and CONH₂;
R² and R³ taken together with the carbons connecting them form a four-membered to seven-membered ring A and R⁴ and R⁵ taken together with the carbons connecting them form a four-membered to seven-membered ring B,
wherein ring A is

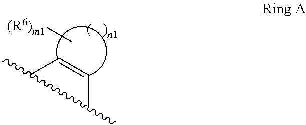

and ring B is

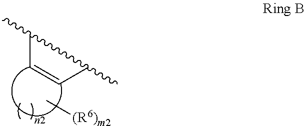

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹¹, oxo, and ═NR³;
or two R⁶ taken together with the atom or atoms connecting them form a 3-to-8-membered carbocyclic or saturated heterocyclic ring containing 1 or 2 heteroatoms independently selected from O, N, and S;
R¹ is selected from H, C₁-C₆ alkyl and C₃-C₆ cycloalkyl, wherein R¹ is optionally substituted with hydroxy, amino or oxo;
R¹⁰ is selected from H, C₁-C₆ alkyl and C₃-C₆ cycloalkyl, wherein R¹⁰ is optionally substituted with hydroxy, amino or oxo;
or R¹ and R¹⁰ taken together with the atoms connecting them form a five-membered, a six-membered, or a seven-membered carbocyclic or heterocyclic ring.

In some embodiments, the compound of Formula III is a compound of Formula IIIa-ii

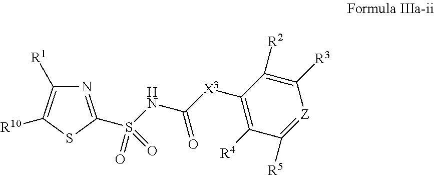

or a pharmaceutically acceptable salt thereof.

In some embodiments, the compound of Formula III is a compound of Formula IIIa-iii

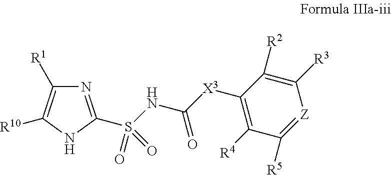

or a pharmaceutically acceptable salt thereof.

In some embodiments of the compound of Formulae IIIa, IIa-i, oIa-ii, IIIa-iii, and IiIb, R¹ is C₁-C₆ alkyl or C₃-C₆ cycloalkyl, wherein R¹ is optionally substituted with one or more substituents each independently selected from hydroxy, amino and oxo. In some embodiments of the compound of Formulae IIIa, IIIa-i, IIIa-i, IIIa-iii, and IIIb, R¹ is C₁-C₆ alkyl optionally substituted with one or more hydroxy. In some embodiments of the compound of Formulae IIIa, IIIa-i, IIIa-ii, IIIa-iii, and IIIb, R¹ is C₁-C₆ alkyl substituted with hydroxy. In some embodiments, the hydroxy is at the carbon of R¹ directly bonded to the five-membered heteroaryl ring in Formulae IIIa, IIIa-i, IIIa-ii, IIIa-iii, and IIIb. In some embodiments of the compound of Formulae IIIa, IIIa-i, IIIa-ii, IIIa-iii, and IIIb, R¹ is 2-hydroxy-2-propyl.

In some embodiments of the compound of Formulae IIIa, IIIa-i, IIIa-ii, IlIa-iii, and IIc, R¹⁰ is C₁-C₆ alkyl or C₃-C₆ cycloalkyl, wherein R¹⁰ is optionally substituted with one or more substituents each independently selected from hydroxy, amino and oxo. In some embodiments of the compound of Formulae IIIa, IIIa-i, IIIa-ii, IIIa-iii, and IIIc, R¹⁰ is C₁-C₆ alkyl optionally substituted with one or more hydroxy. In some embodiments of the compound of Formulae IIIa, IIIa-i, IIIa-ii, IIIa-iii, and IIIc, R¹⁰ is C₁-C₆ alkyl substituted with hydroxy. In some embodiments, the hydroxy is at the carbon of R¹⁰ directly bonded to the five-membered heteroaryl ring in Formulae IIIa, IIIa-i, IIIa-ii, IIIa-iii, and IIIc. In some embodiments of the compound of Formulae IIIa, IIIa-i, IIIa-ii, IIIa-iii, and IIIc, R¹⁰ is 2-hydroxy-2-propyl.

In some embodiments of the compound of Formulae IIIa, IIIa-i, IIIa-ii, and IIIa-iii, R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²; In some embodiments of the compound of Formulae IIIa, IIIa-i, IIIa-ii, and IIIa-iii, R¹ and R¹⁰ taken together with the atoms connecting them form a five-membered carbocyclic ring.

In some embodiments of the compound of Formulae IIIa, IIIa-i, IIIa-ii, and IIIa-iii, R¹ and R¹⁰ taken together with the atoms connecting them form a six-membered carbocyclic ring. In some embodiments of the compound of Formulae IIIa, IIIa-i, IIIa-ii, and IIIa-iii, R¹ and R¹⁰ taken together with the atoms connecting them form a five-membered heterocyclic ring containing 1 or 2 heteroatoms independently selected from O, N, and S. In some embodiments of the compound of Formulae IIIa, IIIa-i, IIIa-ii, and IIIa-iii, R¹ and R¹⁰ taken together with the atoms connecting them form a five-membered heterocyclic ring containing 1 or 2 heteroatoms independently selected from O, N, and S.

In some embodiments of the compound of Formulae IIIa, IIIa-i, IIIa-ii, and IIIa-iii, ring A is a carbocyclic ring and n1 is 3; or ring A is a carbocyclic ring and n1 is 4; or ring A is a heterocyclic ring containing 1 or 2 heteroatoms independently selected from O, N, and S and n1 is 3; or ring A is a heterocyclic ring containing 1 or 2 heteroatoms independently selected from O, N, and S and n1 is 4; or ring B is a carbocyclic ring and n2 is 3; or ring B is a carbocyclic ring and n2 is 4; or ring B is a heterocyclic ring containing 1 or 2 heteroatoms independently selected from O, N, and S and n2 is 3; or ring B is a heterocyclic ring containing 1 or 2 heteroatoms independently selected from O, N, and S and n2 is 4.

In some embodiments, the compound of Formula III is a compound of Formula IIIb

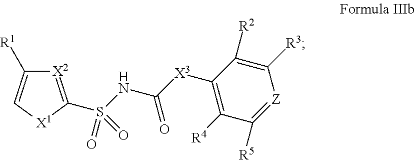

or a compound of Formula IIIc

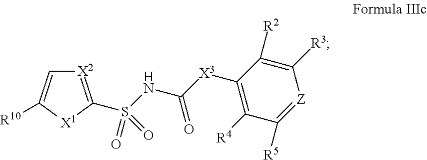

or

or a pharmaceutically acceptable salt thereof, respectively.

In some embodiments, provided herein is a compound of Formula IV

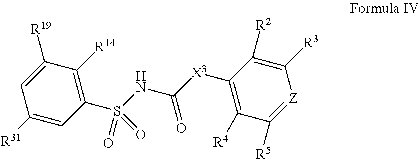

or a pharmaceutically acceptable salt thereof, wherein:
X³ is CH₂;
or X³ and R² taken together with the atoms connecting them form a four-to-seven-membered carbocyclic ring optionally substituted with one or more R¹⁶;
or X³ and R⁴ taken together with the atoms connecting them form a four-to-seven-membered carbocyclic ring optionally substituted with one or more R¹⁶;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, or C₁-C₆ alkyl optionally substituted with hydroxy;
R³ is hydrogen, C₁-C₆ alkoxy, halo, C₁-C₆ haloalkyl, or C₁-C₆ alkyl optionally substituted with hydroxy;
R⁴ is hydrogen, C₁-C₆ alkoxy, halo, C₁-C₆ haloalkyl, or C₁-C₆ alkyl optionally substituted with hydroxy;
R⁵ is hydrogen, C₁-C₆ alkoxy, halo, C₁-C₆ haloalkyl, or C₁-C₆ alkyl optionally substituted with hydroxy;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

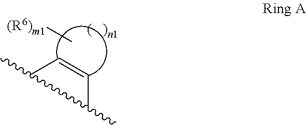

and ring B is

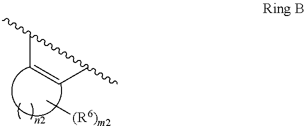

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
R³¹ is selected from H, CN, Cl, or F;
R¹⁴ is selected from H, CN, Cl, or F;
R¹⁹ is selected from C₁-C₆ alkyl, C(R²⁰)₂OH, C(R²⁰)₂NR¹¹R¹², C₃-C₆ cycloalkyl and C₃-C₆ heterocycloalkyl;
wherein, when R¹⁹ is C₁-C₆ alkyl, C₃-C₆ cycloalkyl or C₃-C₆ heterocycloalkyl, R¹⁹ is optionally substituted with one or more substituents each independently selected from ═NR¹³, COOC₁-C₆ alkyl, and CONR¹¹R¹²;
each R²⁰ is the same and is H or C₁-C₆ alkyl;
or two R²⁰ taken together with the carbon connecting them form a three-to-eight-membered heterocyclic ring containing 1 or 2 heteroatoms independently selected from O, N, and S, or a three-membered, six-membered, seven-membered, or eight-membered carbocyclic ring, wherein the heterocyclic ring or carbocyclic ring is optionally substituted with one or more substituents each independently selected from H, C₁-C₆ alkyl, C₁-C₆ alkoxy, NR¹¹R¹², oxo, and ═NR¹³;
R¹³ is C₁-C₆ alkyl;
each of R¹¹ and R¹² at each occurrence is independently selected from hydrogen, C₁-C₆ alkyl, CO₂R¹⁵ and CONR¹⁷R8;
R¹⁵ is C₁-C₆ alkyl;
each of R⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³.

In some embodiments, provided herein is a compound of Formula IV

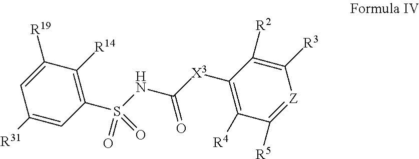

or a pharmaceutically acceptable salt thereof, wherein:
X³ is CH₂;
or X³ and R² taken together with the atoms connecting them form a four-to-seven-membered carbocyclic ring optionally substituted with one or more R16;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, or C₁-C₆ alkyl optionally substituted with hydroxy;
R³ is hydrogen, C₁-C₆ alkoxy, halo, C₁-C₆ haloalkyl, or C₁-C₆ alkyl optionally substituted with hydroxy;
R⁴ is hydrogen, C₁-C₆ alkoxy, halo, C₁-C₆ haloalkyl, or C₁-C₆ alkyl optionally substituted with hydroxy;
R⁵ is hydrogen, C₁-C₆ alkoxy, halo, C₁-C₆ haloalkyl, or C₁-C₆ alkyl optionally substituted with hydroxy;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

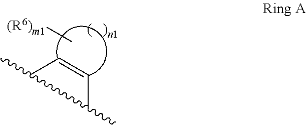

and ring B is

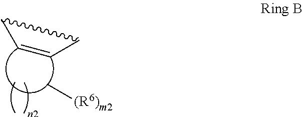

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹¹, oxo, and ═NR³;
or two R⁶ taken together with the atom or atoms connecting them form a 3-to-8-membered carbocyclic or saturated heterocyclic ring containing 1 or 2 heteroatoms independently selected from O, N, and S;
R³¹ is selected from H, CN, Cl, or F;
R¹⁴ is selected from H, CN, Cl, or F;
R¹⁹ is selected from C₁-C₆ alkyl, C(R²⁰)₂OH, C(R²⁰)₂NR¹¹R¹², C₃-C₆ cycloalkyl and C₃-C₆ heterocycloalkyl;
wherein, when R¹⁹ is C₁-C₆ alkyl, C₃-C₆ cycloalkyl or C₃-C₆ heterocycloalkyl, R¹⁹ is optionally substituted with one or more substituents each independently selected from ═NR¹³, COOC₁-C₆ alkyl, and CONR¹¹R¹²;
each R²⁰ is the same and is H or C₁-C₆ alkyl;
R¹³ is C₁-C₆ alkyl;
each of R¹¹ and R¹² at each occurrence is independently selected from hydrogen, C₁-C₆ alkyl, CO₂R¹⁵ and CONR¹⁷R18;
R¹⁵ is C₁-C₆ alkyl;
each of R¹⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³.

In some embodiments, provided herein is a compound of Formula IV

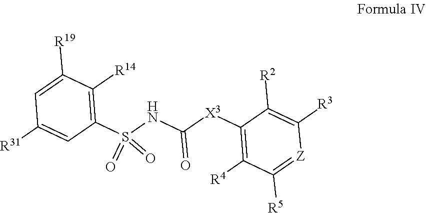

or a pharmaceutically acceptable salt thereof, wherein:
X³ is CH₂;

### Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;
R² is hydrogen, C₁-C₆ alkoxy, halo, C₁-C₆ haloalkyl, or C₁-C₆ alkyl optionally substituted with hydroxy;
R³ is hydrogen, C₁-C₆ alkoxy, halo, C₁-C₆ haloalkyl, or C₁-C₆ alkyl optionally substituted with hydroxy;
R⁴ is hydrogen, C₁-C₆ alkoxy, halo, C₁-C₆ haloalkyl, or C₁-C₆ alkyl optionally substituted with hydroxy;
R⁵ is hydrogen, C₁-C₆ alkoxy, halo, C₁-C₆ haloalkyl, or C₁-C₆ alkyl optionally substituted with hydroxy;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is

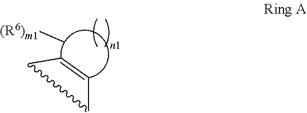

and ring B is

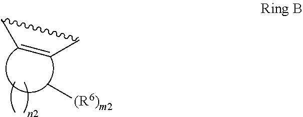

wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹¹, oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
R³¹ is selected from H, CN, Cl, or F;
R¹⁴ is selected from H, CN, Cl, or F;
R¹⁹ is selected from C₁-C₆ alkyl, C(R²⁰)₂OH, C(R²⁰)₂NR¹¹R¹², C₃-C₆ cycloalkyl and C₃-C₆ heterocycloalkyl;
wherein, when R¹⁹ is C₁-C₆ alkyl, C₃-C₆ cycloalkyl or C₃-C₆ heterocycloalkyl, R¹⁹ is optionally substituted with one or more substituents each independently selected from ═NR¹³, COOC₁-C₆ alkyl, and CONR¹¹R¹²;
each R²⁰ is the same and is H or C₁-C₆ alkyl;
each of R¹¹, R¹² and R¹³ at each occurrence is independently selected from hydrogen and C₁-C₆ alkyl.

In some embodiments, provided herein is a compound of Formula IVa

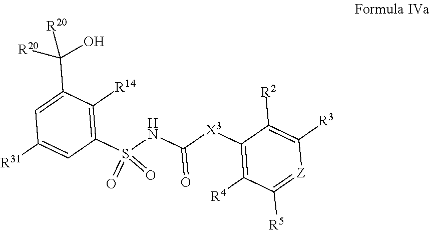

or a pharmaceutically acceptable salt thereof, wherein:
X³ is CH₂;

### Z is N or CR⁸;

R⁸ is selected from H, CN, Cl, F, CO₂C₁-C₆ alkyl and CONH₂;
R² is hydrogen or C₁-C₆ alkyl optionally substituted with hydroxy;
R³ is hydrogen or C₁-C₆ alkyl optionally substituted with hydroxy;
R⁴ is hydrogen or C₁-C₆ alkyl optionally substituted with hydroxy and is the same as R²;
R⁵ is hydrogen or C₁-C₆ alkyl optionally substituted with hydroxy and is the same as R³;
provided that at least one of R², R³, R⁴ and R⁵ is not hydrogen, and that R² and R⁴ are not both hydroxymethyl;
or R² and R³ taken together with the carbons connecting them form a five-membered ring A and R⁴ and R⁵ taken together with the carbons connecting them form a five-membered ring B,
wherein ring A is

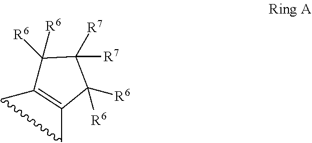

and ring B is

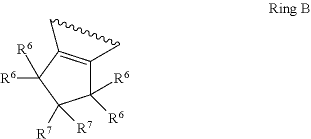

wherein each R⁶ in each ring is the same and is H or C₁-C₆ alkyl, and each R⁷ in each ring is the same and is H or C₁-C₆ alkyl;
R³¹ is selected from H, CN, Cl, or F;
R¹⁴ is selected from H, CN, Cl, or F;
each R²⁰ is the same and is selected from H and C₁-C₆ alkyl.

In some embodiments, provided herein is a compound of Formula IVa

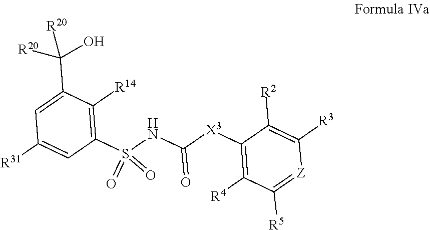

or a pharmaceutically acceptable salt thereof, wherein:
X³ is CH₂;

### Z is N or CR⁸;

R⁸ is selected from H, CN, Cl, F, CO₂C₁-C₆ alkyl and CONH₂;
R² is hydrogen or C₁-C₆ alkyl optionally substituted with hydroxy;
R³ is hydrogen or C₁-C₆ alkyl optionally substituted with hydroxy;
R⁴ is hydrogen or C₁-C₆ alkyl optionally substituted with hydroxy and is the same as R²;
R⁵ is hydrogen or C₁-C₆ alkyl optionally substituted with hydroxy and is the same as R³;
provided that at least one of R², R³, R⁴ and R⁵ is not hydrogen, and that R² and R⁴ are not both hydroxymethyl;
or R² and R³ taken together with the carbons connecting them form a five-membered ring A and R⁴ and R⁵ taken together with the carbons connecting them form a five-membered ring B,
wherein ring A is

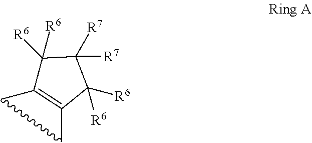

and ring B is

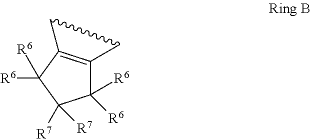

wherein each R⁶ in each ring is the same and is H or C₁-C₆ alkyl, and each R⁷ in each ring is the same and is H or C₁-C₆ alkyl;
R¹ is selected from H, CN, Cl, or F;
R¹⁴ is selected from H, CN, Cl, or F;
each R²⁰ is the same and is selected from H and C₁-C₆ alkyl.

### The Group X³

In some embodiments of one or more formulae herein, X³ is CH₂; or X³ and R² taken together with the atoms connecting them form a four-to-seven-membered carbocyclic ring optionally substituted with one or more R¹⁶; or X³ and R⁴ taken together with the atoms connecting them form a four-to-seven-membered carbocyclic ring optionally substituted with one or more R¹⁶; or X³ and R² taken together with the atoms connecting them form a four-to-seven-membered ring C of the formula

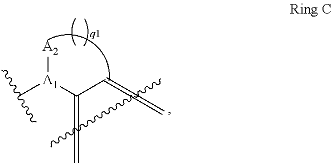

wherein q1 is 0, 1, 2 or 3; A1 is CH; A2 is CH₂; and ring C is optionally substituted with 1 to 8 R¹⁶.
In some embodiments of ring C, A1 is CH and the CH has (R) stereochemistry; or A1 is CH and the CH has (S) stereochemistry; or R¹⁶ is H.

### The Group R16

In some embodiments of one or more formulae herein, R¹⁶ is hydrogen.; or R¹⁶ is C₁-C₆ alkyl; or
In some embodiments of one or more formulae herein, R¹⁶ is C₁-C₆ alkoxy; or R16 is NR¹¹R¹²; or R¹⁶ is oxo; or R¹⁶ is C₁═NR¹³.

### Non-Limiting Combinations

In some embodiments of one or more formulae herein, R² is C₁-C₆ alkyl (e.g., isopropyl), halo (e.g., chloro), or C₃-C₇ cycloalkyl (e.g., cyclopropyl); R³ is hydrogen, halo (e.g., fluoro), or C₁-C₆ alkyl (e.g., isopropyl or methyl); R⁸ is hydrogen, halo (e.g., chloro or fluoro), CN, or C₁-C₆ haloalkyl (e.g., difluoromethyl); R⁵ is hydrogen or halo (e.g., fluoro); R⁴ is halo (e.g., chloro), C₁-C₆ alkyl (e.g., isopropyl), or C₃-C₇ cycloalkyl (e.g., cyclopropyl);

or R² and R³ taken together with the carbons connecting them form a five-membered ring A,

or R⁴ and R⁵ taken together with the carbons connecting them form a five-membered ring B,

or R² and R³ taken together with the carbons connecting them form a five-membered ring A; and R⁴ and R⁵ taken together with the carbons connecting them form a five-membered ring B,

wherein ring A is

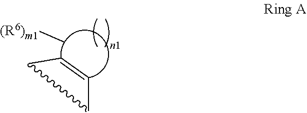

and ring B is

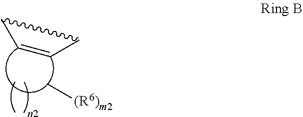

wherein
ring A is a carbocyclic ring;
n1 is 3;
m1 is 6;
wherein ring B is a carbocyclic ring;
n2 is 3;
m2 is 6;
wherein each R⁶ in each ring is the same or different and is selected from H or C₁-C₆ alkyl (e.g., methyl).

In some embodiments of one or more formulae herein, Y is CR², X⁴ is CR⁴, and Z is N.

In some embodiments of one or more formulae herein, R² is C₁-C₆ alkyl (e.g., isopropyl) or halo (e.g., chloro); R³ is hydrogen or C₁-C₆ alkyl (e.g., isopropyl); R⁵ is hydrogen or halo (e.g., fluoro); R⁴ is C₁-C₆ alkyl (e.g., isopropyl);

or R² and R³ taken together with the carbons connecting them form a five-membered ring A,

or R⁴ and R⁵ taken together with the carbons connecting them form a five-membered ring B,

or R² and R³ taken together with the carbons connecting them form a five-membered ring A; and R⁴ and R⁵ taken together with the carbons connecting them form a five-membered ring B,

wherein ring A is

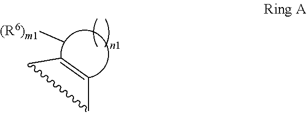

and ring B is

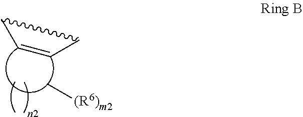

wherein
ring A is a carbocyclic ring;
n1 is 3;
m1 is 6;
wherein ring B is a carbocyclic ring;
n2 is 3;
m2 is 6;
wherein each R⁶ in each ring is the same or different and is selected from H or C₁-C₆ alkyl (e.g., methyl).

### Additional Features of the Embodiments Herein

In some embodiments of one or more formulae herein, the compound is not a compound selected from the group consisting of those mentioned under Proviso (P1) above.

In some embodiments of one or more formulae herein, the compound is not a compound selected from the group consisting of the compounds disclosed in Table 1A and Table 1B.

In some embodiments of one or more formulae herein, the compound is not a compound disclosed in Smith, W. E. et. al. J. Med. Chem. 2016, 59(8), 4342-4351.; or is not a compound disclosed in Ammazzalorso, A. et. al. Synth. Commun. 2015, 2546-2554; or is not a compound disclosed in Shen, S. et. al. Org. Biomol. Chem. 2015, 13(40), 10205-10211; or is not a compound disclosed in Yavari, I. et. al. Synlett. 2014, 25(7), 959-960; or is not a compound disclosed in CN 103159674; or is not a compound disclosed in Luo, Y. et. al. Bioorg. Med. Chem. Lett. 2011, 19(20), 6069-6076; or is not a compound disclosed in Raushel, J. et. al. Org. Lett. 2010, 14(23), 6012-6015; or is not a compound disclosed in Smith, W. E. et. al. J. Med. Chem. 2016, 59(8), 4342-4351; Ammazzalorso, A. et. al. Synth. Commun. 2015, 2546-2554; Shen, S. et. al. Org. Biomol. Chem. 2015, 13(40), 10205-10211; Yavari, I. et. al. Synlett. 2014, 25(7), 959-960; CN 103159674; Luo, Y. et. al. Bioorg. Med. Chem. Lett. 2011, 19(20), 6069-6076; and Raushel, J. et. al. Org. Lett. 2010, 14(23), 6012-6015; or is not a compound from two or more or all disclosures mentioned in the present paragraph.

In one embodiment, provided herein is a combination of a compound of any preceding embodiment, for use in the treatment or the prevention of a condition mediated by TNF-α, in a patient in need thereof, wherein the compound is administered to said patient at a therapeutically effective amount. Preferably, the subject is resistant to treatment with an anti-TNFα agent. Preferably, the condition is a gut disease or disorder.

In one embodiment, provided herein is a pharmaceutical composition of comprising a compound of any preceding embodiment, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any preceding embodiment, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates ton NLRP3 antagonist for use according to any of the above embodiments, wherein the gut disease is IBD.

In one embodiment, the present invention relates to an NLRP3 antagonist for use according to any of the above embodiments, wherein the gut disease is Ulcerative Colitis (UC) or Crohn's Disease (CD).

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist.

In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method according to any of the above embodiments x to xx, wherein the gut disease is UC or CD.

Unless otherwise indicated, when a disclosed compound is named or depicted by a structure without specifying the stereochemistry and has one or more chiral centers, it is understood to represent all possible stereoisomers of the compound.

It is understood that the combination of variables in the formulae herein is such that the compounds are stable.

In some embodiments, provided herein is a compound selected from the group consisting of the compounds in Table 1A below:

TABLE 1A

| Com- | |
|---|---|
| pound | Structure |
| 101 | 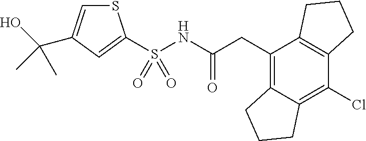 [SMILES: CC(C)(O)c1csc(S(=O)(=O)NC(=O)Cc2c3c(c(Cl)c4c2CCC4)CCC3)c1] |
| 102 | 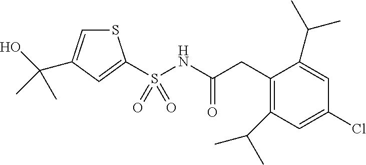 [SMILES: CC(C)c1cc(Cl)cc(C(C)C)c1CC(=O)NS(=O)(=O)c1cc(C(C)(C)O)cs1] |
| 103 | 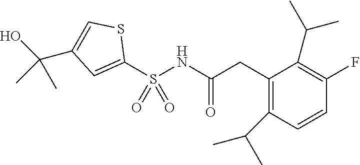 [SMILES: CC(C)c1ccc(F)c(C(C)C)c1CC(=O)NS(=O)(=O)c1cc(C(C)(C)O)cs1] |
| 104 | 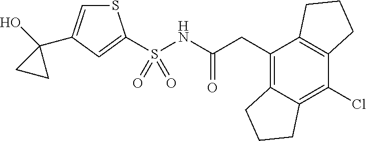 [SMILES: O=C(Cc1c2c(c(Cl)c3c1CCC3)CCC2)NS(=O)(=O)c1cc(C2(O)CC2)cs1] |
| 105 | 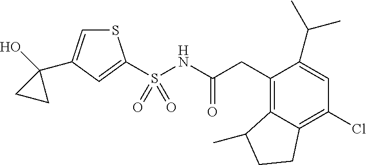 [SMILES: CC(C)c1cc(Cl)c2c(c1CC(=O)NS(=O)(=O)c1cc(C3(O)CC3)cs1)C(C)CC2] |
| 106 | 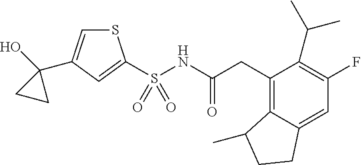 [SMILES: CC(C)c1c(F)cc2c(c1CC(=O)NS(=O)(=O)c1cc(C3(O)CC3)cs1)C(C)CC2] |
| 107 | 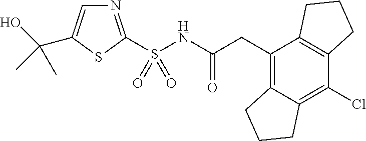 [SMILES: CC(C)(O)c1cnc(S(=O)(=O)NC(=O)Cc2c3c(c(Cl)c4c2CCC4)CCC3)s1] |
| 108 | 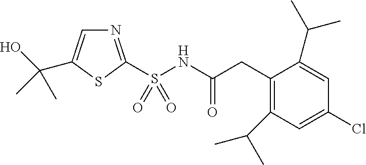 [SMILES: CC(C)c1cc(Cl)cc(C(C)C)c1CC(=O)NS(=O)(=O)c1ncc(C(C)(C)O)s1] |
| 109 | 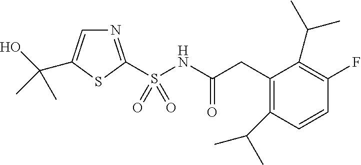 [SMILES: CC(C)c1ccc(F)c(C(C)C)c1CC(=O)NS(=O)(=O)c1ncc(C(C)(C)O)s1] |
| 110 | 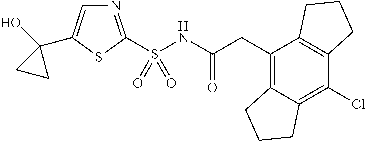 [SMILES: O=C(Cc1c2c(c(Cl)c3c1CCC3)CCC2)NS(=O)(=O)c1ncc(C2(O)CC2)s1] |
| 111 | 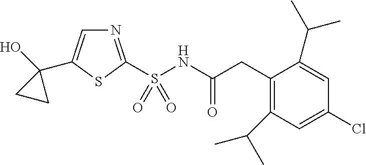 [SMILES: CC(C)c1cc(Cl)cc(C(C)C)c1CC(=O)NS(=O)(=O)c1ncc(C2(O)CC2)s1] |
| 112 | 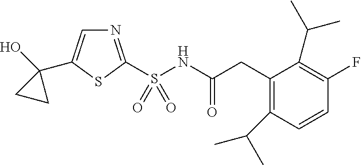 [SMILES: CC(C)c1ccc(F)c(C(C)C)c1CC(=O)NS(=O)(=O)c1ncc(C2(O)CC2)s1] |
| 113 | 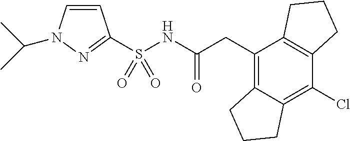 [SMILES: CC(C)n1ccc(S(=O)(=O)NC(=O)Cc2c3c(c(Cl)c4c2CCC4)CCC3)n1] |
| 114 | 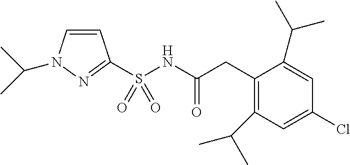 [SMILES: CC(C)c1cc(Cl)cc(C(C)C)c1CC(=O)NS(=O)(=O)c1ccn(C(C)C)n1] |
| 115 | 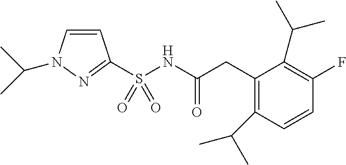 [SMILES: CC(C)c1ccc(F)c(C(C)C)c1CC(=O)NS(=O)(=O)c1ccn(C(C)C)n1] |
| 116 | 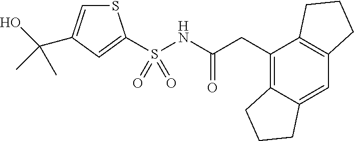 [SMILES: CC(C)(O)c1csc(S(=O)(=O)NC(=O)Cc2c3c(cc4c2CCC4)CCC3)c1] |
| 117 | 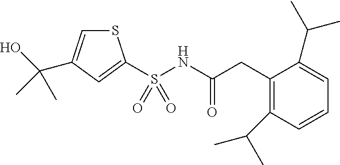 [SMILES: CC(C)c1cccc(C(C)C)c1CC(=O)NS(=O)(=O)c1cc(C(C)(C)O)cs1] |
| 118 | 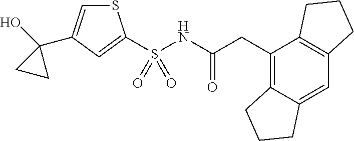 [SMILES: O=C(Cc1c2c(cc3c1CCC3)CCC2)NS(=O)(=O)c1cc(C2(O)CC2)cs1] |
| 119 | 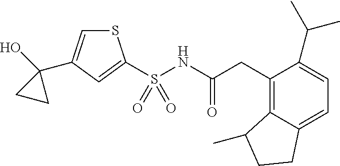 [SMILES: CC(C)c1ccc2c(c1CC(=O)NS(=O)(=O)c1cc(C3(O)CC3)cs1)C(C)CC2] |
| 120 | 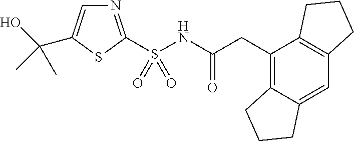 [SMILES: CC(C)(O)c1cnc(S(=O)(=O)NC(=O)Cc2c3c(cc4c2CCC4)CCC3)s1] |
| 121 | 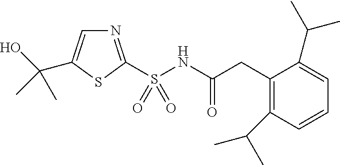 [SMILES: CC(C)c1cccc(C(C)C)c1CC(=O)NS(=O)(=O)c1ncc(C(C)(C)O)s1] |
| 122 | 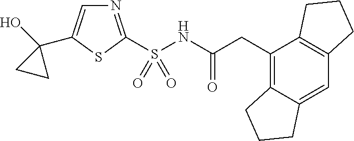 [SMILES: O=C(Cc1c2c(cc3c1CCC3)CCC2)NS(=O)(=O)c1ncc(C2(O)CC2)s1] |
| 123 | 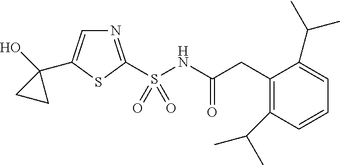 [SMILES: CC(C)c1cccc(C(C)C)c1CC(=O)NS(=O)(=O)c1ncc(C2(O)CC2)s1] |
| 124 | 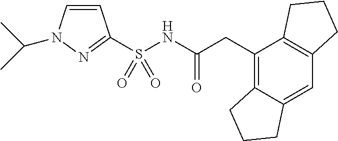 [SMILES: CC(C)n1ccc(S(=O)(=O)NC(=O)Cc2c3c(cc4c2CCC4)CCC3)n1] |
| 125 | 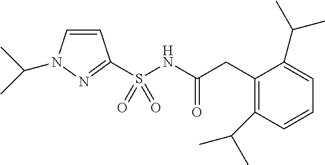 [SMILES: CC(C)c1cccc(C(C)C)c1CC(=O)NS(=O)(=O)c1ccn(C(C)C)n1] |
| 126 | 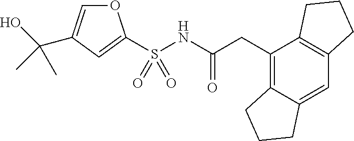 [SMILES: CC(C)(O)c1coc(S(=O)(=O)NC(=O)Cc2c3c(cc4c2CCC4)CCC3)c1] |

and pharmaceutically acceptable salts thereof.

In some embodiments, provided herein is a compound selected from the group consisting of the compounds in Table 1B below:

TABLE 1B

| | |
|---|---|
| 140 | 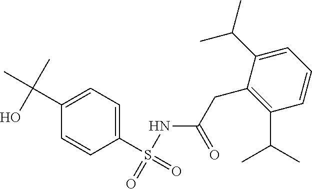 [SMILES: CC(C)c1cccc(C(C)C)c1CC(=O)NS(=O)(=O)c1ccc(C(C)(C)O)cc1] |
| 141 | 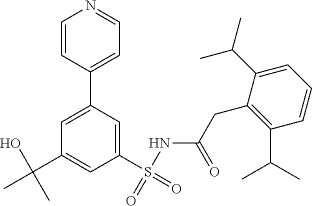 [SMILES: CC(C)c1cccc(C(C)C)c1CC(=O)NS(=O)(=O)c1cc(-c2ccncc2)cc(C(C)(C)O)c1] |
| 142 | 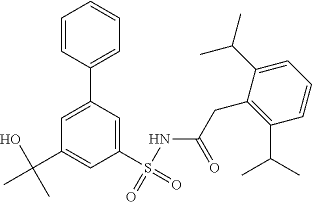 [SMILES: CC(C)c1cccc(C(C)C)c1CC(=O)NS(=O)(=O)c1cc(-c2ccccc2)cc(C(C)(C)O)c1] |
| 143 | 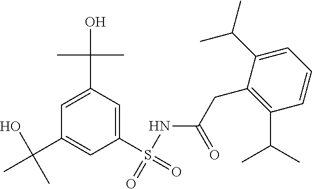 [SMILES: CC(C)c1cccc(C(C)C)c1CC(=O)NS(=O)(=O)c1cc(C(C)(C)O)cc(C(C)(C)O)c1] |
| 144 | 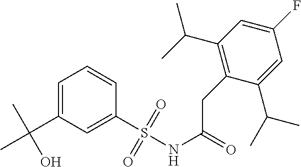 [SMILES: CC(C)c1cc(F)cc(C(C)C)c1CC(=O)NS(=O)(=O)c1cccc(C(C)(C)O)c1] |
| 145 | 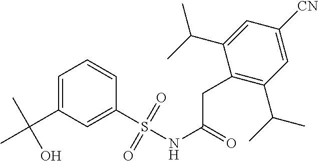 [SMILES: CC(C)c1cc(C#N)cc(C(C)C)c1CC(=O)NS(=O)(=O)c1cccc(C(C)(C)O)c1] |
| 146 | 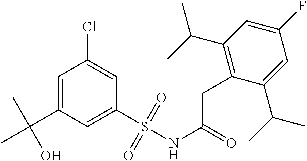 [SMILES: CC(C)c1cc(F)cc(C(C)C)c1CC(=O)NS(=O)(=O)c1cc(Cl)cc(C(C)(C)O)c1] |
| 147 | 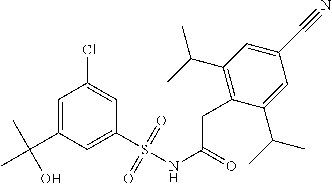 [SMILES: CC(C)c1cc(C#N)cc(C(C)C)c1CC(=O)NS(=O)(=O)c1cc(Cl)cc(C(C)(C)O)c1] |
| 148 | 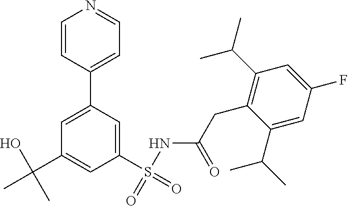 [SMILES: CC(C)c1cc(F)cc(C(C)C)c1CC(=O)NS(=O)(=O)c1cc(-c2ccncc2)cc(C(C)(C)O)c1] |
| 149 | 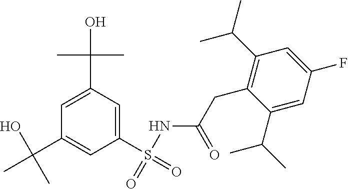 [SMILES: CC(C)c1cc(F)cc(C(C)C)c1CC(=O)NS(=O)(=O)c1cc(C(C)(C)O)cc(C(C)(C)O)c1] |
| 150 | 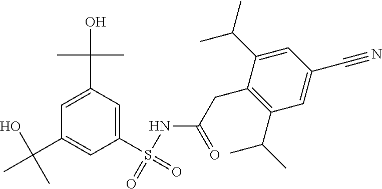 [SMILES: CC(C)c1cc(C#N)cc(C(C)C)c1CC(=O)NS(=O)(=O)c1cc(C(C)(C)O)cc(C(C)(C)O)c1] |
| 151 | 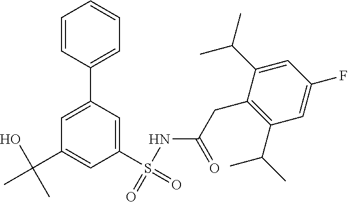 [SMILES: CC(C)c1cc(F)cc(C(C)C)c1CC(=O)NS(=O)(=O)c1cc(-c2ccccc2)cc(C(C)(C)O)c1] |
| 152 | 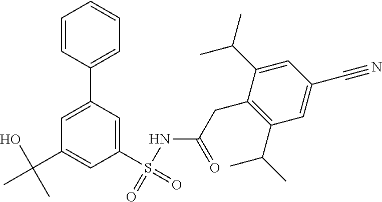 [SMILES: CC(C)c1cc(C#N)cc(C(C)C)c1CC(=O)NS(=O)(=O)c1cc(-c2ccccc2)cc(C(C)(C)O)c1] |
| 153 | 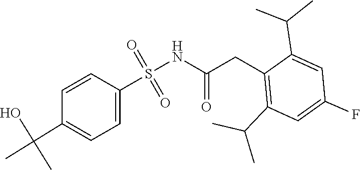 [SMILES: CC(C)c1cc(F)cc(C(C)C)c1CC(=O)NS(=O)(=O)c1ccc(C(C)(C)O)cc1] |
| 154 | 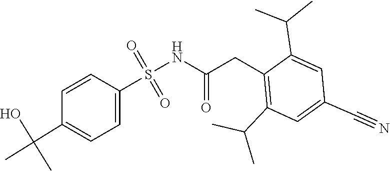 [SMILES: CC(C)c1cc(C#N)cc(C(C)C)c1CC(=O)NS(=O)(=O)c1ccc(C(C)(C)O)cc1] |
| 155 | 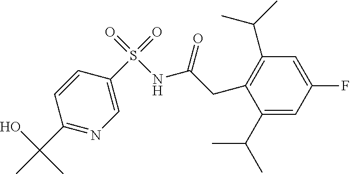 [SMILES: CC(C)c1cc(F)cc(C(C)C)c1CC(=O)NS(=O)(=O)c1ccc(C(C)(C)O)nc1] |
| 156 | 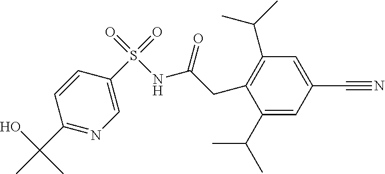 [SMILES: CC(C)c1cc(C#N)cc(C(C)C)c1CC(=O)NS(=O)(=O)c1ccc(C(C)(C)O)nc1] |
| 157 | 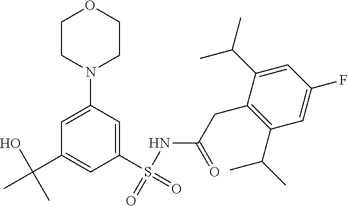 [SMILES: CC(C)c1cc(F)cc(C(C)C)c1CC(=O)NS(=O)(=O)c1cc(N2CCOCC2)cc(C(C)(C)O)c1] |
| 158 | 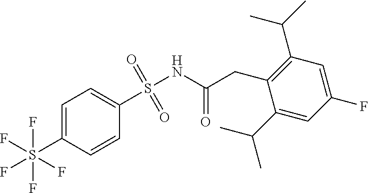 [SMILES: CC(C)c1cc(F)cc(C(C)C)c1CC(=O)NS(=O)(=O)c1ccc(S(F)(F)(F)(F)F)cc1] |
| 159 | 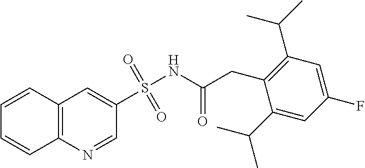 [SMILES: CC(C)c1cc(F)cc(C(C)C)c1CC(=O)NS(=O)(=O)c1cnc2ccccc2c1] |
| 160 | 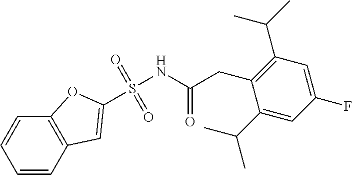 [SMILES: CC(C)c1cc(F)cc(C(C)C)c1CC(=O)NS(=O)(=O)c1cc2ccccc2o1] |
| 161 | 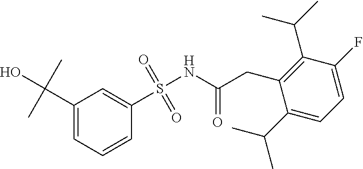 [SMILES: CC(C)c1ccc(F)c(C(C)C)c1CC(=O)NS(=O)(=O)c1cccc(C(C)(C)O)c1] |
| 162 | 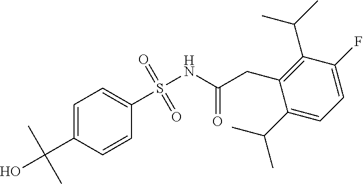 [SMILES: CC(C)c1ccc(F)c(C(C)C)c1CC(=O)NS(=O)(=O)c1ccc(C(C)(C)O)cc1] |
| 163 | 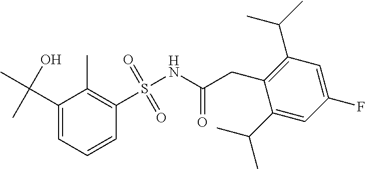 [SMILES: Cc1c(C(C)(C)O)cccc1S(=O)(=O)NC(=O)Cc1c(C(C)C)cc(F)cc1C(C)C] |
| 164 | 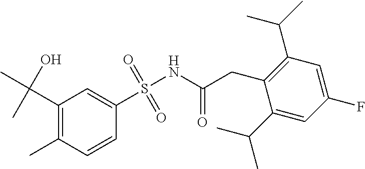 [SMILES: Cc1ccc(S(=O)(=O)NC(=O)Cc2c(C(C)C)cc(F)cc2C(C)C)cc1C(C)(C)O] |
| 165 | 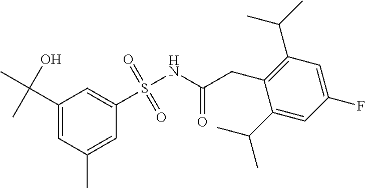 [SMILES: Cc1cc(C(C)(C)O)cc(S(=O)(=O)NC(=O)Cc2c(C(C)C)cc(F)cc2C(C)C)c1] |
| 166 | 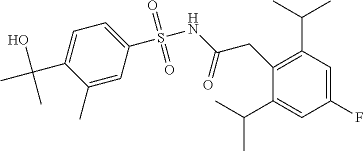 [SMILES: Cc1cc(S(=O)(=O)NC(=O)Cc2c(C(C)C)cc(F)cc2C(C)C)ccc1C(C)(C)O] |
| 167 | 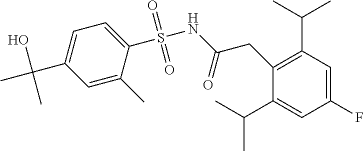 [SMILES: Cc1cc(C(C)(C)O)ccc1S(=O)(=O)NC(=O)Cc1c(C(C)C)cc(F)cc1C(C)C] |
| 168 | 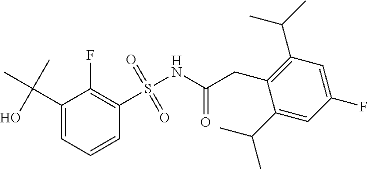 [SMILES: CC(C)c1cc(F)cc(C(C)C)c1CC(=O)NS(=O)(=O)c1cccc(C(C)(C)O)c1F] |
| 169 | 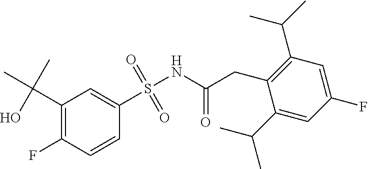 [SMILES: CC(C)c1cc(F)cc(C(C)C)c1CC(=O)NS(=O)(=O)c1ccc(F)c(C(C)(C)O)c1] |
| 170 | 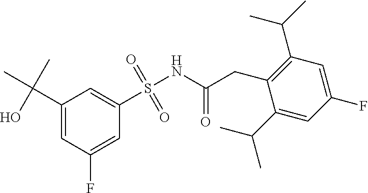 [SMILES: CC(C)c1cc(F)cc(C(C)C)c1CC(=O)NS(=O)(=O)c1cc(F)cc(C(C)(C)O)c1] |
| 171 | 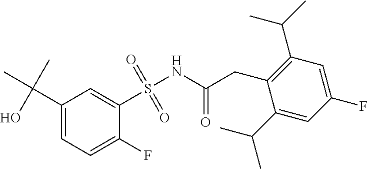 [SMILES: CC(C)c1cc(F)cc(C(C)C)c1CC(=O)NS(=O)(=O)c1cc(C(C)(C)O)ccc1F] |
| 172 | 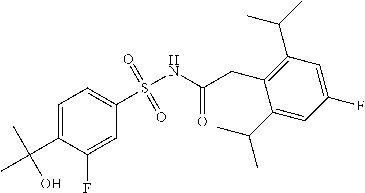 [SMILES: CC(C)c1cc(F)cc(C(C)C)c1CC(=O)NS(=O)(=O)c1ccc(C(C)(C)O)c(F)c1] |
| 173 | 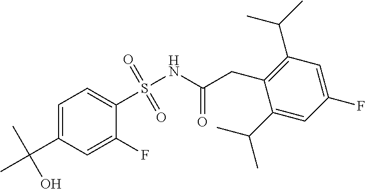 [SMILES: CC(C)c1cc(F)cc(C(C)C)c1CC(=O)NS(=O)(=O)c1ccc(C(C)(C)O)cc1F] |
| 174 | 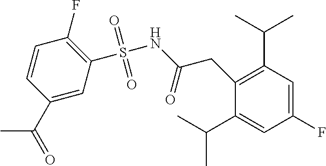 [SMILES: CC(=O)c1ccc(F)c(S(=O)(=O)NC(=O)Cc2c(C(C)C)cc(F)cc2C(C)C)c1] |
| 175 | 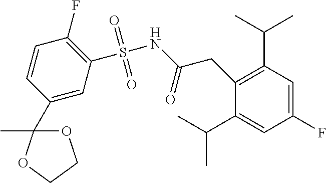 [SMILES: CC(C)c1cc(F)cc(C(C)C)c1CC(=O)NS(=O)(=O)c1cc(C2(C)OCCO2)ccc1F] |
| 176 | 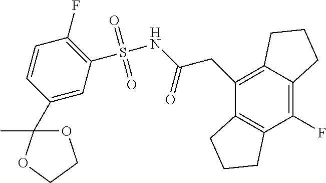 [SMILES: CC1(c2ccc(F)c(S(=O)(=O)NC(=O)Cc3c4c(c(F)c5c3CCC5)CCC4)c2)OCCO1] |
| 177 | 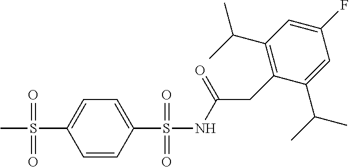 [SMILES: CC(C)c1cc(F)cc(C(C)C)c1CC(=O)NS(=O)(=O)c1ccc(S(C)(=O)=O)cc1] |
| 178 | 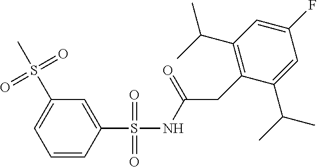 [SMILES: CC(C)c1cc(F)cc(C(C)C)c1CC(=O)NS(=O)(=O)c1cccc(S(C)(=O)=O)c1] |
| 179 | 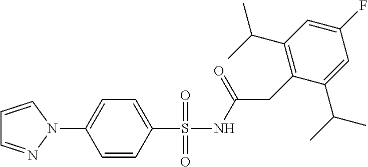 [SMILES: CC(C)c1cc(F)cc(C(C)C)c1CC(=O)NS(=O)(=O)c1ccc(-n2cccn2)cc1] |
| 180 | 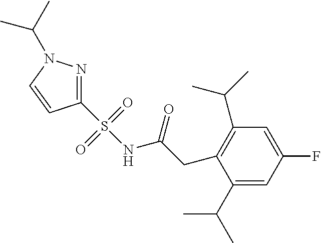 [SMILES: CC(C)c1cc(F)cc(C(C)C)c1CC(=O)NS(=O)(=O)c1ccn(C(C)C)n1] |
| 181 | 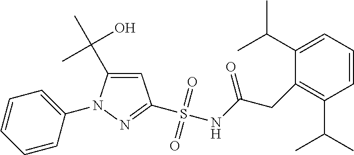 [SMILES: CC(C)c1cccc(C(C)C)c1CC(=O)NS(=O)(=O)c1cc(C(C)(C)O)n(-c2ccccc2)n1] |
| 182 | 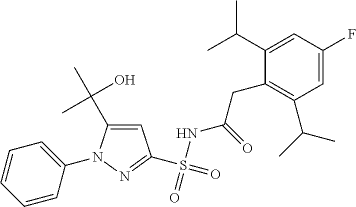 [SMILES: CC(C)c1cc(F)cc(C(C)C)c1CC(=O)NS(=O)(=O)c1cc(C(C)(C)O)n(-c2ccccc2)n1] |
| 183 | 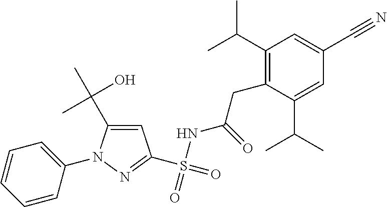 [SMILES: CC(C)c1cc(C#N)cc(C(C)C)c1CC(=O)NS(=O)(=O)c1cc(C(C)(C)O)n(-c2ccccc2)n1] |
| 184 | 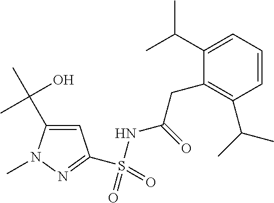 [SMILES: CC(C)c1cccc(C(C)C)c1CC(=O)NS(=O)(=O)c1cc(C(C)(C)O)n(C)n1] |
| 185 | 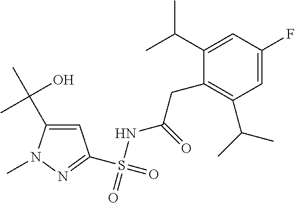 [SMILES: CC(C)c1cc(F)cc(C(C)C)c1CC(=O)NS(=O)(=O)c1cc(C(C)(C)O)n(C)n1] |
| 186 | 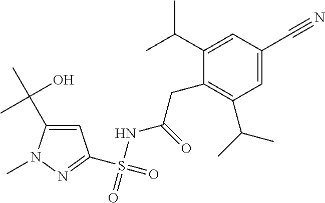 [SMILES: CC(C)c1cc(C#N)cc(C(C)C)c1CC(=O)NS(=O)(=O)c1cc(C(C)(C)O)n(C)n1] |
| 187 | 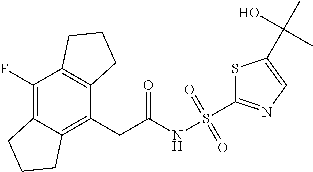 [SMILES: CC(C)(O)c1cnc(S(=O)(=O)NC(=O)Cc2c3c(c(F)c4c2CCC4)CCC3)s1] |
| 188 | 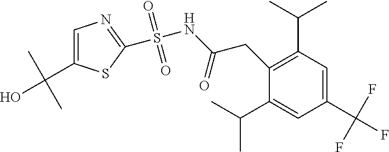 [SMILES: CC(C)c1cc(C(F)(F)F)cc(C(C)C)c1CC(=O)NS(=O)(=O)c1ncc(C(C)(C)O)s1] |
| 189 | 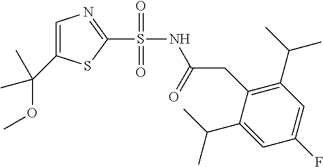 [SMILES: COC(C)(C)c1cnc(S(=O)(=O)NC(=O)Cc2c(C(C)C)cc(F)cc2C(C)C)s1] |
| 190 | 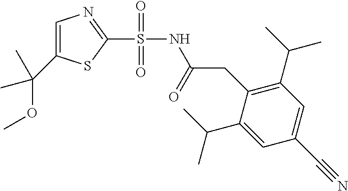 [SMILES: COC(C)(C)c1cnc(S(=O)(=O)NC(=O)Cc2c(C(C)C)cc(C#N)cc2C(C)C)s1] |
| 191 | 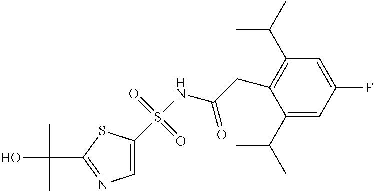 [SMILES: CC(C)c1cc(F)cc(C(C)C)c1CC(=O)NS(=O)(=O)c1cnc(C(C)(C)O)s1] |
| 192 | 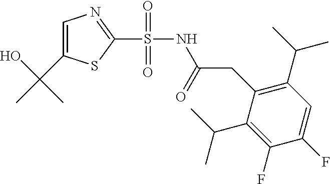 [SMILES: CC(C)c1cc(F)c(F)c(C(C)C)c1CC(=O)NS(=O)(=O)c1ncc(C(C)(C)O)s1] |
| 193 | 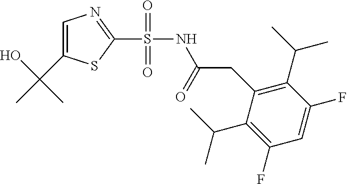 [SMILES: CC(C)c1c(F)cc(F)c(C(C)C)c1CC(=O)NS(=O)(=O)c1ncc(C(C)(C)O)s1] |
| 194 | 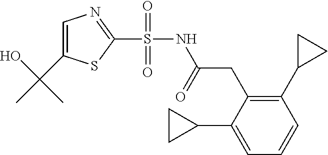 [SMILES: CC(C)(O)c1cnc(S(=O)(=O)NC(=O)Cc2c(C3CC3)cccc2C2CC2)s1] |
| 195 | 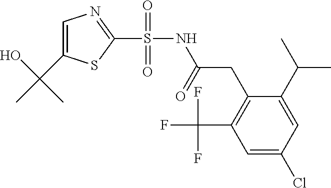 [SMILES: CC(C)c1cc(Cl)cc(C(F)(F)F)c1CC(=O)NS(=O)(=O)c1ncc(C(C)(C)O)s1] |
| 196 | 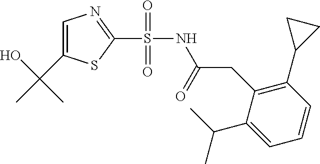 [SMILES: CC(C)c1cccc(C2CC2)c1CC(=O)NS(=O)(=O)c1ncc(C(C)(C)O)s1] |
| 197 | 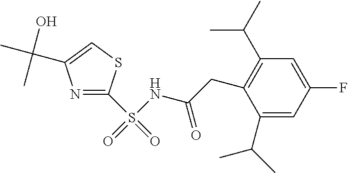 [SMILES: CC(C)c1cc(F)cc(C(C)C)c1CC(=O)NS(=O)(=O)c1nc(C(C)(C)O)cs1] |
| 198 | 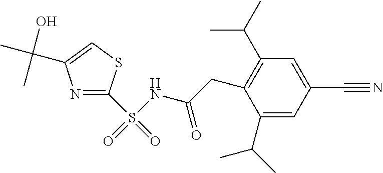 [SMILES: CC(C)c1cc(C#N)cc(C(C)C)c1CC(=O)NS(=O)(=O)c1nc(C(C)(C)O)cs1] |
| 199 | 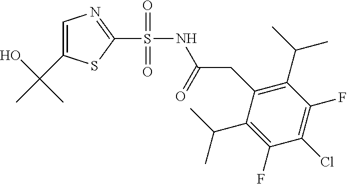 [SMILES: CC(C)c1c(F)c(Cl)c(F)c(C(C)C)c1CC(=O)NS(=O)(=O)c1ncc(C(C)(C)O)s1] |
| 200 | 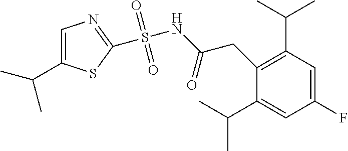 [SMILES: CC(C)c1cnc(S(=O)(=O)NC(=O)Cc2c(C(C)C)cc(F)cc2C(C)C)s1] |
| 201 | 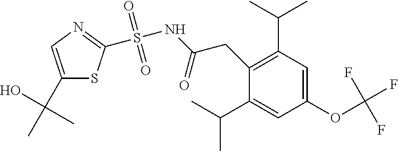 [SMILES: CC(C)c1cc(OC(F)(F)F)cc(C(C)C)c1CC(=O)NS(=O)(=O)c1ncc(C(C)(C)O)s1] |
| 202 | 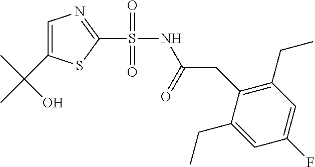 [SMILES: CCc1cc(F)cc(CC)c1CC(=O)NS(=O)(=O)c1ncc(C(C)(C)O)s1] |
| 203 | 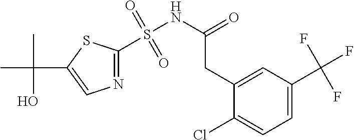 [SMILES: CC(C)(O)c1cnc(S(=O)(=O)NC(=O)Cc2cc(C(F)(F)F)ccc2Cl)s1] |
| 204 | 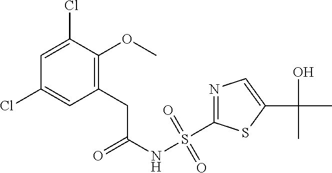 [SMILES: COc1c(Cl)cc(Cl)cc1CC(=O)NS(=O)(=O)c1ncc(C(C)(C)O)s1] |
| 205 | 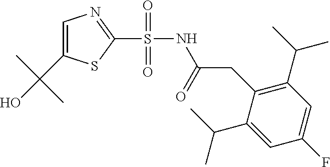 [SMILES: CC(C)c1cc(F)cc(C(C)C)c1CC(=O)NS(=O)(=O)c1ncc(C(C)(C)O)s1] |
| 206 | 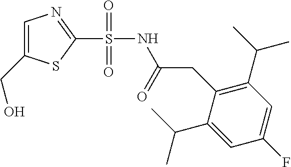 [SMILES: CC(C)c1cc(F)cc(C(C)C)c1CC(=O)NS(=O)(=O)c1ncc(CO)s1] |
| 207 | 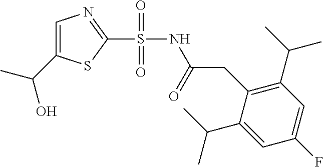 [SMILES: CC(C)c1cc(F)cc(C(C)C)c1CC(=O)NS(=O)(=O)c1ncc(C(C)O)s1] |
| 208 | 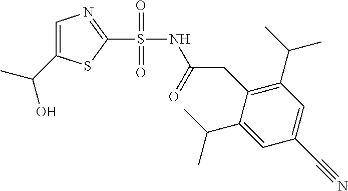 [SMILES: CC(C)c1cc(C#N)cc(C(C)C)c1CC(=O)NS(=O)(=O)c1ncc(C(C)O)s1] |
| 209 | 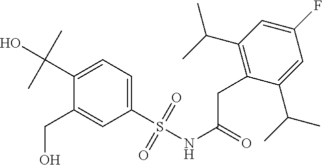 [SMILES: CC(C)c1cc(F)cc(C(C)C)c1CC(=O)NS(=O)(=O)c1ccc(C(C)(C)O)c(CO)c1] |
| 210 | 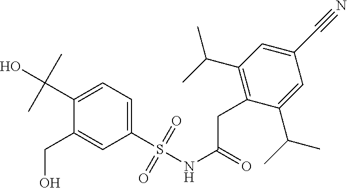 [SMILES: CC(C)c1cc(C#N)cc(C(C)C)c1CC(=O)NS(=O)(=O)c1ccc(C(C)(C)O)c(CO)c1] |
| 211 | 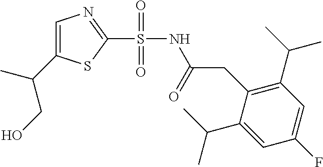 [SMILES: CC(C)c1cc(F)cc(C(C)C)c1CC(=O)NS(=O)(=O)c1ncc(C(C)CO)s1] |
| 212 | 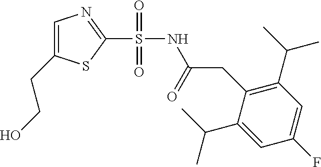 [SMILES: CC(C)c1cc(F)cc(C(C)C)c1CC(=O)NS(=O)(=O)c1ncc(CCO)s1] |
| 213 | 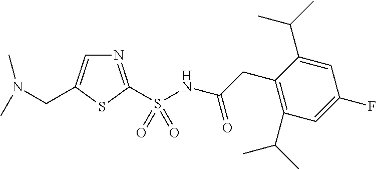 [SMILES: CC(C)c1cc(F)cc(C(C)C)c1CC(=O)NS(=O)(=O)c1ncc(CN(C)C)s1] |
| 214 | 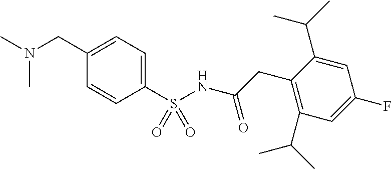 [SMILES: CC(C)c1cc(F)cc(C(C)C)c1CC(=O)NS(=O)(=O)c1ccc(CN(C)C)cc1] |
| 215 | 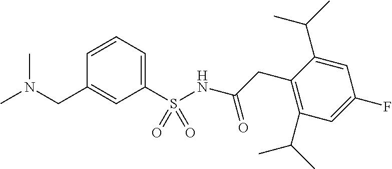 [SMILES: CC(C)c1cc(F)cc(C(C)C)c1CC(=O)NS(=O)(=O)c1cccc(CN(C)C)c1] |
| 216 | 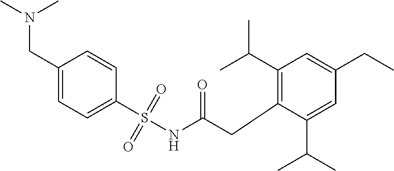 [SMILES: CCc1cc(C(C)C)c(CC(=O)NS(=O)(=O)c2ccc(CN(C)C)cc2)c(C(C)C)c1] |
| 217 | 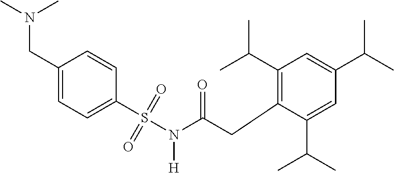 [SMILES: CC(C)c1cc(C(C)C)c(CC(=O)NS(=O)(=O)c2ccc(CN(C)C)cc2)c(C(C)C)c1] |
| 218 | 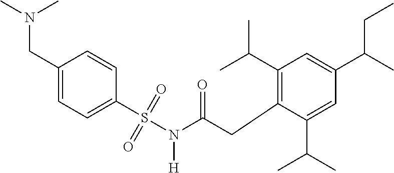 [SMILES: CCC(C)c1cc(C(C)C)c(CC(=O)NS(=O)(=O)c2ccc(CN(C)C)cc2)c(C(C)C)c1] |
| 219 | 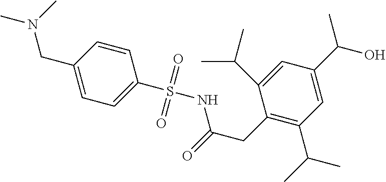 [SMILES: CC(C)c1cc(C(C)O)cc(C(C)C)c1CC(=O)NS(=O)(=O)c1ccc(CN(C)C)cc1] |

and pharmaceutically acceptable salts thereof.

In some embodiments, provided herein is a compound selected from the group consisting of compounds 127-212 above.

In some embodiments, provided herein is a compound selected from the group consisting of compounds 127-215 above.

In some embodiments, provided herein is a compound selected from the group consisting of the compounds below:

TABLE 1C

| | |
|---|---|
| 301 | 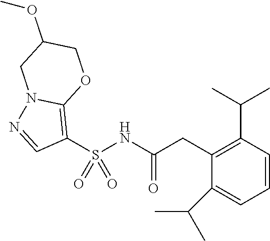 [SMILES: COC1COc2c(S(=O)(=O)NC(=O)Cc3c(C(C)C)cccc3C(C)C)cnn2C1] |
| 302 | 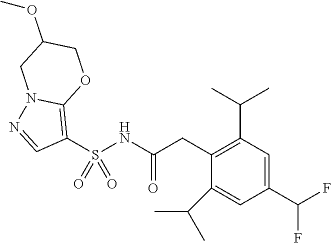 [SMILES: COC1COc2c(S(=O)(=O)NC(=O)Cc3c(C(C)C)cc(C(F)F)cc3C(C)C)cnn2C1] |
| 303 | 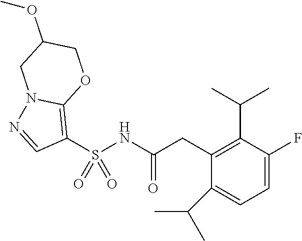 [SMILES: COC1COc2c(S(=O)(=O)NC(=O)Cc3c(C(C)C)ccc(F)c3C(C)C)cnn2C1] |
| 304 | 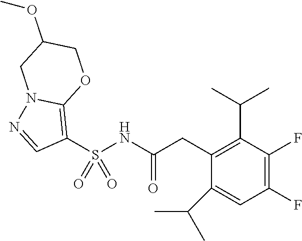 [SMILES: COC1COc2c(S(=O)(=O)NC(=O)Cc3c(C(C)C)cc(F)c(F)c3C(C)C)cnn2C1] |
| 305 | 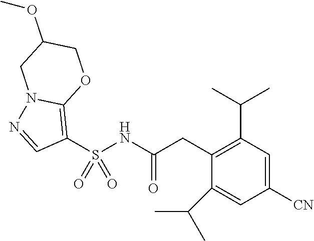 [SMILES: COC1COc2c(S(=O)(=O)NC(=O)Cc3c(C(C)C)cc(C#N)cc3C(C)C)cnn2C1] |
| 306 | 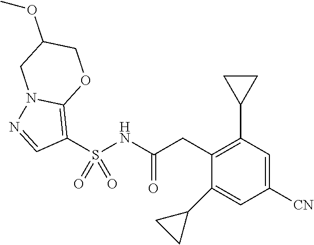 [SMILES: COC1COc2c(S(=O)(=O)NC(=O)Cc3c(C4CC4)cc(C#N)cc3C3CC3)cnn2C1] |
| 307 | 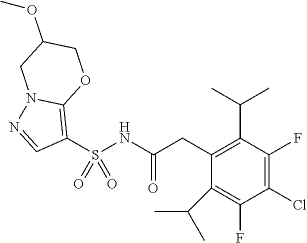 [SMILES: COC1COc2c(S(=O)(=O)NC(=O)Cc3c(C(C)C)c(F)c(Cl)c(F)c3C(C)C)cnn2C1] |
| 308 | 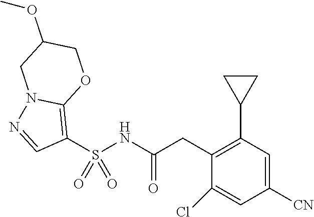 [SMILES: COC1COc2c(S(=O)(=O)NC(=O)Cc3c(Cl)cc(C#N)cc3C3CC3)cnn2C1] |
| 309 | 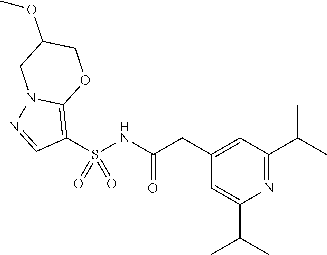 [SMILES: COC1COc2c(S(=O)(=O)NC(=O)Cc3cc(C(C)C)nc(C(C)C)c3)cnn2C1] |
| 310 | 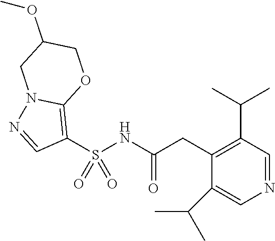 [SMILES: COC1COc2c(S(=O)(=O)NC(=O)Cc3c(C(C)C)cncc3C(C)C)cnn2C1] |
| 311 | 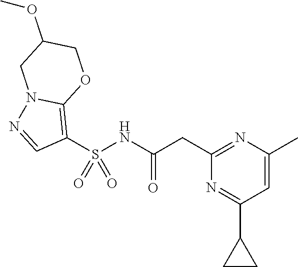 |
| 312 | 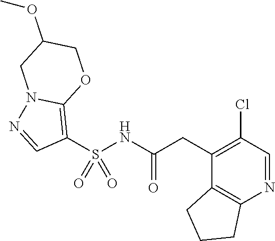 [SMILES: COC1COc2c(S(=O)(=O)NC(=O)Cc3c(Cl)cnc4c3CCC4)cnn2C1] |
| 313 | 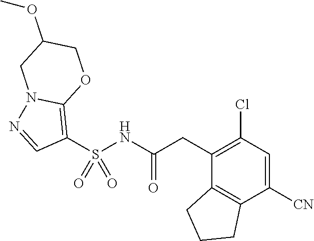 [SMILES: COC1COc2c(S(=O)(=O)NC(=O)Cc3c(Cl)cc(C#N)c4c3CCC4)cnn2C1] |
| 314 | 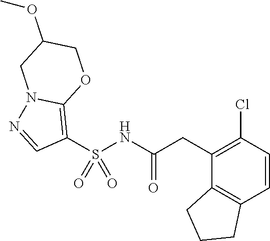 [SMILES: COC1COc2c(S(=O)(=O)NC(=O)Cc3c(Cl)ccc4c3CCC4)cnn2C1] |
| 315 | 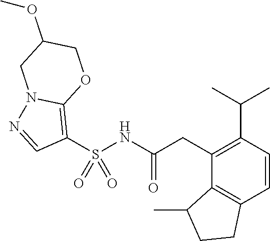 [SMILES: COC1COc2c(S(=O)(=O)NC(=O)Cc3c(C(C)C)ccc4c3C(C)CC4)cnn2C1] |
| 316 | 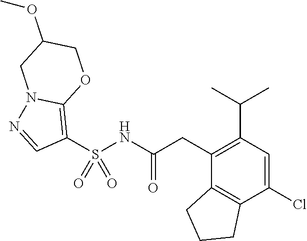 [SMILES: COC1COc2c(S(=O)(=O)NC(=O)Cc3c(C(C)C)cc(Cl)c4c3CCC4)cnn2C1] |
| 317 | 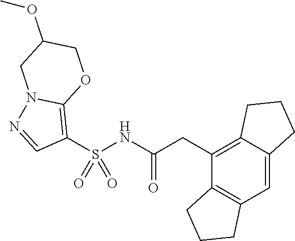 [SMILES: COC1COc2c(S(=O)(=O)NC(=O)Cc3c4c(cc5c3CCC5)CCC4)cnn2C1] |
| 318 | 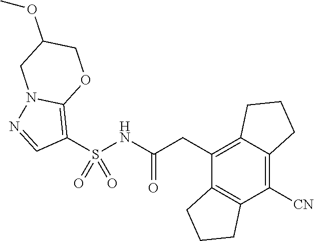 [SMILES: COC1COc2c(S(=O)(=O)NC(=O)Cc3c4c(c(C#N)c5c3CCC5)CCC4)cnn2C1] |
| 319 | 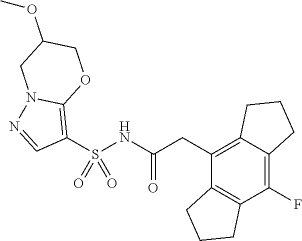 [SMILES: COC1COc2c(S(=O)(=O)NC(=O)Cc3c4c(c(F)c5c3CCC5)CCC4)cnn2C1] |
| 320 | 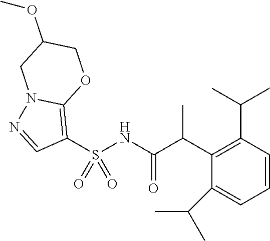 [SMILES: COC1COc2c(S(=O)(=O)NC(=O)C(C)c3c(C(C)C)cccc3C(C)C)cnn2C1] |
| 321 | 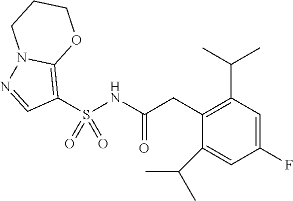 [SMILES: CC(C)c1cc(F)cc(C(C)C)c1CC(=O)NS(=O)(=O)c1cnn2c1OCCC2] |
| 322 | 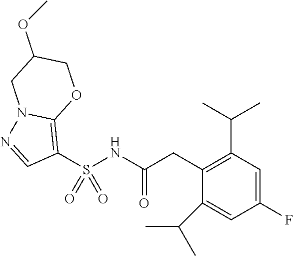 [SMILES: COC1COc2c(S(=O)(=O)NC(=O)Cc3c(C(C)C)cc(F)cc3C(C)C)cnn2C1] |
| 323 | 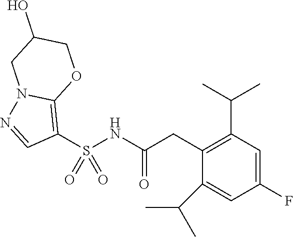 [SMILES: CC(C)c1cc(F)cc(C(C)C)c1CC(=O)NS(=O)(=O)c1cnn2c1OCC(O)C2] |
| 324 | 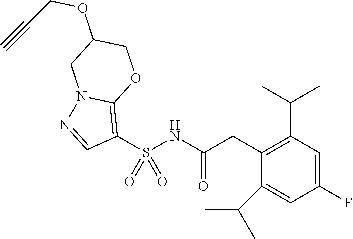 [SMILES: C#CCOC1COc2c(S(=O)(=O)NC(=O)Cc3c(C(C)C)cc(F)cc3C(C)C)cnn2C1] |
| 325 | 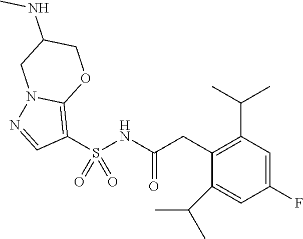 [SMILES: CNC1COc2c(S(=O)(=O)NC(=O)Cc3c(C(C)C)cc(F)cc3C(C)C)cnn2C1] |
| 326 | 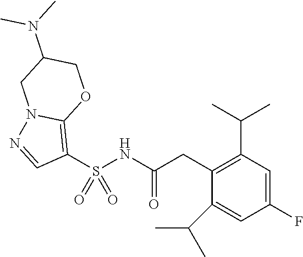 [SMILES: CC(C)c1cc(F)cc(C(C)C)c1CC(=O)NS(=O)(=O)c1cnn2c1OCC(N(C)C)C2] |
| 327 | 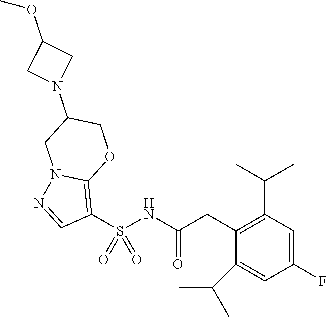 [SMILES: COC1CN(C2COc3c(S(=O)(=O)NC(=O)Cc4c(C(C)C)cc(F)cc4C(C)C)cnn3C2)C1] |
| 328 | 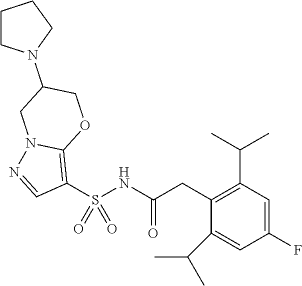 [SMILES: CC(C)c1cc(F)cc(C(C)C)c1CC(=O)NS(=O)(=O)c1cnn2c1OCC(N1CCCC1)C2] |
| 329 | 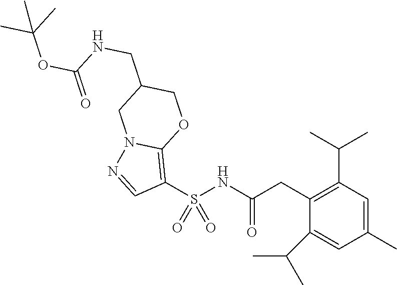 [SMILES: Cc1cc(C(C)C)c(CC(=O)NS(=O)(=O)c2cnn3c2OCC(CNC(=O)OC(C)(C)C)C3)c(C(C)C)c1] |
| 330 | 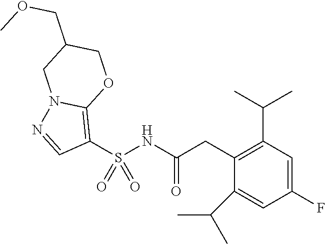 [SMILES: COCC1COc2c(S(=O)(=O)NC(=O)Cc3c(C(C)C)cc(F)cc3C(C)C)cnn2C1] |
| 331 | 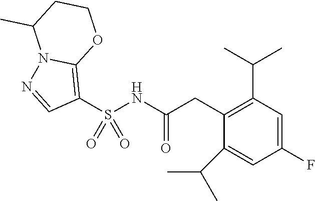 [SMILES: CC(C)c1cc(F)cc(C(C)C)c1CC(=O)NS(=O)(=O)c1cnn2c1OCCC2C] |
| 332 | 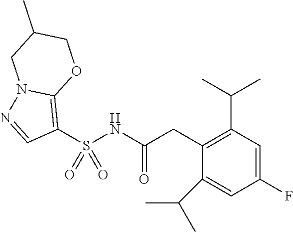 [SMILES: CC1COc2c(S(=O)(=O)NC(=O)Cc3c(C(C)C)cc(F)cc3C(C)C)cnn2C1] |
| 333 | 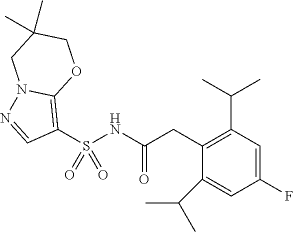 [SMILES: CC(C)c1cc(F)cc(C(C)C)c1CC(=O)NS(=O)(=O)c1cnn2c1OCC(C)(C)C2] |
| 334 | 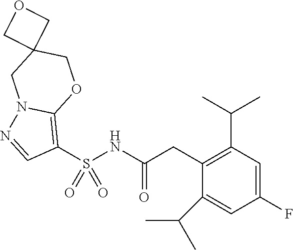 [SMILES: CC(C)c1cc(F)cc(C(C)C)c1CC(=O)NS(=O)(=O)c1cnn2c1OCC1(COC1)C2] |
| 335 | 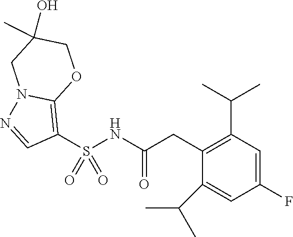 [SMILES: CC(C)c1cc(F)cc(C(C)C)c1CC(=O)NS(=O)(=O)c1cnn2c1OCC(C)(O)C2] |
| 336 | 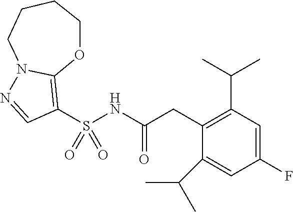 [SMILES: CC(C)c1cc(F)cc(C(C)C)c1CC(=O)NS(=O)(=O)c1cnn2c1OCCCC2] |
| 337 | 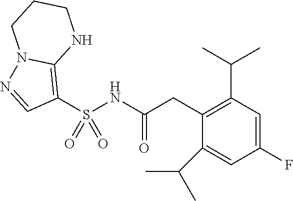 [SMILES: CC(C)c1cc(F)cc(C(C)C)c1CC(=O)NS(=O)(=O)c1cnn2c1NCCC2] |
| 338 | 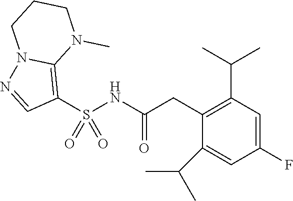 [SMILES: CC(C)c1cc(F)cc(C(C)C)c1CC(=O)NS(=O)(=O)c1cnn2c1N(C)CCC2] |
| 339 | 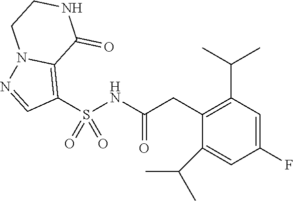 [SMILES: CC(C)c1cc(F)cc(C(C)C)c1CC(=O)NS(=O)(=O)c1cnn2c1C(=O)NCC2] |
| 340 | 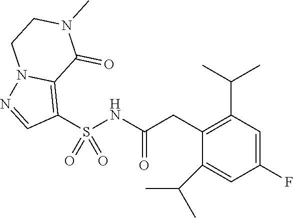 [SMILES: CC(C)c1cc(F)cc(C(C)C)c1CC(=O)NS(=O)(=O)c1cnn2c1C(=O)N(C)CC2] |
| 341 | 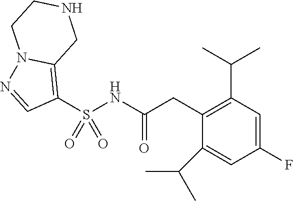 [SMILES: CC(C)c1cc(F)cc(C(C)C)c1CC(=O)NS(=O)(=O)c1cnn2c1CNCC2] |
| 342 | 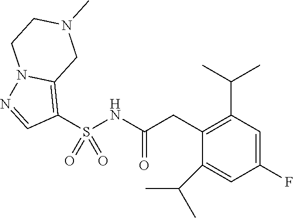 [SMILES: CC(C)c1cc(F)cc(C(C)C)c1CC(=O)NS(=O)(=O)c1cnn2c1CN(C)CC2] |
| 343 | 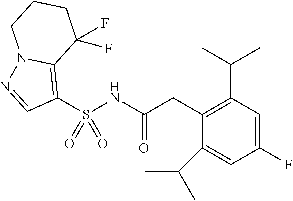 [SMILES: CC(C)c1cc(F)cc(C(C)C)c1CC(=O)NS(=O)(=O)c1cnn2c1C(F)(F)CCC2] |
| 344 | 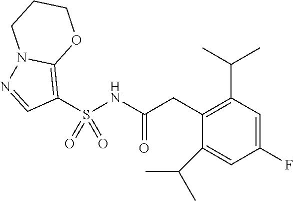 [SMILES: CC(C)c1cc(F)cc(C(C)C)c1CC(=O)NS(=O)(=O)c1cnn2c1OCCC2] |
| 345 | 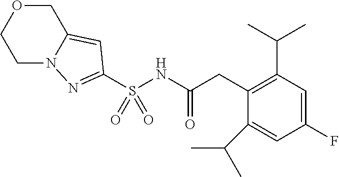 [SMILES: CC(C)c1cc(F)cc(C(C)C)c1CC(=O)NS(=O)(=O)c1cc2n(n1)CCOC2] |
| 346 | 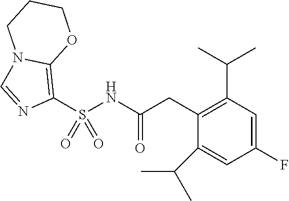 [SMILES: CC(C)c1cc(F)cc(C(C)C)c1CC(=O)NS(=O)(=O)c1ncn2c1OCCC2] |
| 347 | 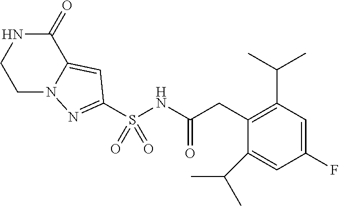 [SMILES: CC(C)c1cc(F)cc(C(C)C)c1CC(=O)NS(=O)(=O)c1cc2n(n1)CCNC2=O] |
| 348 | 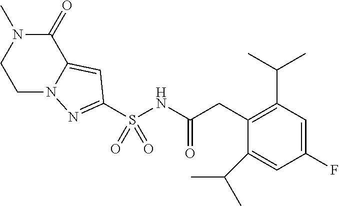 [SMILES: CC(C)c1cc(F)cc(C(C)C)c1CC(=O)NS(=O)(=O)c1cc2n(n1)CCN(C)C2=O] |
| 349 | 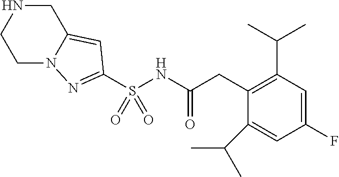 [SMILES: CC(C)c1cc(F)cc(C(C)C)c1CC(=O)NS(=O)(=O)c1cc2n(n1)CCNC2] |
| 350 | 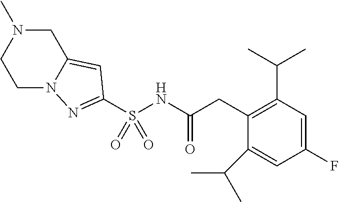 [SMILES: CC(C)c1cc(F)cc(C(C)C)c1CC(=O)NS(=O)(=O)c1cc2n(n1)CCN(C)C2] |
| 351 | 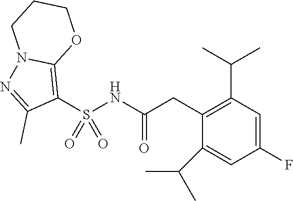 [SMILES: Cc1nn2c(c1S(=O)(=O)NC(=O)Cc1c(C(C)C)cc(F)cc1C(C)C)OCCC2] |
| 352 | 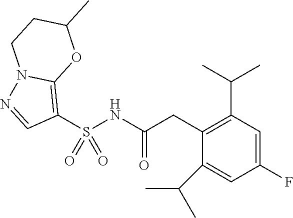 [SMILES: CC1CCn2ncc(S(=O)(=O)NC(=O)Cc3c(C(C)C)cc(F)cc3C(C)C)c2O1] |
| 353 | 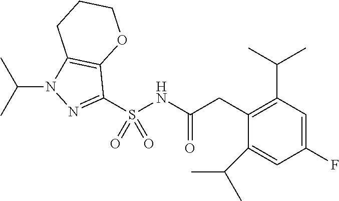 [SMILES: CC(C)c1cc(F)cc(C(C)C)c1CC(=O)NS(=O)(=O)c1nn(C(C)C)c2c1OCCC2] |
| 354 | 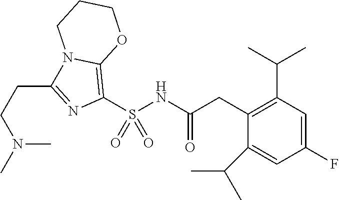 [SMILES: CC(C)c1cc(F)cc(C(C)C)c1CC(=O)NS(=O)(=O)c1nc(CCN(C)C)n2c1OCCC2] |
| 355 | 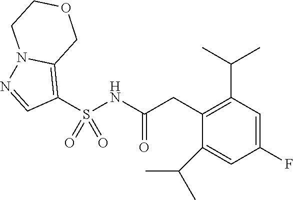 [SMILES: CC(C)c1cc(F)cc(C(C)C)c1CC(=O)NS(=O)(=O)c1cnn2c1COCC2] |
| 356 | 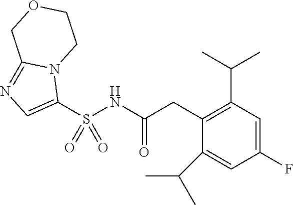 [SMILES: CC(C)c1cc(F)cc(C(C)C)c1CC(=O)NS(=O)(=O)c1cnc2n1CCOC2] |
| 357 | 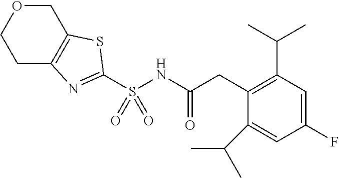 [SMILES: CC(C)c1cc(F)cc(C(C)C)c1CC(=O)NS(=O)(=O)c1nc2c(s1)COCC2] |
| 358 | 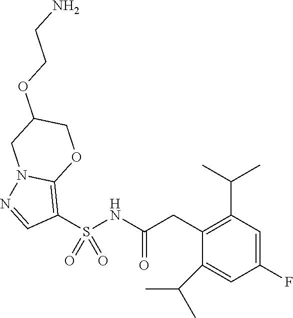 [SMILES: CC(C)c1cc(F)cc(C(C)C)c1CC(=O)NS(=O)(=O)c1cnn2c1OCC(OCCN)C2] |
| 359 | 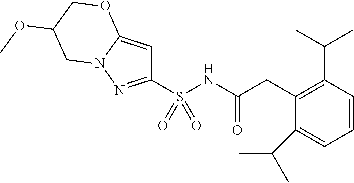 [SMILES: COC1COc2cc(S(=O)(=O)NC(=O)Cc3c(C(C)C)cccc3C(C)C)nn2C1] |
| 360 | 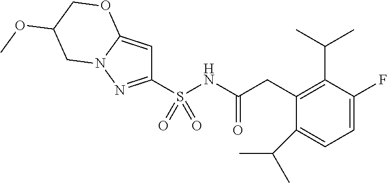 [SMILES: COC1COc2cc(S(=O)(=O)NC(=O)Cc3c(C(C)C)ccc(F)c3C(C)C)nn2C1] |
| 361 | 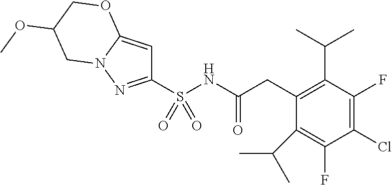 [SMILES: COC1COc2cc(S(=O)(=O)NC(=O)Cc3c(C(C)C)c(F)c(Cl)c(F)c3C(C)C)nn2C1] |
| 362 | 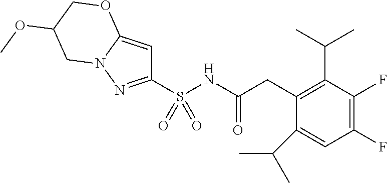 [SMILES: COC1COc2cc(S(=O)(=O)NC(=O)Cc3c(C(C)C)cc(F)c(F)c3C(C)C)nn2C1] |
| 363 | 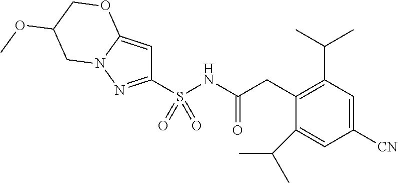 [SMILES: COC1COc2cc(S(=O)(=O)NC(=O)Cc3c(C(C)C)cc(C#N)cc3C(C)C)nn2C1] |
| 364 | 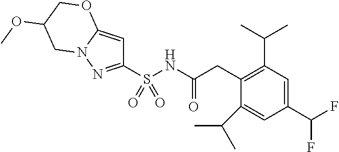 [SMILES: COC1COc2cc(S(=O)(=O)NC(=O)Cc3c(C(C)C)cc(C(F)F)cc3C(C)C)nn2C1] |
| 365 | 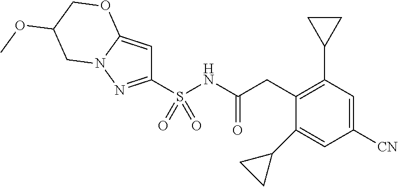 [SMILES: COC1COc2cc(S(=O)(=O)NC(=O)Cc3c(C4CC4)cc(C#N)cc3C3CC3)nn2C1] |
| 366 | 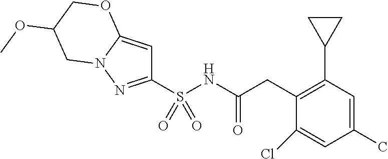 |
| 367 | 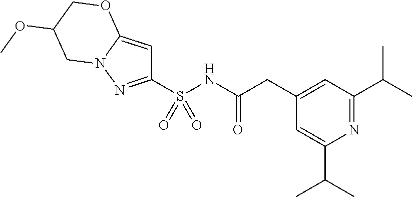 [SMILES: COC1COc2cc(S(=O)(=O)NC(=O)Cc3cc(C(C)C)nc(C(C)C)c3)nn2C1] |
| 368 | 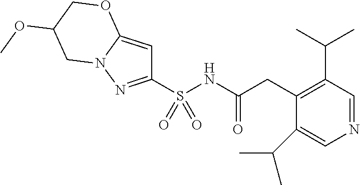 [SMILES: COC1COc2cc(S(=O)(=O)NC(=O)Cc3c(C(C)C)cncc3C(C)C)nn2C1] |
| 369 | 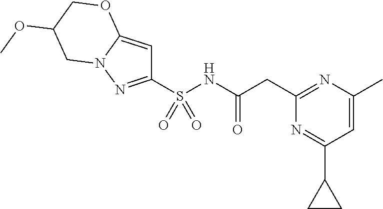 |
| 370 | 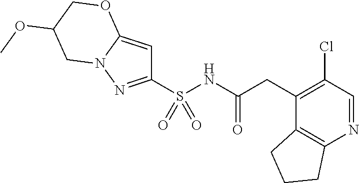 [SMILES: COC1COc2cc(S(=O)(=O)NC(=O)Cc3c(Cl)cnc4c3CCC4)nn2C1] |
| 371 | 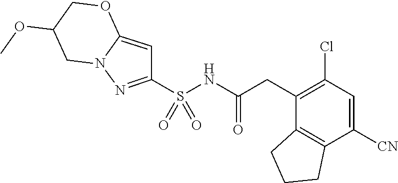 [SMILES: COC1COc2cc(S(=O)(=O)NC(=O)Cc3c(Cl)cc(C#N)c4c3CCC4)nn2C1] |
| 372 | 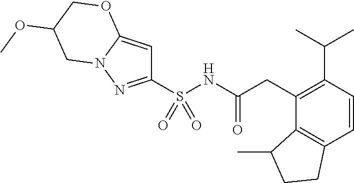 [SMILES: COC1COc2cc(S(=O)(=O)NC(=O)Cc3c(C(C)C)ccc4c3C(C)CC4)nn2C1] |
| 373 | 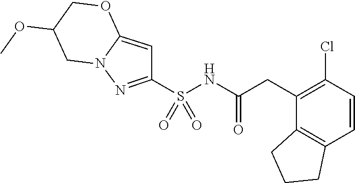 [SMILES: COC1COc2cc(S(=O)(=O)NC(=O)Cc3c(Cl)ccc4c3CCC4)nn2C1] |
| 374 | 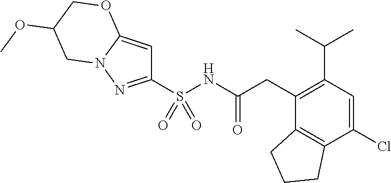 [SMILES: COC1COc2cc(S(=O)(=O)NC(=O)Cc3c(C(C)C)cc(Cl)c4c3CCC4)nn2C1] |
| 375 | 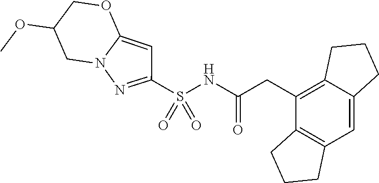 [SMILES: COC1COc2cc(S(=O)(=O)NC(=O)Cc3c4c(cc5c3CCC5)CCC4)nn2C1] |
| 376 | 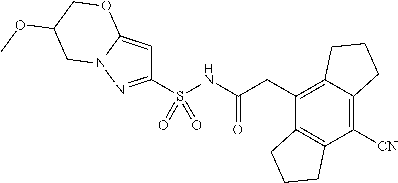 [SMILES: COC1COc2cc(S(=O)(=O)NC(=O)Cc3c4c(c(C#N)c5c3CCC5)CCC4)nn2C1] |
| 377 | 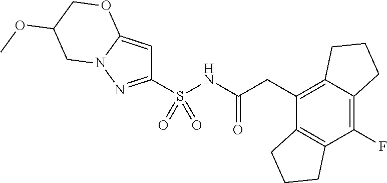 [SMILES: COC1COc2cc(S(=O)(=O)NC(=O)Cc3c4c(c(F)c5c3CCC5)CCC4)nn2C1] |
| 378 | 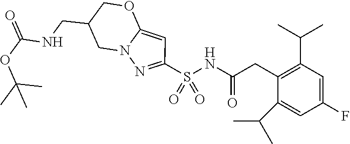 [SMILES: CC(C)c1cc(F)cc(C(C)C)c1CC(=O)NS(=O)(=O)c1cc2n(n1)CC(CNC(=O)OC(C)(C)C)CO2] |
| 379 | 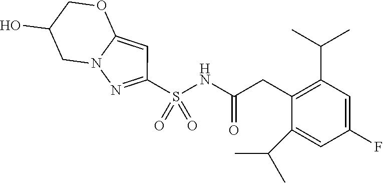 [SMILES: CC(C)c1cc(F)cc(C(C)C)c1CC(=O)NS(=O)(=O)c1cc2n(n1)CC(O)CO2] |
| 380 | 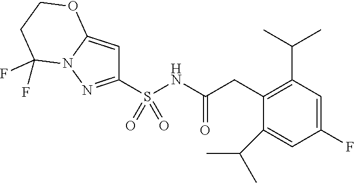 [SMILES: CC(C)c1cc(F)cc(C(C)C)c1CC(=O)NS(=O)(=O)c1cc2n(n1)C(F)(F)CCO2] |
| 381 | 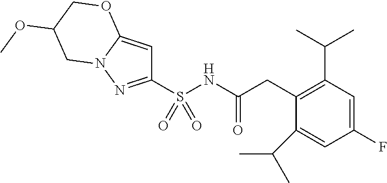 [SMILES: COC1COc2cc(S(=O)(=O)NC(=O)Cc3c(C(C)C)cc(F)cc3C(C)C)nn2C1] |
| 382 | 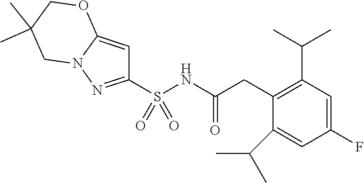 [SMILES: CC(C)c1cc(F)cc(C(C)C)c1CC(=O)NS(=O)(=O)c1cc2n(n1)CC(C)(C)CO2] |
| 383 | 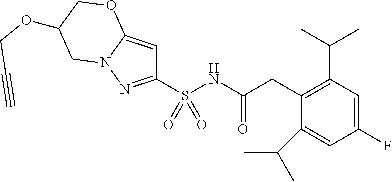 [SMILES: C#CCOC1COc2cc(S(=O)(=O)NC(=O)Cc3c(C(C)C)cc(F)cc3C(C)C)nn2C1] |
| 384 | 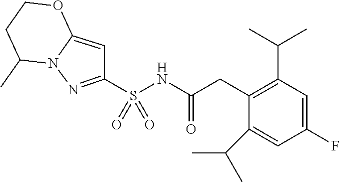 [SMILES: CC(C)c1cc(F)cc(C(C)C)c1CC(=O)NS(=O)(=O)c1cc2n(n1)C(C)CCO2] |
| 385 | 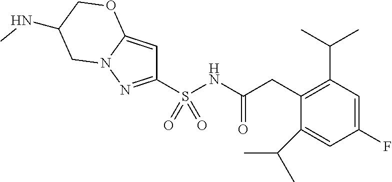 [SMILES: CNC1COc2cc(S(=O)(=O)NC(=O)Cc3c(C(C)C)cc(F)cc3C(C)C)nn2C1] |
| 386 | 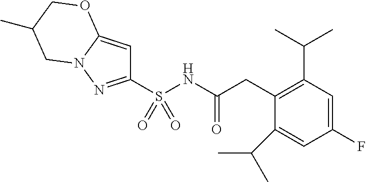 [SMILES: CC1COc2cc(S(=O)(=O)NC(=O)Cc3c(C(C)C)cc(F)cc3C(C)C)nn2C1] |
| 387 | 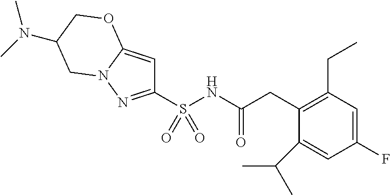 [SMILES: CCc1cc(F)cc(C(C)C)c1CC(=O)NS(=O)(=O)c1cc2n(n1)CC(N(C)C)CO2] |
| 388 | 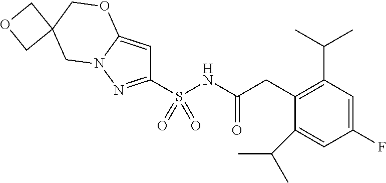 [SMILES: CC(C)c1cc(F)cc(C(C)C)c1CC(=O)NS(=O)(=O)c1cc2n(n1)CC1(COC1)CO2] |
| 389 | 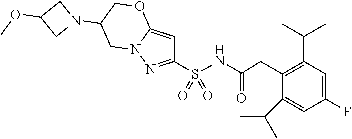 [SMILES: COC1CN(C2COc3cc(S(=O)(=O)NC(=O)Cc4c(C(C)C)cc(F)cc4C(C)C)nn3C2)C1] |
| 390 | 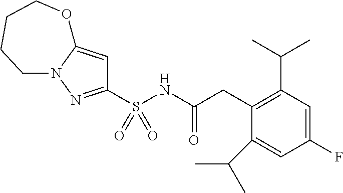 [SMILES: CC(C)c1cc(F)cc(C(C)C)c1CC(=O)NS(=O)(=O)c1cc2n(n1)CCCCO2] |
| 391 | 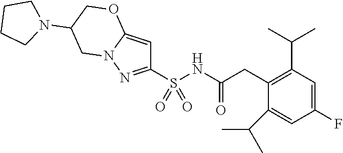 [SMILES: CC(C)c1cc(F)cc(C(C)C)c1CC(=O)NS(=O)(=O)c1cc2n(n1)CC(N1CCCC1)CO2] |
| 392 | 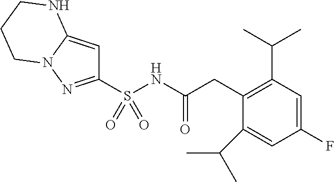 [SMILES: CC(C)c1cc(F)cc(C(C)C)c1CC(=O)NS(=O)(=O)c1cc2n(n1)CCCN2] |
| 393 | 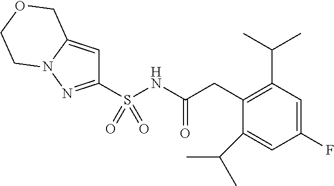 [SMILES: CC(C)c1cc(F)cc(C(C)C)c1CC(=O)NS(=O)(=O)c1cc2n(n1)CCOC2] |
| 394 | 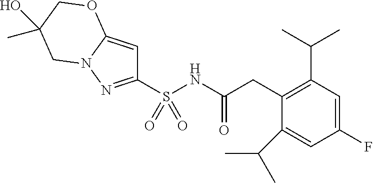 [SMILES: CC(C)c1cc(F)cc(C(C)C)c1CC(=O)NS(=O)(=O)c1cc2n(n1)CC(C)(O)CO2] |
| 395 | 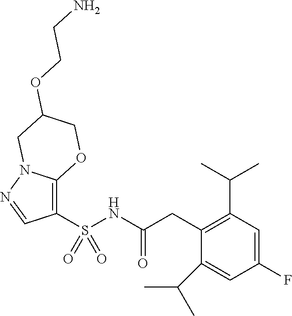 [SMILES: CC(C)c1cc(F)cc(C(C)C)c1CC(=O)NS(=O)(=O)c1cnn2c1OCC(OCCN)C2] |

and pharmaceutically acceptable salts thereof.

In some embodiments, provided herein is a compound that is not a compound selected from compounds 101 to 126.

In some embodiments, provided herein is a compound that is not a compound selected from compounds 127 to 215.

In some embodiments, provided herein is a compound selected from the group consisting of the compounds in Table 1D:

TABLE 1D

| | |
|---|---|
| 401 | 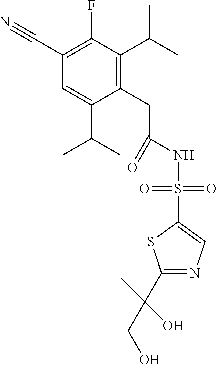 [SMILES: CC(C)c1cc(C#N)c(F)c(C(C)C)c1CC(=O)NS(=O)(=O)c1cnc(C(C)(O)CO)s1] |
| 402 | 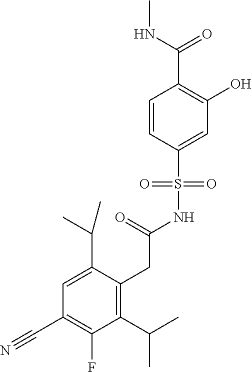 [SMILES: CNC(=O)c1ccc(S(=O)(=O)NC(=O)Cc2c(C(C)C)cc(C#N)c(F)c2C(C)C)cc1O] |
| 403 | 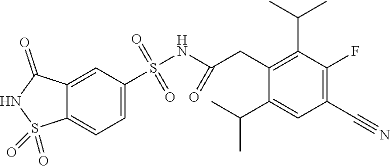 [SMILES: CC(C)c1cc(C#N)c(F)c(C(C)C)c1CC(=O)NS(=O)(=O)c1ccc2c(c1)C(=O)NS2(=O)=O] |
| 404 | 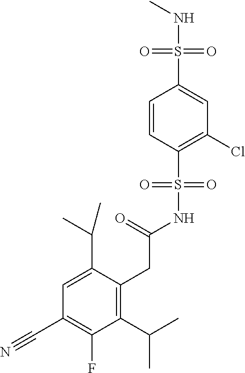 [SMILES: CNS(=O)(=O)c1ccc(S(=O)(=O)NC(=O)Cc2c(C(C)C)cc(C#N)c(F)c2C(C)C)c(Cl)c1] |
| 405 | 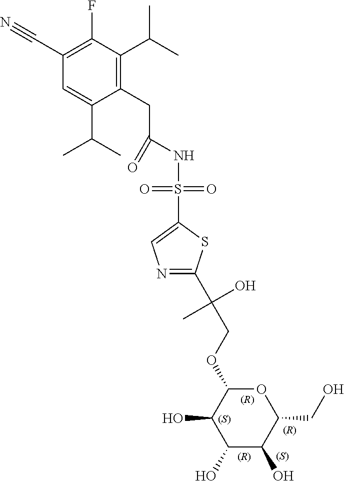 |
| 406 | 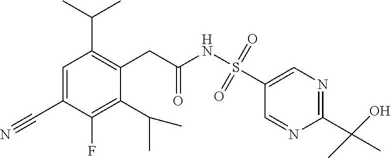 [SMILES: CC(C)c1cc(C#N)c(F)c(C(C)C)c1CC(=O)NS(=O)(=O)c1cnc(C(C)(C)O)nc1] |
| 407 | 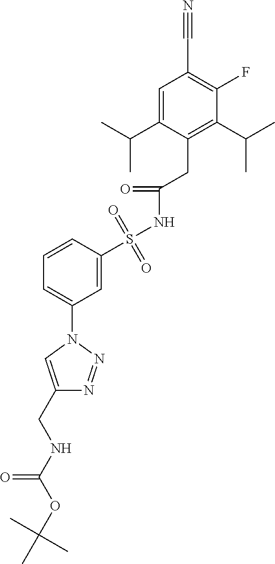 [SMILES: CC(C)c1cc(C#N)c(F)c(C(C)C)c1CC(=O)NS(=O)(=O)c1cccc(-n2cc(CNC(=O)OC(C)(C)C)nn2)c1] |
| 408 | 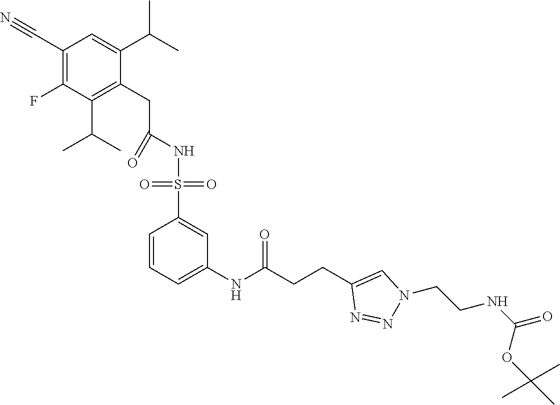 [SMILES: CC(C)c1cc(C#N)c(F)c(C(C)C)c1CC(=O)NS(=O)(=O)c1cccc(NC(=O)CCc2cn(CCNC(=O)OC(C)(C)C)nn2)c1] |
| 409 | 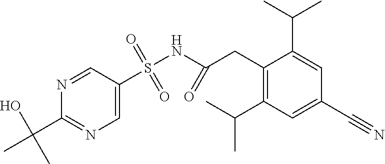 [SMILES: CC(C)c1cc(C#N)cc(C(C)C)c1CC(=O)NS(=O)(=O)c1cnc(C(C)(C)O)nc1] |
| 410 | 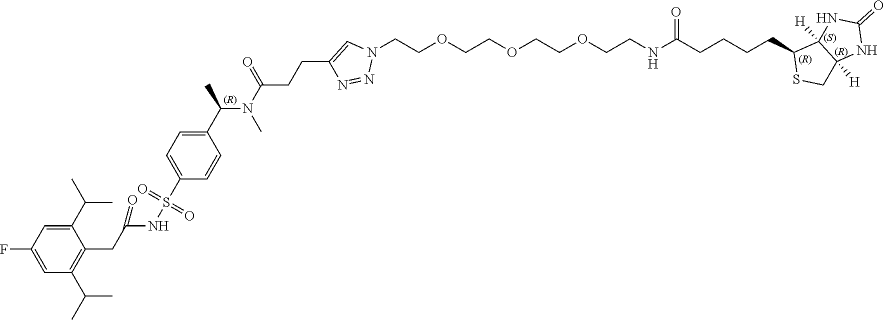 |
| 411 | 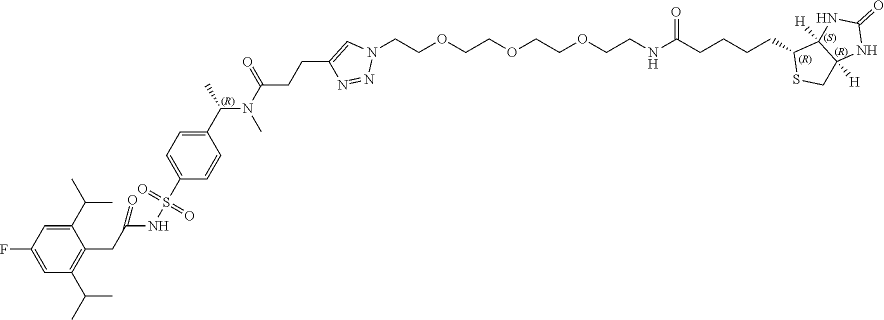 |
| 412 | 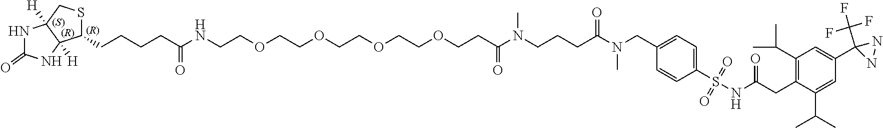 |
| 413 | 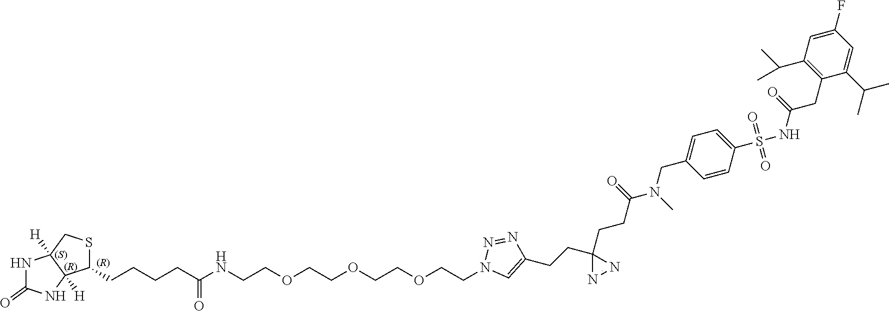 |
| 414 | 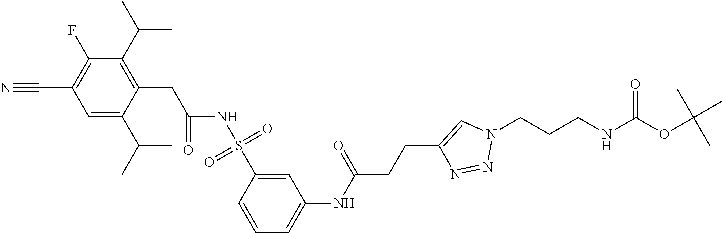 [SMILES: CC(C)c1cc(C#N)c(F)c(C(C)C)c1CC(=O)NS(=O)(=O)c1cccc(NC(=O)CCc2cn(CCCNC(=O)OC(C)(C)C)nn2)c1] |
| 415 | 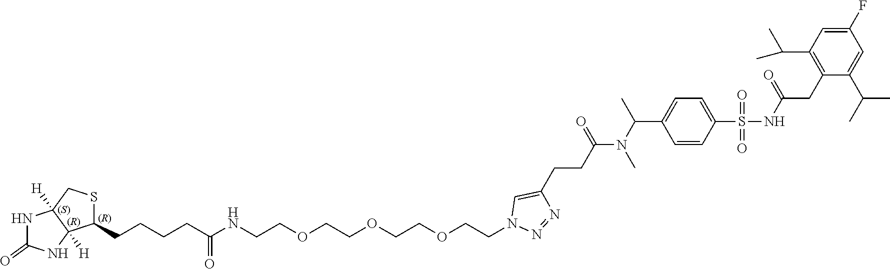 |
| 416 | 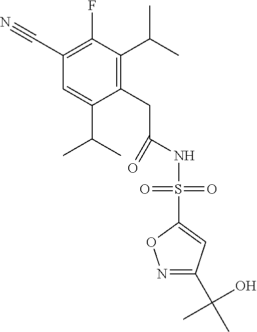 [SMILES: CC(C)c1cc(C#N)c(F)c(C(C)C)c1CC(=O)NS(=O)(=O)c1cc(C(C)(C)O)no1] |
| 417 | 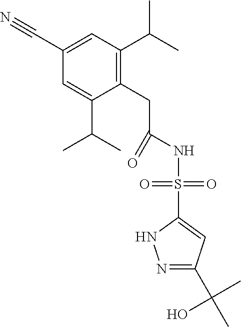 [SMILES: CC(C)c1cc(C#N)cc(C(C)C)c1CC(=O)NS(=O)(=O)c1cc(C(C)(C)O)n[nH]1] |
| 418 | 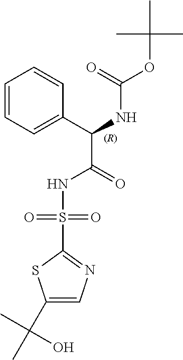 |
| 419 | 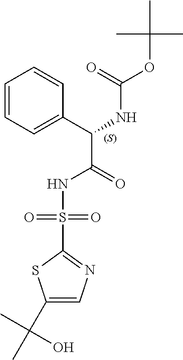 |
| 420 | 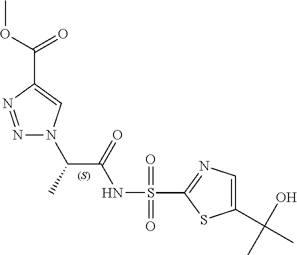 |
| 421 | 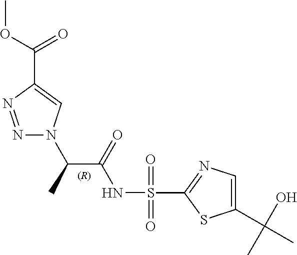 |
| 422 | 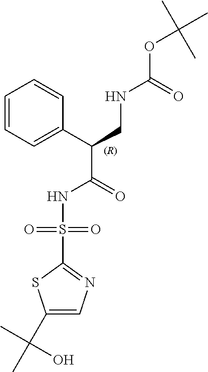 |
| 423 | 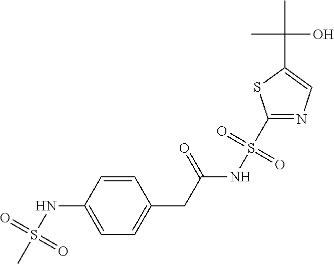 [SMILES: CC(C)(O)c1cnc(S(=O)(=O)NC(=O)Cc2ccc(NS(C)(=O)=O)cc2)s1] |

In one embodiment, provided herein is a pharmaceutical composition of comprising a compound of Table 1A, 1B, 1C and 1D, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.
In one embodiment, provided herein is a pharmaceutical combination of a compound of Table 1A, 1B, 1C and 1D, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

Pharmaceutical Compositions and Administration

General

In some embodiments, a chemical entity (e.g., a compound that modulates (e.g., antagonizes) NLRP1 or NLRP3 or both NLRP1 and NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, an NLRP3 antagonist and/or an anti-TNFα agent disclosed herein is administered as a pharmaceutical composition that includes the NLRP3 antagonist and/or anti-TNFα agent and one or more pharmaceutically acceptable excipients, and optionally one or more additional therapeutic agents as described herein. Preferably the pharmaceutical composition that includes an NLRP3 antagonist and an anti-TNFα agent.

Preferably the above pharmaceutical composition embodiments comprise an NLRP3 antagonist disclosed herein. More preferably the above pharmaceutical composition embodiments comprise an NLRP3 antagonist and an anti-TNFα agent disclosed herein.

In some embodiments, an NLRP3 antagonist and/or an anti-TNFα agent is administered as a pharmaceutical composition that includes the NLRP3 antagonist and/or anti-TNFα agent and one or more pharmaceutically acceptable excipients, and optionally one or more additional therapeutic agents as described herein.

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

Methods of Treatment

In some embodiments, methods for treating a subject having condition, disease or disorder in which a decrease or increase in NLRP1 or NLRP3 or both NLRP1 and NLRP3 activity (e.g., an increase, e.g., NLRP1/3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity; or is Type 2 Diabetes; or is NASH; or is Alzheimer's disease; or is gout; or is SLE; or is rheumatoid arthritis; or is IBD; or is multiple sclerosis; or is COPD; or is asthma; or

In some embodiments, the condition, disease or disorder is scleroderma; or is pulmonary fibrosis; or is age related macular degeneration (AMD); or is cystic fibrosis; or is Muckle Wells syndrome; or is familial cold autoinflammatory syndrome (FCAS); or is chronic neurologic cutaneous and articular syndrome; or In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis; or further from acute myeloid leukemia (AML) and chronic myeloid leukemia (CML).

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH; or is multiple myeloma; or is promyelocytic leukemia; or is gastric cancer; or

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20

In some embodiments, the methods described herein further include the step of identifying a subject (e.g., a patient) in need of treatment for an indication related to NLRP1 activity, such as an indication related NLRP1 polymorphism.

In some embodiments, the methods described herein further include the step of identifying a subject (e.g., a patient) in need of treatment for an indication related to NLRP1 activity, such as an indication related to NLRP1 where polymorphism is a gain of function

In some embodiments, the methods described herein further include the step of identifying a subject (e.g., a patient) in need of treatment for an indication related to NLRP1 activity, such as an indication related NLRP1 polymorphism found in vitiligo Vitiligo-Associated Autoimmune Disease.

In some embodiments, the methods described herein further include the step of identifying a subject (e.g., a patient) in need of treatment for an indication related to NLRP1 activity, such as an indication related where NLRP1 polymorphism is VAR_033239 (L155H)

In some embodiments, the methods described herein further include the step of identifying a subject (e.g., a patient) in need of treatment for an indication related to NLRP1 activity, such as an indication related where NLRP1 polymorphism is a natural variant reported in http://www.uniprot.org/uniprot/Q9C₀₀₀

In some embodiments, the methods described herein further include the step of identifying a subject (e.g., a patient) in need of treatment for an indication related to NLRP1/3 activity, such as an indication related to point mutation of NLRP1/3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibitreduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (INK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSFlA associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), INK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, INK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, INK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)₂, a minibody, or a BiTE.

In some embodiments, an antibody can be a crossmab, a diabody, a scDiabody, a scDiabody-CH₃, a Diabody-CH₃, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)—IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH₃ KIH, a Fab-scFv, a scFv-CH-CL-scFv, a F(ab′)₂-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFvl-PEG-scFv2.

Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or IYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, or less than 1×10⁻¹² M), e.g., as measured in phosphate buffered saline using surface plasmon resonance (SPR).

In some embodiments, any of the antibodies or antigen-binding fragments described herein has a K_{D} of about 1×10⁻² M to about 1×10⁻⁵ M, about 0.5×10⁻¹¹ M to about 1×10⁻⁵ M; about 1×10⁻¹ M to about 1×10⁻⁵ M; about 0.5×10⁻¹⁰ M to about 1×10⁻⁵ M; about 1×10⁻¹⁰ M to about 1×10⁻⁵ M; about 0.5×10⁻⁹ M to about 1×10⁻⁵ M; about 1 about 0.5×10⁻⁸ M to about 1×10⁻⁵ M; about 1×10⁻⁸ M to about 1×10⁻⁵ M, or about 0.5×10⁻⁵ M to about 1×10⁻⁵ M (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is fully or partially complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., fully or partially complementary to all or a part of any one of the sequences presented in Table E).

TABLE E

| | mRNA GenBank accession |
|---|---|
| Human gene | number(s) |
| Tumor necrosis factor (TNF, a.k.a. TNF- | NM_000594 |
| alpha) | |
| TNF receptor superfamily member 1A | NM_001065 |
| (TNFRSF1A) (a.k.a. TNFR1) | NM_001346091 |
| | NM_001346092 |
| TNF receptor superfamily member 1B | NM_001066 |
| (TNFRSF1B) (a.k.a. TNFR2) | XM_011542060 |
| | XM_011542063 |
| | XM_017002214 |
| | XM_017002215 |
| | XM_017002211 |
| TNFRSF1A associated via death domain | NM_003789 |
| (TRADD) | NM_001323552 |
| | XM_005256213 |
| | XM_017023815 |
| TNF receptor associated factor 2 (TRAF2) | NM_021138 |
| | XM_011518976 |
| | XM_011518977 |
| | XM_011518974 |
| JunD proto-oncogene, AP-1 transcription | NM_001286968 |
| factor subunit (JUND) | NM_005354 |
| Mitogen-activated protein kinase kinase | NM_005923 |
| kinase 5 (MAP3K5) (a.k.a. ASK1) | XM_017010875 |
| | XM_017010872 |
| | XM_017010873 |
| | XM_017010877 |
| | XM_017010874 |
| | XM_017010871 |
| | XM_017010870 |
| | XM_017010876 |
| | XM_011535839 |
| CD14 | NM_000591 |
| | NM_001040021 |
| | NM_001174104 |
| | NM_001174105 |
| Mitogen-activated protein kinase 3 | NM_001040056 |
| (MAPK3) (a.k.a. ERK1) | NM_001109891 |
| | NM_002746 |
| Mitogen-activated protein kinase 1 | NM_002745 |
| (MAPK1) (a.k.a. ERK2) | NM_138957 |
| Inhibitor of nuclear factor kappa B kinase | NM_001190720 |
| subunit beta (IKBKB) | NM_001242778 |
| | NM_001556 |
| | XM_005273491 |
| | XM_005273496 |
| | XM_005273493 |
| | XM_005273498 |
| | XM_011544518 |
| | XM_005273492 |
| | XM_005273490 |
| | XM_005273494 |
| | 12XM_017013396 |
| | XM_011544521 |
| | XM_011544522 |
| | XM_005273495 |
| | XM_011544517 |
| | XM_011544520 |
| | XM_011544519 |
| NFKB inhibitor alpha (NFKBIA) | NM_020529 |
| Interleukin 1 receptor associated kinase 1 | NM_001025242 |
| (IRAK1) | NM_001025243 |
| | NM_001569 |
| | XM_005274668 |
| Mitogen-activated protein kinase 8 | NM_001278547 |
| (MAPK8) (a.k.a. JNK) | NM_001278548 |
| | NM_001323302 |
| | NM_001323320 |
| | NM_001323321 |
| | NM_001323322 |
| | NM_001323323 |
| | NM_001323324 |
| | NM_001323325 |
| | NM_001323326 |
| | NM_001323327 |
| | NM_001323328 |
| | NM_001323329 |
| | NM_001323330 |
| | NM_001323331 |
| | NM_139046 |
| | NM_139049 |
| | XM_024448079 |
| | XM_024448080 |
| Lipopolysaccharide binding protein (LBP) | NM_004139 |
| Mitogen-activated protein kinase kinase 1 | NM_002755 |
| (MAP2K1) (a.k.a. MEK1) | XM_017022411 |
| | XM_011521783 |
| | XM_017022412 |
| | XM_017022413 |
| Mitogen-activated protein kinase kinase 2 | NM_030662 |
| (MAP2K2) (a.k.a. MEK2) | XM_006722799 |
| | XM_017026990 |
| | XM_017026989 |
| | XM_017026991 |
| Mitogen-activated protein kinase kinase 3 | NM_001316332 |
| (MAP2K3) (a.k.a. MEK3) | NM_002756 |
| | NM_145109 |
| | XM_017024859 |
| | XM_005256723 |
| | XM_017024857 |
| | XM_011523959 |
| | XM_017024858 |
| | XM_011523958 |
| Mitogen-activated protein kinase kinase 6 | NM_001330450 |
| (MAP2K6) (a.k.a. MEK6) | NM_002758 |
| | XM_005257516 |
| | XM_011525027 |
| | XM_011525026 |
| | XM_006721975 |
| Mitogen-activated protein kinase kinase | NM_005921 |
| kinase 1 (MAP3K1) (a.k.a. MEKK1) | XM_017009485 |
| | XM_017009484 |
| Mitogen-activated protein kinase kinase | NM_001330431 |
| kinase 3 (MAP3K3) (a.k.a. MEKK3) | NM_001363768 |
| | NM_002401 |
| | NM_203351 |
| | XM_005257378 |
| Mitogen-activated protein kinase kinase | NM_001291958 |
| kinase 4 (MAP3K4) (a.k.a. MEKK4) | NM_001301072 |
| | NM_001363582 |
| | NM_005922 |
| | NM_006724 |
| | XM_017010869 |
| Mitogen-activated protein kinase kinase | NM_001297609 |
| kinase 6 (MAP3K6) (a.k.a. MEKK6) | NM_004672 |
| | XM_017002771 |
| | XM_017002772 |
| Mitogen-activated protein kinase kinase | NM_003188 |
| kinase 7 (MAP3K7) (a.k.a. MEKK7) | NM_145331 |
| | NM_145332 |
| | NM_145333 |
| | XM_006715553 |
| | XM_017011226 |
| MAPK activated protein kinase 2 | NM_004759 |
| (MAPKAPK2) (a.k.a. MK2) | NM_032960 |
| | XM_005273353 |
| | XM_017002810 |
| MYD88, innate immune signal transduction | NM_001172566 |
| adaptor (MYD88) | NM_001172567 |
| | NM_001172568 |
| | NM_001172569 |
| | NM_001365876 |
| | NM_001365877 |
| | NM_002468 |
| Nuclear factor kappa B subunit 1 (NFKB1) | NM_001165412 |
| | NM_001319226 |
| | NM_003998 |
| | XM_024454069 |
| | XM_024454067 |
| | XM_011532006 |
| | XM_024454068 |
| Mitogen-activated protein kinase kinase | NM_003954 |
| kinase 14 (MAP3K14) (a.k.a. NIK) | XM_011525441 |
| Mitogen-activated protein kinase 14 | NM_001315 |
| (MAPK14) (a.k.a. p38) | NM_139012 |
| | NM_139013 |
| | NM_139014 |
| | XM_011514310 |
| | XM_017010300 |
| | XM_017010299 |
| | XM_017010301 |
| | XM_017010304 |
| | XM_017010303 |
| | XM_017010302 |
| | XM_006714998 |
| Eukaryotic translation initiation factor 2 | NM_001135651 |
| alpha kinase 2 (EIF2AK2) (a.k.a. PKR) | NM_001135652 |
| | NM_002759 |
| | XM_011532987 |
| | XM_017004503 |
| AKT serine/threonine kinase 1 (AKT1) | NM_001014431 |
| (a.k.a. RAC) | NM_001014432 |
| | NM_005163 |
| Zinc fingers and homeoboxes 2 (ZHX2) | NM_001362797 |
| (a.k.a. RAF) | NM_014943 |
| | XM_011516932 |
| | XM_005250836 |
| KRAS proto-oncogene, GTPase (KRAS) | NM_001369786 |
| | NM_001369787 |
| | NM_004985 |
| | NM_033360 |
| NRAS proto-oncogene, GTPase (NRAS) | NM_002524 |
| Receptor interacting serine/threonine kinase | NM_001317061 |
| 1 (RIPK1) (a.k.a. RIP) | NM_001354930 |
| | NM_001354931 |
| | NM_001354932 |
| | NM_001354933 |
| | NM_001354934 |
| | NM_003804 |
| | XM_017011405 |
| | XM_006715237 |
| | XM_017011403 |
| | XM_017011404 |
| TNF receptor associated factor 6 (TRAF6) | NM_004620 |
| | NM_145803 |
| | XM_017018220 |
| ZFP36 ring finger protein (ZFP36) (a.k.a. | NM_003407 |
| TTP) | |

An antisense nucleic acid molecule can be fully or partially complementary to all or part of a non-coding region of the coding strand of a nucleotide sequence encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-cB, NIK, p38, PKR, rac, ras, raf, RIP, TNFα, TNFR1, TNFR2, TRADD, TRAF2, TRAF6, or TTP protein. Non-coding regions (5′ and 3′ untranslated regions) are the 5′ and 3′ sequences that flank the coding region in a gene and are not translated into amino acids.

Based upon the sequences disclosed herein (e.g., in Table E), one of skill in the art can easily choose and synthesize any of a number of appropriate antisense nucleic acids to target a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-cB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-cB, NIK, p38, PKR, rac, ras, raf, RIP, TNFα, TNFR1, TNFR2, TRADD, TRAF2, TRAF6, or TTP protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an u-anomeric nucleic acid molecule. An u-anomeric nucleic acid molecule forms specific double-stranded hybrids with complementary RNA in which, contrary to the usual, -units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of sequences presented in Table E). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein (e.g., in Table E). For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C₈₇ (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, INK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, RO5126766 (CH5126766), PLX7904, and MLN2480).

In some examples, the anti-TNFα agent TNFα inhibitor is a small molecule that inhibits the activity of one of MK2 (PF 3644022 and PHA 767491), INK (e.g., AEG 3482, BI 78D3, CEP 1347, c-JUN peptide, IQ iS, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15nucleotides to about 40 nucleotides, about 15nucleotides to about 35 nucleotides, about 15 nucleotides to about 30 nucleotides, about 15nucleotides to about 28 nucleotides, about 15nucleotides to about 26 nucleotides, about 15nucleotides to about 25 nucleotides, about 15nucleotides to about 24 nucleotides, about 15nucleotides to about 22 nucleotides, about 15nucleotides to about 20 nucleotides, about 15nucleotides to about 18 nucleotides, about 15nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds herein may be prepared, for example, as shown in Scheme 1.

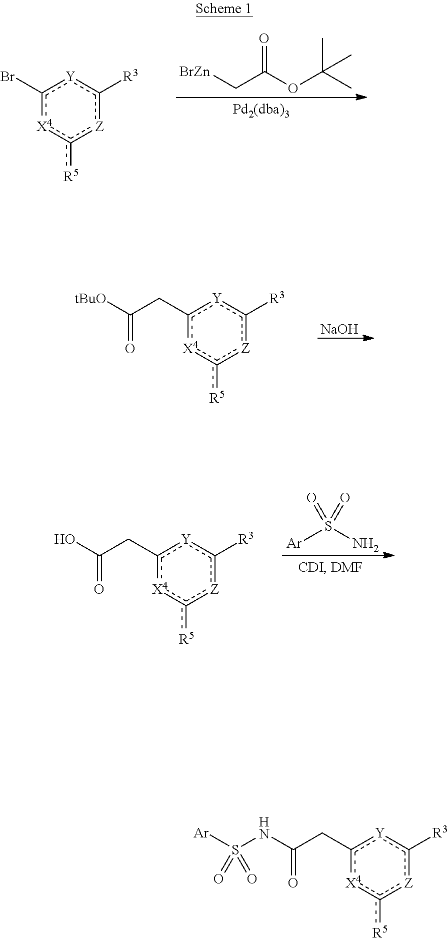

### PREPARATIVE EXAMPLES

The following abbreviations have the indicated meanings:
ACN=acetonitrile
AcOH=acetic acid
BINAP=(=)-2,2′-bis(diphenylphosphino)-1,1-binaphthyl
CDI=carbonyldiimidazole
DBU=1,8-diazabicycloundec-7-ene
DCM=dichloromethane
Dess-Martin=(1,1,1-triacetoxy)-1,1-dihydro-1,2-benziodoxol-3(1H)-one

### DIEA=N,N-diisopropylethylamine

### DMAP=4-(dimethylamino)pyridine

DMEDA=N,N′-dimethylethylenediamine

### DMF=N,N-dimethylformarnide

EDCI=N-(3-dimethylamninopropyl)-N′-ethylcarbodiinide hydrochloride
Et=ethyl
EtOH=ethanol
HATU=O-(7-azabenzotriazol-1-yl)-N,N,N′,N′-tetramethyluronium hexafluorophosphate
HBTU=O-benzotriazole-N,N,N′,N′-tetramethyluroniun-hexafliorophosphate
HOBt=l-hydroxybenzotrizole
LC-MS=liquid chromatography—mass spectrometry
LiHMDS=lithium bis(trimethylsilyl)amid
Me=methyl
MeOH=methanol

### NBS=N-bromosuccinimide

### NCS=N-chlorosuccinimide

NMR=nuclear magnetic resonance
Pd(dppf)Cl₂=dichloro[1,1′-bis(diphenylphosphino)ferrocene]palladium
Pd₂(dba)₃=tris(dibenzylideneacetone)dipalladium
Ph=phenyl
HPLC=high performance liquid chromatography
Py=pyridine
RT=room temperature
TBAF=tetrabutylammonium fluoride
TBDMSCI=tert-butyldimethylsilyl chloride
TBDPSCi=tert-butyldiphenylsilyl chloride
TEA triethylamine
TFA=trifluoroacetic acid
THF=tetrahydrofuran
Ti(i-PrO)₄=tetraisopropyl titanate
TLC=thin layer chromatography
TsOH=p-toluenesulfonicacidmonohydrate
X-phos=2-(Dicyclohexylphosphino)-2′,4′,6′-triisopropylbiphenyl
The progress of reactions was often monitored by TLC or LC-MS. The idenrity of the products was often confirmed by LC-MS The LC-MS was recorded using one of the following methods.
Method A: Shim-pack XR-ODS, C18, 3×50 mm, 2.5 urn column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.
Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with ACN and water (0.5% NH₄HCO₃), 2 minute total run time.
Method C: Shim-pack XR-ODS, C18, 3×50 mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time.
Method D: Kinetex EVO, C18, 3×50 mm, 2.2 um column, 1.0 uL injection, 1.5 mL/min flow rate, 90-900 amu scan range, 190-400 nm LV range, 10-95% (2.1 min), 95% (0.6 min) gradient with ACN and water (0.5% NH₄HCO₃), 3 minute total run time.
The final targets were purified by Prep-HPLC, The Prep-HPLC was carried out using the following method.
Method E: Pre-HPLC: Column, XBridge Shield RP18 OBD (19×250 mm, 10 um); mobile phase, Water (10 mmol/L NH₄HCO₃) and ACN, UV detection 254/210 nm.
NMR was recorded on BRUKER NMR 300.03 Mz, DUL-C-H, ULTRASHIELD™300, AVANCE II 300 B-ACS™120 or BRUKER NMR 400.13 Mz, BBFO, ULTRASHIELD™400, AVANCE III 400, B-ACS™120.
Scheme of final targets: Schemes A-E illustrate several conditions used for coupling of acid 1 and sulfonamide 2 to afford acyl sulfonamide 3.

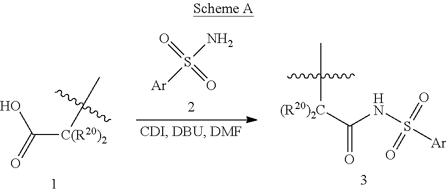

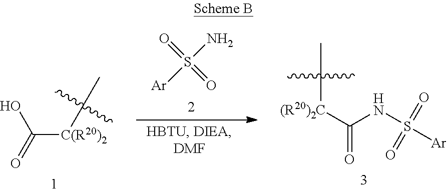

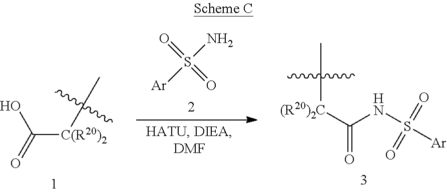

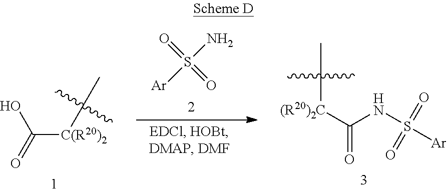

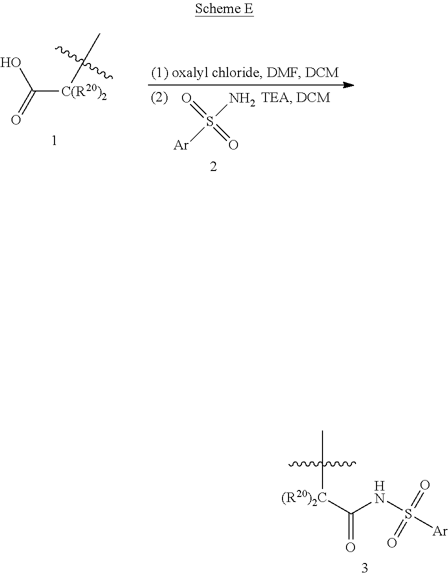

Scheme of Sulfonamides Intermediates: Schemes F-Z illustrate the preparation of sulfonamide intermediates. It is understood that the numbering used in the schemes below refers only to the intermediates and that the intermediates are distinct from compounds of formula A, I, and/or II. that may have the same numerical designation. Thus, by way of example, intermediate number “101” in Scheme AE below—that is, the compound

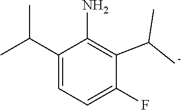

is distinct from compound 101 disclosed herein, that is,

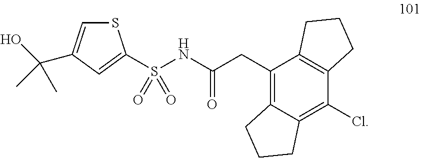 [SMILES: CC(C)(O)c1csc(S(=O)(=O)NC(=O)Cc2c3c(c(Cl)c4c2CCC4)CCC3)c1]

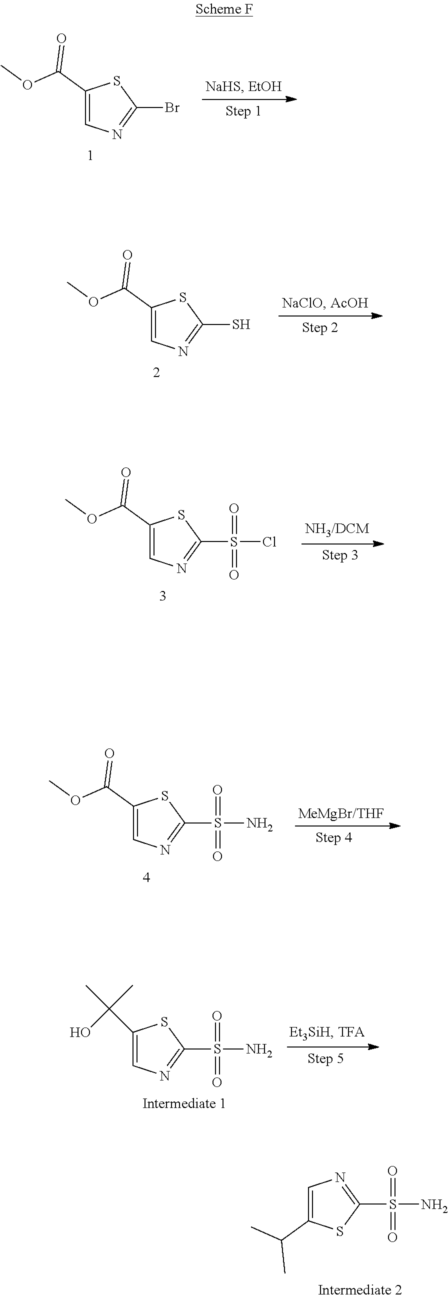

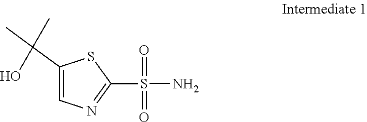

### 5-(2-Hydroxypropan-2-yl)thiazole-2-sulfonamide

### Step 1: Methyl 2-mercaptothiazole-5-carboxylate

Into a 250-mL round-bottom flask, was placed methyl 2-bromothiazole-5-carboxylate (10 g, 45 mmol), EtOH (100 mL), sodium hydrogensulfide (5 g, 89 mmol). The resulting solution was stirred for 2 h at 80° C. and then was cooled to 0° C. with a water/ice bath. The pH value of the solution was adjusted to 3 with hydrogen chloride (1 N). The solids were collected by filtration. This resulted in 6 g (76%) of the title compound as a light yellow solid. MS-ESI: 176.0 (M+1).

### Step 2: Methyl 2-(chlorosulfonyl)thiazole-5-carboxylate

Into a 250-mL round-bottom flask, was placed methyl 2-mercaptothiazole-5-carboxylate (6 g, 34 mmol), acetic acid (60 mL). This was followed by the addition of sodium hypochloride (60 mL, 8%-10% wt) in portions at 0° C. The resulting solution was stirred for 1 h at RT and then was diluted with 100 mL of water. The solution was extracted with 3×50 mL of DCM and the organic layers combined and dried over anhydrous Na₂SO₄, then concentrated under vacuum. This resulted in 5 g (crude, 60%) of the title compound as yellow oil. The crude product was used in the next step.

### Step 3: Methyl 2-sulfamoylthiazole-5-carboxylate

Into a 250-mL round-bottom flask, was placed methyl 2-(chlorosulfonyl)thiazole-5-carboxylate (5 g, 21 mmol), DCM (50 mL). This was followed by the addition of a saturated solution of ammonia in DCM (10 mL) in portions at RT. The resulting solution was stirred for 2 h at RT and then was concentrated under vacuum. The residue was applied onto a silica gel column and eluted with ethyl acetate/petroleum ether (1:5 to 1:3). This resulted in 3 g (65%) of the title compound as a white solid. MS-ESI: 223.0 (M+1).

### Step 4: 5-(2-Hydroxypropan-2-yl)thiazole-2-sulfonamide

Into a 250-mL round-bottom flask purged with and maintained under nitrogen, was placed a solution of methyl 2-sulfamoylthiazole-5-carboxylate (3 g, 13.5 mmol) in THF (25 mL). This was followed by the addition of MeMgBr/THF (3 M, 18 mL) dropwise with stirring at 0° C. The resulting solution was stirred for 14 h at RT and then was quenched by the addition of 20 mL of NH₄Cl (sat.). The resulting solution was extracted with 3×30 mL of DCM and the organic layers combined and dried over anhydrous Na₂SO₄, then concentrated under vacuum. The residue was applied onto a silica gel column and eluted with ethyl acetate/petroleum ether (1:5 to 1:3). This resulted in 2.3 g (78%) of the title compound as a white solid. MS-ESI: 223.0 (M+1), 221.0 (M-1).

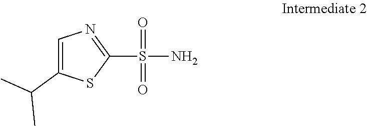

### 5-Isopropylthiazole-2-sulfonamide

### Step 5: 5-Isopropylthiazole-2-sulfonamide

Into a 40-mL sealed tube, was placed 5-(2-hydroxypropan-2-yl)thiazole-2-sulfonamide (500 mg, 2.25 mmol) in TFA (5 mL), Et₃SiH (5 mL). The resulting solution was stirred for 4 h at 70° C. and then was concentrated under vacuum. The residue was applied onto a silica gel column and eluted with ethyl acetate/petroleum ether (1:4 to 1:2). This resulted in 380 mg (82%) of the title compound as a yellow solid. MS-ESI: 205.0 (M-1).

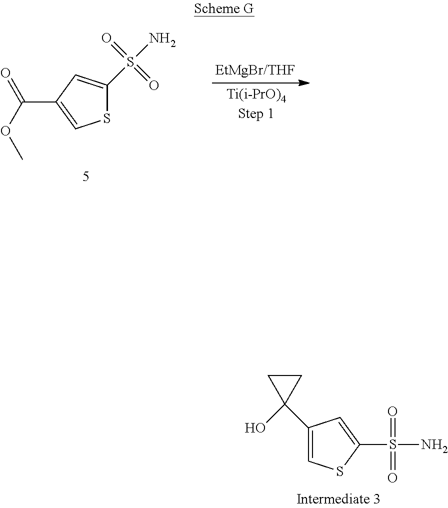

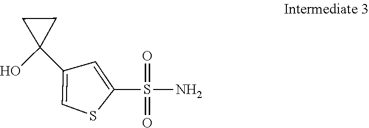 [SMILES: NS(=O)(=O)c1cc(C2(O)CC2)cs1]

### 4-(1-Hydroxvcyclopropyl)thiophene-2-sulfonamide

### Step 1: 4-(1-Hydroxycyclopropyl)thiophene-2-sulfonamide

Into a 500-mL 3-necked round-bottom flask purged with and maintained under nitrogen, was placed methyl 5-sulfamoylthiophene-3-carboxylate (5.525 g, 24.97 mmol), THF (80 mL), Ti(i-PrO)₄ (1.5 mL). This was followed by the addition of EtMgBr/THF (3 M, 21 mL) dropwise with stirring at 0° C. The resulting solution was stirred for 2 h at RT and then was quenched by the addition of 30 mL of NH₄Cl (sat.). The resulting solution was extracted with 3×40 mL of ethyl acetate and the organic layers combined and concentrated under vacuum. The residue was applied onto a silica gel column and eluted with ethyl acetate/petroleum ether (1:3 to 1:1). This resulted in 662 mg (12%) of the title compound as a light yellow solid. MS-ESI: 218.0 (M-1).

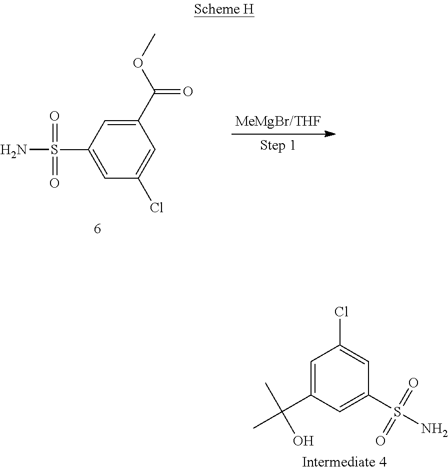

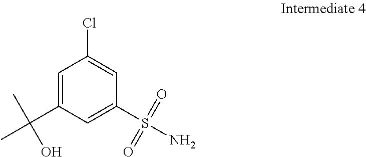

### 3-Chloro-5-(2-hydroxypropan-2-vl)benzenesulfonamide

### Step 1: 3-Chloro-5-(2-hydroxypropan-2-yl)benzenesulfonamide

Into a 100-mL 3-necked round-bottom flask purged with and maintained under nitrogen, was placed a solution of methyl 3-chloro-5-sulfamoylbenzoate (579 mg, 2.32 mmol) in THF (30 mL). This was followed by the addition of MeMgBr/THF (3 M, 3.5 mL) dropwise with stirring at 0° C. The resulting solution was stirred for 12 h at RT and then was quenched by the addition of 20 mL of NH₄Cl (sat.). The solution was extracted with 3×20 mL of ethyl acetate and the organic layers combined and concentrated under vacuum. The residue was applied onto a silica gel column and eluted with ethyl acetate/petroleum ether (1:3 to 1:1). This resulted in 415 mg (72%) of the title compound as a light yellow solid. MS-ESI: 248.0, 250.0 (M-1).

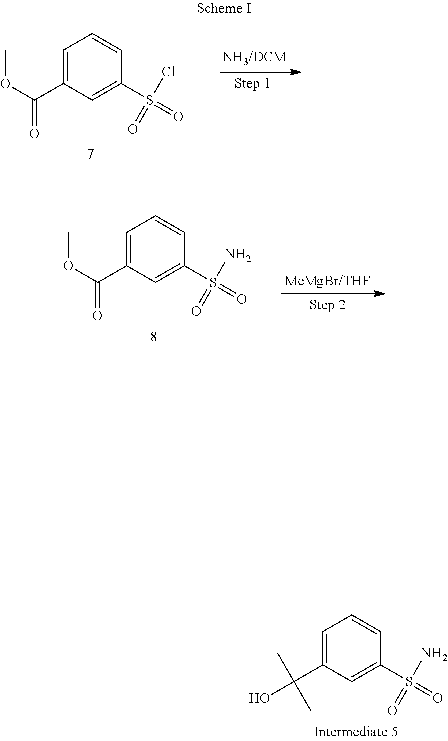

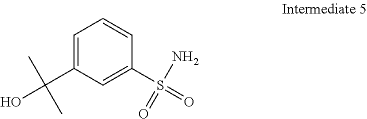

### 3-(2-Hydroxypropan-2-yl)benzenesulfonamide

### Step 1: Methyl 3-sulfamoylbenzoate

Into a 100-mL round-bottom flask, was placed a solution of methyl 3-(chlorosulfonyl)benzoate (2 g, 8.5 mmol) in DCM (35 mL). To the above was added a saturated solution of ammonia in DCM (15 mL). The resulting solution was stirred for 2 h at RT and then was concentrated under vacuum. The residue was applied onto a silica gel column and eluted with ethyl acetate/petroleum ether (1:3 to 1:1). This resulted in 1.753 g (93%) of the title compound as a white solid. MS-ESI: 214.0 (M-1).

### Step 2: 3-(2-Hydroxypropan-2-yl)benzenesulfonamide

Into a 250-mL 3-necked round-bottom flask purged with and maintained under nitrogen, was placed a solution of methyl 3-sulfamoylbenzoate (1.753 g, 8.14 mmol) in THF (70 mL). This was followed by the addition of MeMgBr/THF (3 M, 12.2 mL) dropwise with stirring at 0° C. The resulting solution was stirred for 12 h at RT and then was quenched by the addition of 30 mL of NH₄Cl (sat.). The resulting solution was extracted with 5×30 mL of ethyl acetate and the organic layers combined and concentrated under vacuum. The residue was applied onto a silica gel column and eluted with ethyl acetate/petroleum ether (1:3 to 1:1). This resulted in 1.642 g (94%) of the title compound as a white solid. MS-ESI: 214.0 (M-1).

TABLE 2
The Intermediates in the following Table were prepared using the similar procedure for
converting compound 7 to compound 8 shown in Scheme I.

| Intermediate # | Structure | IUPAC Name | Mass Spec[M + H]⁺ |
|---|---|---|---|
| Intermediate 6 | 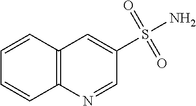 [SMILES: NS(=O)(=O)c1cnc2ccccc2c1] | quinoline-3- sulfonamide | 209.0 (M + 1) |
| Intermediate 7 | 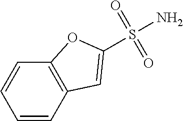 [SMILES: NS(=O)(=O)c1cc2ccccc2o1] | benzofuran-2- sulfonamide | 196.0 (M − 1) |

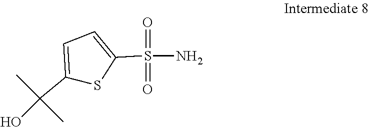

### 5-(2-Hydroxypropan-2-yl)thiophene-2-sulfonamide

Intermediate 8 was prepared using the similar procedures for converting compound 7 to Intermediate 5 shown in Scheme I MS-ESI 220.0 (M-1).

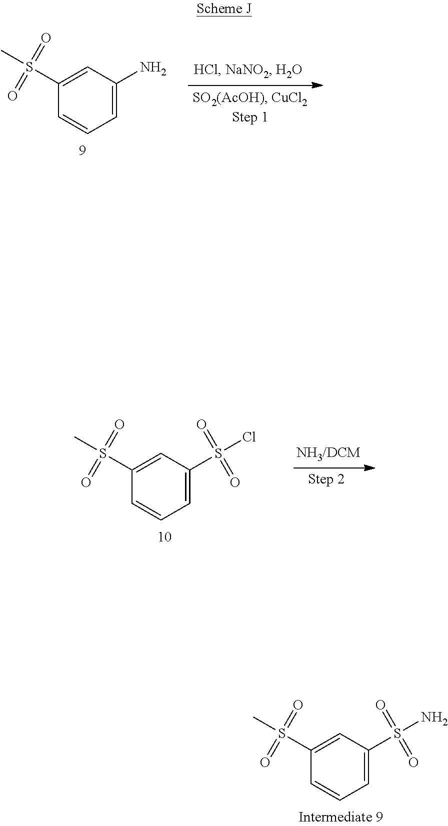

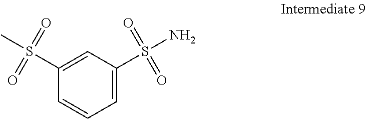

### 3-(Methylsulfonyl)benzenesulfonamide

### Step 1: 3-(Methylsulfonyl)benzene-1-sulfonyl chloride

Into a 100-mL round-bottom flask, was placed a solution of 3-(methylsulfonyl)benzenamine (200 mg, 1.17 mmol) in HCl (6 M, 5 mL). This was followed by the addition of a solution of NaNO₂ (97 mg, 1.41 mmol) in water (0.5 mL) dropwise with stirring at 0° C. The resulting solution was stirred for 20 min at 0° C. The above mixture was added to a saturated solution of SO₂ in AcOH (5 mL) dropwise with stirring at 0° C. Then to the above was added CuCl₂ (157 mg, 1.17 mmol). The resulting solution was stirred for 1 h at RT and then was quenched by the addition of 10 mL of water. The resulting solution was extracted with 3×10 mL of DCM and the organic layers combined and dried over anhydrous Na₂SO₄, then concentrated under vacuum. This resulted in 250 mg (84%) of the title compound as a light yellow solid. The crude product was used in the next step.

### Step 2: 3-(Methylsulfonyl)benzenesulfonamide

Into a 50-mL round-bottom flask, was placed 3-(methylsulfonyl)benzene-1-sulfonyl chloride (250 mg, 0.98 mmol), DCM (3 mL). To the above was added a saturated solution of ammonia in DCM (5 mL). The resulting solution was stirred for 1 h at RT and then was diluted with 5 mL of water. The resulting solution was extracted with 3×10 mL of ethyl acetate and the organic layers combined and dried over anhydrous Na₂SO₄, then concentrated under vacuum. This resulted in 220 mg (crude, 95%) of the title compound as a white solid. MS-ESI: 234.0 (M-1).

TABLE 3
The Intermediates in the following Table were prepared using the similar procedures
for converting compound 9 to Intermediate 9 shown in Scheme J.

| | | | Mass |
|---|---|---|---|
| Intermediate # | Structure | IUPAC Name | Spec[M − H]⁻ |
| Intermediate 10 | 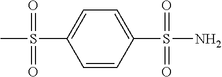 | (methylsulfonyl) benzenesulfonamide | 234.0 |
| Intermediate 11 | 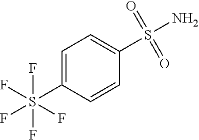 [SMILES: NS(=O)(=O)c1ccc(S(F)(F)(F)(F)F)cc1] | 4- pentafluorobenzenesulfonamide | 282.0 |
| Intermediate 12 | 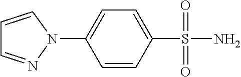 [SMILES: NS(=O)(=O)c1ccc(-n2cccn2)cc1] | 4-(1H-pyrazol-1-yl) benzenesulfonamide | 222.0 |

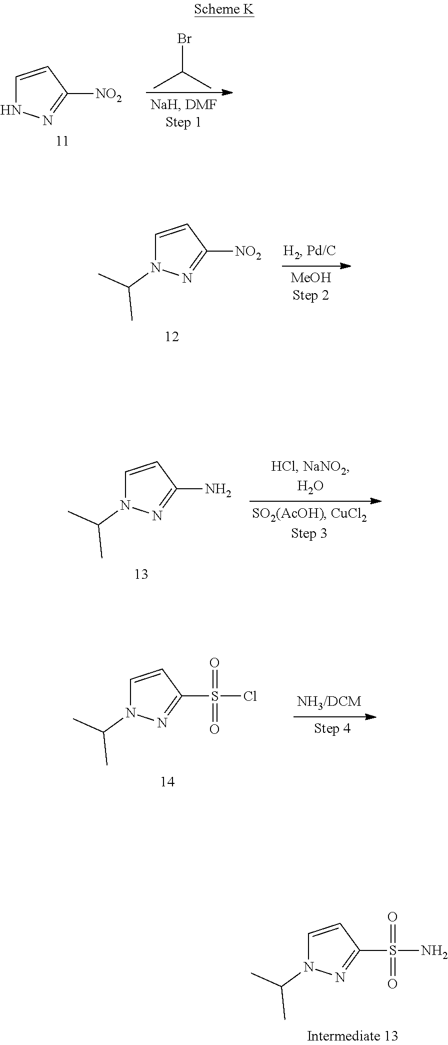

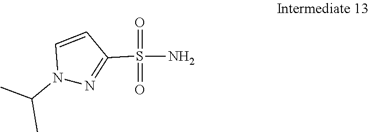

### 1-Isopropyl-1H-pyrazole-3-sulfonamide

### Step 1: 1-Isopropyl-3-nitro-1H-pyrazole

Into a 250-mL round-bottom flask, was placed a solution of 3-nitro-1H-pyrazole (10 g, 88.4 mmol) in DMF (100 mL). This was followed by the addition of NaH (60%, 3.9 g) in portions at 0° C. The resulting solution was stirred for 0.5 h at 0° C. This was followed by the addition of 2-bromopropane (14.1 g, 114.6 mmol) dropwise with stirring at 0° C. in 10 min. The resulting solution was stirred for 16 h at RT and then was quenched by the addition of 100 mL of water. The resulting solution was extracted with 3×100 mL of ethyl acetate and the organic layers combined and dried over anhydrous Na₂SO₄, then concentrated under vacuum. The residue was applied onto a silica gel column and eluted with ethyl acetate/petroleum ether (1:5 to 1:3). This resulted in 11.8 g (86%) of the title compound as yellow oil. MS-ESI: 156.1 (M+1).

### Step 2: 3-Amino-i-(propan-2-yl)-1H-pyrazole

Into a 250-mL round-bottom flask, was placed a solution of 1-isopropyl-3-nitro-1H-pyrazole (10.8 g, 69.6 mmol) in MeOH (100 mL). Then Pd/C (10% wt, 1.5 g) was added. The flask was evacuated and flushed three times with hydrogen. The mixture was stirred for 24 h at RT under an atmosphere of hydrogen. The solids were filtered out. The resulting mixture was concentrated under vacuum. This resulted in 7.27 g (83%) of the title compound as yellow oil. MS-ESI: 126.1 (M+1).

Steps 3-4 used similar procedures for converting compound 9 to Intermediate 9 shown in Scheme J to afford Intermediate 13. MS-ESI: 188.0 (M-1).

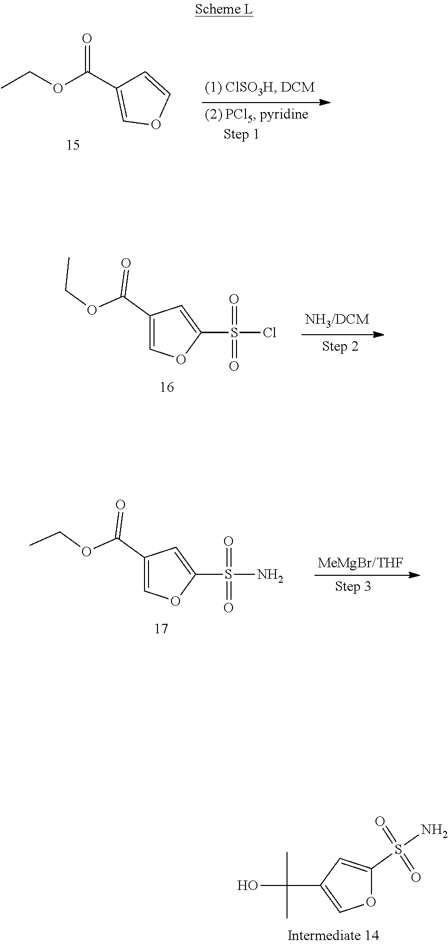

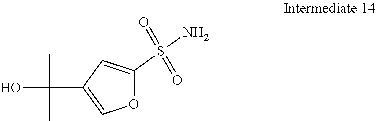

### 4-(2-Hydroxypropan-2-yl)furan-2-sulfonamide

### Step 1: Ethyl 5-(chlorosulfonyl)furan-3-carboxylate

Into a 500-mL 3-necked round-bottom flask, was placed ethyl furan-3-carboxylate (7 g, 50 mmol), DCM (200 mL). This was followed by the addition of chloranesulfonic acid (5.8 g, 49.8 mmol) dropwise with stirring at −10° C. Then the reaction was stirred for 48 h at RT and the system was cooled to −10° C. Then to the above was added pyridine (3.96 g, 50.1 mmol), phosphorus pentachloride (11.46 g, 55.0 mmol). The resulting solution was stirred for 12 h at RT and then was quenched by the addition of 200 mL of water. The resulting solution was extracted with 3×200 mL of DCM and the organic layers combined and dried over anhydrous Na₂SO₄, then concentrated under vacuum. This resulted in 7.13 g (60%) of the title compound as light brown oil. The crude product was used in the next step.

### Step 2: Ethyl 5-sulfamoylfuran-3-carboxylate

Into a 250-mL round-bottom flask, was placed a solution of ethyl 5-(chlorosulfonyl)furan-3-carboxylate (6.111 g, 25.61 mmol) in DCM (60 mL). To the above was added a saturated solution of ammonia in DCM (40 mL). The resulting solution was stirred for 3 h at RT and then was concentrated under vacuum. The residue was applied onto a silica gel column and eluted with ethyl acetate/petroleum ether (1:4 to 1:2). This resulted in 3.698 g (66%) of the title compound as a light yellow solid. MS-ESI: 218.0 (M-1).

### Step 3: 4-(2-Hydroxypropan-2-yl)furan-2-sulfonamide

Into a 250-mL 3-necked round-bottom flask purged with and maintained under nitrogen, was placed a solution of ethyl 5-sulfamoylfuran-3-carboxylate (3.698 g, 16.87 mmol) in THF (100 mL). This was followed by the addition of MeMgBr/THF (3 M, 25 mL) dropwise with stirring at −10° C. The resulting solution was stirred for 10 h at RT and then was quenched by the addition of 50 mL of NH₄Cl (sat.). The resulting solution was extracted with 3×50 mL of ethyl acetate and the organic layers combined and dried over anhydrous Na₂SO₄, then concentrated under vacuum. The residue was applied onto a silica gel column and eluted with ethyl acetate/petroleum ether (1:3 to 1:1). This resulted in 2.6 g (75%) of the title compound as a light yellow solid. MS-ESI: 204.0 (M-1).

TABLE 4
The Intermediates in the following Table were prepared using the similar procedures
for converting compound 15 to Intermediate 14 shown in Scheme L.

| Intermediate # | Structure | IUPAC Name | Mass Spec[M − H]⁻ |
|---|---|---|---|
| Intermediate 15 | 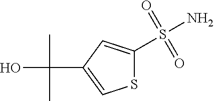 | 4-(2-hydroxypropan-2-yl) thiophene-2-sulfonamide | 220.0 |
| Intermediate 16 | 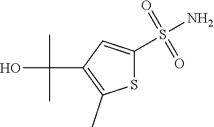 | 4-(2-hydroxypropan-2-yl)-5- methylthiophene-2- sulfonamide | 234.0 |
| Intermediate 17 | 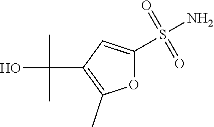 | 4-(2-hydroxypropan-2-yl)-5- methylfuran-2-sulfonamide | 218.1 |
| Intermediate 18 | 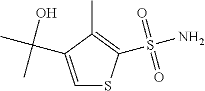 | 4-(2-hydroxypropan-2-yl)-3- methylthiophene-2- sulfonamide | 234.1 |

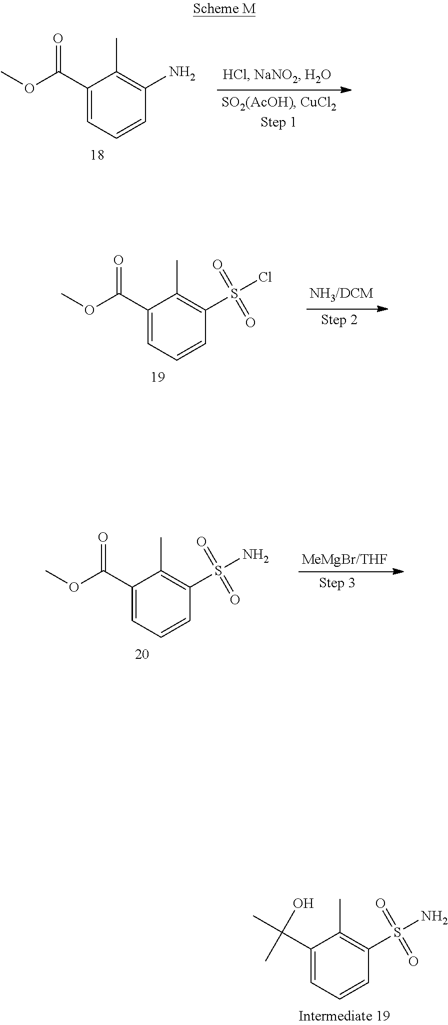

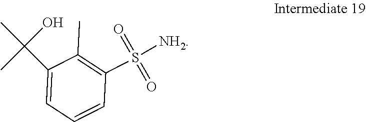

### 3-(2-Hydroxypropan-2-yl)-2-methylbenzenesulfonamide

### Step 1: Methyl 3-(chlorosulfonyl)-2-methylbenzoate

Into a 100-mL round-bottom flask, was placed methyl methyl 3-amino-2-methylbenzoate (2 g, 12.1 mmol), HCl (6 M, 10 mL). This was followed by the addition of a solution of NaNO2 (1 g, 14.5 mmol) in water (5 mL) dropwise with stirring at 0° C. The resulting solution was stirred for 20 min at 0° C. The above mixture was added to a saturated solution of SO₂ in AcOH (15 mL) dropwise with stirring at 0° C. Then to the above was added CuCl₂ (1.63 g, 12.1 mmol). The resulting solution was stirred for 1 h at RT and then was quenched by the addition of 15 mL of water. The resulting solution was extracted with 2×20 mL of DCM and the organic layers combined and dried over anhydrous Na₂SO₄, then concentrated under vacuum. This resulted in 2 g (66%) of the title compound as a light yellow solid. The crude product was used in the next step.

### Step 2: Methyl 2-methyl-3-sulfamoylbenzoate

Into a 100-mL round-bottom flask, was placed a solution of methyl 3-(chlorosulfonyl)-2-methylbenzoate (2 g, 8.04 mmol) in DCM (10 mL). To the above was added a saturated solution of ammonia in DCM (15 mL). The resulting solution was stirred for 1 h at RT and then was concentrated under vacuum. The residue was applied onto a silica gel column and eluted with ethyl acetate/petroleum ether (1:3 to 1:1). This resulted in 1.2 g (65%) of the title compound as a white solid. MS-ESI: 228.0 (M-1).

### Step 3: 3-(2-Hydroxypropan-2-yl)-2-methylbenzenesulfonamide

Into a 100-mL 3-necked round-bottom flask purged with and maintained under nitrogen, was placed a solution of methyl 2-methyl-3-sulfamoylbenzoate (1.2 g, 5.23 mmol) in THF (20 mL). This was followed by the addition MeMgBr/THF (3 M, 8.7 mL) dropwise with stirring at 0° C. The resulting solution was stirred for 12 h at RT. The reaction was then quenched by the addition of 15 mL of NH₄Cl (sat.). The resulting solution was extracted with 3×20 mL of ethyl acetate and the organic layers combined and dried over anhydrous Na₂SO₄, then concentrated under vacuum. This resulted in 1.1 g (crude, 92%) of the title compound as an off-white solid. MS-ESI: 228.1 (M-1).

TABLE 5
The Intermediates in the following Table were prepared using the similar procedures
for converting compound 18 to Intermediate 19 shown in Scheme M.

| Intermediate # | Structure | IUPAC Name | Mass Spec[M − H]⁻ |
|---|---|---|---|
| Intermediate 20 | 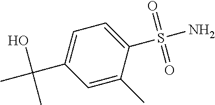 | 4-(2-hydroxypropan-2-yl)-2- methylbenzenesulfonamide | 228.1 |
| Intermediate 21 | 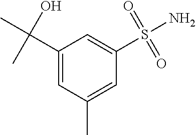 | 3-(2-hydroxypropan-2-yl)-5- methylbenzenesulfonamide | 228.1 |
| Intermediate 22 | 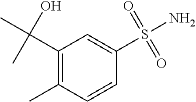 | 3-(2-hydroxypropan-2-yl)-4- methylbenzenesulfonamide | 228.1 |
| Intermediate 23 | 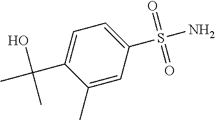 | 4-(2-hydroxypropan-2-yl)-3- methylbenzenesulfonamide | 228.1 |
| Intermediate 24 | 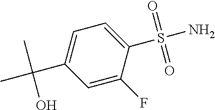 | 2-fluoro-4-(2- hydroxypropan-2- yl)benzenesulfonamide | 232.1 |
| Intermediate 25 | 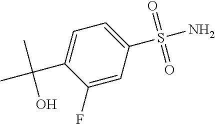 | 3-fluoro-4-(2- hydroxypropan-2- yl)benzenesulfonamide | 232.1 |
| Intermediate 26 | 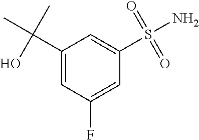 | 3-fluoro-5-(2- hydroxypropan-2- yl)benzenesulfonamide | 232.1 |
| Intermediate 27 | 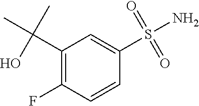 | 4-fluoro-3-(2- hydroxypropan-2- yl)benzenesulfonamide | 232.1 |
| Intermediate 28 | 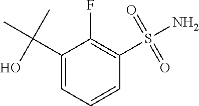 | 2-fluoro-3-(2- hydroxypropan-2- yl)benzenesulfonamide | 232.1 |
| Intermediate 29 | 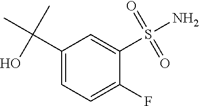 | 2-fluoro-5-(2- hydroxypropan-2- yl)benzenesulfonamide | 232.1 |
| Intermediate 30 | 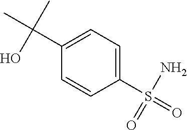 | 4-(2-hydroxypropan-2-yl) benzenesulfonamide | 214.1 |
| Intermediate 31 | 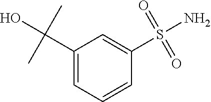 | 3-(2-hydroxypropan-2-yl) benzenesulfonamide | 214.1 |
| Intermediate 32 | 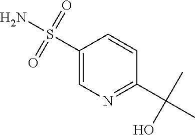 | 6-(2-hydroxypropan-2-yl) pyridine-3-sulfonamide | 217.1 (M + 1) |
| Intermediate 33 | 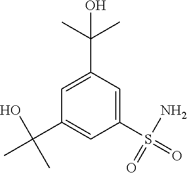 [SMILES: CC(C)(O)c1cc(C(C)(C)O)cc(S(N)(=O)=O)c1] | 3,5-bis(2-hydroxypropan-2- yl) benzenesulfonamide | 272.1 |

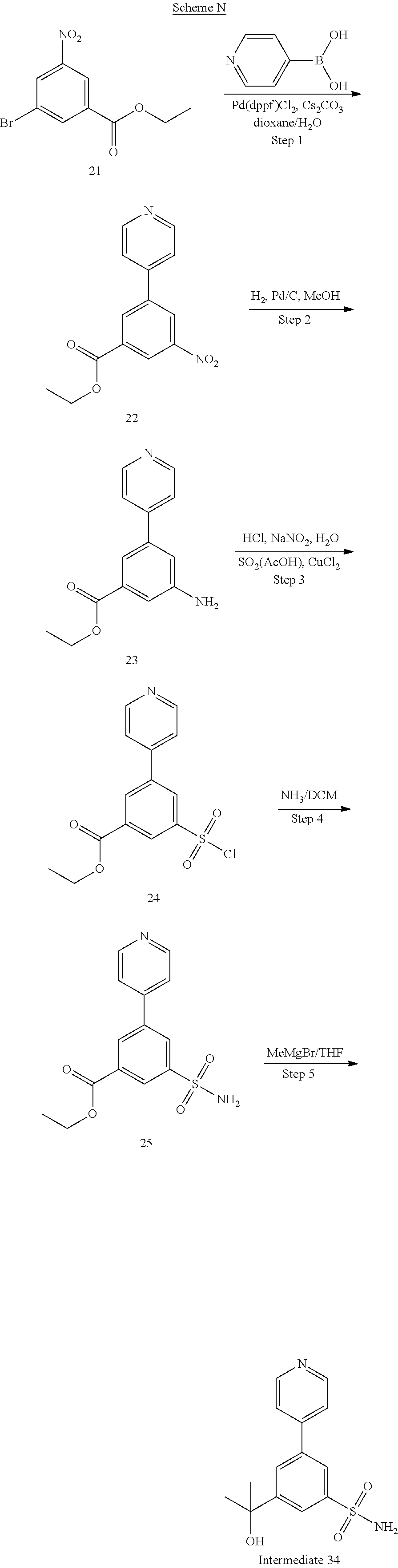

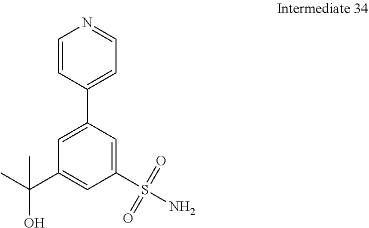 [SMILES: CC(C)(O)c1cc(-c2ccncc2)cc(S(N)(=O)=O)c1]

### 3-(2-Hydroxypropan-2-yl)-5-(pyridin-4-yl)benzenesulfonamide

### Step 1: Ethyl 3-nitro-5-(pyridin-4-yl)benzoate

Into a 500-mL round-bottom flask purged with and maintained under nitrogen, was placed ethyl 3-bromo-5-nitrobenzoate (5.5 g, 20.1 mmol), dioxane (250 mL), water(50 mL), (pyridin-4-yl)boronic acid (3.0 g, 24.4 mmol), Cs₂CO₃ (12.7 g, 38.98 mmol), Pd(dppf)Cl₂ (600 mg, 0.82 mmol). The resulting solution was stirred for 12 h at 90° C. and then was concentrated under vacuum. The residue was applied onto a silica gel column and eluted with ethyl acetate/petroleum ether (1:1 to 3:1). This resulted in 4.2 g (77%) of the title compound as a white solid. MS-ESI: 273.1 (M+1).

### Step 2: Ethyl 3-amino-5-(pyridin-4-yl)benzoate

Into a 250-mL round-bottom flask, was placed ethyl 3-nitro-5-(pyridin-4-yl)benzoate (4.2 g, 15.4 mmol), MeOH (150 mL). Then Pd/C (10% wt, 500 mg) was added. The flask was evacuated and flushed three times with hydrogen. The resulting solution was stirred for 2 days at RT under an atmosphere of hydrogen. The solids were filtered out. The resulting solution was concentrated under vacuum. This resulted in 3.7 g (99%) of the title compound as a white solid. MS-ESI: 243.1 (M+1).

Steps 3-5 used similar procedures for converting compound 18 to Intermediate 19 shown in Scheme M to afford Intermediate 34. MS-ESI: 293.1 (M+1), 291.1 (M-1).

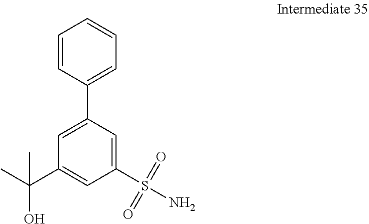 [SMILES: CC(C)(O)c1cc(-c2ccccc2)cc(S(N)(=O)=O)c1]

### 5-(2-Hydroxypropan-2-yl)biphenyl-3-sulfonamide

Intermediate 35 was prepared using the similar procedures for converting compound 21 to Intermediate 34 shown in Scheme N. MS-ESI: 290.1 (M-1)

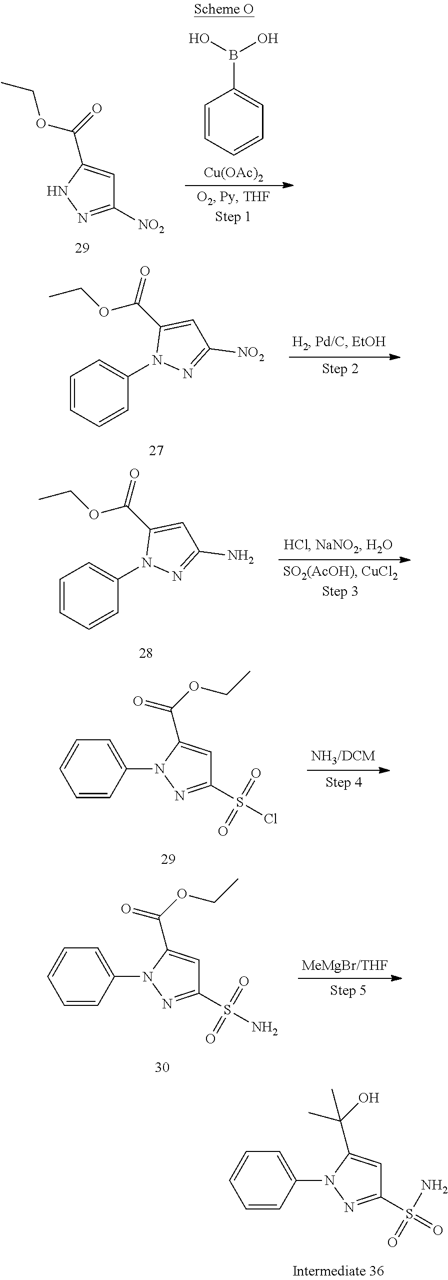

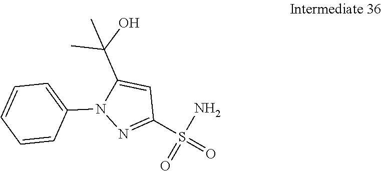 [SMILES: CC(C)(O)c1cc(S(N)(=O)=O)nn1-c1ccccc1]

### 5-(2-Hydroxypropan-2-yl)-1-phenyl-1H-pyrazole-3-sulfonamide

### Step 1: Ethyl 3-nitro-1-phenyl-1H-pyrazole-5-carboxylate

Into a 500-mL round-bottom flask, was placed ethyl 3-nitro-1H-pyrazole-5-carboxylate (5 g, 27.0 mmol), THF (150 mL), phenylboronic acid (6.59 g, 54.1 mmol), Cu(OAc)₂ (7.36 g, 40.5 mmol), pyridine (8.54 g, 108 mmol). The resulting solution was stirred for 14 h at 55° C. and then was concentrated under vacuum. The residue was applied onto a silica gel column and eluted with ethyl acetate/petroleum ether (1:7 to 1:4). This resulted in 2 g (28%) of the title compound as an off-white solid. MS-ESI: 262.1 (M+1).

### Step 2: Ethyl 3-amino-I-phenyl-1H-pyrazole-5-carboxylate

Into a 100-mL round-bottom flask, was placed ethyl 3-nitro-1-phenyl-1H-pyrazole-5-carboxylate (2 g, 7.66 mmol), EtOH (50 mL). Then Pd/C (10% wt, 200 mg) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:3 to 1:1). This resulted in 1 g (56%) of the title compound as a light yellow solid. MS-ESI: 232.1 (M+1).

Steps 3-5 used similar procedures for converting compound 18 to Intermediate 19 shown in Scheme M to afford Intermediate 36. MS-ESI: 280.1 (M-1).

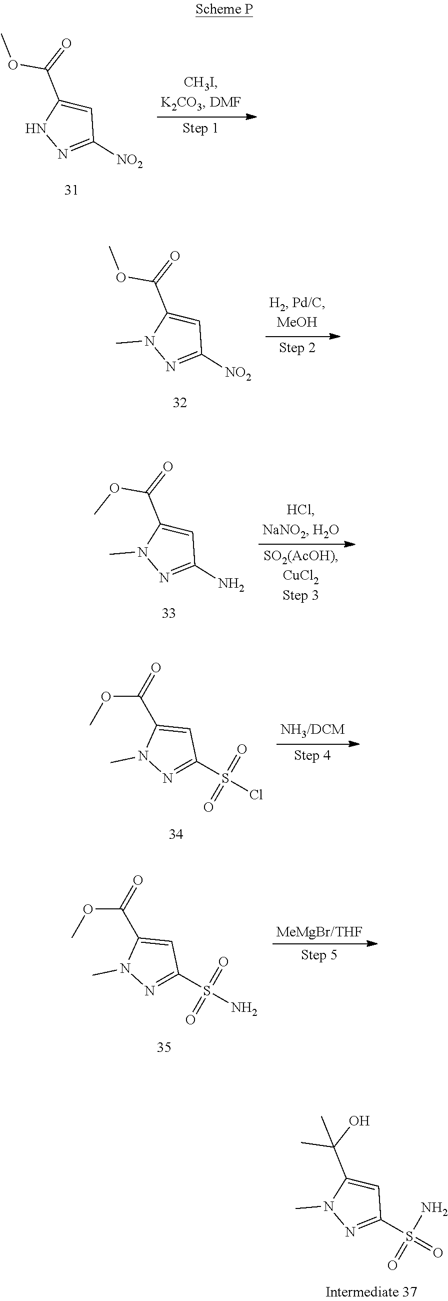

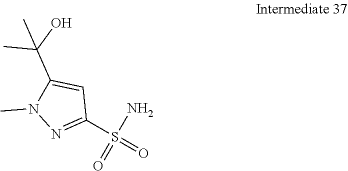

### 5-(2-Hydroxypropan-2-yl)-1-methyl-1H-pyrazole-3-sulfonamide

### Step 1: Methyl 1-methyl-3-nitro-1H-pyrazole-5-carboxylate

Into a 250-mL round-bottom flask purged with and maintained under nitrogen, was placed methyl 3-nitro-1H-pyrazole-5-carboxylate (15 g, 87.7 mmol), DMF (50 mL), potassium carbonate (22.4 g, 162 mmol), CH₃I (18.5 g, 130 mmol). The resulting solution was stirred for 15 h at RT and then was quenched by the addition of 50 mL of water. The resulting solution was extracted with 3×40 mL of ethyl acetate and the organic layers combined and dried over anhydrous Na₂SO₄, then concentrated under vacuum. This resulted in 17 g (crude) of the title compound as a yellow solid. MS-ESI: 186.0 (M+1).

### Step 2: Methyl 3-amino-I-methyl-1H-pyrazole-5-carboxylate

Into a 500-mL round-bottom flask, was placed methyl 1-methyl-3-nitro-1H-pyrazole-5-carboxylate (17 g, 91.8 mmol), MeOH (100 mL). Then Pd/C (10% wt, 2 g) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:4 to 2:3). This resulted in 11.6 g (81%) of the title compound as a yellow solid. MS-ESI: 156.1 (M+1).

Steps 3-5 used similar procedures for converting compound 18 to Intermediate 19 shown in Scheme M to afford Intermediate 37. MS-ESI: 218.0 (M-1).

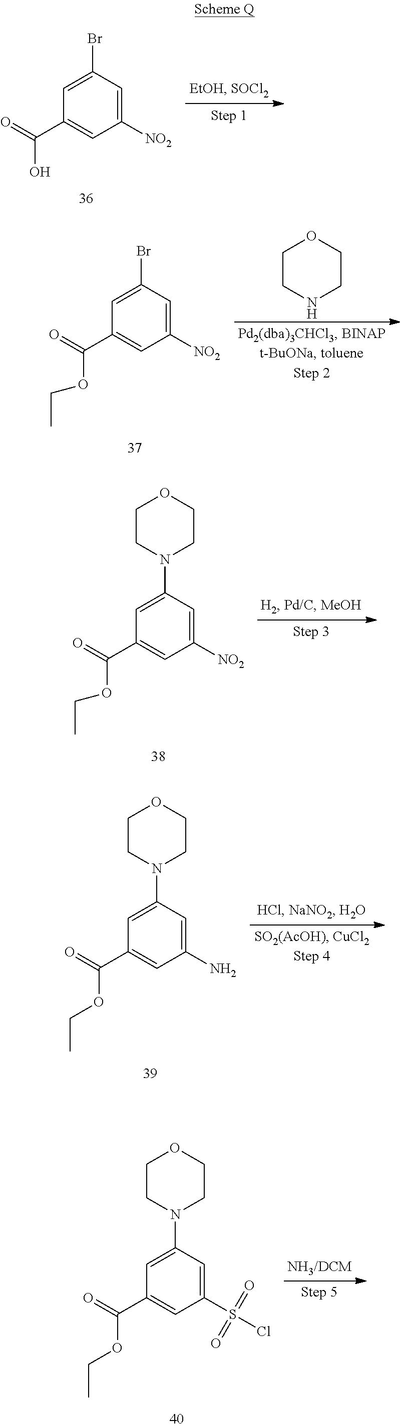 [SMILES: O=C(O)c1cc(Br)cc([N+](=O)[O-])c1 ; CCOC(=O)c1cc(Br)cc([N+](=O)[O-])c1 ; C1COCCN1 ; CCOC(=O)c1cc(N2CCOCC2)cc([N+](=O)[O-])c1 ; CCOC(=O)c1cc(N)cc(N2CCOCC2)c1 ; CCOC(=O)c1cc(N2CCOCC2)cc(S(=O)(=O)Cl)c1]

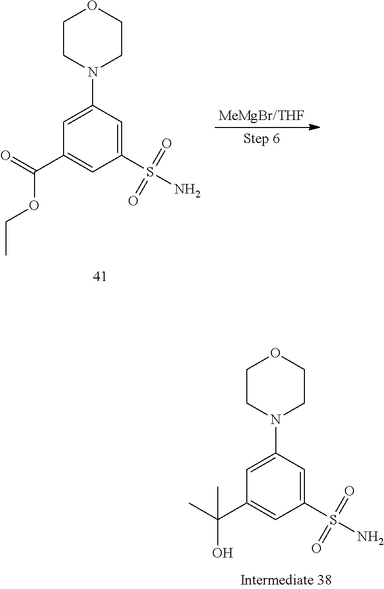 [SMILES: CCOC(=O)c1cc(N2CCOCC2)cc(S(N)(=O)=O)c1 ; CC(C)(O)c1cc(N2CCOCC2)cc(S(N)(=O)=O)c1]

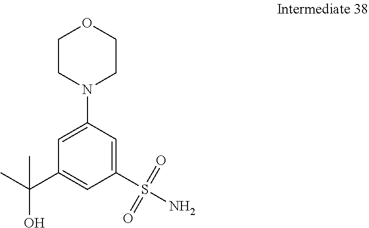 [SMILES: CC(C)(O)c1cc(N2CCOCC2)cc(S(N)(=O)=O)c1]

### 3-(2-Hydroxypropan-2-yl)-5-morpholinobenzenesulfonamide

### Step 1: Ethyl 3-bromo-5-nitrobenzoate

Into a 500-mL round-bottom flask, was placed 3-bromo-5-nitrobenzoic acid (25 g, 101.6 mmol), EtOH (200 mL). This was followed by the addition of thionyl chloride (15 mL) dropwise with stirring at 0° C. The resulting solution was stirred for 4 h at 80° C. and then was quenched by the addition of 50 mL water. The resulting solution was extracted with 3×50 mL of DCM and the organic layers combined and concentrated under vacuum. The residue was applied onto a silica gel column and eluted with ethyl acetate/petroleum ether (1:20 to 1:10). This resulted in 27.5 g (99%) of the title compound as a white solid.

### Step 2: Ethyl 3-(morpholin-4-yl)-5-nitrobenzoate

Into a 500-mL round-bottom flask purged with and maintained under nitrogen, was placed ethyl 3-bromo-5-nitrobenzoate (10 g, 36.5 mmol), toluene (250 mL), morpholine (4.6 g, 52.8 mmol), t-BuONa (5 g, 52.0 mmol), Pd₂(dba)₃CHCl₃ (1.9 g, 1.93 mmol), BINAP (1.2 g, 1.93 mmol). The resulting solution was stirred for 18 h at 60° C. and then was concentrated under vacuum. The residue was applied onto a silica gel column and eluted with ethyl acetate/petroleum ether (1:30 to 1:10). This resulted in 2.8 g (27%) of the title compound as a yellow solid. MS-ESI: 281.1 (M+1).

### Step 3: Ethyl 3-amino-5-(morpholin-4-yl)benzoate

Into a 250-mL round-bottom flask, was placed ethyl 3-(morpholin-4-yl)-5-nitrobenzoate (3.0 g, 10.7 mmol), MeOH (100 mL). Then Pd/C (10% wt, 300 mg) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:5 to 1:3). This resulted in 2.6 g (97%) of the title compound as a yellow solid. MS-ESI: 251.1 (M+1).

Steps 4-6 used similar procedures for converting compound 18 to Intermediate 19 shown in Scheme M to afford Intermediate 38. MS-ESI: 299.1 (M-1).

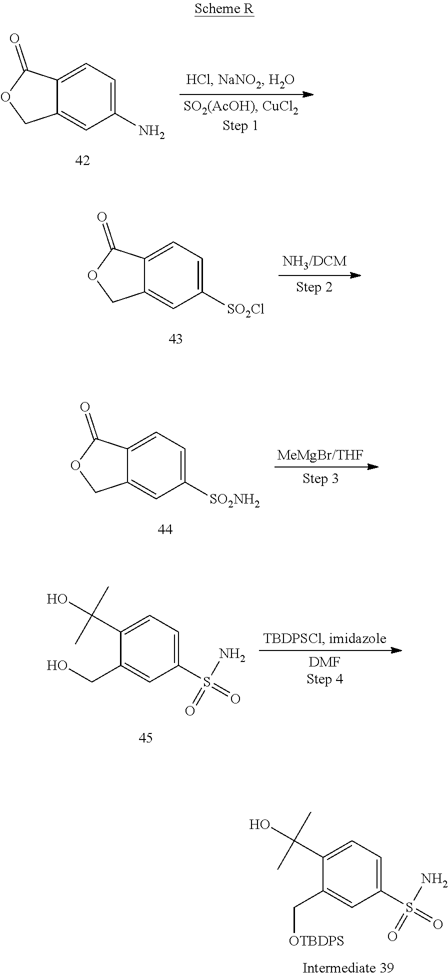

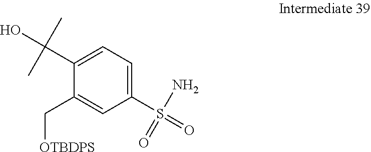

### 3-((Tert-butyldiphenylsilyloxy)methyl)-4-(2-hydroxypropan-2-yl)benzenesulfonamide

Steps 1-3 used similar procedures for converting compound 18 to Intermediate 19 shown in Scheme M to afford compound 45. MS-ESI: 212.1 (M-1).

### Step 4: 3-((Tert-butyldiphenylsilyloxy)methyl)-4-(2-hydroxypropan-2-yl)benzenesulfonamide

Into a 100-mL round-bottom flask, was placed 3-(hydroxymethyl)-4-(2-hydroxypropan-2-yl)benzenesulfonamide (1.9 g, 7.75 mmol), DMF (20 mL), imidazole (1.06 g, 15.57 mmol), TBDPSCl (3.2 g, 11.64 mmol). The resulting solution was stirred overnight at RT and then was diluted with 20 mL of water. The resulting solution was extracted with 2×20 mL of DCM and the organic layers combined and dried over anhydrous Na₂SO₄, then concentrated under vacuum. The crude product was purified by Flash-Prep-HPLC with the following conditions (IntelFlash-1): Column, C18 silica gel; mobile phase, ACN/H₂O (10 mmol/NH₄HCO₃)=1:4 increasing to ACN/H₂O (10 mmol/NH₄HCO₃)=4:1 within 30 min; Detector, UV 210 nm. This resulted in 1.4 g (37%) of the title compound as an off-white solid. MS-ESI: 482.2 (M-1).

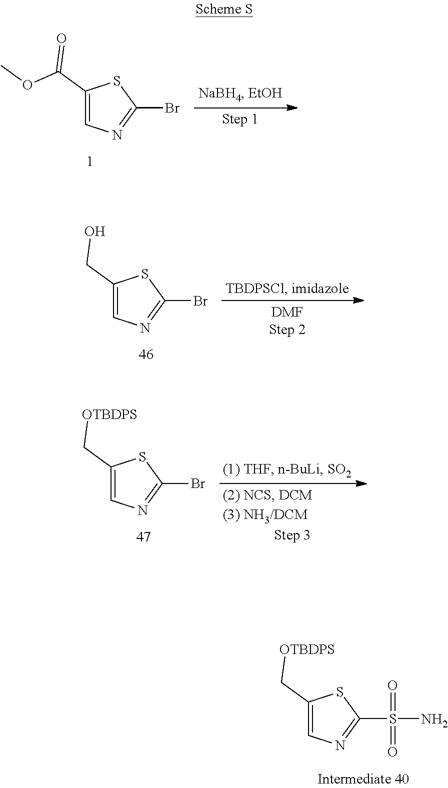

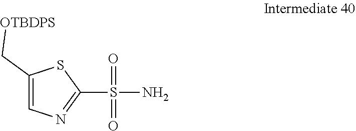

### 5-((Tert-butyldiphenylsilyloxy)methyl)thiazole-2-sulfonamide

### Step 1: (2-Bromothiazol-5-yl)methanol

Into a 250-mL round-bottom flask, was placed a solution of methyl 2-bromothiazole-5-carboxylate (15 g, 67.55 mmol) in EtOH (100 mL). This was followed by the addition of sodium borohydride (5.13 g, 139.3 mmol) in portions at 0° C. The resulting solution was stirred for 12 h at RT and then was quenched by the addition of 100 mL of water. The resulting solution was extracted with 3×50 mL of DCM and the organic layers combined and dried over anhydrous Na₂SO₄, then concentrated under vacuum. This resulted in 10 g (crude, 76%) of the title compound as a light yellow oil. MS-ESI: 195.9, 193.9 (M+1).

### Step 2: 2-Bromo-5-((tert-butyldiphenylsilyloxy)methyl)thiazole

Into a 250-mL round-bottom flask, was placed (2-bromothiazol-5-yl)methanol (8 g, 41.2 mmol), DMF (50 mL), TBDPSCl (12.5 g, 45.5 mmol), imidazole (5.6 g, 82.4 mmol). The resulting solution was stirred for 2 h at RT and then was concentrated under vacuum. The residue was applied onto a silica gel column and eluted with ethyl acetate/petroleum ether (1:100 to 1:80). This resulted in 15 g (84%) of the title compound as a light yellow solid. MS-ESI: 434.0, 432.0 (M+1).

### Step 3: 5-((Tert-butyldiphenylsilyloxy)methyl)thiazole-2-sulfonamide

Into a 500-mL 3-necked round-bottom flask purged with and maintained under nitrogen, was placed a solution of 2-bromo-5-((tert-butyldiphenylsilyloxy)methyl)thiazole (15 g, 34.7 mmol) in THF (200 mL). This was followed by the addition of n-BuLi (2.5 M, 16.7 mL) dropwise with stirring at −78° C. The resulting solution was stirred for 30 min at −78° C. To the above SO₂ was introduced. The reaction was warmed to RT and stirred for 30 min and then was concentrated under vacuum. The residue diluted in DCM (150 mL) and then NCS (5.7 g, 42.69 mmol) was added. The resulting solution was stirred for 30 min at RT. To the above was added a saturated solution of ammonia in DCM (100 mL). The resulting solution was stirred for 2 h at RT and then was concentrated under vacuum. The residue was applied onto a silica gel column and eluted with ethyl acetate/petroleum ether (1:20 to 1:10). This resulted in 7.5 g (50%) of the title compound as a light yellow solid. MS-ESI: 431.1 (M-1).

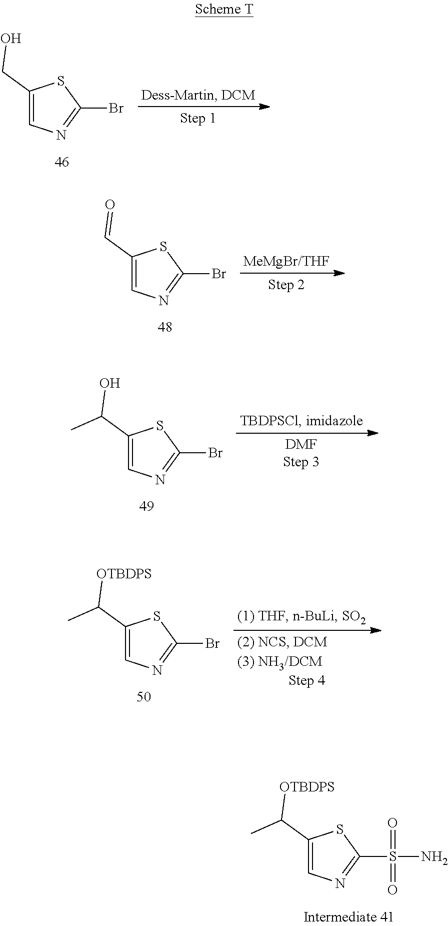

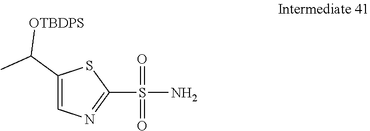

### 5-(1-(Tert-butyldiphenylsilyloxy)ethyl)thiazole-2-sulfonamide

### Step 1: 2-Bromothiazole-5-carbaldehyde

Into a 500-mL round-bottom flask, was placed (2-bromothiazol-5-yl)methanol (20 g, 103 mmol), DCM (200 mL). This was followed by the addition of Dess-Martin reagent (46 g, 103 mmol) in portions at 0° C. The resulting solution was stirred for 2 h at RT and then was concentrated under vacuum. The residue was applied onto a silica gel column and eluted with ethyl acetate/petroleum ether (1:20 to 1:10). This resulted in 18 g (91%) of the title compound as a white solid. MS-ESI: 193.9, 191.9 (M+1).

### Step 2: 1-(2-Bromothiazol-5-yl)ethanol

Into a 500-mL 3-necked round-bottom flask purged with and maintained under nitrogen, was placed a solution of 2-bromothiazole-5-carbaldehyde (18 g, 93.7 mmol) in THF (200 mL). This was followed by the addition of MeMgBr/THF (3 M, 33 mL) dropwise with stirring at 0° C. The resulting solution was stirred for 0.5 h at 0° C. The reaction was then quenched by the addition of 200 mL of NH₄Cl (sat.). The resulting solution was extracted with 2×200 mL of DCM and the organic layers combined and concentrated under vacuum. The residue was applied onto a silica gel column and eluted with ethyl acetate/petroleum ether (1:20 to 1:15). This resulted in 15 g (77%) of the title compound as colorless oil. MS-ESI: 209.9, 207.9 (M+1).

Steps 3-4 used similar procedures for converting compound 46 to Intermediate 40 shown in Scheme S to afford Intermediate 41. MS-ESI: 445.1 (M-1).

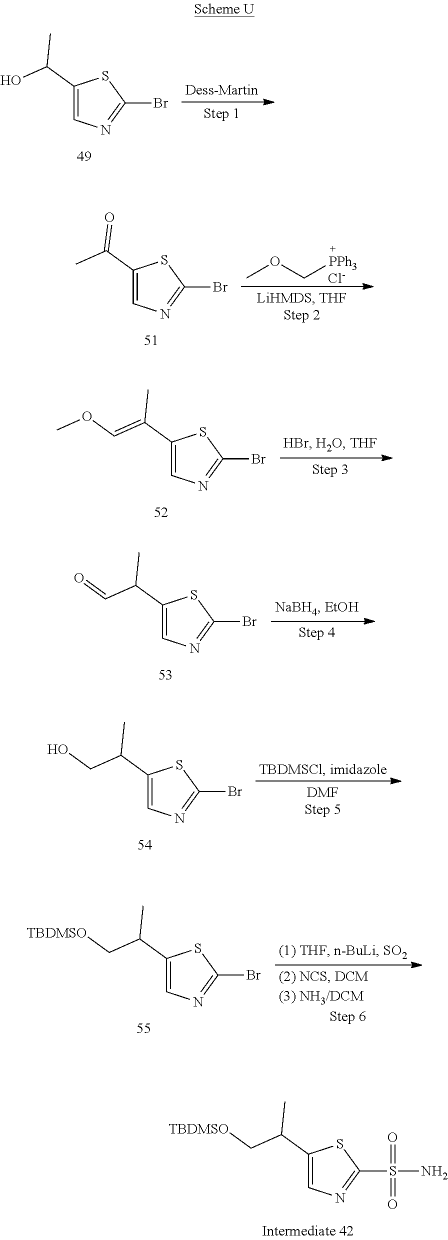

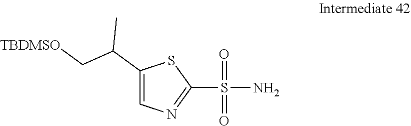 [SMILES: CC(CO[Si](C)(C)C(C)(C)C)c1cnc(S(N)(=O)=O)s1]

### 5-(1-(Tert-butyldimethylsilyloxy)propan-2-yl)thiazole-2-sulfonamide

### Step 1: 1-(2-Bromothiazol-5-yl)ethanone

Into a 250-mL round-bottom flask, was placed 1-(2-bromothiazol-5-yl)ethanol (5.792 g, 27.84 mmol), DCM (150 mL), and Dess-Martin reagent (17.72 g, 41.78 mmol). The resulting solution was stirred for 2 h at RT and then was concentrated under vacuum. The residue was applied onto a silica gel column and eluted with ethyl acetate/petroleum ether (1:10 to 1:5). This resulted in 5.29 g (92%) of the title compound as an off-white solid. MS-ESI: 207.9, 205.9 (M+1).

### Step 2: 2-Bromo-5-(1-methoxyprop-1-en-2-yl)thiazole

Into a 250-mL 3-necked round-bottom flask purged with and maintained under nitrogen, was placed (methoxymethyl)triphenylphosphanium chloride (13.16 g, 38.39 mmol), THF (100 mL). This was followed by the addition of LiHMDS (1 M, 38.52 mL) dropwise with stirring at 0° C. The resulting solution was stirred for 0.5 h at 0° C. To this was added a solution of 1-(2-bromothiazol-5-yl)ethanone (5.29 g, 25.67 mmol) in THF (30 mL) dropwise with stirring at 0° C. The resulting solution was stirred for 1 h at RT and then was quenched by the addition of 100 mL of NH₄Cl (sat.). The resulting solution was extracted with 3×80 mL of DCM and the organic layers combined and dried over anhydrous Na₂SO₄, then concentrated under vacuum. The residue was applied onto a silica gel column and eluted with ethyl acetate/petroleum ether (1:5 to 1:3). This resulted in 4.38 g (73%) of the title compound as light yellow oil. MS-ESI: 235.9, 234.0 (M+1).

### Step 3: 2-(2-Bromothiazol-5-yl)propanal

Into a 250-mL round-bottom flask, was placed 2-bromo-5-(1-methoxyprop-1-en-2-yl)thiazole (4.38 g, 18.7 mmol), THF (30 mL), water (50 mL), HBr (47% wt, 50 mL). The resulting solution was stirred for 4 h at 70° C. and then was diluted with 30 mL of water. The resulting solution was extracted with 3×50 mL of DCM and the organic layers combined and dried over anhydrous Na₂SO₄, then concentrated under vacuum. This resulted in 3.79 g (crude, 92%) of the title compound as light yellow oil. MS-ESI: 221.9, 219.9 (M+1).

### Step 4: 2-(2-Bromothiazol-5-yl)propan-1-ol

Into a 250-mL round-bottom flask, was placed 2-(2-bromothiazol-5-yl)propanal (4 g, 18.2 mmol), EtOH (60 mL). This was followed by the addition of NaBH₄ (1.38 g, 36.5 mmol) in portions at 0° C. The resulting solution was stirred overnight at RT and then was quenched by the addition of 50 mL of water. The resulting solution was extracted with 3×50 mL of DCM and the organic layers combined and dried over anhydrous Na₂SO₄, then concentrated under vacuum.

This resulted in 3.79 g (94%) of the title compound as light yellow oil. MS-ESI: 223.9, 222.0 (M+1).

### Step 5: 2-Bromo-5-(1-(tert-butyldimethylsilyloxy)propan-2-yl)thiazole

Into a 100-mL round-bottom flask, was placed 2-(2-bromothiazol-5-yl)propan-1-ol (3.79 g, 17.1 mmol), DMF (25 mL), imidazole (2.33 g, 34.2 mmol), TBDMSCl (3.87 g, 25.7 mmol). The resulting solution was stirred overnight at RT and then was diluted with 30 mL of water. The resulting solution was extracted with 3×30 mL of DCM and the organic layers combined and dried over anhydrous Na₂SO₄, then concentrated under vacuum. The residue was applied onto a silica gel column and eluted with ethyl acetate/petroleum ether (1:15 to 1:10). This resulted in 3.12 g (54%) of the title compound as a white solid. MS-ESI: 338.0, 336.0 (M+1).

Step 6 used similar procedure for converting compound 47 to Intermediate 40 shown in Scheme S to afford Intermediate 42. MS-ESI: 335.1 (M-1).

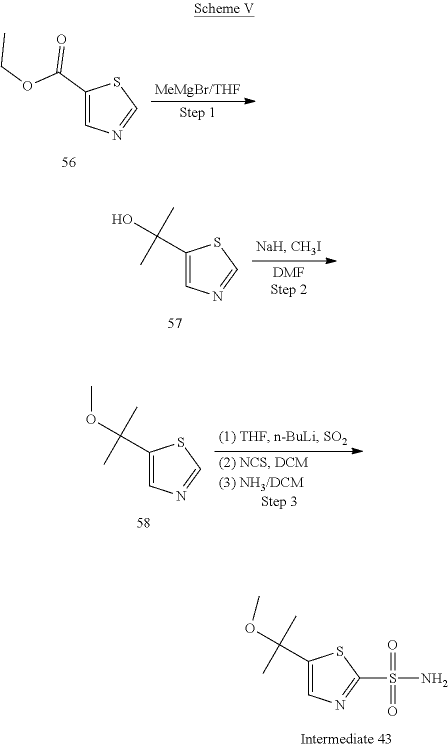

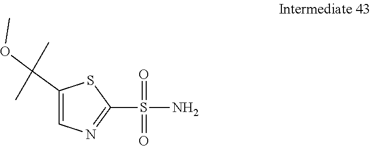

### 5-(2-Methoxypropan-2-yl)thiazole-2-sulfonamide

### Step 1: 2-(Thiazol-5-yl)propan-2-ol

Into a 250-mL 3-necked round-bottom flask purged with and maintained under nitrogen, was placed a solution of ethyl ethyl thiazole-5-carboxylate (3.75 g, 23.9 mmol) in THF (50 mL). This was followed by the addition of MeMgBr/THF (3 M, 40 mL) dropwise with stirring at 0° C. The resulting solution was stirred for 2 h at RT and then was quenched by the addition of 50 mL of NH₄Cl (sat.). The resulting solution was extracted with 3×80 mL of DCM and the organic layers combined and dried over anhydrous Na₂SO₄, then concentrated under vacuum. The residue was applied onto a silica gel column and eluted with ethyl acetate/petroleum ether (1:3 to 1:1). This resulted in 2.1 g (61%) of the title compound as yellow oil. MS-ESI: 144.0 (M+1).

### Step 2: 5-(2-Methoxypropan-2-yl)thiazole

Into a 100-mL round-bottom flask, was placed a solution of 2-(thiazol-5-yl)propan-2-ol (2.06 g, 14.4 mmol) in DMF (20 mL). This was followed by the addition of NaH (60%, 1.15 g, 28.8 mmol) in portions at 0° C. To this was added CH₃I (3.07 g, 21.6 mmol) dropwise with stirring at 0° C. The resulting solution was stirred for 1 h at RT and then was quenched by the addition of 20 mL of water. The resulting solution was extracted with 3×30 mL of ethyl acetate and the organic layers combined and dried over anhydrous Na₂SO₄, then concentrated under vacuum. The residue was applied onto a silica gel column and eluted with ethyl acetate/petroleum ether (1:5 to 1:3). This resulted in 1.42 g (63%) of the title compound as yellow oil. MS-ESI: 158.1 (M+1). Step 3 used similar procedure for converting compound 47 to Intermediate 40 shown in Scheme S to afford Intermediate 43. MS-ESI: 235.0 (M-1).

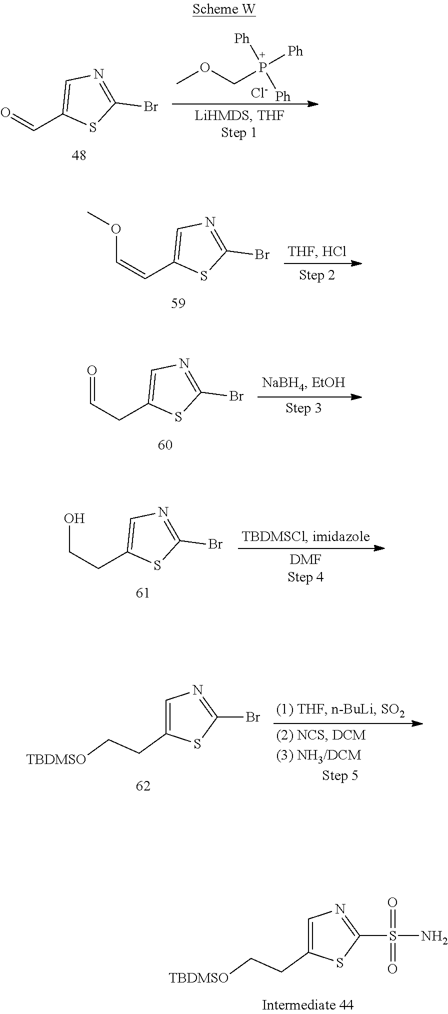

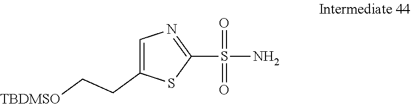 [SMILES: CC(C)(C)[Si](C)(C)OCCc1cnc(S(N)(=O)=O)s1]

### 5-(2-(Tert-butyldimethylsilyloxy)ethyl)thiazole-2-sulfonamide

### Step 1: 2-Bromo-5-(2-methoxyvinyl)thiazole

Into a 100-mL 3-necked round-bottom flask purged with and maintained under nitrogen, was placed (methoxymethyl)triphenylphosphanium chloride (3.2 g, 9.33 mmol), THF (15 mL). This was followed by the addition of LiHMDS (1 M, 9.4 mL) dropwise with stirring at 0° C. To this was added a solution of 2-bromo-1,3-thiazole-5-carbaldehyde (1.5 g, 7.81 mmol) in THF (10 mL) dropwise with stirring at 0° C. The resulting solution was stirred for 0.5 h at 0° C. and then was quenched by the addition of 50 mL of NH₄Cl (sat.). The resulting solution was extracted with 3×50 mL of DCM and the organic layers combined and concentrated under vacuum. The residue was applied onto a silica gel column and eluted with ethyl acetate/petroleum ether (1:100 to 1:80). This resulted in 1.3 g (76%) of the title compound as brown oil. The crude product was used in the next step.

### Step 2: 2-(2-Bromo-1,3-thiazol-5-yl)acetaldehyde

Into a 50-mL round-bottom flask purged with and maintained under nitrogen, was placed 2-bromo-5-(2-methoxyvinyl)thiazole (1.3 g, 5.91 mmol), THF (10 mL). This was followed by the addition of aqueous hydrogen chloride (4 M, 5 mL) dropwise with stirring at 0° C. The resulting solution was stirred for 4 h at 60° C. The resulting solution was extracted with 3×30 mL of DCM and the organic layers combined and dried over anhydrous Na₂SO₄, then concentrated under vacuum. This resulted in 1.1 g (90%) of the title compound as light yellow oil. MS-ESI: 205.9, 207.9 (M+1).

### Step 3: 2-(2-Bromo-1,3-thiazol-5-yl)ethan-1-ol

Into a 50-mL round-bottom flask, was placed 2-(2-bromo-1,3-thiazol-5-yl)acetaldehyde (1.1 g, 5.34 mmol), EtOH (10 mL), sodium borohydride (200 mg, 5.43 mmol). The resulting solution was stirred for 2 h at RT and then was quenched by the addition of 20 mL of water. The resulting solution was extracted with 3×30 mL of DCM and the organic layers combined and dried over anhydrous Na₂SO₄, then concentrated under vacuum. This resulted in 1.0 g (90%) of the title compound as light yellow oil. MS-ESI: 207.9, 209.9 (M+1).

### Step 4: 2-Bromo-5-(2-(tert-butyldimethylsilyloxy)ethyl)thiazole

Into a 50-mL round-bottom flask, was placed 2-(2-bromo-1,3-thiazol-5-yl)ethan-1-ol (1.0 g, 4.81 mmol), DMF (10 mL), imidazole (650 mg, 9.56 mmol), TBDMSCl (1.1 g, 7.30 mmol). The resulting solution was stirred for 2 h at RT and then was diluted with 20 mL of water. The resulting solution was extracted with 2×20 mL of DCM and the organic layers combined and concentrated under vacuum. This resulted in 1.2 g (77%) of the title compound as light yellow oil. MS-ESI: 324.0, 322.0 (M+1).

Step 5 used similar procedure for converting compound 47 to Intermediate 40 shown in Scheme S to afford Intermediate 44. MS-ESI: 321.1 (M-1).

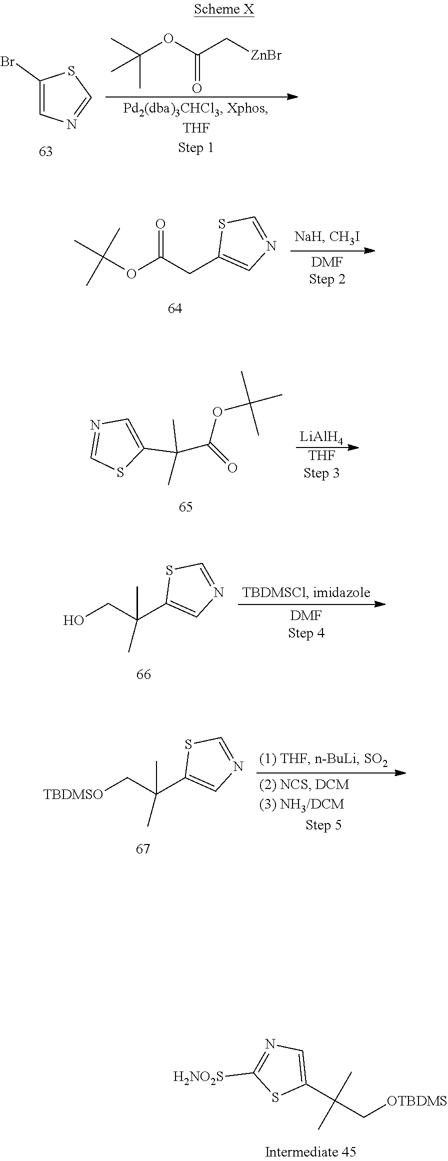

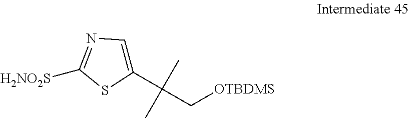 [SMILES: CC(C)(CO[Si](C)(C)C(C)(C)C)c1cnc(S(N)(=O)=O)s1]

### 5-(1-(Tert-butyldimethylsilyloxy)-2-methylpropan-2-yl)thiazole-2-sulfonamide

### Step 1: Tert-butyl 2-(thiazol-5-yl)acetate

Into a 100-mL 3-necked round-bottom flask purged with and maintained under nitrogen, was placed 5-bromothiazole (3 g, 18.29 mmol), THF (30 mL), X-phos (1.74 g, 3.66 mmol), Pd₂(dba)₃CHCl₃ (950 mg, 0.91 mmol). The resulting solution was stirred for 0.5 h at RT. To the above was added tert-butyl 2-(bromozincio)acetate (7.13 g, 27.37 mmol). The resulting solution was stirred for 4 h at 70° C. and then was concentrated under vacuum. The residue was applied onto a silica gel column and eluted with ethyl acetate/petroleum ether (1:10 to 1:3). This resulted in 2.4 g (66%) of the title compound as brown oil. MS-ESI: 200.1 (M+1).

### Step 2: Tert-butyl 2-methyl-2-(thiazol-5-yl)propanoate

Into a 100-mL round-bottom flask purged with and maintained under nitrogen, was placed tert-butyl 2-(thiazol-5-yl)acetate (1 g, 5.02 mmol), DMF (20 mL). This was followed by the addition of NaH (60%, 600 mg, 25.00 mmol) in portions at 0° C. The solution was stirred for 0.5 h at 0° C. This was followed by the addition of CH₃I (2.13 g, 15.06 mmol) dropwise with stirring at 0° C. The resulting solution was stirred for 2 h at RT and then was quenched by the addition of 40 mL of NH₄Cl (sat.). The resulting solution was extracted with 3×50 mL of DCM and the organic layers combined and concentrated under vacuum. The residue was applied onto a silica gel column and eluted with ethyl acetate/petroleum ether (1:10 to 1:3). This resulted in 0.7 g (61%) of the title compound as light yellow oil. MS-ESI: 228.1 (M+1).

### Step 3: 2-Methyl-2-(thiazol-5-yl)propan-1-ol

Into a 100-mL round-bottom flask, was placed tert-butyl 2-methyl-2-(thiazol-5-yl)propanoate (700 mg, 3.08 mmol), THF (20 mL). This was followed by the addition of LiAlH₄ (200 mg, 5.27 mmol) in portions at 0° C. and was stirred for 2 h at 0° C. and then was quenched by the addition of 1 mL of water. The solids were filtered out. The resulting mixture was concentrated under vacuum. This resulted in 400 mg (83%) of the title compound as brown oil. MS-ESI: 158.1 (M+1).

Steps 4-5 used similar procedures for converting compound 54 to Intermediate 42 shown in in Scheme U to afford Intermediate 45. MS-ESI: 349.1 (M-1).

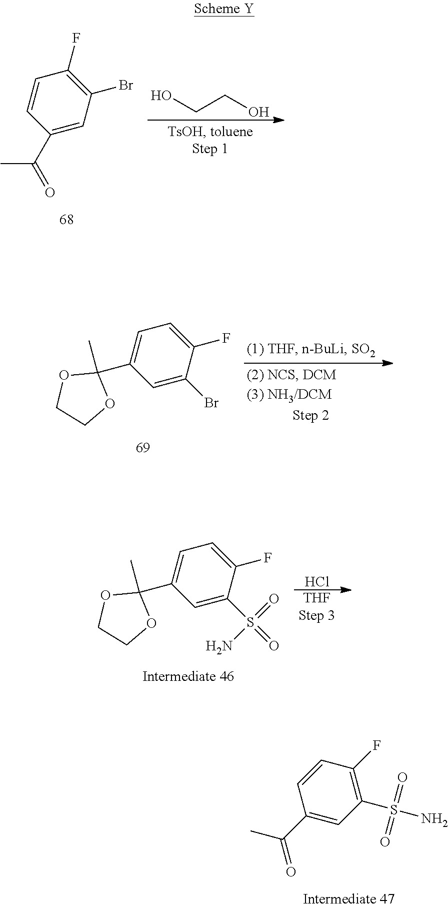

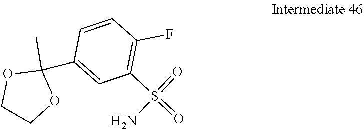 [SMILES: CC1(c2ccc(F)c(S(N)(=O)=O)c2)OCCO1]

### 2-Fluoro-5-(2-methyl-1,3-dioxolan-2-yl)benzenesulfonamide

### Step 1: 2-(3-Bromo-4-fluorophenyl)-2-methyl-1,3-dioxolane

Into a 250-mL round-bottom flask, was placed a solution of 1-(3-bromo-4-fluorophenyl)ethan-1-one (5 g, 23.0 mmol) in toluene (50 mL), ethane-1,2-diol (4 mL), TsOH (200 mg, 1.16 mmol).

The resulting solution was stirred for 6 h at 120° C. The reaction was then quenched by the addition of 100 mL of water. The resulting solution was extracted with 3×100 mL of ethyl acetate and the organic layers combined and dried over anhydrous Na₂SO₄, then concentrated under vacuum. The residue was applied onto a silica gel column and eluted with ethyl acetate/petroleum ether (1:5 to 1:4). This resulted in 5.5 g (91%) of the title compound as yellow oil.

Step 2 used similar procedure for converting compound 47 to Intermediate 40 shown in Scheme S to afford Intermediate 46. MS-ESI: 260.0 (M-1).

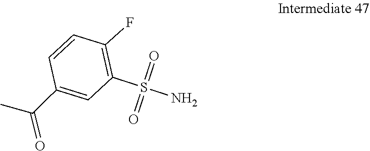

### 5-Acetyl-2-fluorobenzenesulfonamide

### Step 3: 5-Acetyl-2-fluorobenzenesulfonamide

Into a 50-mL round-bottom flask, was placed 2-fluoro-5-(2-methyl-1,3-dioxolan-2-yl)benzene-1-sulfonamide (300 mg, 1.15 mmol), THF (5 mL), hydrogen chloride (1 N, 5 mL). The resulting solution was stirred for 12 h at RT. The pH value of the solution was adjusted to 7-8 with NaOH (2 N). The resulting solution was extracted with 3×30 mL of ethyl acetate and the organic layers combined and concentrated under vacuum. This resulted in 240 mg (crude, 96%) of the title compound as a light yellow solid. MS-ESI: 216.0 (M-1).

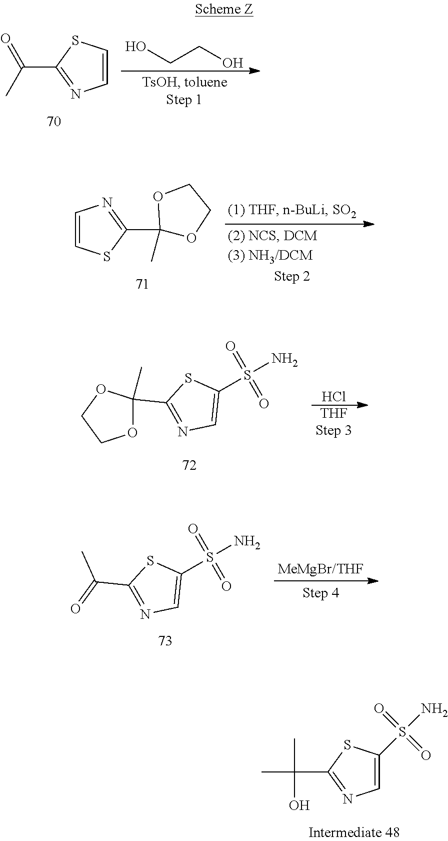

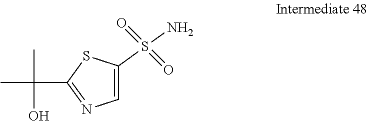

### 2-(2-Hydroxypropan-2-yl)thiazole-5-sulfonamide

Compound 73 was prepared using similar procedures for converting compound 68 to Intermediate 47 shown in Scheme Y.

### Step 4: 2-(2-Hydroxypropan-2-yl)thiazole-5-sulfonamide

Into a 100-mL 3-necked round-bottom flask purged with and maintained under nitrogen, was placed 2-acetylthiazole-5-sulfonamide (1 g, 4.85 mmol), THF (20 mL). This was followed by the addition of MeMgBr (3 M, 7 mL) dropwise with stirring at 0° C. The resulting solution was stirred for 14 h at RT and then was quenched by the addition of 20 mL of NH₄Cl (sat.). The resulting solution was extracted with 2×30 mL of DCM and the organic layers combined and concentrated under vacuum. The residue was applied onto a silica gel column and eluted with ethyl acetate/petroleum ether (1:5 to 1:3). This resulted in 580 mg (54%) of the title compound as a light yellow solid. MS-ESI: 221.0 (M-1).

Schemes for phenylacetic acids Intermediates: Schemes AA-AQ illustrate the phenylacetic acid intermediates preparation.

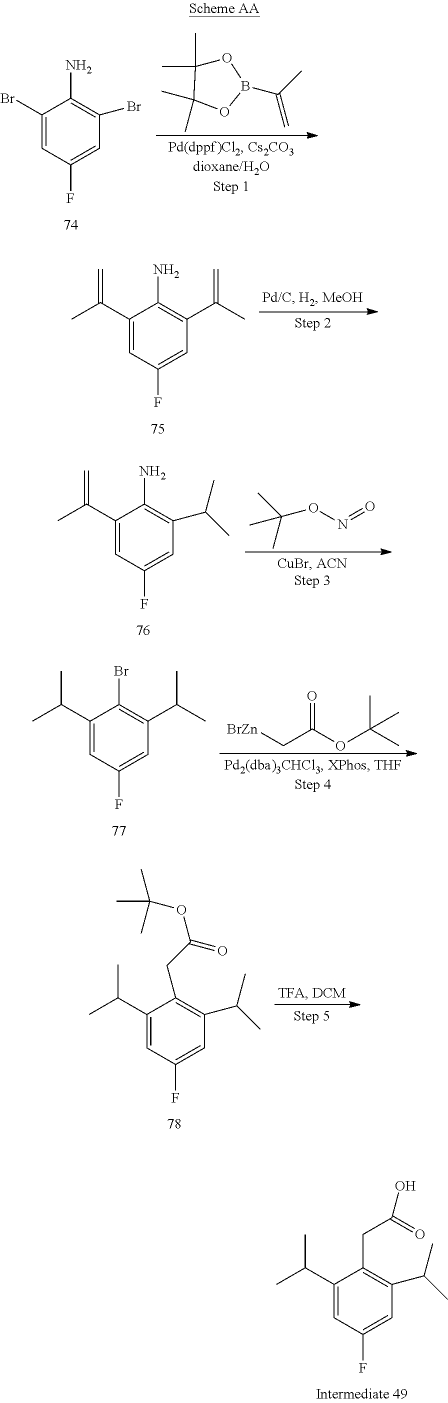

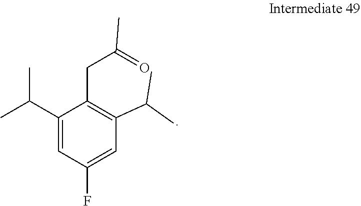 [SMILES: CC(=O)Cc1c(C(C)C)cc(F)cc1C(C)C]

### 2-(4-Fluoro-2,6-diisopropylphenyl)acetic acid

### Step 1: 4-Fluoro-2,6-bis(prop-1-en-2-yl)aniline

Into a 500-mL round-bottom flask purged with and maintained under nitrogen, was placed 2,6-dibromo-4-fluoroaniline (15 g, 55.8 mmol), dioxane (150 mL), water(15 mL), Cs₂CO₃ (55 g, 169 mmol), 4,4,5,5-tetramethyl-2-(prop-1-en-2-yl)-1,3,2-dioxaborolane (25 g, 149 mmol), Pd(dppf)Cl₂ (4 g, 5.47 mmol). The resulting solution was stirred for 15 h at 100° C. and then was concentrated under vacuum. The residue was applied onto a silica gel column and eluted with ethyl acetate/petroleum ether (1:10 to 1:8). This resulted in 9.2 g (86%) of the title compound as brown oil. MS-ESI: 192.1 (M+1).

### Step 2: 4-Fluoro-2,6-bis(propan-2-yl)aniline

Into a 500-mL round-bottom flask, was placed 4-fluoro-2,6-bis(prop-1-en-2-yl)aniline (9.2 g, 48.1 mmol), MeOH (200 mL). Then Pd/C (10% wt, 900 mg) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:10 to 1:8). This resulted in 7.2 g (77%) of the title compound as brown oil. MS-ESI: 196.1 (M+1).

### Step 3: 2-Bromo-5-fluoro-1,3-bis(propan-2-yl)benzene

Into a 500-mL round-bottom flask purged with and maintained under nitrogen, was placed 4-fluoro-2,6-bis(propan-2-yl)aniline (7 g, 35.9 mmol), ACN (300 mL), CuBr (7.71 g, 53.9 mmol). This was followed by the addition of tert-butyl nitrite (5.55 g, 53.8 mmol) dropwise with stirring at 0° C. The resulting solution was stirred for 3 h at 60° C. and then was concentrated under vacuum. The residue was applied onto a silica gel column with petroleum ether. This resulted in 3.0 g (32%) of the title compound as yellow oil. ¹H NMR (400 MHz, DMSO-d6): δ 7.09 (d, J=9.8 Hz, 2H), 3.40 (hept, J=6.9 Hz, 2H), 1.20 (d, J=6.8 Hz, 12H).

### Step 4: Tert-butyl 2-[4-fluoro-2,6-bis(propan-2-yl)phenyl]acetate

Into a 250-mL 3-necked round-bottom flask purged with and maintained under nitrogen, was placed 2-bromo-5-fluoro-1,3-bis(propan-2-yl)benzene (3.0 g, 11.6 mmol), THF(150 mL), X-phos (553 mg, 1.16 mmol), Pd₂(dba)₃CHCl₃ (600 mg, 0.58 mmol). The resulting solution was stirred for 0.5 h at RT. Then to the above tert-butyl 2-(bromozincio)acetate (6.0 g, 23.04 mmol) was added. The resulting solution was stirred for 5 h at 70° C. and then was quenched by the addition of 100 mL of NH₄Cl (sat.). The resulting solution was extracted with 3×100 mL of ethyl acetate and the organic layers combined and concentrated under vacuum. The residue was applied onto a silica gel column and eluted with ethyl acetate/petroleum ether (1:100 to 3:97). This resulted in 3.14 g (92%) of the title compound as yellow oil. ¹H NMR (400 MHz, DMSO-d₆) δ 6.93 (d, J=10.4 Hz, 2H), 3.67 (s, 2H), 3.19-3.07 (m, 2H), 1.39 (s, 9H), 1.15 (d, J=6.7 Hz, 12H).

### Step 5: 2-(4-Fluoro-2,6-diisopropylphenyl)acetic Acid

Into a 50-mL round-bottom flask, was placed tert-butyl 2-[4-fluoro-2,6-bis(propan-2-yl)phenyl]acetate (1.56 g, 5.30 mmol), DCM (10 mL), TFA (10 mL). The resulting solution was stirred for 3 h at RT and then was concentrated under vacuum. This resulted in 1.36 g (crude, 108%) of the title compound as a light yellow solid. MS-ESI: 237.1 (M-1).

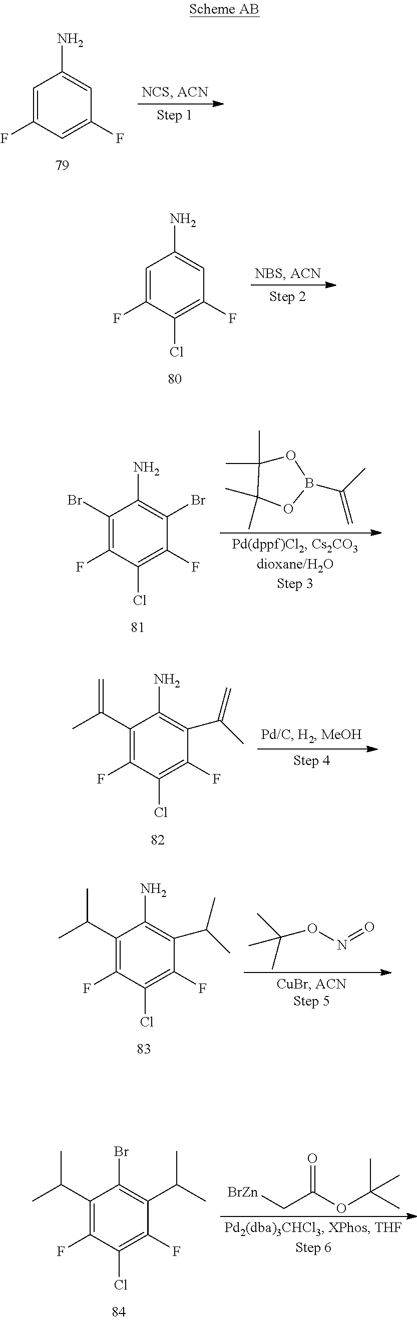

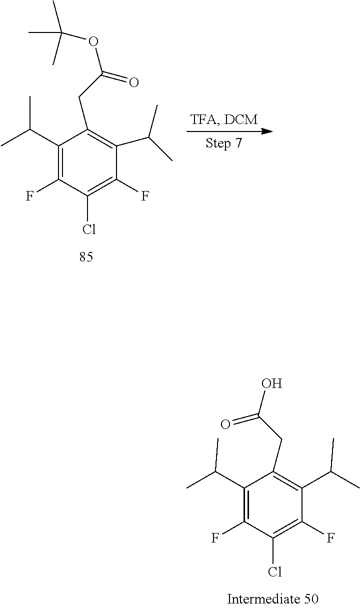 [SMILES: CC(C)c1c(F)c(Cl)c(F)c(C(C)C)c1CC(=O)OC(C)(C)C ; CC(C)c1c(F)c(Cl)c(F)c(C(C)C)c1CC(=O)O]

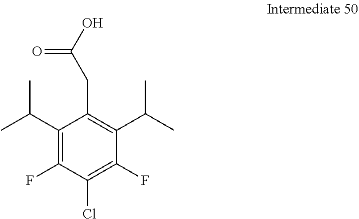 [SMILES: CC(C)c1c(F)c(Cl)c(F)c(C(C)C)c1CC(=O)O]

### 2-(4-Chloro-3,5-difluoro-2,6-diisopropylphenyl)acetic Acid

### Step 1: 4-Chloro-3,5-difluorobenzenamine

Into a 500-mL round-bottom flask, was placed 3,5-difluorobenzenamine (10.3 g, 79.8 mmol), ACN (100 mL), NCS (10.8 g, 80.9 mmol). The resulting solution was stirred for 5 h at 80° C. and then was concentrated under vacuum. The residue was applied onto a silica gel column and eluted with ethyl acetate/petroleum ether (1:3 to 1:1). This resulted in 7.1 g (54%) of the title compound as a gray solid. 164.0, 166.0 (M+1).

### Step 2: 2,6-Dibromo-4-chloro-3,5-difluorobenzenamine

Into a 250-mL round-bottom flask, was placed 4-chloro-3,5-difluorobenzenamine (4.0 g, 24.5 mmol), ACN (100 mL), NBS (13.0 g, 73.0 mmol). The resulting solution was stirred for 1 h at RT and then was concentrated under vacuum. The residue was applied onto a silica gel column and eluted with ethyl acetate/petroleum ether (1:6 to 1:4). This resulted in 7.4 g (94%) of the title compound as a yellow solid. MS-ESI: 319.8, 321.8, 323.8 (M+1).

Steps 3-7 used similar procedures for converting compound 74 to Intermediate 49 shown in Scheme AA to afford Intermediate 50. MS-ESI: 289.1, 291.1 (M-1).

Compound 84: ¹H NMR (400 MHz, CDCl₃-d) S 3.67 (hept, J=7.2 Hz, 2H), 1.33 (d, J=7.2 Hz, 12H).

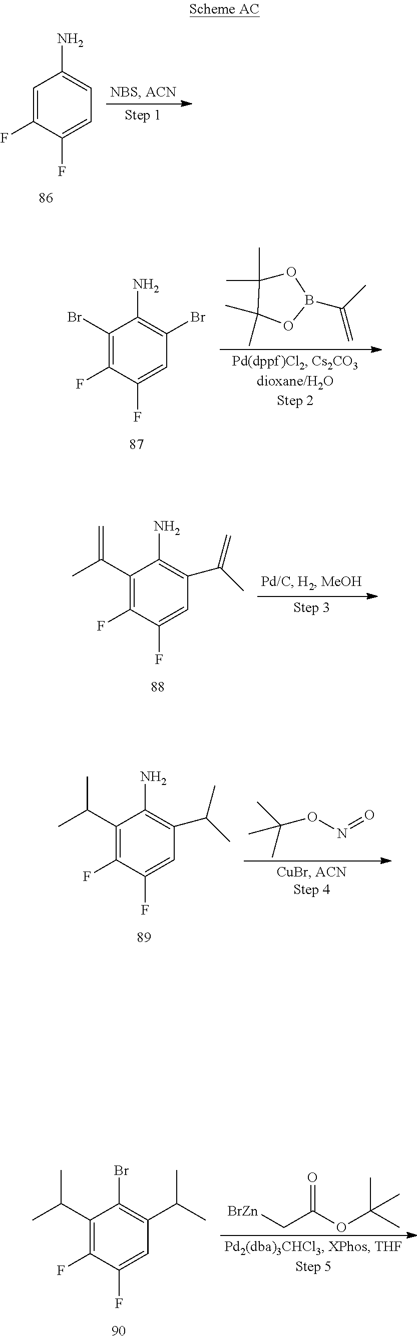

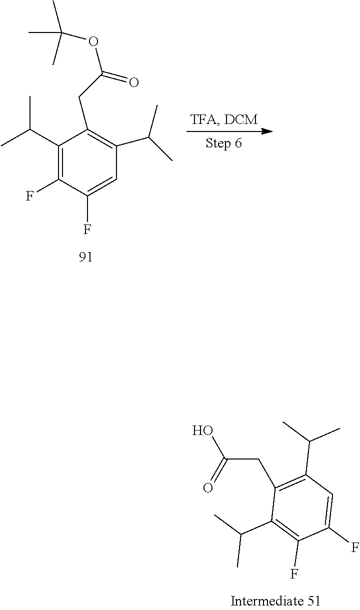 [SMILES: CC(C)c1cc(F)c(F)c(C(C)C)c1CC(=O)OC(C)(C)C ; CC(C)c1cc(F)c(F)c(C(C)C)c1CC(=O)O]

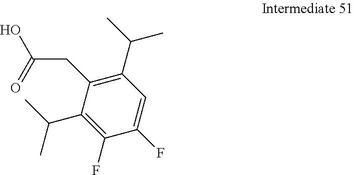 [SMILES: CC(C)c1cc(F)c(F)c(C(C)C)c1CC(=O)O]

### 2-(3,4-Difluoro-2,6-diisopropylphenyl)acetic acid

### Step 1: 2,6-Dibromo-3,4-difluorobenzenamine

Into a 250-mL round-bottom flask, was placed 3,4-difluorobenzenamine (5 g, 38.7 mmol), ACN (100 mL), NBS (16.2 g, 91.0 mmol). The resulting solution was stirred for 16 h at 85° C. and then was concentrated under vacuum. The residue was applied onto a silica gel column and eluted with ethyl acetate/petroleum ether (1:6 to 1:4). This resulted in 5.49 g (49%) of the title compound as a yellow solid. MS-ESI: 287.9, 285.9, 289.9 (M+1).

Steps 2-6 used similar procedures for converting compound 74 to Intermediate 49 shown in Scheme AA to afford Intermediate 51. MS-ESI: 255.1 (M-1).

Compound 90: ¹H NMR (300 MHz, MeOD-d₄) δ 7.10 (dd, J=11.7, 8.4 Hz, 1H), 3.79-3.70 (m, 1H), 3.48-3.29 (m, 1H), 1.32 (dd, J=6.8, 1.8 Hz, 6H), 1.18 (d, J=6.8 Hz, 6H).

Compound 91: ¹H NMR (300 MHz, DMSO-d₆) δ 7.13 (dd, J=12.3, 8.3 Hz, 1H), 3.65 (s, 2H), 3.21-3.00 (m, 2H), 1.35 (s, 9H), 1.28-1.05 (m, 12H).

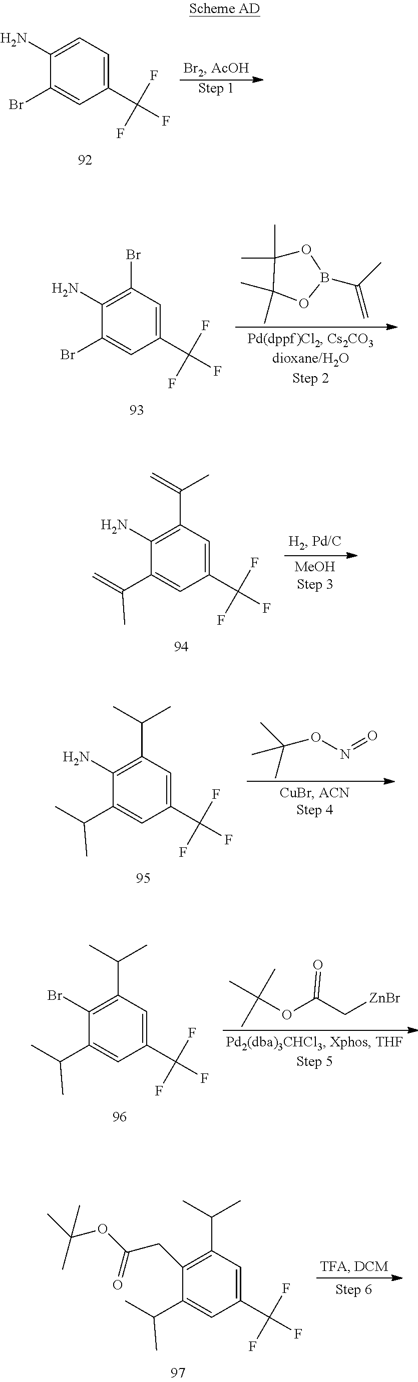

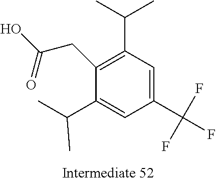 [SMILES: CC(C)c1cc(C(F)(F)F)cc(C(C)C)c1CC(=O)O]

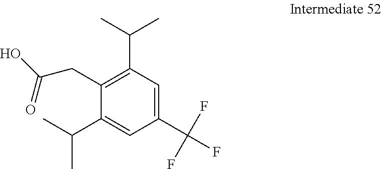 [SMILES: CC(C)c1cc(C(F)(F)F)cc(C(C)C)c1CC(=O)O]

### 2-(2,6-Diisopropyl-4-(trifluoromethyl)phenyl)acetic acid

### Step 1: 2,6-Dibromo-4-(trifluoromethyl)benzenamine

Into a 100-mL round-bottom flask purged with and maintained under nitrogen, was placed 2-bromo-4-(trifluoromethyl)benzenamine (5 g, 20.8 mmol), AcOH (50 mL), Br₂ (1.3 mL). The resulting solution was stirred for 3 h at RT and then was quenched by the addition of 50 mL of Na₂S₂O₃ (sat.). The resulting solution was extracted with 3×50 mL of DCM and the organic layers combined and dried over anhydrous Na₂SO₄, then concentrated under vacuum. This resulted in 5 g (75%) of the title compound as light yellow oil. MS-ESI: 319.9, 317.9, 321.9 (M+1).

Steps 2-6 used similar procedures for converting compound 74 to Intermediate 49 shown in Scheme AA to afford Intermediate 52. MS-ESI: 287.1 (M-1).

Compound 97: ¹H NMR (300 MHz, DMSO-d₆) δ 7.39 (s, 2H), 3.29 (s, 2H), 3.16 (hept, J=6.8 Hz, 2H), 1.37 (s, 9H), 1.16 (d, J=6.7 Hz, 12H).

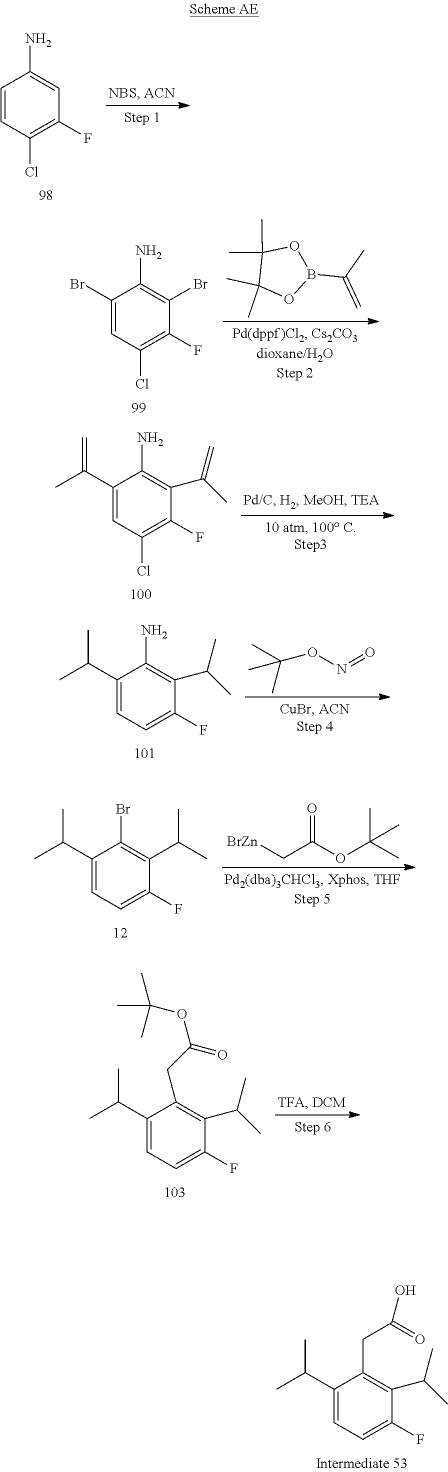

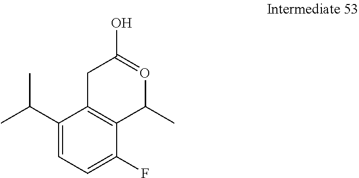

### 2-(3-Fluoro-2,6-diisopropylphenyl)acetic acid

### Step 1: 2,6-Dibromo-4-chloro-3-fluoroaniline

Into a 500-mL round-bottom flask, was placed 4-chloro-3-fluoroaniline (5.08 g, 34.9 mmol), ACN (200 mL), NBS (18.69 g, 105.0 mmol). The resulting solution was stirred for 12 h at RT and then was concentrated under vacuum. The residue was applied onto a silica gel column and eluted with ethyl acetate/petroleum ether (1:200 to 1:100). This resulted in 9.7 g (92%) of the title compound as a light yellow solid. MS-ESI: 303.8, 305.8, 301.8 (M+1).

### Step 2: 4-Chloro-3-fluoro-2,6-bis(prop-1-en-2-yl)aniline

Into a 500-mL round-bottom flask purged with and maintained under nitrogen, was placed 2,6-dibromo-4-chloro-3-fluoroaniline (9.03 g, 29.8 mmol), dioxane (200 mL), water (20 mL), 4,4,5,5-tetramethyl-2-(prop-1-en-2-yl)-1,3,2-dioxaborolane (15.12 g, 89.98 mmol), Cs₂CO₃ (29.34 g, 90.05 mmol), Pd(dppf)Cl₂ (2.20 g, 3.01 mmol). The resulting solution was stirred for 12 h at 90° C. and then was concentrated under vacuum. The residue was applied onto a silica gel column and eluted with ethyl acetate/petroleum ether (1:30 to 1:20). This resulted in 4.3 g (64%) of the title compound as yellow oil. MS-ESI: 226.1, 228.1 (M+1).

### Step 3: 3-Fluoro-2,6-bis(propan-2-yl)aniline

Into a 250-mL pressure tank reactor (10 atm) purged with and maintained under nitrogen, was placed 4-chloro-3-fluoro-2,6-bis(prop-1-en-2-yl)aniline (4.3 g, 19.1 mmol), MeOH (100 mL), TEA (2.0 g, 19.8 mmol). Then Pd/C (10% wt, 0.5 g) was added. The flask was evacuated and flushed three times with hydrogen. The resulting solution was stirred for 7 days at 100° C. under an atmosphere of hydrogen. The solids were filtered out. The resulting mixture was concentrated under vacuum. The residue was applied onto a silica gel column and eluted with ethyl acetate/petroleum ether (1:5 to 1:3). This resulted in 3.6 g (97%) of the title compound as light yellow oil. MS-ESI: 196.1 (M+1).

Steps 4-6 used similar procedures for converting compound 76 to Intermediate 49 shown in Scheme AA to afford Intermediate 53. MS-ESI: 237.1 (M-1).

Compound 102: ¹H NMR (400 MHz, DMSO-d6) δ 7.28 (dd, J=8.7, 5.9 Hz, 1H), 7.18 (dd, J=11.3, 8.7 Hz, 1H), 3.64 (hept, J=6.9 Hz, 1H), 3.36 (hept, J=6.9 Hz, 1H), 1.30 (dd, J=6.9, 1.9 Hz, 6H), 1.19 (d, J=6.8 Hz, 6H).

Compound 103: ¹H NMR (400 MHz, DMSO-d6) δ 7.16 (dd, J=8.6, 5.6 Hz, 1H), 7.00 (dd, J=11.9, 8.7 Hz, 1H), 3.72 (s, 2H), 3.23-3.00 (m, 2H), 1.40 (s, 9H), 1.28 (d, J=6.9 Hz, 6H), 1.15 (d, J=6.8 Hz, 6H).

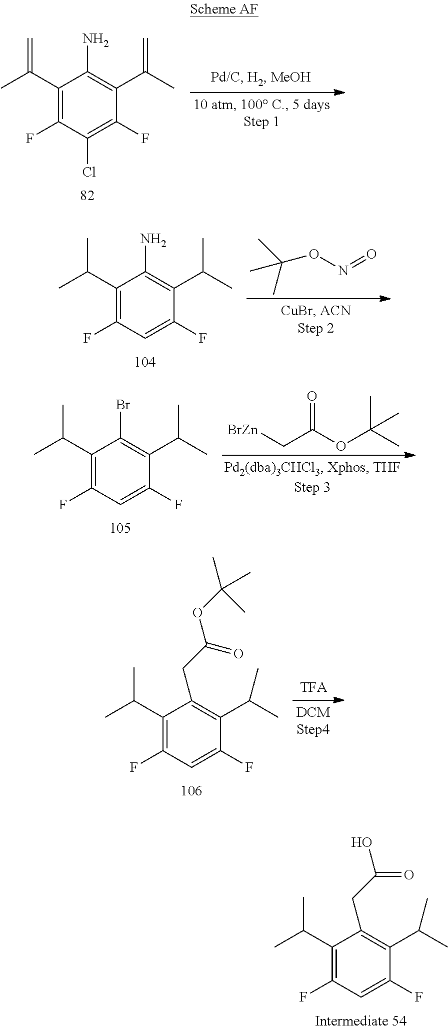

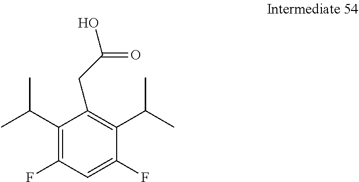 [SMILES: CC(C)c1c(F)cc(F)c(C(C)C)c1CC(=O)O]

### 2-(3,5-Difluoro-2,6-diisopropylphenyll)acetic acid

### Step 1: 3,5-Difluoro-2,6-bis(propan-2-yl)aniline

Into a 100-mL pressure tank reactor (10 atm), was placed 4-chloro-3,5-difluoro-2,6-bis(prop-1-en-2-yl) aniline (1.6 g, 6.57 mmol), MeOH (60 mL), TEA (0.2 mL). Then Pd/C (10% wt, 800 mg) was added. The flask was evacuated and flushed three times with hydrogen. The resulting solution was stirred for 5 days at 100° C. under an atmosphere of hydrogen. The solids were filtered out. The resulting mixture was concentrated under vacuum. The residue was applied onto a silica gel column and eluted with ethyl acetate/petroleum ether (1:5 to 1:3). This resulted in 1.2 g (86%) of the title compound as light yellow oil. MS-ESI: 214.1 (M+1).

Steps 2-4 used similar procedures for converting compound 76 to Intermediate 49 shown in Scheme AA to afford Intermediate 54. MS-ESI: 255.1 (M-1).

Compound 105: ¹H NMR (300 MHz, CDCl₃-d) δ 6.71 (t, J=11.4 Hz, 1H), 3.64 (hept, J=7.0 Hz, 2H), 1.29 (d, J=7.0 Hz, 12H).

Compound 106: ¹H NMR (300 MHz, CDCl3-d) δ 6.64 (t, J=11.8 Hz, 1H), 3.67 (s, 2H), 3.16 (hept, J=7.0 Hz, 2H), 1.43 (s, 9H), 1.30 (d, J=7.0 Hz, 12H).

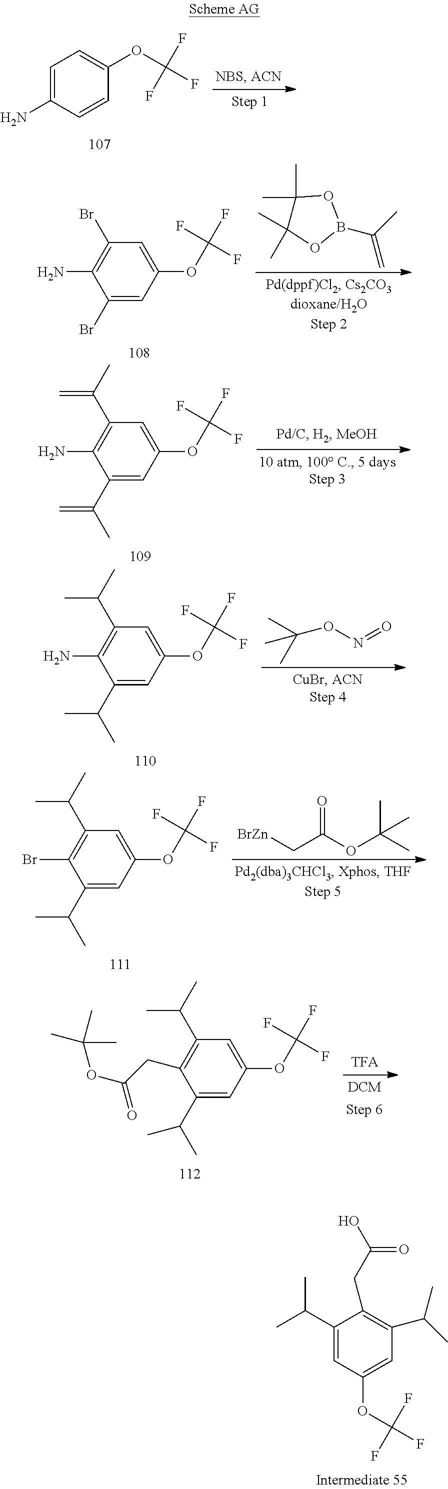

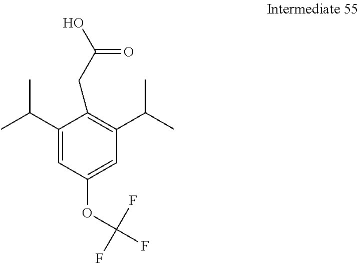 [SMILES: CC(C)c1cc(OC(F)(F)F)cc(C(C)C)c1CC(=O)O]

### 2-(2,6-Diisopropyl-4-(trifluoromethoxy)phenyl)acetic acid

### Step 1: 2,6-Dibromo-4-(trifluoromethoxy)aniline

Into a 500-mL round-bottom flask, was placed 4-(trifluoromethoxy)aniline (7.15 g, 40.4 mmol), ACN (300 mL), NBS (18 g, 101 mmol). The resulting solution was stirred for 12 h at RT and then was concentrated under vacuum. The residue was applied onto a silica gel column and eluted with ethyl acetate/petroleum ether (1:15 to 1:10). This resulted in 12 g (89%) of the title compound as a white solid. MS-ESI: 335.9, 333.9, 337.9 (M+1).

### Step 2: 2,6-Bis(prop-1-en-2-yl)-4-(trifluoromethoxy)aniline

Into a 500-mL 3-necked round-bottom flask purged with and maintained under nitrogen, was placed 2,6-dibromo-4-(trifluoromethoxy)aniline (2.67 g, 7.97 mmol), dioxane (40 mL), water (4 mL), Cs₂CO₃ (8 g, 24.8 mol), 4,4,5,5-tetramethyl-2-(prop-1-en-2-yl)-1,3,2-dioxaborolane (3.06 g, 18.2 mmol), Pd(dppf)Cl₂ (656 mg, 0.80 mmol). The resulting solution was stirred overnight at 90° C. and then concentrated under vacuum. The residue was applied onto a silica gel column and eluted with ethyl acetate/petroleum ether (1:15 to 1:10). This resulted in 1.15 g (56%) of the title compound as light yellow oil. MS-ESI: 258.1 (M+1).

Steps 3-6 used similar procedures for converting compound 82 to Intermediate 54 shown in Scheme AF to afford Intermediate 55. MS-ESI: 303.1 (M-1).

Compound 111: ¹H NMR (300 MHz, MeOD-d₄) δ 7.10-7.03 (s, 2H), 3.55 (hept, J=6.8 Hz, 2H), 1.25 (d, J=6.8 Hz, 12H).

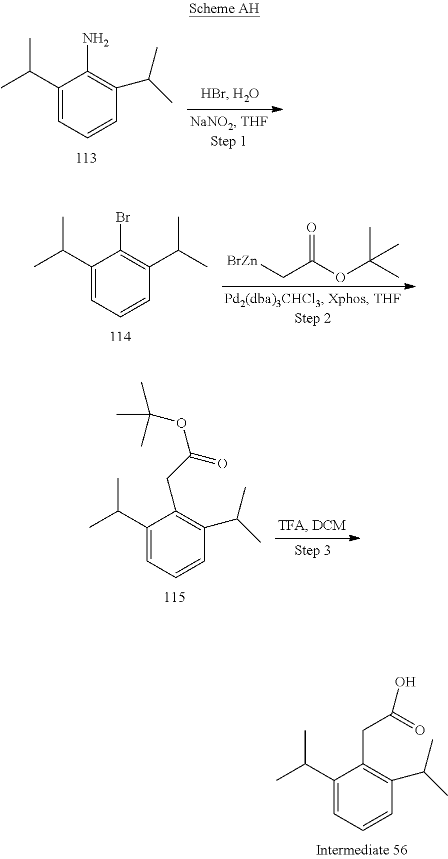

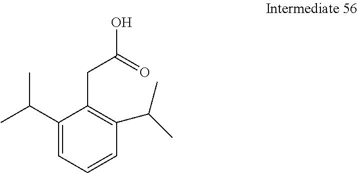 [SMILES: CC(C)c1cccc(C(C)C)c1CC(=O)O]

### 2-(2,6-Diisopropylphenyl)acetic Acid

### Step 1: 2-Bromo-1,3-bis(propan-2-yl)benzene

Into a 500-mL round-bottom flask, was placed 2,6-diisopropylbenzenamine (10 g, 56.4 mmol). This was followed by the addition of HBr (47% wt, 51 mL) dropwise with stirring at RT during 5 min. The white suspension was cooled down to −56° C. and 23.6 g (0.34 mol) of NaNO₂ (6.65 g, 96.4 mmol) was added in portions during 10 min and stirred continued at the same temperature for 1 h. Then 70 mL of ice-cold THF was slowly added during 10 min and the temperature let slowly rising to −15° C. during 2 h until no more gas evolved. The temperature was decreased again to −56° C. and 24 mL of water was added followed by the addition of sodium carbonate decahydrate (33.38 g, 11.67 mmol) giving a brown suspension. The temperature was let raising to RT during 3 h. The mixture was stirred for 16 h at RT. The resulting solution was extracted with 3×50 mL of DCM and the organic layers combined and concentrated under vacuum. The residue was applied onto a silica gel column and eluted with ethyl acetate/petroleum ether (1:15 to 1:10). This resulted in 11 g (81%) of the title compound as yellow oil.

Steps 2-3 used similar procedures for converting compound 77 to Intermediate 49 shown in Scheme AA to afford Intermediate 56. MS-ESI: 219.1 (M-1).

Compound 115: ¹H NMR (400 MHz, DMSO-d₆) δ 7.21-7.09 (m, 3H), 3.69 (s, 2H), 3.12 (hept, J=6.8 Hz, 2H), 1.39 (s, 9H), 1.18 (d, J=6.8 Hz, 12H).

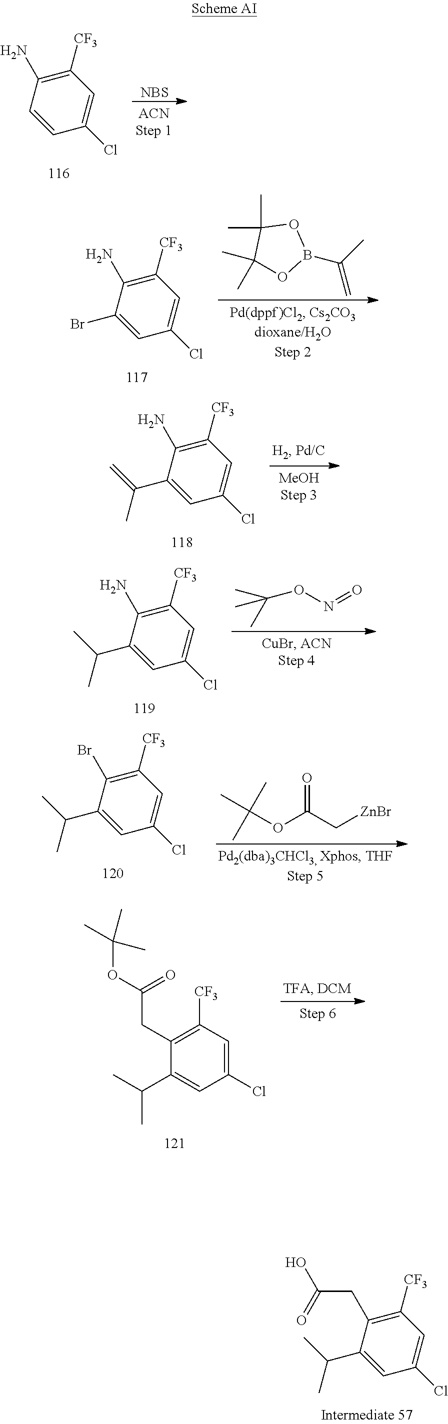

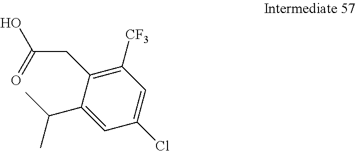 [SMILES: CC(C)c1cc(Cl)cc(C(F)(F)F)c1CC(=O)O]

### 2-(4-Chloro-2-isopropyl-6-(trifluoromethyl)phenyl)acetic acid

### Step 1: 2-Bromo-4-chloro-6-(trifluoromethyl)aniline

Into a 250-mL round-bottom flask, was placed 4-chloro-2-(trifluoromethyl)aniline (5 g, 25.6 mmol), ACN (150 mL), NBS (9.2 g, 51.7 mmol). The resulting solution was stirred overnight at RT and then was concentrated under vacuum. The residue was applied onto a silica gel column and eluted with ethyl acetate/petroleum ether (1:15 to 1:10). This resulted in 6 g (86%) of the title compound as a white solid. MS-ESI: 275.9, 273.9 (M+1).

Steps 2-6 used similar procedures for converting compound 74 to Intermediate 49 shown in Scheme AA to afford Intermediate 57. MS-ESI: 279.0 (M-1).

Compound 121: ¹H NMR (300 MHz, DMSO-d₆) δ 7.70 (s, 1H), 7.58 (s, 1H), 3.77 (s, 2H), 3.11-2.97 (m, 1H), 1.35 (s, 9H), 1.17 (d, J=6.8 Hz, 6H).

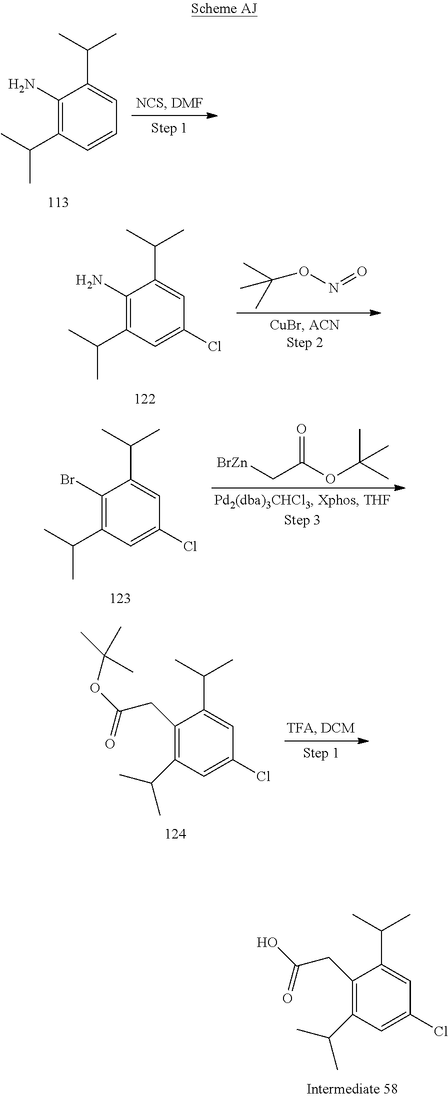

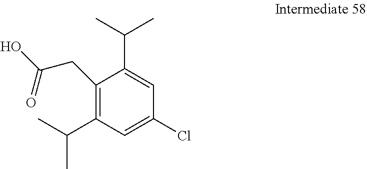 [SMILES: CC(C)c1cc(Cl)cc(C(C)C)c1CC(=O)O]

### 2-(4-Chloro-2,6-diisopropylphenyl)acetic acid

### Step 1: 4-Chloro-2,6-bis(propan-2-yl)aniline

Into a 100-mL round-bottom flask, was placed 2,6-bis(propan-2-yl)aniline (5 g, 28.2 mmol), DMF (20 mL), NCS (4.9 g, 36.7 mmol). The resulting solution was stirred for 15 h at RT and then was diluted with 20 mL of water. The resulting solution was extracted with 3×20 mL of DCM and the organic layers combined and concentrated under vacuum. The residue was applied onto a silica gel column and eluted with ethyl acetate/petroleum ether (1:10 to 1:5). This resulted in 3.7 g (62%) of the title compound as brown oil. MS-ESI: 212.1, 214.1 (M+1). Steps 2-4 used similar procedures for converting compound 76 to Intermediate 49 shown in Scheme AA to afford Intermediate 58. MS-ESI: 253.1, 255.1 (M-1).

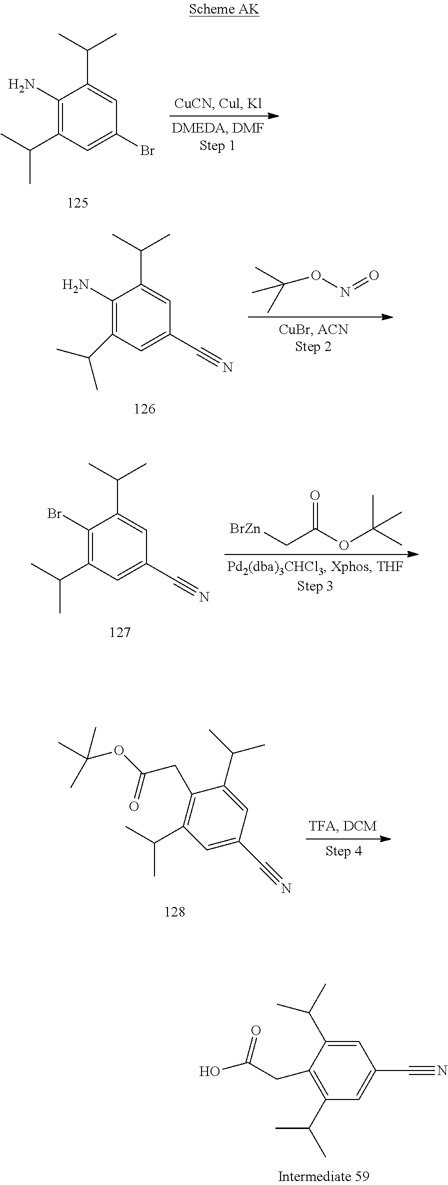

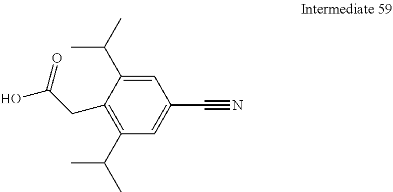 [SMILES: CC(C)c1cc(C#N)cc(C(C)C)c1CC(=O)O]

### 2-(4-Cyano-2,6-diisopropylphenyl)acetic acid

### Step 1: 4-Amino-3,5-bis(propan-2-yl)benzonitrile

Into a 100-mL round-bottom flask purged with and maintained under nitrogen, was placed 4-bromo-2,6-bis(propan-2-yl)aniline (5.1 g, 19.9 mmol), DMF (30 mL), CuCN (2.16 g, 23.9 mmol), CuI (380 mg, 2.00 mmol), KI (664 mg, 3.98 mmol), DMEDA (2.0 mL). The resulting solution was stirred for 24 h at 100° C. and then was diluted with 20 mL of water. The solution was extracted with 3×30 mL of ethyl acetate and the organic layers combined and concentrated under vacuum. The residue was applied onto a silica gel column and eluted with ethyl acetate/petroleum ether (1:30 to 1:20). This resulted in 1.2 g (30%) of the title compound as a yellow solid. MS-ESI: 203.1 (M+1).

Steps 2-4 used similar procedures for converting compound 76 to Intermediate 49 shown in Scheme AA to afford Intermediate 59. MS-ESI: 244.1 (M-1). ¹H NMR (400 MHz, DMSO-d₆) δ 12.54 (s, 1H), 7.56 (s, 2H), 3.79 (s, 2H), 3.12 (hept, J=6.8 Hz, 2H), 1.15 (d, J=6.7 Hz, 12H).

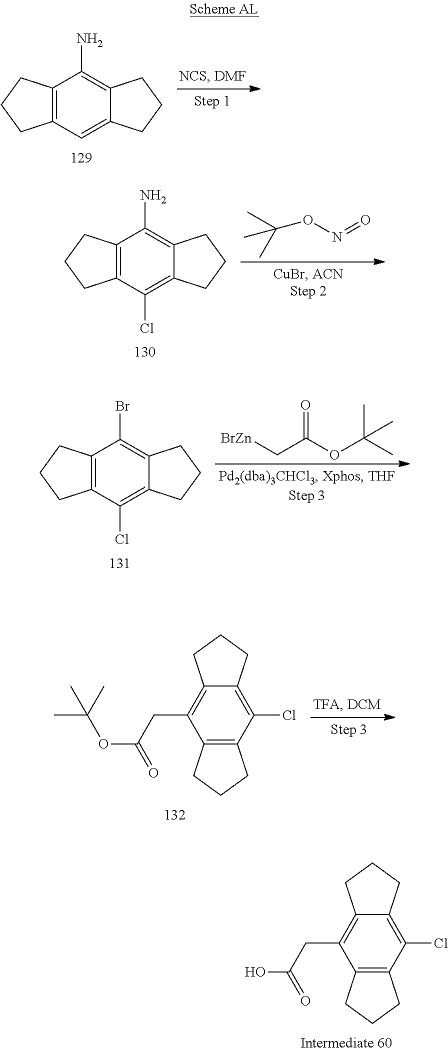

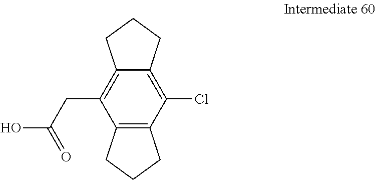 [SMILES: O=C(O)Cc1c2c(c(Cl)c3c1CCC3)CCC2]

### 2-(8-Chloro-1,2,3,5,6,7-hexahydros-indacen-4-yl)acetic acid

### Step 1: 8-Chloro-1,2,3,5,6,7-hexahydros-indacen-4-amine

Into a 100-mL round-bottom flask, was placed 1,2,3,5,6,7-hexahydros-indacen-4-amine (1.73 g, 9.99 mmol), DMF (10 mL), NCS (1.47 g, 11.0 mmol). The resulting solution was stirred for 12 h at RT and then was diluted with 50 mL of DCM. The resulting mixture was washed with 3×10 mL of water. The resulting mixture was concentrated under vacuum. The residue was applied onto a silica gel column and eluted with ethyl acetate/petroleum ether (1:5 to 1:10). This resulted in 1.88 g (91%) of the title compound as a yellow solid. MS-ESI: 208.1, 210.1 (M+1). Steps 2-4 used similar procedures for converting compound 76 to Intermediate 49 shown in Scheme AA to afford Intermediate 60. MS-ESI: 249.1, 251.1 (M-1).

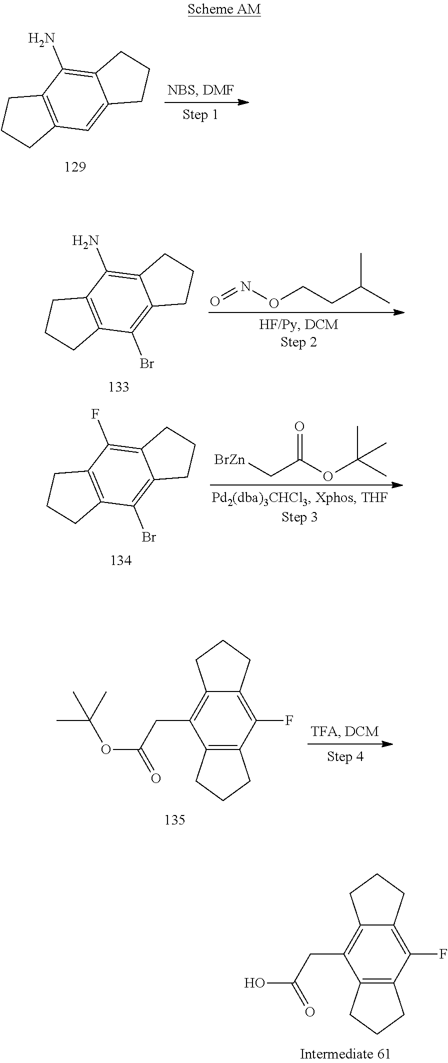

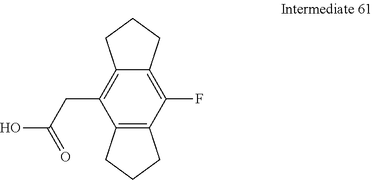 [SMILES: O=C(O)Cc1c2c(c(F)c3c1CCC3)CCC2]

### 2-(8-Fluoro-1,2,3,5,6,7-hexahydros-indacen-4-yl)acetic acid

### Step 1: 8-Bromo-1,2,3,5,6,7-hexahydro-s-indacen-4-amine

Into a 100-mL round-bottom flask, was placed 1,2,3,5,6,7-hexahydro-s-indacen-4-amine (2.6 g, 15.0 mmol), DMF (30 mL), NBS (2.9 g, 16.3 mmol). The resulting solution was stirred for 12 h at RT and then was diluted with 80 mL of ethyl acetate. The resulting mixture was washed with 3×20 mL of water. The resulting mixture was concentrated under vacuum. The residue was applied onto a silica gel column and eluted with ethyl acetate/petroleum ether (1:30 to 1:20).

This resulted in 3.0 g (79%) of the title compound as a brown solid. MS-ESI: 252.0, 254.0 (M+1).

### Step 2: 4-Bromo-8-fluoro-1,2,3,5,6,7-hexahydros-indacene

Into a 100-mL round-bottom flask, was placed 8-bromo-1,2,3,5,6,7-hexahydro-s-indacen-4-amine (1.5 g, 5.95 mmol), DCM (40 mL), HF/Py (70%, 4 mL), 3-methylbutyl nitrite (1.05 g, 8.96 mmol). The resulting solution was stirred for 2 h at RT and then was diluted with 50 mL of DCM. The resulting mixture was washed with 3×10 mL of water. The resulting mixture was concentrated under vacuum. The residue was applied onto a silica gel column and eluted with petroleum ether. This resulted in 1.2 g (79%) of the title compound as an off-white solid. ¹H NMR (400 MHz, DMSO-d₆) δ 3.00-2.80 (m, 8H), 2.15-2.00 (m, 4H).

Steps 3-4 used similar procedures for converting compound 77 to Intermediate 49 shown in Scheme AA to afford Intermediate 61. ¹H NMR (400 MHz, DMSO-d₆) δ 12.23 (s, 1H), 3.44 (s, 2H), 2.80 (dt, J=15.0, 7.5 Hz, 8H), 2.04-2.02 (m, 4H).

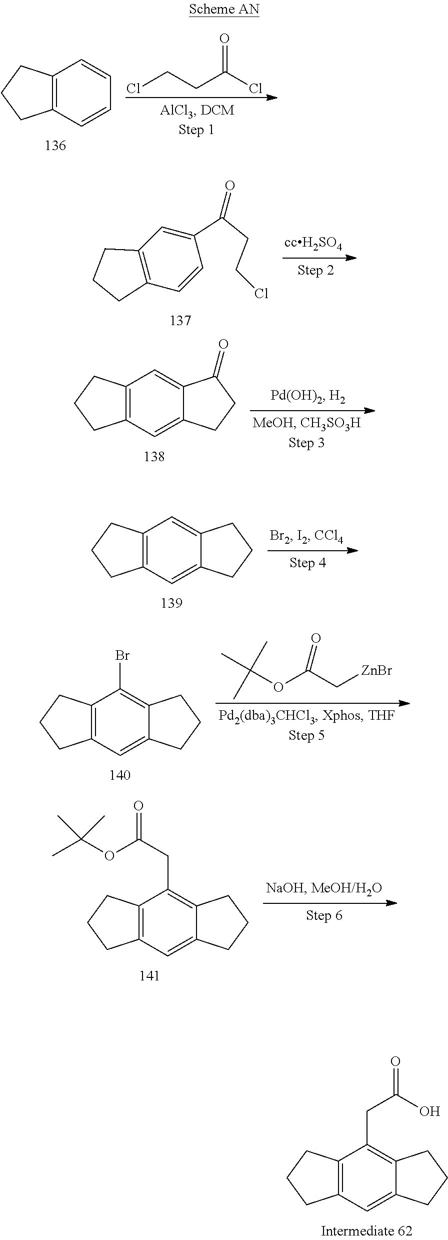

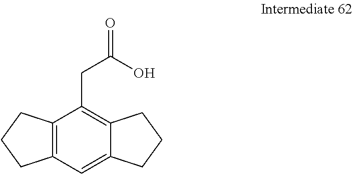 [SMILES: O=C(O)Cc1c2c(cc3c1CCC3)CCC2]

### 2-(1,2,3,5,6,7-Hexahydros-indacen-4-yl)acetic Acid

### Step 1: 3-Chloro-1-(2,3-dihydro-1H-inden-5-yl)propan-1-one

Into a 1000-mL round-bottom flask, was placed a solution of AlCl₃ (37 g, 278 mmol) in DCM (400 mL). This was followed by the addition of a solution of 2,3-dihydro-1H-indene (30 g, 254 mmol) and 3-chloropropanoyl chloride (32.1 g, 253 mmol) in DCM (100 mL) dropwise with stirring at −10° C. in 30 min. The resulting solution was stirred for 16 h at RT. Then the reaction mixture was added dropwise to cold HCl (3 N, 400 mL) over 45 min at −10° C. The resulting solution was extracted with 3×200 mL of DCM and the organic layers combined and dried over anhydrous Na₂SO₄, then concentrated under vacuum. This resulted in 53.5 g (crude) of the title compound as a yellow solid.

### Step 2: 1,2,3,5,6,7-Hexahydros-indacen-1-one

Into a 1000-mL round-bottom flask, was placed a solution of 3-chloro-1-(2,3-dihydro-1H-inden-5-yl)propan-1-one (53.5 g, 253 mmol) in cc. H2SO₄ (300 mL). The resulting solution was stirred for 16 h at 55° C. and then was quenched by the addition of 1500 mL of water/ice. The solids were collected by filtration and then was dried over infrared lamp for 24 h. This resulted in 37.4 g (85%) of the title compound as a yellow solid.

### Step 3: 1,2,3,5,6,7-Hexahydros-indacene

Into a 1000-mL round-bottom flask, was placed a solution of 1,2,3,5,6,7-hexahydros-indacen-1-one (37.2 g, 216.00 mmol), MeOH (300 mL), CH₃SO₃H (42 g). Then Pd(OH)₂/C (20% wt, 8 g) was added. The flask was evacuated and flushed three times with hydrogen. The resulting solution was stirred for 16 h at RT under an atmosphere of hydrogen. The solids were filtered out. The resulting mixture was concentrated under vacuum. The residue was applied onto a silica gel column and eluted with ethyl acetate/petroleum ether (1:150 to 1:100). This resulted in 27.1 g (79%) of the title compound as a white solid.

### Step 4: 4-Bromo-1,2,3,5,6,7-hexahydros-indacene

Into a 500-mL 3-necked round-bottom flask purged with and maintained under nitrogen, was placed a solution of 1,2,3,5,6,7-hexahydros-indacene (15 g, 94.8 mmol) in CCl₄ (200 mL). Then I₂ (1.2 g, 4.72 mmol) was added. This was followed by the addition of a solution of Br₂ (16 g, 100 mmol) in CCl₄ (50 mL) dropwise with stirring at 0° C. in 10 min. The resulting solution was stirred for 2 h at 0° C. The reaction was then quenched by the addition of 150 mL of NH₄Cl (sat.).

The resulting solution was extracted with 3×150 mL of DCM and the organic layers combined and dried over anhydrous Na₂SO₄, then concentrated under vacuum. This resulted in 23.3 g (crude) of the title compound as yellow oil. ¹H NMR (300 MHz, DMSO-d₆) δ 7.02 (s, 1H), 2.95-2.75 (m, 8H), 2.03-2.01 (m, 4H)

### Step 5: Tert-butyl 2-(1,2,3,5,6,7-hexahydros-indacen-4-yl)acetate

Into a 100-mL round-bottom flask purged with and maintained under nitrogen, was placed a solution of 4-bromo-1,2,3,5,6,7-hexahydros-indacene (1 g, 4.2 mmol) in THF (20 mL). Then X-phos (200 mg, 0.42 mmol) and Pd₂(dba)₃CHCl₃ (220 mg, 0.21 mmol) were added. The resulting solution was stirred for 10 min at RT. This was followed by the addition of tert-butyl 2-(bromozincio)acetate (2.2 g, 8.45 mmol). The resulting solution was stirred for 4 h at 80° C. and then was quenched by the addition of 50 mL of NH₄Cl (sat.). The resulting solution was extracted with 3×100 mL of DCM and the organic layers combined and dried over anhydrous Na₂SO₄, then concentrated under vacuum. This resulted in 1.4 g (crude) of the title compound as brown oil. ¹H NMR (400 MHz, DMSO-d₆) δ 6.96 (s, 1H), 3.47 (s, 2H), 2.79 (dt, J=17.6, 7.5 Hz, 8H), 2.01-1.99 (m, 4H), 1.39 (s, 9H).

### Step 6: 2-(1,2,3,5,6,7-hexahydros-indacen-4-yl)acetic acid

Into a 40-mL sealed tube, was placed a solution of tert-butyl 2-(1,2,3,5,6,7-hexahydros-indacen-4-yl)acetate (1.4 g, 5.14 mmol) in 6 M sodium hydroxide/MeOH (4/6 mL). The resulting solution was stirred for 16 h at 100° C. The reaction was then quenched by the addition of 20 mL of water. The resulting solution was extracted with 2×30 mL of DCM and the aqueous layers combined. The pH value of the solution was adjusted to 2 with hydrogen chloride (1 N). The resulting solution was extracted with 3×50 mL of ethyl acetate and the organic layers combined and dried over anhydrous Na₂SO₄, then concentrated under vacuum. This resulted in 180 mg (16%) of the title compound as a yellow solid. MS-ESI: 215.1 (M-1).

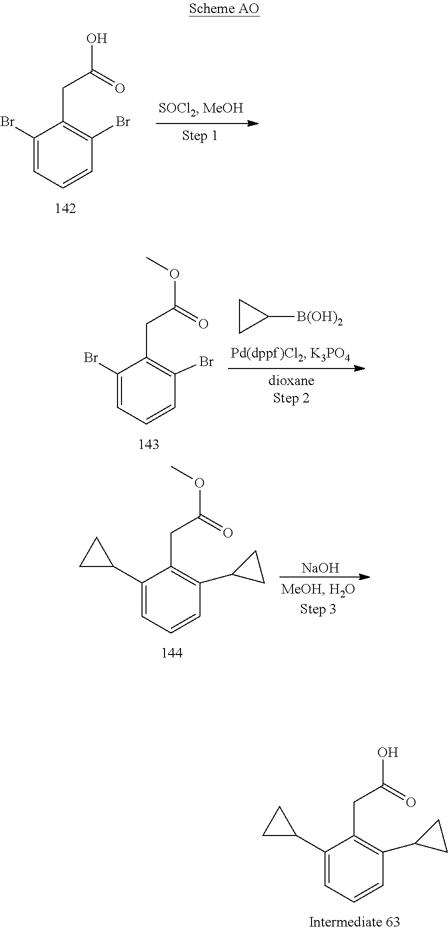

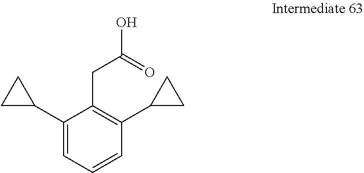 [SMILES: O=C(O)Cc1c(C2CC2)cccc1C1CC1]

### 2-(2,6-Dicyclopropylphenyl)acetic acid

### Step 1: Methyl 2-(2,6-dibromophenyl)acetate

Into a 250-mL round-bottom flask, was placed 2-(2,6-dibromophenyl)acetic acid (5 g, 17.0 mmol), methanol (50 mL). This was followed by the addition of sulfuroyl dichloride (4.1 g, 34.5 mmol) dropwise with stirring at 0° C. The resulting solution was stirred for 3 h at 60° C. and then was concentrated under vacuum. The residue was applied onto a silica gel column and eluted with ethyl acetate/petroleum ether (1:15 to 1:10). This resulted in 4.5 g (86%) of the title compound as light yellow oil. MS-ESI: 308.9, 306.9, 310.9 (M+1).

### Step 2: Methyl 2-(2,6-dicyclopropylphenyl)acetate

Into a 50-mL round-bottom flask purged with and maintained under nitrogen, was placed methyl 2-(2,6-dibromophenyl)acetate (600 mg, 1.95 mmol), dioxane (20 mL), cyclopropylboronic acid (688 mg, 8.01 mmol), K₃PO₄ (2.1 g, 9.89 mmol), Pd(dppf)Cl₂ (146 mg, 0.20 mmol). The resulting solution was stirred for 4 h at 100° C. and then was concentrated under vacuum. The residue was applied onto a silica gel column and eluted with ethyl acetate/petroleum ether (1:30 to 1:20). This resulted in 440 mg (98%) of the title compound as yellow oil. MS-ESI: 231.1 (M+1).

### Step 3: 2-(2,6-Dicyclopropylphenyl)acetic acid

Into a 50-mL round-bottom flask, was placed methyl 2-(2,6-dicyclopropylphenyl)acetate (440 mg, 1.91 mmol). Then to the above was added a solution of sodium hydroxide (228 mg, 5.70 mmol) in MeOH (15 mL) and water (4 mL). The resulting solution was stirred for 2 days at 50° C. The resulting solution was extracted with 20 mL of ethyl acetate and the aqueous layers combined. The pH value of the solution was adjusted to 4 with hydrogen chloride (6 N). The resulting solution was extracted with 3×20 mL of ethyl acetate and the organic layers combined dried over anhydrous Na₂SO₄, then concentrated under vacuum. This resulted in 260 mg (63%) of the title compound as a yellow solid. MS-ESI: 215.1 (M-1).

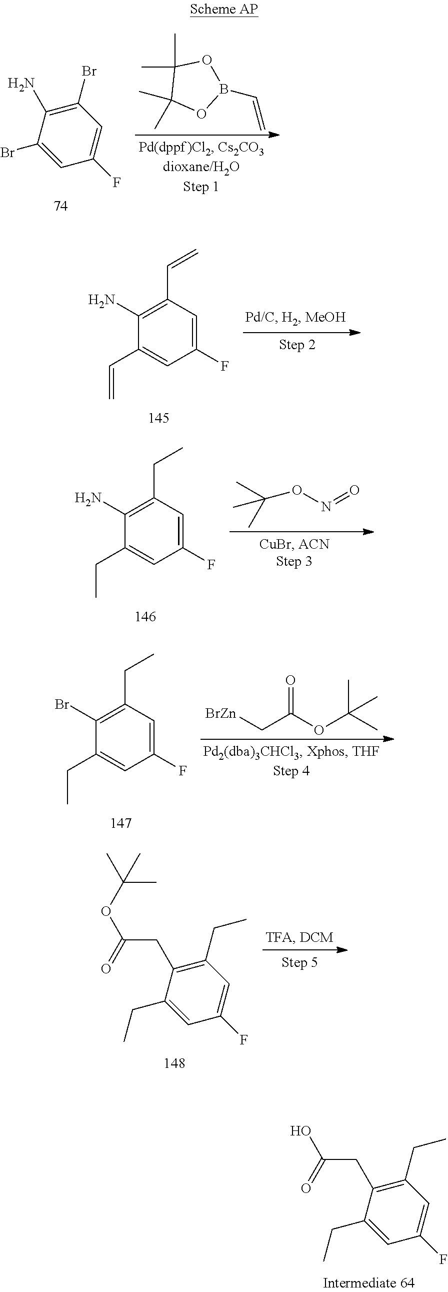

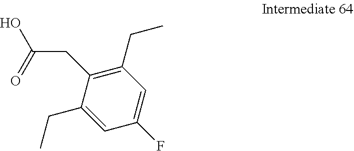

### 2-(2,6-Diethyl-4-fluorophenyl)acetic acid

Intermediate 64 was prepared using the similar procedures for converting compound 74 to 5 Intermediate 49 shown in Scheme AA. MS-ESI: 209.1 (M-1).

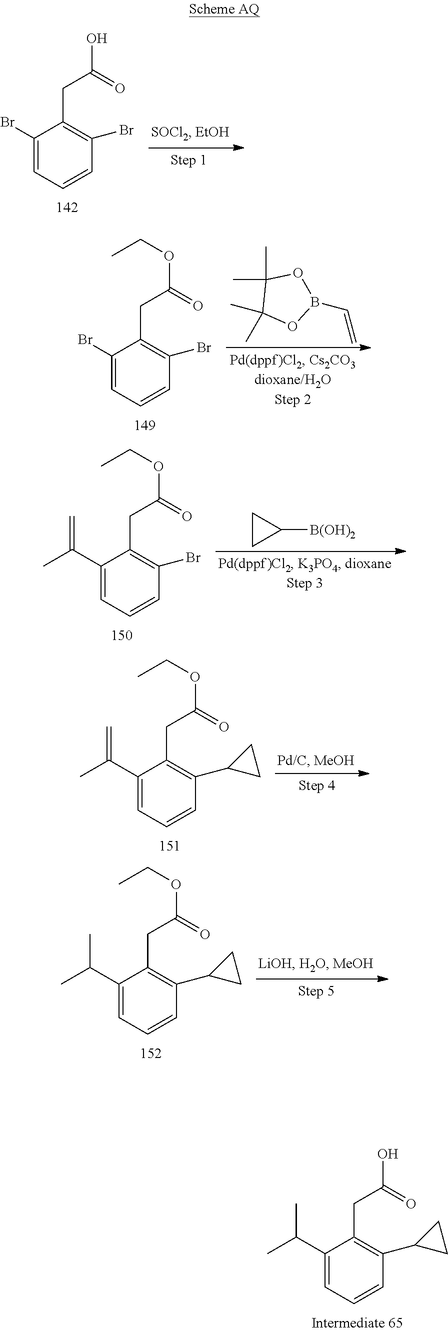

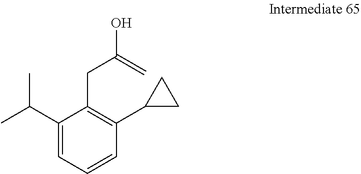 [SMILES: C=C(O)Cc1c(C(C)C)cccc1C1CC1]

### 2-(2-Cyclopropyl-6-isopropylphenyl)acetic acid

### Step 1: Ethyl 2-(2,6-dibromophenyl)acetate

Into a 250-mL round-bottom flask, was placed 2-(2,6-dibromophenyl)acetic acid (3.1 g, 10.55 mmol), EtOH (80 mL). This was followed by the addition of sulfuroyl dichloride (4 g, 33.61 mmol) dropwise with stirring at 0° C. The resulting solution was stirred overnight at 60° C. and then was concentrated under vacuum. The residue was applied onto a silica gel column and eluted with ethyl acetate/petroleum ether (1:3 to 1:1). This resulted in 3.4 g (crude) of the title compound as colorless oil. MS-ESI: 322.9, 320.9, 324.9 (M+1).

### Step 2: Ethyl 2-(2-bromo-6-(prop-1-en-2-yl)phenyl)acetate

Into a 250-mL round-bottom flask purged with and maintained under nitrogen, was placed ethyl 2-(2,6-dibromophenyl)acetate (3.4 g, 10.6 mmol), dioxane (90 mL), water (20 mL), Cs₂CO₃ (3.6 g, 11.1 mmol), 4,4,5,5-tetramethyl-2-(prop-1-en-2-yl)-1,3,2-dioxaborolane (2.06 g, 12.3 mmol), Pd(dppf)Cl₂ (320 mg, 0.44 mmol). The resulting solution was stirred for 7.5 h at 50° C. and then quenched by the addition of 30 mL of water. The resulting solution was extracted with 3×50 mL of ethyl acetate and the organic layers combined and dried over anhydrous Na₂SO₄, and then concentrated under vacuum. The residue was applied onto a silica gel column and eluted with ethyl acetate/petroleum ether (1:5 to 1:3). This resulted in 920 mg (31%) of the title compound as light yellow oil. MS-ESI: 283.0, 285.0 (M+1).

### Step 3: Ethyl 2-(2-cyclopropyl-6-(prop-1-en-2-yl)phenyl)acetate

Into a 100-mL round-bottom flask purged with and maintained under nitrogen, was placed ethyl 2-(2-bromo-6-(prop-1-en-2-yl)phenyl)acetate (300 mg, 1.06 mmol), dioxane (10 mL), cyclopropylboronic acid (180 mg, 2.10 mmol), K₃PO₄ (429 mg, 2.02 mmol), Pd(dppf)Cl₂ (43 mg, 0.06 mmol). The resulting solution was stirred for 5 h at 110° C. and then was quenched by the addition of 30 mL of water. The resulting solution was extracted with 3×50 mL of ethyl acetate and the organic layers combined and dried over anhydrous Na₂SO₄, and then concentrated under vacuum. The residue was applied onto a silica gel column and eluted with ethyl acetate/petroleum ether (1:3 to 1:1). This resulted in 228 mg (88%) of the title compound as light yellow oil. MS-ESI: 245.1 (M+1).

### Step 4: Ethyl 2-(2-cyclopropyl-6-isopropylphenyl)acetate

Into a 250-mL round-bottom, was placed ethyl 2-(2-cyclopropyl-6-(prop-1-en-2-yl)phenyl)acetate (228 mg, 0.93 mmol), MeOH (10 mL). Then Pd/C (10% wt, 50 mg) was added. The flask was evacuated and flushed three times with hydrogen. The resulting solution was stirred for 3.5 h at RT under an atmosphere of hydrogen. The solids were filtered out. The resulting mixture was concentrated under vacuum. This resulted in 162 mg (70%) of the title compound as colorless oil. MS-ESI: 247.1 (M+1).

### Step 5: 2-(2-Cyclopropyl-6-isopropylphenyl)acetic acid

Into a 100-mL round-bottom flask, was placed ethyl 2-(2-cyclopropyl-6-isopropylphenyl)acetate (162 mg, 0.66 mmol), MeOH (10 mL), water (2 mL), LiOH (200 mg, 8.35 mmol). The resulting solution was stirred for 5 h at RT and then was concentrated under vacuum. The resulting solution was diluted with 10 mL of 1 N hydrogen chloride. The resulting solution was extracted with 3×10 mL of DCM and the organic layers combined and dried over anhydrous Na₂SO₄, and then concentrated under vacuum. This resulted in 140 mg (98%) of the title compound as a light yellow solid. MS-ESI: 217.1 (M-1).

### Example 1

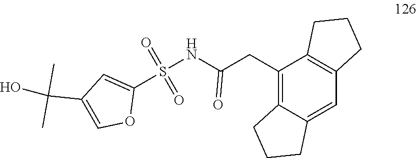 [SMILES: CC(C)(O)c1coc(S(=O)(=O)NC(=O)Cc2c3c(cc4c2CCC4)CCC3)c1]

### 2-(1,2,3,5,6,7-Hexahydros-indacen-4-yl)-N-(4-(2-hydroxypropan-2-yl)furan-2-ylsulfonyl)acetamide (Scheme A)

Into a 50-mL round-bottom flask purged with and maintained under nitrogen, was placed 2-(1,2,3,5,6,7-hexahydros-indacen-4-yl)acetic acid (125 mg, 0.58 mmol), DMF (5 mL), CDI (113 mg, 0.70 mmol). The resulting solution was stirred for 1 h at RT and then to the above was added 4-(2-hydroxypropan-2-yl)furan-2-sulfonamide (119 mg, 0.58 mmol), DBU (0.11 mL). The resulting solution was stirred for 3 h at RT and then was diluted with 10 mL of water. The resulting solution was extracted with 3×10 mL of ethyl acetate and the organic layers combined and concentrated under vacuum. The crude product was purified by Prep-HPLC using method E eluted with a gradient of 30˜40% ACN. This resulted in 59.9 mg (26%) of the title compound as a white solid. MS-ESI: 402.0 (M-1). ¹H NMR (400 MHz, MeOD-d₄) δ 7.44 (s, 1H), 6.86 (s, 1H), 6.84 (s, 1H), 3.48 (s, 2H), 2.89-2.65 (m, 8H), 2.10-1.90 (m, 4H), 1.45 (s, 6H).

### Example 2

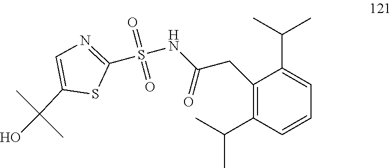 [SMILES: CC(C)c1cccc(C(C)C)c1CC(=O)NS(=O)(=O)c1ncc(C(C)(C)O)s1]

### 2-(2,6-Diisopropylphenyl)-N-(5-(2-hydroxypropan-2-yl)thiazol-2-ylsulfonyl)acetamide(Scheme B)

Into a 50-mL round-bottom flask, was placed 2-(2,6-diisopropylphenyl)acetic acid (60 mg, 0.27 mmol), DMF (5 mL), HBTU (124 mg, 0.33 mmol), DIEA (105 mg, 0.81 mmol), 5-(2-hydroxypropan-2-yl)thiazole-2-sulfonamide (67 mg, 0.30 mmol). The resulting solution was stirred overnight at RT and then was concentrated under vacuum. The crude product was purified by Prep-HPLC using method E eluted with a gradient of 21-43% ACN. This resulted in 44.3 mg (38%) of the title compound as a white solid. MS-ESI. 423.2 (M-1). ¹H NMR (300 MHz, MeOD-d₄) δ 7.60 (s, 1H), 7.18-7.00 (m, 3H), 3.76 (s, 2H), 3.14 (hept, J=6.6 Hz, 2H), 1.59 (s, 6H), 1.14 (d, J=6.6 Hz, 12H).

### Example 3

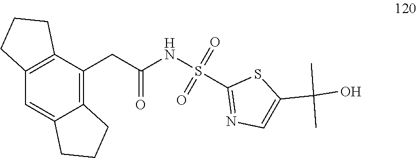 [SMILES: CC(C)(O)c1cnc(S(=O)(=O)NC(=O)Cc2c3c(cc4c2CCC4)CCC3)s1]

### 2-(1,2,3,5,6,7-Hexahydros-indacen-4-yl)-N-(5-(2-hydroxypropan-2-yl)thiazol-2-ylsulfonyl)acetamide (Scheme C)

Into a 50-mL round-bottom flask purged with and maintained under nitrogen, was placed 2-(1,2,3,5,6,7-hexahydros-indacen-4-yl)acetic acid (500 mg, 2.31 mmol), DCM (20 mL), DIEA (900 mg, 6.96 mmol), HATU (1.06 g, 2.79 mmol). The resulting solution was stirred for 0.5 h at RT and then to the above was added 5-(2-hydroxypropan-2-yl)thiazole-2-sulfonamide (570 mg, 2.56 mmol). The resulting solution was stirred for 2 h at RT and then was quenched by the addition of 15 mL of water. The resulting solution was extracted with 2×30 mL of DCM and the organic layers combined and dried over anhydrous Na₂SO₄, and then concentrated under vacuum. The crude product was purified by Prep-HPLC using method E eluted with a gradient of 25˜50% ACN. This resulted in 293.2 mg (30%) of the title compound as a yellow solid. MS-ESI: 421.1 (M+1). ¹H NMR (300 MHz, MeOD-d₄) δ 7.61 (s, 1H), 6.84 (s, 1H), 3.50 (s, 2H), 2.86-2.66 (m, 8H), 2.10-1.90 (m, 4H), 1.57 (s, 6H).

### Example 4

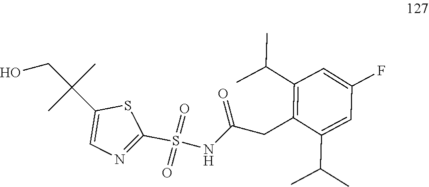 [SMILES: CC(C)c1cc(F)cc(C(C)C)c1CC(=O)NS(=O)(=O)c1ncc(C(C)(C)CO)s1]

### 2-(4-Fluoro-2,6-diisopropylphenyl)-N-(5-(1-hydroxy-2-methylpropan-2-yl)thiazol-2-ylsulfonyl)acetamide (Scheme D)

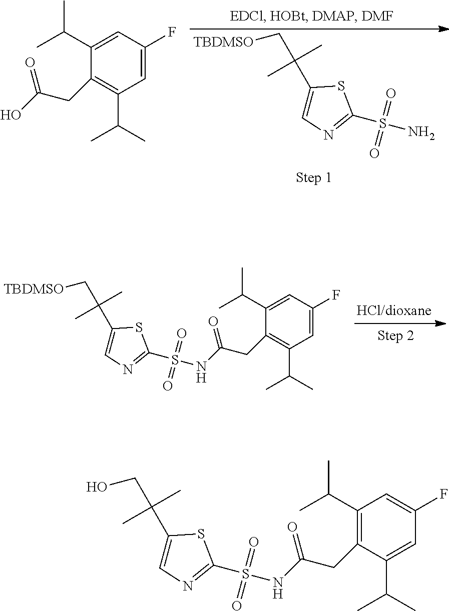 [SMILES: CC(C)c1cc(F)cc(C(C)C)c1CC(=O)O ; CC(C)(CO[Si](C)(C)C(C)(C)C)c1cnc(S(N)(=O)=O)s1 ; CC(C)c1cc(F)cc(C(C)C)c1CC(=O)NS(=O)(=O)c1ncc(C(C)(C)CO[Si](C)(C)C(C)(C)C)s1 ; CC(C)c1cc(F)cc(C(C)C)c1CC(=O)NS(=O)(=O)c1ncc(C(C)(C)CO)s1]

### Step 1: N-(5-(1-(tert-butyldimethylsilyloxy)-2-methylpropan-2-yl)thiazol-2-ylsulfonyl)-2-(4-fluoro-2,6-diisopropylphenyl)acetamide

Into a 50-mL round-bottom flask, was placed 2-(4-fluoro-2,6-diisopropylphenyl)acetic acid (100 mg, 0.42 mmol), DMF (5 mL), EDCI (121 mg, 0.63 mmol), HOBt (85 mg, 0.63 mmol), DMAP (5 mg, 0.04 mmol). The resulting solution was stirred for 20 min at RT and then to the above was added 5-(1-(tert-butyldimethylsilyloxy)-2-methylpropan-2-yl)thiazole-2-sulfonamide (147 mg, 0.42 mmol). The resulting solution was stirred for 3 h at RT and then was diluted with 10 mL of water. The resulting solution was extracted with 2×10 mL of DCM and the organic layers combined and dried over anhydrous Na₂SO₄, and then concentrated under vacuum. This resulted in 150 mg (crude, 63%) of the title compound as brown oil. MS-ESI: 569.2 (M-1).

### Step 2: 2-(4-Fluoro-2,6-diisopropylphenyl)-N-(5-(1-hydroxy-2-methylpropan-2-yl)thiazol-2-ylsulfonyl) acetamide

Into a 50-mL round-bottom flask, was placed N-(5-(1-(tert-butyldimethylsilyloxy)-2-methylpropan-2-yl)thiazol-2-ylsulfonyl)-2-(4-fluoro-2,6-diisopropylphenyl)acetamide (150 mg, 0.26 mmol), HCl/dioxane (4 M, 5 mL). The resulting solution was stirred for 3 h at RT and then was concentrated under vacuum. The crude product was purified by Prep-HPLC using method E eluted with a gradient of 15˜60% ACN. This resulted in 117.3 mg (78%) of the title compound as a white solid. MS-ESI: 455.1 (M-1). ¹H NMR (300 MHz, MeOD-d₄) δ 7.64 (s, 1H), 6.74 (d, J=10.2 Hz, 2H), 3.73 (s, 2H), 3.45 (s, 2H), 3.10-2.90 (m, 2H), 1.33 (s, 6H), 1.09 (d, J=6.9 Hz, 12H).

### Example 5

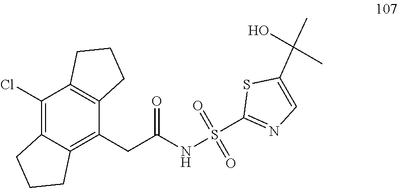 [SMILES: CC(C)(O)c1cnc(S(=O)(=O)NC(=O)Cc2c3c(c(Cl)c4c2CCC4)CCC3)s1]

### 2-(8-Chloro-1,2,3,5,6,7-hexahydros-indacen-4-yl)-N-(5-(2-hydroxypropan-2-yl)thiazol-2-ylsulfonyl) acetamide (Scheme E)

Into a 50-mL round-bottom flask, was placed 2-(8-chloro-1,2,3,5,6,7-hexahydros-indacen-4-yl)acetic acid (60 mg, 0.27 mmol), DCM (3 mL), DMF (0.05 mL). This was followed by the addition of oxalic dichloride (0.5 mL) dropwise with stirring at RT. The resulting solution was stirred for 30 min at RT and then was concentrated under vacuum. The above mixture diluted in DCM (1 mL) was added to a solution of 5-(2-hydroxypropan-2-yl)thiazole-2-sulfonamide (60 mg, 0.27 mmol) and TEA (0.2 mL) in DCM (3 mL) dropwise with stirring at RT. The resulting solution was stirred for 2 h at RT and then was concentrated under vacuum. The crude product was purified by Prep-HPLC using method E eluted with a gradient of 30˜50% ACN. This resulted in 26.7 mg (37%) of the title compound as a white solid. MS-ESI. 455.1 (M+1). ¹H NMR (300 MHz, MeOD-d₄) δ 7.66 (s, 1H), 3.51 (s, 2H), 2.95-2.78 (m, 8H), 2.15-1.95 (m, 4H), 1.61 (s, 6H).

TABLE 6
Example in the following table was prepared using
similar conditions as described in Example 3 and
Scheme C from appropriate starting materials.

| | Final | | Mass |
|---|---|---|---|
| Example | Target | | Spec |
| # | Number | IUPAC Name | [M + H]⁺ |
| 6 | 128 | 2-(4-cyano-2,6- | 450.1 |
| | | diisopropylphenyl)-N-(5-(2- | |
| | | hydroxypropan-2-yl)thiazol-2- | |
| | | ylsulfonyl)acetamide | |

TABLE 7
Examples in the following table were prepared using
similar conditions as described in Example 5 and
Scheme E from appropriate starting materials.

| | Final | | Mass |
|---|---|---|---|
| Example | Target | | Spec |
| # | Number | IUPAC Name | [M − H]⁻ |
| 7 | 116 | 2-(1,2,3,5,6,7-hexahydros- | 418.1 |
| | | indacen-4-yl)-N-(4-(2- | |
| | | hydroxypropan-2-yl)thiophen-2- | |
| | | ylsulfonyl)acetamide | |
| 8 | 117 | 2-(2,6-diisopropylphenyl)-N-(4-(2- | 422.1 |
| | | hydroxypropan-2-yl)thiophen-2- | |
| | | ylsulfonyl)acetamide | |
| 9 | 129 | 2-(4-fluoro-2,6- | 440.1 |
| | | diisopropylphenyl)-N-(4-(2- | |
| | | hydroxypropan-2-yl)thiophen- | |
| | | 2-ylsulfonyl)acetamide | |
| 10 | 130 | 2-(4-cyano-2,6- | 447.2 |
| | | diisopropylphenyl)-N-(4-(2- | |
| | | hydroxypropan-2-yl)thiophen- | |
| | | 2-ylsulfonyl)acetamide | |
| 11 | 103 | 2-(3-fluoro-2,6- | 440.0 |
| | | diisopropylphenyl)-N-(4-(2- | |
| | | hydroxypropan-2-yl)thiophen- | |
| | | 2-ylsulfonyl)acetamide | |
| 12 | 131 | 2-(4-chloro-3,5-difluoro-2,6- | 492.1 |
| | | diisopropylphenyl)-N-(4-(2- | |
| | | hydroxypropan-2-yl)thiophen- | |
| | | 2-ylsulfonyl)acetamide | |
| 13 | 132 | 2-(4-fluoro-2,6- | 440.1 |
| | | diisopropylphenyl)-N-(5-(2- | |
| | | hydroxypropan-2-yl)thiophen-2- | |
| | | ylsulfonyl)acetamide | |
| 14 | 133 | 2-(2,6-diisopropylphenyl)-N-(4-(1- | 420.2 |
| | | hydroxycyclopropyl)thiophen-2- | |
| | | ylsulfonyl)acetamide | |
| 15 | 134 | 2-(4-fluoro-2,6- | 438.1 |
| | | diisopropylphenyl)-N-(4-(1- | |
| | | hydroxycyclopropyl)thiophen- | |
| | | 2-ylsulfonyl)acetamide | |
| 16 | 135 | 2-(4-cyano-2,6- | 445.2 |
| | | diisopropylphenyl)-N-(4-(1- | |
| | | hydroxycyclopropyl)thiophen- | |
| | | 2-ylsulfonyl)acetamide | |
| 17 | 136 | 2-(4-fluoro-2,6- | 454.1 |
| | | diisopropylphenyl)-N-(4-(2- | |
| | | hydroxypropan-2-yl)-5- | |
| | | methylthiophen-2- | |
| | | ylsulfonyl)acetamide | |
| 18 | 137 | 2-(4-fluoro-2,6- | 438.2 |
| | | diisopropylphenyl)-N-(4-(2- | |
| | | hydroxypropan-2-yl)-5-methylfuran- | |
| | | 2-ylsulfonyl)acetamide | |
| 19 | 138 | 2-(4-fluoro-2,6- | 454.1 |
| | | diisopropylphenyl)-N-(4-(2- | |
| | | hydroxypropan-2-yl)-3- | |
| | | methylthiophen-2- | |
| | | ylsulfonyl)acetamide | |
| 20 | 139 | 2-(4-chloro-3,5-difluoro-2,6- | 476.1 |
| | | diisopropylphenyl)-N-(4-(2- | |
| | | hydroxypropan-2-yl)furan-2- | |
| | | ylsulfonyl)acetamide | |

TABLE 8
Examples in the following table were prepared using
similar conditions as described in Example 5 and
Scheme E from appropriate starting materials.

| | Final | | Mass |
|---|---|---|---|
| Example | Target | | Spec |
| # | Number | IUPAC Name | [M − H]⁻ |
| 21 | 140 | 2-(2,6-diisopropylphenyl)-N-(4-(2- | 418.3 |
| | | hydroxypropan-2- | (M + 1) |
| | | yl)phenylsulfonyl)acetamide | |
| 22 | 141 | 2-(2,6-diisopropylphenyl)-N-(3-(2- | 493.2 |
| | | hydroxypropan-2-yl)-5-(pyridin-4- | |
| | | yl)phenylsulfonyl)acetamide | |
| 23 | 142 | 2-(2,6-diisopropylphenyl)-N-(5-(2- | 492.2 |
| | | hydroxypropan-2-yl)biphenyl- | |
| | | 3-ylsulfonyl)acetamide | |
| 24 | 143 | N-(3,5-bis(2-hydroxypropan-2- | 440.1 |
| | | yl)phenylsulfonyl)-2- | (M − |
| | | (2,6-diisopropylphenyl)acetamide | 2OH) |
| 25 | 144 | 2-(4-fluoro-2,6- | 434.0 |
| | | diisopropylphenyl)-N-(3-(2- | |
| | | hydroxypropan-2- | |
| | | yl)phenylsulfonyl)acetamide | |
| 26 | 145 | 2-(4-cyano-2,6- | 441.2 |
| | | diisopropylphenyl)-N-(3-(2- | |
| | | hydroxypropan-2- | |
| | | yl)phenylsulfonyl)acetamide | |
| 27 | 146 | N-(3-chloro-5-(2-hydroxypropan-2- | 468.1 |
| | | yl)phenylsulfonyl)-2-(4-fluoro-2,6- | |
| | | diisopropylphenyl)acetamide | |
| 28 | 147 | N-(3-chloro-5-(2-hydroxypropan-2- | 475.2 |
| | | yl)phenylsulfonyl)-2-(4-cyano-2,6- | |
| | | diisopropylphenyl)acetamide | |
| 29 | 148 | 2-(4-fluoro-2,6- | 511.2 |
| | | diisopropylphenyl)-N-(3-(2- | |
| | | hydroxypropan-2-yl)-5-(pyridin-4- | |
| | | yl)phenylsulfonyl)acetamide | |
| 30 | 149 | N-(3,5-bis(2-hydroxypropan-2- | 492.2 |
| | | yl)phenylsulfonyl)-2-(4-fluoro-2,6- | |
| | | diisopropylphenyl)acetamide | |
| 31 | 150 | N-(3,5-bis(2-hydroxypropan-2- | 499.2 |
| | | yl)phenylsulfonyl)-2-(4-cyano-2,6- | |
| | | diisopropylphenyl)acetamide | |
| 32 | 151 | 2-(4-fluoro-2,6- | 510.2 |
| | | diisopropylphenyl)-N-(5-(2- | |
| | | hydroxypropan-2-yl)biphenyl-3- | |
| | | ylsulfonyl)acetamide | |
| 33 | 152 | 2-(4-cyano-2,6- | 517.3−1 |
| | | diisopropylphenyl)-N-(5-(2- | |
| | | hydroxypropan-2-yl)biphenyl-3- | |
| | | ylsulfonyl)acetamide | |
| 34 | 153 | 2-(4-fluoro-2,6- | 434.2 |
| | | diisopropylphenyl)-N-(4-(2- | |
| | | hydroxypropan-2- | |
| | | yl)phenylsulfonyl)acetamide | |
| 35 | 154 | 2-(4-cyano-2,6- | 441.2 |
| | | diisopropylphenyl)-N-(4-(2- | |
| | | hydroxypropan-2- | |
| | | yl)phenylsulfonyl)acetamide | |
| 36 | 155 | 2-(4-fluoro-2,6- | 435.1 |
| | | diisopropylphenyl)-N-(6-(2- | |
| | | hydroxypropan-2-yl)pyridin-3- | |
| | | ylsulfonyl)acetamide | |
| 37 | 156 | 2-(4-cyano-2,6- | 442.2 |
| | | diisopropylphenyl)-N-(6-(2- | |
| | | hydroxypropan-2-yl)pyridin-3- | |
| | | ylsulfonyl)acetamide | |
| 38 | 157 | 2-(4-fluoro-2,6- | 521.3 |
| | | diisopropylphenyl)-N-(3-(2- | (M + 1) |
| | | hydroxypropan-2-yl)-5- | |
| | | morpholinophenylsulfonyl)acetamide | |
| 39 | 158 | N-(4-pentafluorophenylsulfonyl)-2- | 502.1 |
| | | (4-fluoro-2,6- | |
| | | diisopropylphenyl)acetamide | |
| 40 | 159 | 2-(4-fluoro- | 427.1 |
| | | 2,6-diisopropylphenyl)-N- | |
| | | (quinolin-3-ylsulfonyl)acetamide2- | |
| | | (4-fluoro-2,6-diisopropylphenyl)-N- | |
| | | (quinolin-3-ylsulfonyl)acetamide | |
| 41 | 160 | N-(benzofuran-2-ylsulfonyl)-2- | 416.1 |
| | | (4-fluoro-2,6- | |
| | | diisopropylphenyl)acetamide | |
| 42 | 161 | 2-(3-fluoro-2,6- | 434.2 |
| | | diisopropylphenyl)-N-(3-(2- | |
| | | hydroxypropan-2- | |
| | | yl)phenylsulfonyl)acetamide | |
| 43 | 162 | 2-(3-fluoro-2,6- | 434.2 |
| | | diisopropylphenyl)-N-(4-(2- | |
| | | hydroxypropan-2- | |
| | | yl)phenylsulfonyl)acetamide | |
| 44 | 163 | 2-(4-fluoro-2,6- | 448.2 |
| | | diisopropylphenyl)-N-(3-(2- | |
| | | hydroxypropan-2-yl)-2- | |
| | | methylphenylsulfonyl)acetamide | |
| 45 | 164 | 2-(4-fluoro-2,6- | 448.2 |
| | | diisopropylphenyl)-N-(3-(2- | |
| | | hydroxypropan-2-yl)-4- | |
| | | methylphenylsulfonyl)acetamide | |
| 46 | 165 | 2-(4-fluoro-2,6- | 448.2 |
| | | diisopropylphenyl)-N-(3-(2- | |
| | | hydroxypropan-2-yl)-5- | |
| | | methylphenylsulfonyl)acetamide | |
| 47 | 166 | 2-(4-fluoro-2,6- | 450.2 |
| | | diisopropylphenyl)-N-(4-(2- | (M + 1) |
| | | hydroxypropan-2-yl)-3- | |
| | | methylphenylsulfonyl)acetamide | |
| 48 | 167 | 2-(4-fluoro-2,6- | 448.2 |
| | | diisopropylphenyl)-N-(4-(2- | |
| | | hydroxypropan-2-yl)-2- | |
| | | methylphenylsulfonyl)acetamide | |
| 49 | 168 | 2-(4-fluoro-2,6- | 452.3 |
| | | diisopropylphenyl)-N-(1- | |
| | | fluoro-3-(2-hydroxypropan-2- | |
| | | yl)phenylsulfonyl)acetamide | |
| 50 | 169 | 2-(4-fluoro-2,6- | 452.3 |
| | | diisopropylphenyl)-N-(2- | |
| | | fluoro-3-(2-hydroxypropan-2- | |
| | | yl)phenylsulfonyl)acetamide | |
| 51 | 170 | 2-(4-fluoro-2,6- | 452.3 |
| | | diisopropylphenyl)-N-(3- | |
| | | fluoro-5-(2-hydroxypropan-2- | |
| | | yl)phenylsulfonyl)acetamide | |
| 52 | 171 | 2-(4-fluoro-2,6- | 452.3 |
| | | diisopropylphenyl)-N-(2- | |
| | | fluoro-5-(2-hydroxypropan-2- | |
| | | yl)phenylsulfonyl)acetamide | |
| 53 | 172 | 2-(4-fluoro-2,6- | 452.2 |
| | | diisopropylphenyl)-N-(3- | |
| | | fluoro-4-(2-hydroxypropan-2- | |
| | | yl)phenylsulfonyl)acetamide | |
| 54 | 173 | 2-(4-fluoro-2,6- | 452.2 |
| | | diisopropylphenyl)-N-(2- | |
| | | fluoro-4-(2-hydroxypropan-2- | |
| | | yl)phenylsulfonyl)acetamide | |
| 55 | 174 | N-(5-acetyl-2- | 438.2 |
| | | fluorophenylsulfonyl)-2-fluoro- | (M + 1) |
| | | 2,6-diisopropylphenyl)acetamide | |
| 56 | 175 | 2-(4-fluoro-2,6- | 482.3 |
| | | diisopropylphenyl)-N-(2- | (M + 1) |
| | | fluoro-5-(2-methyl-1,3-dioxolan-2- | |
| | | yl)phenylsulfonyl)acetamide | |
| 57 | 176 | 2-(8-fluoro-1,2,3,5,6,7-hexahydros- | 478.4 |
| | | indacen-4-yl)-N-(2-fluoro-5-(2- | (M + 1) |
| | | methyl-1,3-dioxolan-2- | |
| | | yl)phenylsulfonyl)acetamide | |
| 58 | 177 | 2-(4-fluoro-2,6- | 454.1 |
| | | diisopropylphenyl)-N-(4-(methyl- | |
| | | sulfonyl)phenylsulfonyl)acetamide | |
| 59 | 178 | 2-(4-fluoro-2,6- | 454.1 |
| | | diisopropylphenyl)-N-(3-(methyl- | |
| | | sulfonyl)phenylsulfonyl)acetamide | |
| 60 | 179 | N-(4-(1H-pyrazol-1- | 444.2 |
| | | yl)phenylsulfonyl)-2-(4-fluoro-2,6- | (M + 1) |
| | | diisopropylphenyl)acetamide | |

TABLE 9
Examples in the following table were prepared using
similar conditions as described in Example 5 and
Scheme E from appropriate starting materials.

| | Final | | Mass |
|---|---|---|---|
| Example | Target | | Spec |
| # | Number | IUPAC Name | [M − H]⁻ |
| 61 | 114 | 2-(4-chloro-2,6- | 424.0 |
| | | diisopropylphenyl)-N-(1-isopropyl-1H- | |
| | | pyrazol-3-ylsulfonyl)acetamide | |
| 62 | 180 | 2-(4-fluoro-2,6- | 408.2 |
| | | diisopropylphenyl)-N-(1-isopropyl-1H- | |
| | | pyrazol-3-ylsulfonyl)acetamide | |
| 63 | 181 | 2-(2,6-diisopropylphenyl)-N-(5-(2- | 482.2 |
| | | hydroxypropan-2-yl)-1-phenyl-1H- | |
| | | pyrazol-3-ylsulfonyl)acetamide | |
| 64 | 182 | 2-(4-fluoro-2,6- | 502.2 |
| | | diisopropylphenyl)-N-(5-(2- | (M + 1) |
| | | hydroxypropan-2-yl)-1-phenyl-1H- | |
| | | pyrazol-3-ylsulfonyl)acetamide | |
| 65 | 183 | 2-(4-cyano-2,6- | 507.2 |
| | | diisopropylphenyl)-N-(5-(2- | |
| | | hydroxypropan-2-yl)-1-phenyl-1H- | |
| | | pyrazol-3-ylsulfonyl)acetamide | |
| 66 | 184 | 2-(2,6-diisopropylphenyl)-N-(5-(2- | 422.2 |
| | | hydroxypropan-2-yl)-1-methyl-1H- | (M + 1) |
| | | pyrazol-3-ylsulfonyl)acetamide | |
| 67 | 185 | 2-(4-fluoro-2,6- | 438.2 |
| | | diisopropylphenyl)-N-(5-(2- | |
| | | hydroxypropan-2-yl)-1-methyl-1H- | |
| | | pyrazol-3-ylsulfonyl)acetamide | |
| 68 | 186 | 2-(4-cyano-2,6- | 445.2 |
| | | diisopropylphenyl)-N-(5-(2- | |
| | | hydroxypropan-2-yl)-1-methyl-1H- | |
| | | pyrazol-3-ylsulfonyl)acetamide | |

TABLE 10
Examples in the following table were prepared using
similar conditions as described in Example 5 and
Scheme E from appropriate starting materials.

| | Final | | Mass |
|---|---|---|---|
| Example | Target | | Spec |
| # | Number | IUPAC Name | [M − H]⁻ |
| 69 | 187 | 2-(8-fluoro-1,2,3,5,6,7-hexahydros- | 439.1 |
| | | indacen-4-yl)-N-(5-(2-hydroxypropan-2- | (M + 1) |
| | | yl)thiazol-2-ylsulfonyl)acetamide | |
| 70 | 108 | 2-(4-chloro-2,6- | 459.1 |
| | | diisopropylphenyl)-N-(5-(2- | (M + 1) |
| | | hydroxypropan-2-yl)thiazol-2- | |
| | | ylsulfonyl)acetamide | |
| 71 | 109 | 2-(3-fluoro-2,6- | 443.1 |
| | | diisopropylphenyl)-N-(5-(2- | (M + 1) |
| | | hydroxypropan-2-yl)thiazol-2- | |
| | | ylsulfonyl)acetamide | |
| 72 | 188 | 2-(2,6-diisopropyl-4- | 491.1 |
| | | (trifluoromethyl)phenyl)-N-(5-(2- | |
| | | hydroxypropan-2-yl)thiazol-2- | |
| | | ylsulfonyl)acetamide | |
| 73 | 189 | 2-(4-fluoro-2,6- | 457.0 |
| | | diisopropylphenyl)-N-(5-(2- | (M + 1) |
| | | methoxypropan-2-yl)thiazol-2- | |
| | | ylsulfonyl)acetamide | |
| 74 | 190 | 2-(4-cyano-2,6- | 464.1 |
| | | diisopropylphenyl)-N-(5-(2- | (M + 1) |
| | | methoxypropan-2-yl)thiazol-2- | |
| | | ylsulfonyl)acetamide | |
| 75 | 191 | 2-(4-fluoro-2,6- | 441.1 |
| | | diisopropylphenyl)-N-(2-(2- | |
| | | hydroxypropan-2-yl)thiazol-5- | |
| | | ylsulfonyl)acetamide | |
| 76 | 192 | 2-(3,4-difluoro-2,6- | 461.0 |
| | | diisopropylphenyl)-N-(5-(2- | (M + 1) |
| | | hydroxypropan-2-yl)thiazol-2- | |
| | | ylsulfonyl)acetamide | |
| 77 | 193 | 2-(3,5-difluoro-2,6- | 459.1 |
| | | diisopropylphenyl)-N-(5-(2- | |
| | | hydroxypropan-2-yl)thiazol-2- | |
| | | ylsulfonyl)acetamide | |
| 78 | 194 | 2-(2,6-dicyclopropylphenyl)-N- | 420.9 |
| | | (5-(2-hydroxypropan-2-yl)thiazol-2- | (M + 1) |
| | | ylsulfonyl)acetamide | |
| 79 | 195 | 2-(4-chloro-2-isopropyl-6- | 483.1 |
| | | (trifluoromethyl)phenyl)-N-(5-(2- | |
| | | hydroxypropan-2-yl)thiazol-2- | |
| | | ylsulfonyl)acetamide | |
| 80 | 196 | 2-(2-cyclopropyl-6-isopropylphenyl)- | 421.1 |
| | | N-(5-(2-hydroxypropan-2-yl)thiazol-2- | |
| | | ylsulfonyl)acetamide | |
| 81 | 197 | 2-(4-fluoro-2,6- | 441.1 |
| | | diisopropylphenyl)-N-(4-(2- | |
| | | hydroxypropan-2-yl)thiazol-2- | |
| | | ylsulfonyl)acetamide | |
| 82 | 198 | 2-(4-cyano-2,6- | 448.1 |
| | | diisopropylphenyl)-N-(4-(2- | |
| | | hydroxypropan-2-yl)thiazol-2- | |
| | | ylsulfonyl)acetamide | |
| 83 | 199 | 2-(4-chloro-3,5-difluoro-2,6- | 493.1 |
| | | diisopropylphenyl)-N-(5-(2- | |
| | | hydroxypropan-2-yl)thiazol-2- | |
| | | ylsulfonyl)acetamide | |
| 84 | 200 | 2-(4-fluoro-2,6- | 427.3 |
| | | diisopropylphenyl)-N-(5- | (M + 1) |
| | | isopropylthiazol-2- | |
| | | ylsulfonyl)acetamide | |
| 85 | 201 | 2-(2,6-diisopropyl-4- | 507.1 |
| | | (trifluoromethoxy)phenyl)-N-(5-(2- | |
| | | hydroxypropan-2-yl)thiazol-2- | |
| | | ylsulfonyl)acetamide | |
| 86 | 202 | 2-(2,6-diethyl-4-fluorophenyl)-N-(5- | 415.1 |
| | | (2-hydroxypropan-2-yl)thiazol-2- | (M + 1) |
| | | ylsulfonyl)acetamide | |
| 87 | 203 | 2-(2-chloro-5- | 442.9 |
| | | (trifluoromethyl)phenyl)-N-(5-(2- | (M + 1) |
| | | hydroxypropan-2-yl)thiazol-2- | |
| | | ylsulfonyl)acetamide | |
| 88 | 204 | 2-(3,5-dichloro-2- | 438.9 |
| | | methoxyphenyl)-N-(5-(2- | (M + 1) |
| | | hydroxypropan-2-yl)thiazol-2- | |
| | | ylsulfonyl)acetamide | |

### Example 89

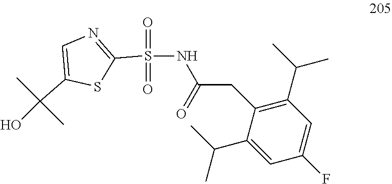 [SMILES: CC(C)c1cc(F)cc(C(C)C)c1CC(=O)NS(=O)(=O)c1ncc(C(C)(C)O)s1]

### 2-(4-Fluoro-2,6-diisopropylphenyl)-N-(5-(2-hydroxypropan-2-yl)thiazol-2-ylsulfonyl)acetamide(Scheme E)

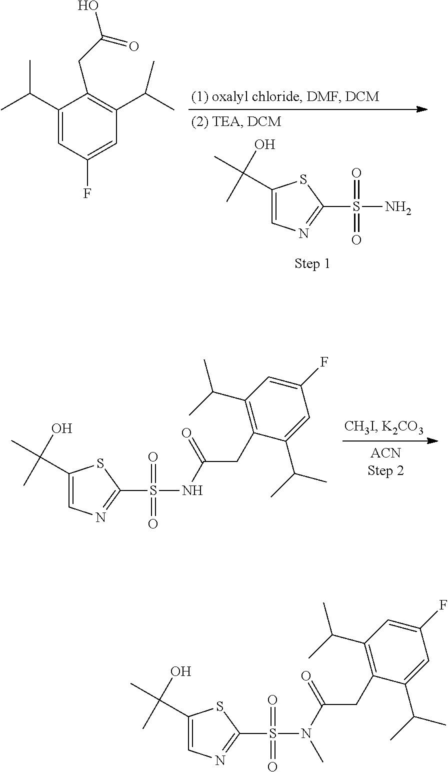

### Step 1: 2-(4-Fluoro-2,6-diisopropylphenyl)-N-(5-(2-hydroxypropan-2-yl)thiazol-2-ylsulfonyl)acetamide

Into a 50-mL round-bottom flask was placed 2-(4-fluoro-2,6-diisopropylphenyl)acetic acid (80 mg, 0.34 mmol), DCM (4 mL), DMF (0.05 mL). This was followed by the addition of oxalyl chloride (0.5 mL) dropwise with stirring at RT. The solution was stirred for 30 min at RT and then was concentrated under vacuum. The above mixture diluted in DCM (1 mL) was added to a solution of 5-(2-hydroxypropan-2-yl)thiazole-2-sulfonamide (80 mg, 0.36 mmol) and TEA (0.2 mL) in DCM (3 mL) dropwise with stirring at RT. The resulting solution was stirred for 2 h at RT and then was concentrated under vacuum. The crude product was purified by Prep-HPLC using method E eluted with a gradient of 19˜68% ACN. This resulted in 82.5 mg (56%) of Example 89 as a white solid. MS-ESI: 443.2 (M+1). ¹H NMR (300 MHz, MeOD-d₄) δ 7.79 (s, 1H), 6.77 (d, J=10.2 Hz, 2H), 3.80 (s, 2H), 3.00-2.80 (m, 2H), 1.58 (s, 6H), 1.08 (d, J=6.6 Hz, 12H).

### Step 2: 2-(4-Fluoro-2,6-diisopropylphenyl)-N-(5-(2-hydroxypropan-2-yl)thiazol-2-ylsulfonyl)-N-methyl acetamide

Into a 50-mL round-bottom flask purged with and maintained under nitrogen, was placed 2-(4-fluoro-2,6-diisopropylphenyl)-N-(5-(2-hydroxypropan-2-yl)thiazol-2-ylsulfonyl)acetamide (80 mg, 0.18 mmol), ACN (5 mL), potassium carbonate (50 mg, 0.36 mmol), CH₃I (50 mg, 0.35 mmol). The resulting solution was stirred for 4 h at 80° C. and then was concentrated under vacuum. The crude product was purified by Prep-HPLC using method E eluted with a gradient of 55˜80% ACN. This resulted in 22.9 mg (28%) of Example 90 as a yellow solid. MS-ESI: 457.0 (M+1). ¹H NMR (300 MHz, MeOD-d₄) δ 7.89 (s, 1H), 6.80 (d, J=10.2 Hz, 2H) 4.30 (s, 2H), 3.37 (s, 3H), 2.90-2.70 (m, 2H), 1.63 (s, 6H), 1.09 (d, J=6.6 Hz, 12H).

### Example 91

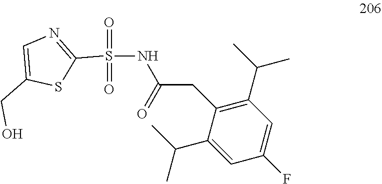 [SMILES: CC(C)c1cc(F)cc(C(C)C)c1CC(=O)NS(=O)(=O)c1ncc(CO)s1]

### 2-(4-Fluoro-2,6-diisopropylphenyl)-N-(5-(hydroxymethyl)thiazol-2-ylsulfonyl)acetamide

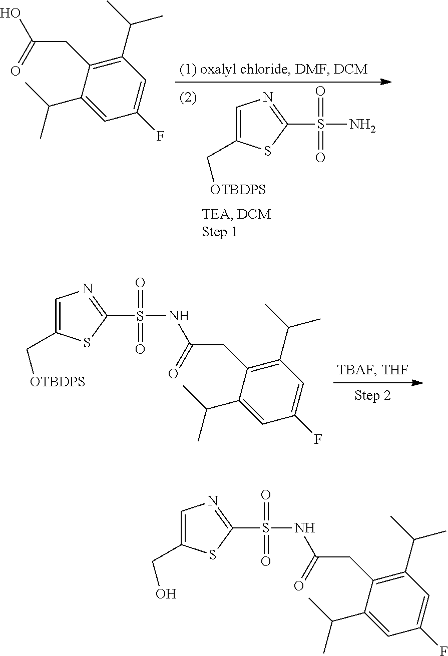

### Step 1: N-(5-((tert-butyldiphenylsilyloxy)methyl)thiazol-2-ylsulfonyl)-2-(4-fluoro-2,6-diisopropylphenyl) acetamide

Into a 50-mL round-bottom flask, was placed 2-(4-fluoro-2,6-diisopropylphenyl)acetic acid (93 mg, 0.39 mmol), DCM (5 mL), DMF (0.05 mL). This was followed by the addition of oxalyl chloride (0.5 mL) dropwise with stirring at RT. The solution was stirred for 30 min at RT and then was concentrated under vacuum. The above mixture diluted in DCM (1 mL) was added to a solution of 5-((tert-butyldiphenylsilyloxy)methyl)thiazole-2-sulfonamide (169 mg, 0.39 mmol) and TEA (0.2 mL) in DCM (3 mL) dropwise with stirring at RT. The resulting solution was stirred for 2 h at RT and diluted with 5 mL of water. The resulting solution was extracted with 3×5 mL of ethyl acetate and the organic layers combined and dried over anhydrous Na₂SO₄, and then concentrated under vacuum. This resulted in 200 mg (78%) of the title compound as a yellow solid. MS-ESI: 651.2 (M-1).

### Step 2: 2-(4-Fluoro-2,6-diisopropylphenyl)-N-(5-(hydroxymethyl)thiazol-2-ylsulfonyl)acetamide

Into a 50-mL round-bottom flask, was placed N-(5-((tert-butyldiphenylsilyloxy)methyl)thiazol-2-ylsulfonyl)-2-(4-fluoro-2,6-diisopropylphenyl)acetamide (200 mg, 0.31 mmol), THF (5 mL), TBAF (160 mg, 0.61 mmol). The resulting solution was stirred for 5 h at RT and then was concentrated under vacuum. The residue was applied onto a silica gel column and eluted with DCM/MeOH (50:1 to 20:1). The crude product was purified by Prep-HPLC using method eluted with a gradient of 20˜55% o ACN. This resulted in 33.0 mg (26% o) of the title compound as a white solid. MS-ESL: 413.1 (M-1). ¹H NMR (300 MHz, MeOD-d₄) δ 7.69 (s, 1H), 6.75 (d, J=13.6 Hz, 2H), 4.78 (s, 2H), 3.74 (s, 2H), 3.20-3.00 (m, 2H), 1.12 (d, J=7.2 Hz, 12H)

TABLE 11
Examples in the following table were prepared using
similar conditions as described in Example 91 and
Scheme E from appropriate starting materials.

| | Final | | Mass |
|---|---|---|---|
| Example | Target | | Spec |
| # | Number | IUPAC Name | [M − H]⁻ |
| 92 | 207 | 2-(4-fluoro-2,6- | 429.1 |
| | | diisopropylphenyl)-N-(5-(1- | (M + 1) |
| | | hydroxyethyl)thiazol-2- | |
| | | ylsulfonyl)acetamide | |
| 93 | 208 | 2-(4-cyano-2,6- | 436.1 |
| | | diisopropylphenyl)-N-(5-(1- | (M + 1) |
| | | hydroxyethyl)thiazol-2- | |
| | | ylsulfonyl)acetamide | |
| 94 | 209 | 2-(4-fluoro-2,6- | 464.2 |
| | | diisopropylphenyl)-N-(3- | |
| | | (hydroxymethyl)-4-(2- | |
| | | hydroxypropan-2- | |
| | | yl)phenylsulfonyl)acetamide | |
| 95 | 210 | 2-(4-cyano-2,6- | 471.2 |
| | | diisopropylphenyl)-N-(3- | |
| | | (hydroxymethyl)-4-(2- | |
| | | hydroxypropan-2- | |
| | | yl)phenylsulfonyl)acetamide | |

### Example 96

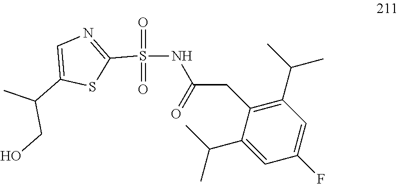 [SMILES: CC(C)c1cc(F)cc(C(C)C)c1CC(=O)NS(=O)(=O)c1ncc(C(C)CO)s1]

### 2-(4-Fluoro-2,6-diisopropylphenyl)-N-(5-(1-hydroxypropan-2-yl)thiazol-2-ylsulfonyl)acetamide

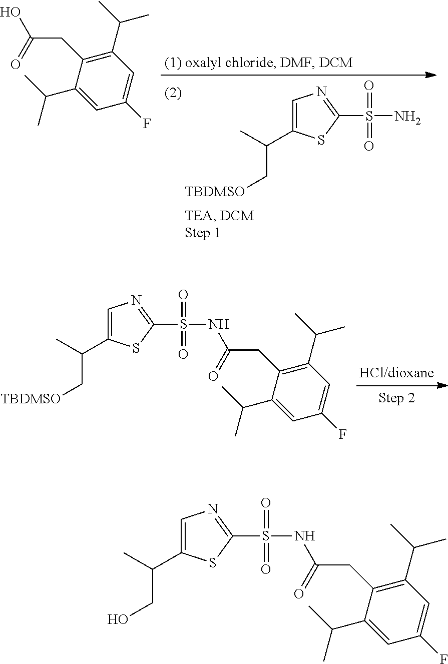 [SMILES: CC(C)c1cc(F)cc(C(C)C)c1CC(=O)O ; CC(CO[Si](C)(C)C(C)(C)C)c1cnc(S(N)(=O)=O)s1 ; CC(C)c1cc(F)cc(C(C)C)c1CC(=O)NS(=O)(=O)c1ncc(C(C)CO[Si](C)(C)C(C)(C)C)s1 ; CC(C)c1cc(F)cc(C(C)C)c1CC(=O)NS(=O)(=O)c1ncc(C(C)CO)s1]

### Step 1: N-(5-(1-(tert-butyldimethylsilyloxy)propan-2-yl)thiazol-2-ylsulfonyl)-2-(4-fluoro-2,6-diisopropyl phenyl)acetamide

Into a 50-mL round-bottom flask, was placed 2-(4-fluoro-2,6-diisopropylphenyl)acetic acid (57 mg, 0.24 mmol), DCM (2 mL), and DMF (0.05 mL). This was followed by the addition of oxalic dichloride (0.5 mL) dropwise with stirring at RT. The resulting solution was stirred for 30 min at RT and then was concentrated under vacuum. The mixture diluted in DCM (1 mL) was added to a solution of 5-(1-(tert-butyldimethylsilyloxy)propan-2-yl)thiazole-2-sulfonamide (80 mg, 0.24 mmol) and TEA (0.2 mL) in DCM (2 mL) dropwise with stirring at RT. The resulting solution was stirred for 1 h at RT and then was diluted with 5 mL of water. The resulting solution was extracted with 3×5 mL of ethyl acetate and the organic layers combined and dried over anhydrous Na₂SO₄, and then concentrated under vacuum. This resulted in 120 mg (90%) of the title compound as a white solid. MS-ESI: 555.2 (M-1).

### Step 2: 2-(4-Fluoro-2,6-diisopropylphenyl)-N-(5-(1-hydroxypropan-2-yl)thiazol-2-ylsulfonyl)acetamide

Into a 50-mL round-bottom flask, was placed N-(5-(1-(tert-butyldimethylsilyloxy)propan-2-yl)thiazol-2-ylsulfonyl)-2-(4-fluoro-2,6-diisopropylphenyl)acetamide (120 mg, 0.22 mmol), HCl/dioxane (4 M, 3 mL). The resulting solution was stirred for 2 h at RT and then was concentrated under vacuum. The crude product was purified by Prep-HPLC using method E eluted with a gradient of 25˜50% ACN. This resulted in 29.4 mg (31%) of the title compound as a white solid. MS-ESI: 443.2 (M+1). ¹H NMR (400 MHz, MeOD-d₄) δ 7.80 (s, 1H), 6.80 (d, J=10.0 Hz, 2H), 3.82 (s, 2H), 3.62-3.72 (m, 1H), 3.62-3.53 (m, 1H), 3.30-3.20 (m, 1H), 3.00-2.80 (m, 2H), 1.34 (d, J=7.2 Hz, 3H), 1.10 (d, J=7.2 Hz, 12H).

TABLE 12
Example in the following table was prepared using
similar conditions as described in Example 96
and Scheme E from appropriate starting materials.

| | Final | | Mass |
|---|---|---|---|
| Example | Target | | Spec |
| # | Number | IUPAC Name | [M − H]⁻ |
| 97 | 212 | 2-(4-fluoro-2,6- | 427.1 |
| | | diisopropylphenyl)-N-(5-(2- | |
| | | hydroxyethyl)thiazol-2- | |
| | | ylsulfonyl)acetamide | |

The following compounds were prepared using procedures analogous to those described herein for other compounds using functional group transformations that are known to the skilled artisan:

| Final | | | |
|---|---|---|---|
| Target | | | Mass |
| Number | Structure | IUPAC Name | Spec |
| 213 | 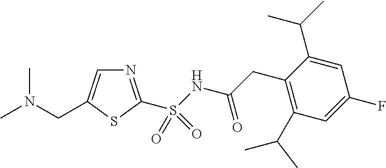 [SMILES: CC(C)c1cc(F)cc(C(C)C)c1CC(=O)NS(=O)(=O)c1ncc(CN(C)C)s1] | 2-(4-fluoro-2,6-diisopropylphenyl)- N-(5-(dimethylaminomethyl)thiazol- 2-ylsulfonyl)acetamide | 442.2 |
| 214 | 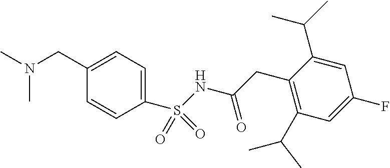 [SMILES: CC(C)c1cc(F)cc(C(C)C)c1CC(=O)NS(=O)(=O)c1ccc(CN(C)C)cc1] | 2-(4-fluoro-2,6-diisopropylphenyl)-N- (4-dimethylaminomethyl) phenylsulfonyl)acetamide | 435.2 |
| 215 | 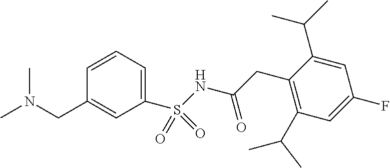 [SMILES: CC(C)c1cc(F)cc(C(C)C)c1CC(=O)NS(=O)(=O)c1cccc(CN(C)C)c1] | 2-(4-fluoro-2,6-diisopropylphenyl)-N- (3-dimethylaminomethyl) phenylsulfonyl)acetamide | 435.2 |

### Examples 97-99

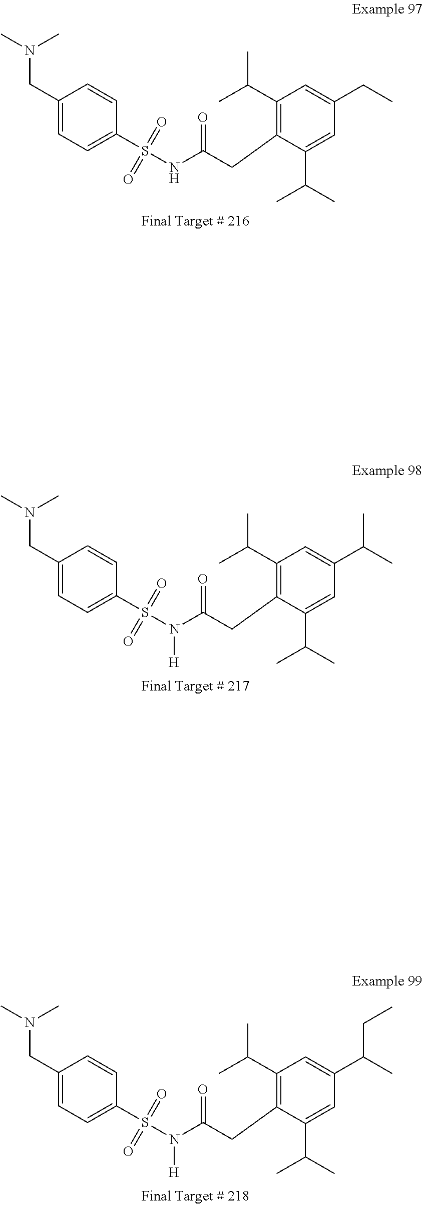

### Preparation of 2-(4-bromo-2,6-diisopropylphenyl)acetic Acid

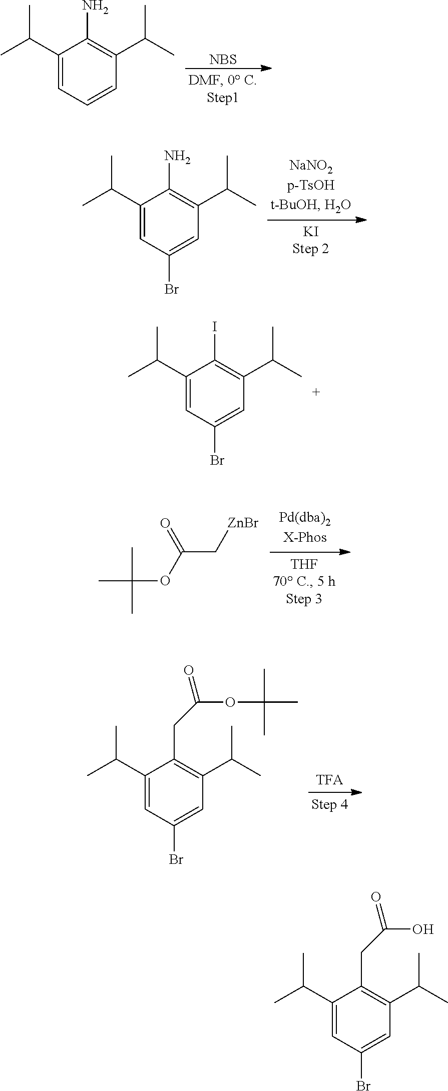

### Step 1: Preparation of 4-bromo-2,6-diisopropylaniline

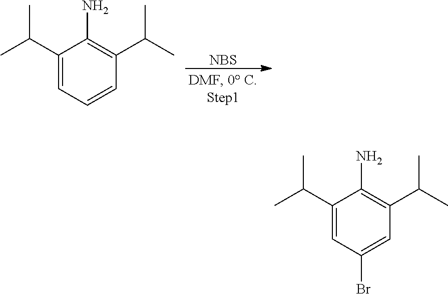

To a stirred solution of 2,6-diisopropylaniline (5.05 g, 28.4 mmol, 1.00 equiv) in N,N-dimethylformamide (70 mL) at 0° C. was added a solution of NBS (5.05 g, 28.4 mmol, 1.00 equiv) in N,N-dimethylformamide (30 mL) dropwise over 60 min. The reaction was stirred for another hour at 0° C., at which time water (300 mL) was added. The resulting mixture was extracted with ethyl acetate (2×300 mL), and the combined organic layers were washed with saturated NH₄C₁ solution (3×100 mL) followed by water (100 mL) and dried over anhydrous sodium sulfate. Concentration of the solution under vacuum afforded 4-bromo-2,6-diisopropylaniline (6.5 g, 88% yield). LCMS (Method A): 256.1 [M+H]⁺, retention time 2.97 min.

### Step 2: Preparation of 5-bromo-2-iodo-1,3-diisopropylbenzene

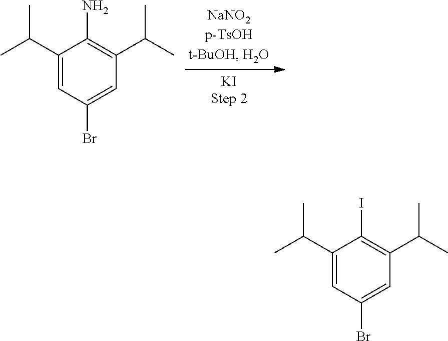

4-Bromo-2,6-diisopropylaniline (35.6 g, 138.96 mmol) was added to a suspension of p-TsOH monohydrate (118.95 g, 625.34 mmol) in a mixture of t-BuOH (500 mL) and water (30 mL). The mixture was cooled to 0° C. in an ice bath and a solution of sodium nitrite (28.76 g, 416.89 mmol) and potassium iodide (86.51 g, 521.11 mmol) in water (70 mL) was then added dropwise over 2.5 h, keeping the temperature of the mixture at 10-15° C. Following the addition, the temperature was then allowed to rise to 25° C., and the mixture was stirred for an additional 1.5 h. The reaction mixture was poured into water and extracted with Et₂O. The ether layer was then washed with 10% sodium bisulfite solution to remove iodine related by-products. The organic layer was dried over MgSO₄ and concentrated under reduced pressure. The residue was flushed through the silica gel plug eluting with hexane/EtOAc (100/0 to 90/10). Fractions containing the desired product were combined and concentrated under reduced pressure to afford pure 5-bromo-2-iodo-1,3-diisopropylbenzene (34.5 g, 67% yield). LCMS (Method A): 366.0 [M+], retention time 4.31 min.

### Step 3: Preparation of tert-butyl 2-(4-bromo-2,6-diisopropylphenyl)acetate

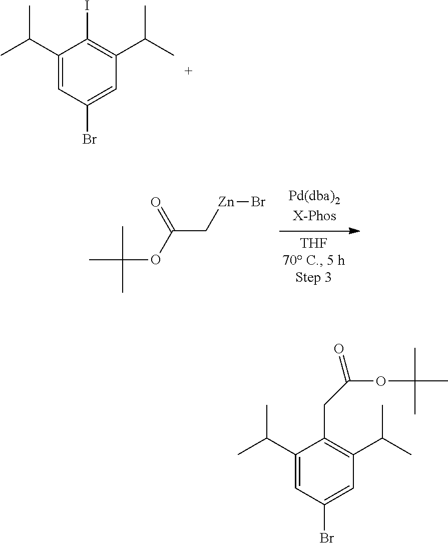

(2-(tert-butoxy)-2-oxoethyl)zinc(II) bromide solution: Under a N2 atmosphere, to a 500 mL round-bottom flask were added zinc powder (32.19 g, 492.17 mmol), dry THF (200 mL), and TMSCl (2.08 mL, 16.41 mmol). The suspension was warmed to 56° C. and stirred at this temperature for 30 min. A solution of t-butyl bromoacetate (32 g, 164.06 mmol) in THF (50 mL) was added dropwise to the suspension. After insoluble matter precipitated, the light yellow supernatant solution was decanted and used for subsequent experiment as is.

tert-Butyl 2-(4-bromo-2,6-diisopropylphenyl)acetate: Into a 5 L 3-necked round-bottom flask purged with and maintained under nitrogen, was placed 5-bromo-2-iodo-1,3-diisopropylbenzene (34.5 g, 93.99 mmol), THF (150 mL), X-phos (4.48 g, 9.4 mmol), Pd₂(dba)₃CHCl₃ (3.2 g, 4.7 mmol). The resulting solution was stirred for 0.5 h at room temperature, at which time the previously prepared solution of 2-(tert-butoxy)-2-oxoethyl)zinc(II) bromide was added. The resulting solution was stirred for 3 h at 76° C. and then quenched by the addition of 200 mL of NH₄Cl (saturated). The organic layer was separated and the aqueous layer was back extracted with EtOAc (200 mL). The combined organic layers were washed with brine, dried over anhydrous sodium sulfate, filtered and concentrated. The crude product was purified by percolation through silica gel, eluting with mixtures of hexane and EtOAc. The product was analyzed on HPLC and had a retention time of 4.11 min using method A. Fractions containing pure product were concentrated and used in the next step without further purification.

### Step 4: Preparation of 2-(4-bromo-2,6-diisopropylphenyl) acetic Acid

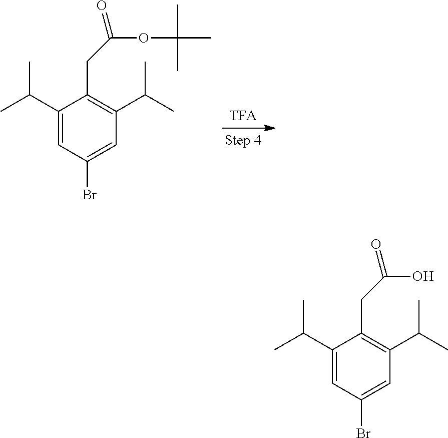 [SMILES: CC(C)c1cc(Br)cc(C(C)C)c1CC(=O)OC(C)(C)C ; CC(C)c1cc(Br)cc(C(C)C)c1CC(=O)O]

tert-Butyl 2-(4-bromo-2,6-diisopropylphenyl)acetate from the previous step was dissolved in dichloromethane (60 mL) and treated with TFA (35 mL). The reaction mixture was stirred overnight at room temperature and then concentrated and partially purified by silica gel flash chromatography. The majority of the desired product eluted with 100% hexanes, but further fractions collected from 1-20% EtOAc/hexanes also contained product. Those fractions which contained product were combined, concentrated in vacuo, and then partitioned between hexane and aqueous 10% Na₂CO₃. The product-containing aqueous layer was washed once with hexanes and then acidified to pH˜1 using 2N HCl. The product was extracted into EtOAc (150 mL×3). The combined organic layers were washed with brine, dried over anhydrous sodium sulfate, filtered and concentrated to give desired product 2-(4-bromo-2,6-diisopropylphenyl)acetic acid as white solid (7 g, 25% yield over two steps).

The product does not have discernible [M+H]⁺ but does have a UV and ELSD signal. The retention time was 3.2 min on an LCMS run using method A. ¹H NMR (250 MHz, DMSO-d₆): 7.26 (s, 2H), 3.68 (s, 2H), 3.08 (m, 2H), 1.13 (d, J=7.5 Hz, 12H).

### Preparation of 2-(4-bromo-2,6-diisopropylphenyl)-N-((4-((dimethylamino)methyl)phenyl) sulfonyl) acetamide)

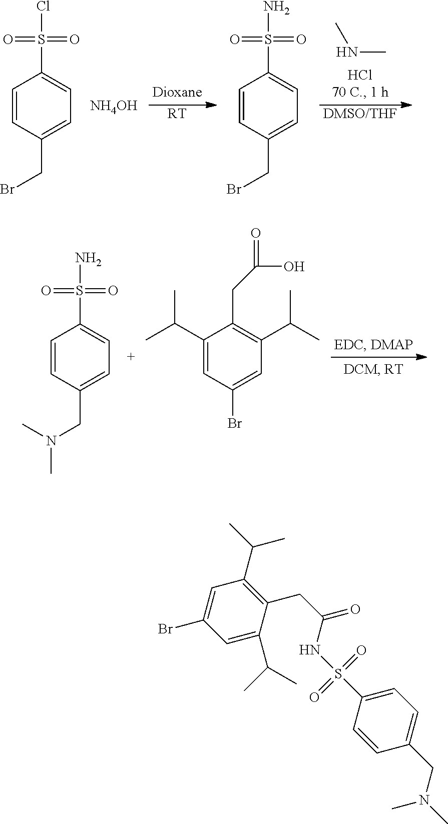

### Step 1: Preparation of 4-(bromomethyl)benzenesulfonamide

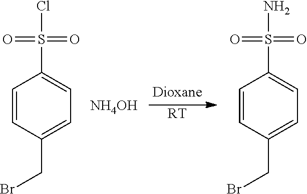

4-(bromomethyl)benzenesulfonyl chloride (2.5 g, 9.3 mmol) was dissolved in dioxane (20 mL). To this solution was added concentrated NH₄OH (5 mL). The solution was stirred at room temperature for 5 min. After the initial exotherm, the solution was poured into the water and extracted with EtOAc several times. The combined organic extracts were dried over MgSO₄, filtered and concentrated under reduced pressure. The resulting sulfonamide was used without further purification. Product does not ionize on LCMS but has a UV (254 nm) signal at 2.0 min (Method A).

### Step 2: Preparation of 4-((dimethylamino)methyl)benzenesulfonamide

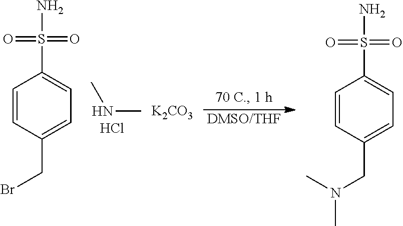

To a solution of 4-(bromomethyl)benzenesulfonamide (2.5 g, 10 mmol) in DMSO (10 mL) was added dimethylamine hydrochloride followed by K₂CO₃. The reaction mixture was heated at 70° C. for 1 h. LCMS showed complete conversion of the starting material and the mixture was poured into the water and extracted with EtOAc several times. The product in the combined organic layers was extracted with 1M HCl. The aqueous phase was washed with EtOAc and dichloromethane to remove impurities and the aqueous layer was basified with 2M NaOH and extracted with EtOAc. The solution was dried over MgSO₄, filtered and concentrated under reduced pressure to afford pure 4-((dimethylamino)methyl) benzenesulfonamide as white crystals (0.800 g, 37% yield over two steps). LCMS (Method A): 215.1 [M+H]⁺, retention time 0.86 min. ¹H NMR (250 MHz, DMSO-d₆): 7.77 (d, J=7.5 Hz, 2H), 7.46 (d, J=7.5 Hz, 2H), 3.45 (s, 2H), 2.15 (s, 6H).

### Step 3: Preparation of 2-(4-bromo-2,6-diisopropylphenyl)-N-((4-((dimethylamino)methyl)phenyl)-sulfonyl)acetamide

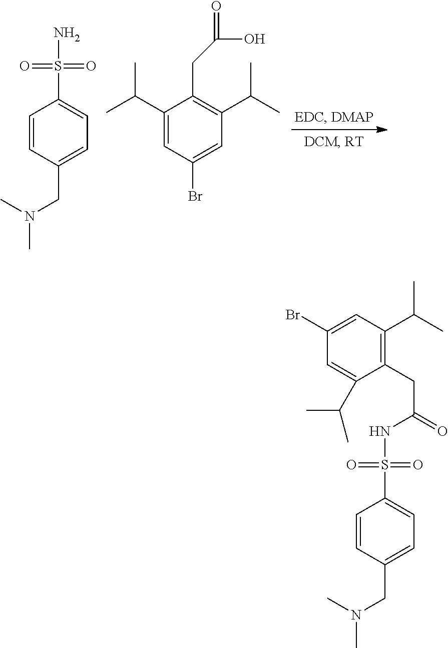

A solution of 2-(4-bromo-2,6-diisopropylphenyl)acetic acid (0.598 g, 2 mmol), 4-((dimethylamino)-methyl) benzene sulfonamide (0.643 g, 3 mmol, 1.5 eq.), 4-dimethyaminopyridine (DMAP, 0.489 g, 4 mmol, 2 equiv), and 1-[3-(dimethyamino)-propyl]-3-ethylcarbodiimide hydrochloride (EDCI, 0.767 g, 4 mmol, 2 eq.) in CH₂Cl₂ (15 mL) was stirred at room temperature for 1 h. After LCMS showed complete conversion of the acid, the reaction was quenched by the addition of water, and aqueous phase was extracted with DCM (3×50 mL). The combined organic layers were washed with brine and dried over MgSO₄, and evaporated in vacuo to afford 2-(4-bromo-2,6-diisopropylphenyl)-N-((4-((dimethylamino) methyl)phenyl)-sulfonyl)acetamide (0.891 g, 90% yield) of sufficient purity to be used as a scaffold for the cross coupling reactions without further purification. An analytically pure sample was obtained when the product was purified on HPLC with TFA buffer. LCMS (Method A): 497.3, 495.3 [M+H]⁺, retention time 2.63 min. ¹H NMR (250 MHz, DMSO-d₆): 7.79 (d, J=7.5 Hz, 2H), 7.46 (d, J=7.5 Hz, 2H), 7.14 (s, 2H), 3.86 (s, 2H), 3.58 (s, 2H), 2.97 (m, 2H), 2.41 (s, 6H), 0.97 (d, J=7.5 Hz, 12H).

### Preparation of 2-[2,6-bis(propan-2-yl)-4-(tetramethyl-1,3,2-dioxaborolan-2-yl)phenyl]-N-{4-[(dimethylamino)methyl]benzenesulfonyl}acetamide

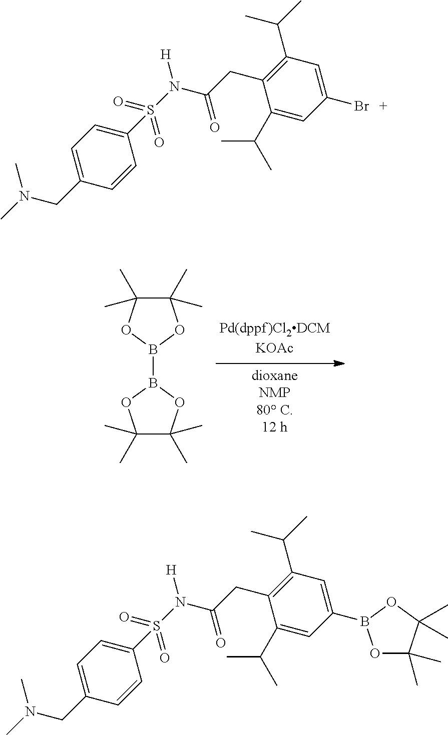

To a solution of 2-(4-bromo-2,6-diisopropylphenyl)-N-((4-((dimethylamino)methyl)phenyl)-sulfonyl)acetamide (530 mg, 1.06 mmol) in dioxane (20 mL) and NMP (2 mL) was added 4,4,5,5-tetramethyl-2-(4,4,5,5-tetramethyl-1,3,2-dioxaborolan-2-yl)-1,3,2-dioxaborolane (410 mg, 1.61 mmol), Pd(dppf)Cl₂.DCM (86 mg, 0.106 mmol) and potassium acetate (312 mg, 3.18 mmol). The resulting mixture was stirred at 80° C. for 12 h. Reaction mixture was brought to room temperature, filtered through a pad of celite, diluted with water (50 mL) and extracted with EtOAc (3×30 mL). The combined organic layer was washed water and brine, dried over anhydrous sodium sulfate, filtered and concentrated in vacuo to provide titled compound (437 mg, 75%) as light buff color solid.

### General Procedure for Reverse Suzuki Coupling of 2-[2,6-bis(propan-2-yl)-4-(tetramethyl-1,3,2-dioxaborolan-2-yl)phenyl]-N-{4-[(dimethylamino)methyl]benzenesulfonyl}acetamide with Orgnobromides/Chlorides

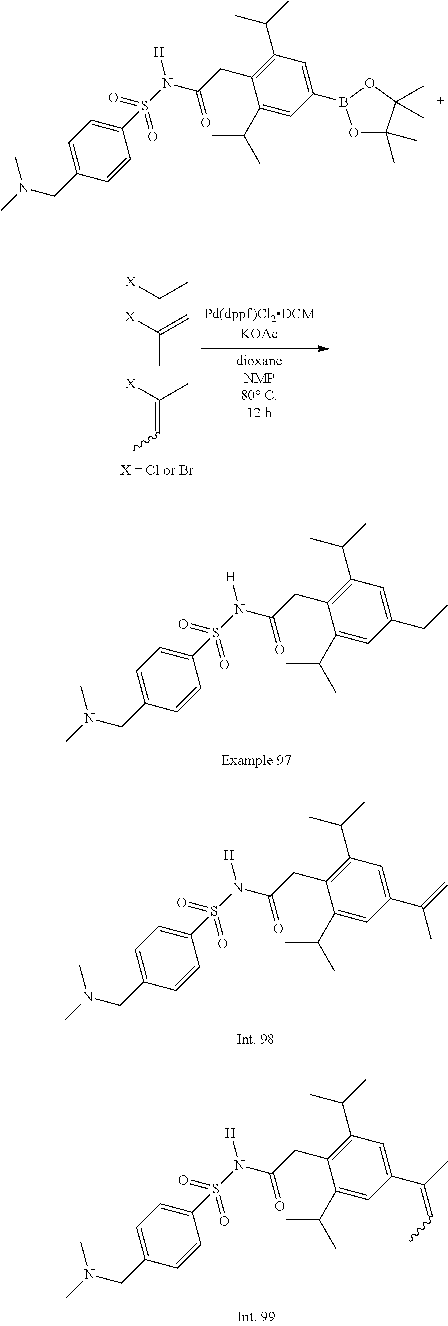

To a solution of 2-[2,6-bis(propan-2-yl)-4-(tetramethyl-1,3,2-dioxaborolan-2-yl)phenyl]-N-{4-[(dimethylamino)methyl]benzenesulfonyl}acetamide (0.1 mmol) in dioxane (1.5 mL) was added the appropriate alkyl or alkenyl halide (e.g., X═Br) (0.2 mmol), Pd(dppf)Cl₂.DCM (0.01 mmol) and 1M aqueous cesium carbonate solution (0.3 mL). The resulting mixture was stirred at 80° C. for 12 h. Reaction mixture was brought to room temperature, filtered through a pad of celite and rinsed with EtOAc (5 mL). Filtrates were concentrated in vacuo and purified by prep-HPLC to obtain desired product.

N-((4-((dimethylamino)methyl)phenyl)sulfonyl)-2-(4-ethyl-2,6-diisopropylphenyl)acetamide (Example 97) was synthesized according to the method above. LC/MS (m+z)=445.28 [M+H], retention time (LC/MS Method B): 2.15 min.

2-(2,6-diisopropyl-4-(prop-1-en-2-yl)phenyl)-N-((4-((dimethylamino)methyl)phenyl)sulfonyl)-acetamide (Int. 98); and

2-(4-(but-2-en-2-yl)-2,6-diisopropylphenyl)-N-((4-((dimethylamino)methyl)phenyl)sulfonyl)-acetamide (Int. 99) were prepared according to the method above.

### General Procedure for the Hydrogenation of Unsaturated Compounds

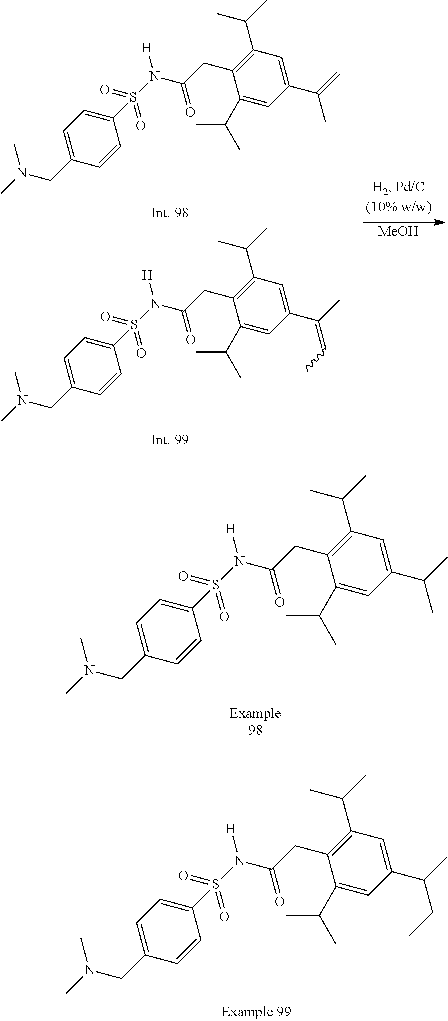

### Method A:

To a solution of unsaturated starting material (0.1 mmol) in MeOH (15 mL) was added 10 weight % Pd/C (10% w/w), trifluoroacetic acid (0.5 mL) and the resulting mixture was hydrogenated at 45 psi for 12 h. Reaction mixture was filtered through a pad of celite, filtrates were concentrated and purified by prep-HPLC.

### Method B:

To a solution of unsaturated starting material (crude obtained either from Suzuki or Sonogashira coupling at 0.1 mmol scale) in ethyl acetate (15 mL) was added Pd(OH)₂ (22 mg) and trifluoroacetic acid (0.2 mL) and the resulting mixture was hydrogenated at 45 psi for 12 h. Reaction mixture was filtered through a pad of celite, filtrates were concentrated and purified by prep-HPLC.

and were synthesized via hydrogenation of Int. 98 and Int. 99 as described above.

N-((4-((dimethylamino)methyl)phenyl)sulfonyl)-2-(2,4,6-triisopropylphenyl)acetamide (Example 98): LC/MS (m/z)=459.38 [M+H], retention time (LC/MS Method B): 2.24 min.

2-(4-(sec-butyl)-2,6-diisopropylphenyl)-N-((4-((dimethylamino)methyl)phenyl)sulfonyl)-acetamide (Example 99): LC/MS (m/z)=473.19 [M+H], retention time (LC/MS Method B): 2.40 min.

### Example 100 (Final Target No. 219)

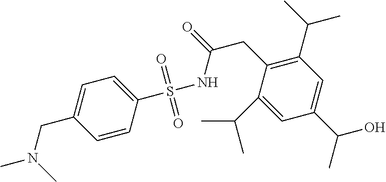 [SMILES: CC(C)c1cc(C(C)O)cc(C(C)C)c1CC(=O)NS(=O)(=O)c1ccc(CN(C)C)cc1]

### Preparation of N-{4-[(dimethylamino)methyl]benzenesulfonyl}-2-[4-(1-hydroxyethyl)-2,6-bis(propan-2-yl)phenyl]acetamide (Example 100, Final Target No. 219)

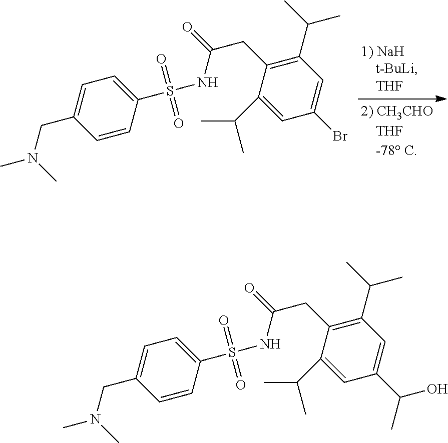

To a solution of 2-(4-bromo-2,6-diisopropylphenyl)-N-((4-((dimethylamino)methyl)phenyl)-sulfonyl)acetamide (43 mg, 0.087 mmol) in anhydrous THF (2 mL) was added NaH (3.5 mg, 0.087 mmol, 60% dispersed in oil) under nitrogen at −78° C. t-BuLi (0.056 mL, 0.095 mmol, 1.7M in pentane) was added slowly and after 3 min acetaldehyde (0.15 mL) was added quickly. Reaction mixture was further stirred at −78° C. for 5 min before quenching with water (5 mL). Reaction mixture froze. Dry ice/acetone cooling bath was removed. The reaction mixture was gradually warmed up to room temperature and extracted with EtOAc (3×5 mL). Combined organic layer was concentrated in vacuo to obtain crude material which was purified by prep-HPLC to obtain titled compound (4.1 mg, 10%). LCMS (Method A): 461.18 [M+H]*, retention time 1.97 min.

The following protocols are suitable for testing the activity of the compounds dislcosed herein. Bioassay 1:

IL-1β Production in PMA-Differentiated THP-1 Cells Stimulated with Gramicidin.

Cell culture-THP-1 cells were purchased from the American Type Culture Collection and sub-cultured according to instructions from the supplier. Prior to experiments, cells were cultured in RPMI 1640 containing 10% heat inactivated FBS, penicillin (100 units/ml) and streptomycin (100 pg/ml), and maintained in log phase prior to experimental setup. Prior to the experiment THP-1 were treated with PMA (Phorbol 12-myristate 13-acetate) (10 g/ml) for 24 hours. The day of the experiment the media was removed and attaching cells were treated with trypsin for 2 minutes, cells were then collected, washed with PBS (phosphate buffer saline), spin down, resuspended in 2% heat inactivated FBS with RPMI at a concentration of 1×106 cells/ml, and 100 ul was plated in a 96 well plate. Cells were incubated with compounds for 1 hours and then stimulated with Gramicidin (5 μM) (Enzo) for 2 hours. Cell free supernatant was collected and the production of IL-10 was evaluated by ELISA. Compounds were dissolved in dimethyl sulfoxide (DMSO) and added to the culture medium to achieve desired concentration (e.g. 100, 30, 10, 3, 1, 0.3 or 0.1 μM). A vehicle only control was run concurrently with each experiment. Final DMSO concentration was 1%. Compounds exhibit a dose-related inhibition of IL-1β production in PMA-differentiated THP-1 cells.

### Bioassay 2:

IL-1β Production in PMA-Differentiated THP-1 Cells Stimulated with Gramicidin.

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 pg/ml)), and maintained in log phase prior to experimental setup. Prior to the experiment THP-1 were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 pg/ml). The cells were plated in a 384-well plate at a density of 50,000 cells/well (final assay volume 50 pl). Compounds were dissolved in dimethyl sulfoxide (DMSO) and added to the culture medium to achieve desired concentration (e.g. 100, 33, 11, 3.7, 1.2, 0.41, 0.14, 0.046, 0.015, 0.0051, 0.0017 pM). Cells were incubated with compounds for 1 hour and then stimulated with gramicidin (5 μM) (Enzo) for 2 hours. Cell free supernatant was collected and the production of IL-1 was evaluated by HTRF (cisbio). A vehicle only control was run concurrently with each experiment. Final DMSO concentration was 0.38%. Compounds exhibited a concentration-dependent inhibition of IL-1 production in PMA-differentiated THP-1 cells.

Compounds tested with protocols 1 and 2 provided IC₅₀ values that are within the variability of the assay.

Tables 13 and 14 show the biological activity of compounds in hTHP-1 assay containing 2% bovine serum: <1 μM=“++++”; ≥1 and <5 μM=“+++”; ≥5 and <15 μM=“++”; ≥15 and <60 μM=“+”.

TABLE 13
Average IC₅₀ of compounds in hTHP-1 assay

| Example # | Average IC₅₀ |
|---|---|
| 1 | ++ |
| 2 | +++ |
| 3 | + |
| 4 | ++ |
| 5 | + |
| 6 | +++ |
| 7 | ++ |
| 8 | ++++ |
| 9 | ++++ |
| 10 | ++++ |
| 11 | ++++ |
| 12 | ++++ |
| 13 | +++ |
| 14 | +++ |
| 15 | +++ |
| 16 | ++++ |
| 17 | +++ |
| 18 | +++ |
| 19 | +++ |
| 20 | ++++ |
| 21 | +++ |
| 22 | + |
| 23 | +++ |
| 24 | + |
| 25 | +++ |
| 26 | +++ |
| 27 | +++ |
| 28 | +++ |
| 29 | + |
| 30 | ++ |
| 31 | ++ |
| 32 | ++ |
| 33 | ++ |
| 34 | +++ |
| 35 | +++ |
| 36 | +++ |
| 37 | ++ |
| 38 | ++ |
| 39 | + |
| 40 | + |
| 41 | ++ |
| 42 | ++ |
| 43 | +++ |
| 44 | ++ |
| 45 | +++ |
| 46 | +++ |
| 47 | +++ |
| 48 | ++++ |
| 49 | ++ |
| 50 | +++ |
| 51 | ++ |
| 52 | +++ |
| 53 | ++++ |
| 54 | ++++ |
| 55 | +++ |
| 56 | ++++ |
| 57 | + |
| 58 | +++ |
| 59 | + |
| 61 | ++ |
| 62 | ++ |
| 63 | ++ |
| 64 | +++ |
| 65 | ++ |
| 66 | + |
| 67 | +++ |
| 68 | ++ |
| 69 | + |
| 70 | ++++ |
| 71 | +++ |
| 72 | +++ |
| 73 | +++ |
| 74 | ++ |
| 75 | ++++ |
| 76 | ++++ |
| 77 | +++ |
| 78 | ++ |
| 79 | + |
| 80 | ++ |
| 81 | + |
| 82 | + |
| 83 | ++++ |
| 84 | ++ |
| 85 | +++ |
| 86 | ++ |
| 87 | + |
| 88 | + |
| 89 | +++ |
| 91 | + |
| 92 | +++ |
| 93 | +++ |
| 94 | +++ |
| 95 | +++ |
| 96 | ++ |
| 97 | ++ |

TABLE 14
Average IC₅₀ of compounds in hTHP-1 assay

| Final Target Number | Average IC₅₀ |
|---|---|
| 213 | ++ |
| 214 | ++++ |
| 215 | ++ |
| 401 | +++ |
| 402 | + |
| 404 | ++++ |
| 405 | ++ |
| 406 | ++ |
| 407 | + |
| 408 | >30 |
| 409 | ++ |
| 410 | >30 |
| 411 | >30 |
| 412 | >30 |
| 415 | >30 |
| 416 | ++++ |

### Study Example 1

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-1β, by processing pro-IL-1β into mature secreted IL-1β. In addition to its role in the inflammasome, CARD8 is also a potent inhibitor of nuclear factor NF-κB. NF-κB activation is essential for the production of pro-IL-1ϑ. Since over-production of IL-1β and dyregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-1β, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at rs2043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-1β are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

### Experimental Design

Human subjects and tissue:
   Endoscopic or surgical biopsies from areas of disease in patients with Crohn's disease and ulcerative colitis who are either anti-TNFα treatment naive or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Endpoints to be measured:
   Before ex vivo treatment—NLRP3 RNA level
   After ex vivo treatment-inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist treatment decreases processed IL-I P, processed caspase-1 or secreted IL-1P, and inflammatory cytokine RNA levels.
   Stratify response data according to treatment status at biopsy and the presence of genetic mutations in the NLRP3 gene, CARD8 gene, and ATG16L1 gene (e.g., any of the exemplary genetic mutations of these genes described herein).

### Study Example 2. Treatment of Anti-TNFα Resistant Patients with NLRP3 Antagonists

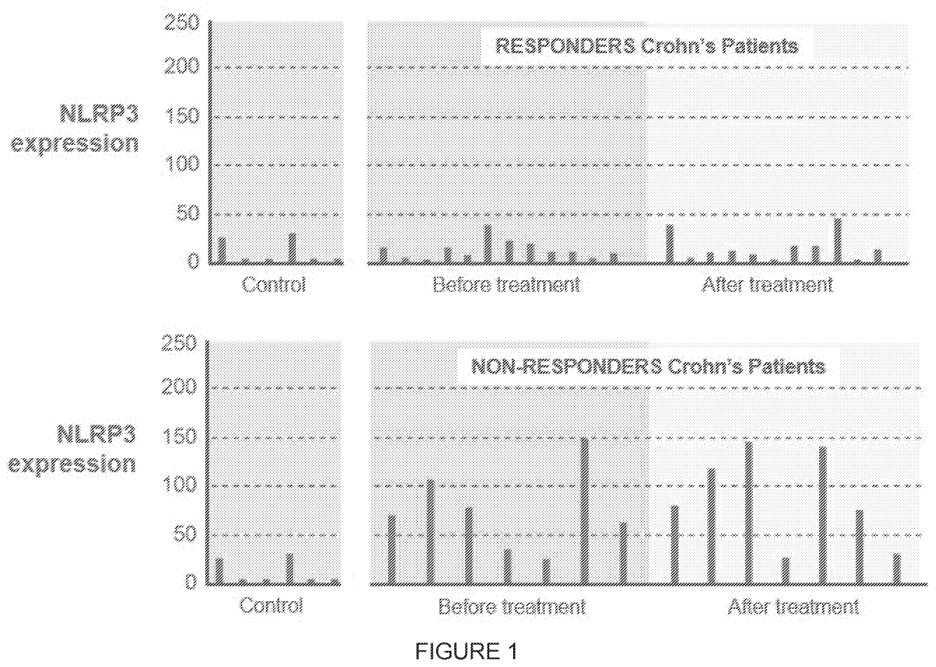

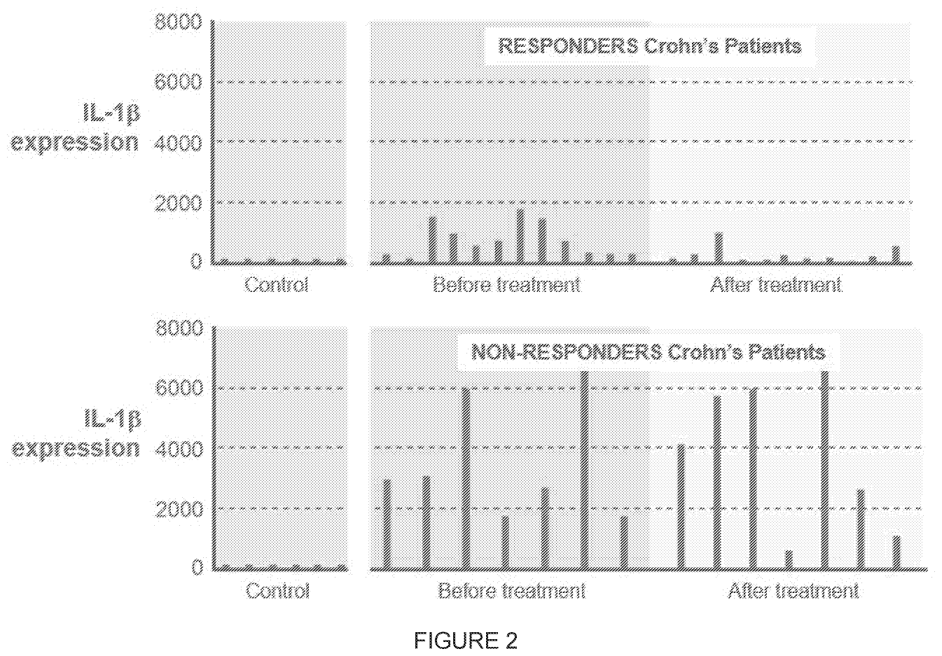

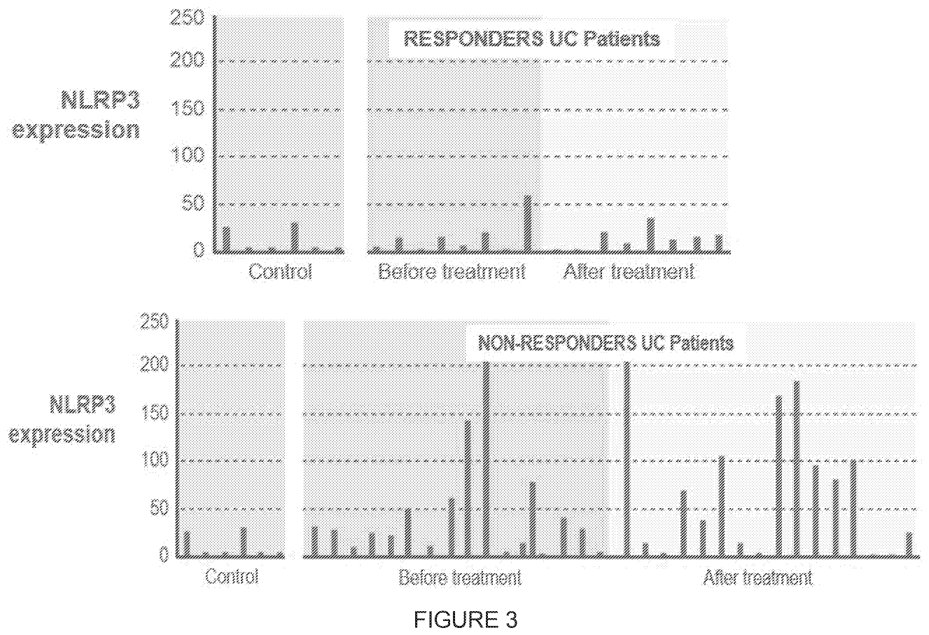

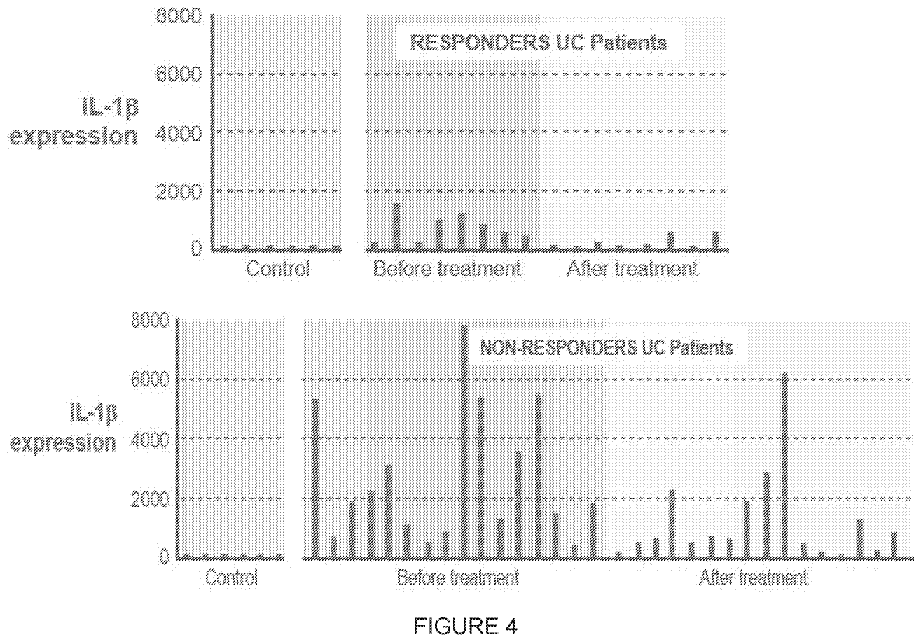

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (htps://www.ncbi.nlm.nih.gov/geo/geo2r/?acc=GSE16879). Expression levels of RNA encoding NLRP3 and IL-1β were determined using GEO2R (a tool available on the same website), based on probe sets 207075_at and 205067_at, respectively. It was surprisingly found that in Crohn's disease patients that are non-responsive to the infliximab (an anti-TNFα agent) have higher expression of NLRP3 and IL-1β RNA than responsive patients (FIGS. 1 and 2). Similar surprising results of higher expression of NLRP3 and IL-1β RNA in UC patients that are non-responsive to infliximab (an anti-TNFα agent) compared to infliximab (an anti-TNFα agent) responsive patients (FIGS. 3 and 4) were found.

Said higher levels of NLRP3 and IL-1β RNA expression levels in anti-TNFα agent non-responders, is hypothesised herein to lead to NLRP3 activation which in turns leads ot release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα□agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination threatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systemtic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

Proposed Experiment:

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Study Example 3

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
   Endoscopic or surgical biopsies from areas of disease in patients with Crohn's disease and ulcerative colitis who are either anti-TNFα treatment naive or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Ex vivo Treatments:
   NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.
Endpoints to be measured:
   Before ex vivo treatment—NLRP3 RNA level
   After ex vivo treatment-inflammasome activity (either processed IL-I P, processed caspase-1, or secreted IL-1β); RNA for inflammatory cytokines (Nanostring); viable T cell number and/or T cell apoptosis.
Data Analysis Plan:
   Determine if NLRP3 antagonist co-treatment increases T cell apoptosis/deletion in response to anti-TNF.
   Determine if the level of NLRP3 RNA expression is greater in TNF-resistant Crohn's disease and ulcerative colitis samples compared to anti-TNF treatment-naive samples.
   Determine if NLRP3 antagonist treatment decreases processed IL-1β, processed caspase-1 or secreted IL-1, and inflammatory cytokine RNA levels.

### Biological Assay—Nigericin-Stimulated IL-1β Secretion Assay in THP-1 Cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000x stock)+Pen-Strep). Cells were differentiated in bulk with 0.5 pM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 pM (final concentration) Nigericin (Enzo Life Sciences, #BML-CA421-0005), and cells were incubated for 3 hours. 10 pL supernatant was removed, and IL-1β levels were monitored using an HTRF assay (CisBio, #62IL1PEC) according to manufacturers' instructions. Viability and pyroptosis was monitored with the addition of PrestoBlue cell viability reagent (Life Technologies, #A13261) directly to the cell culture plate.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of Formula A 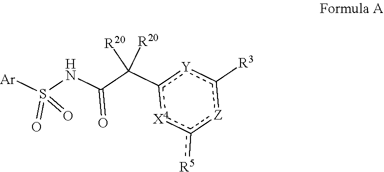
or a pharmaceutically acceptable salt thereof, wherein:
Ar is a heteroaryl group 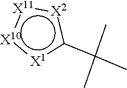
or an aryl or heteroaryl group 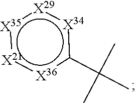
X¹ is O, S, N, CR⁴¹ or NR;
X¹⁰ is O, S, N, CR¹⁰ or NR¹⁰;
X¹¹ is O, S, N, CR¹ or NR¹;
X² is O, S, N, CR⁴² or NR⁴²; 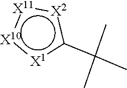
comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴; 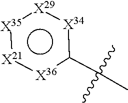
comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;
Y is N or CR²;
Z is N or CR;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A; and R⁴ and R⁵ taken together with the carbons connecting them form a four-membered to seven-membered ring B,
wherein ring A is 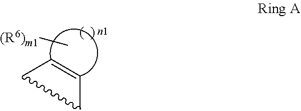
and ring B is 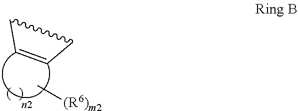
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is independently selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and
each of R¹⁷ and R¹⁸ at each occurrence is independently selected from hydrogen and C₁-C₆ alkyl.

2. A compound of Formula A 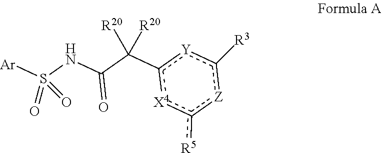
or a pharmaceutically acceptable salt thereof, wherein:
Ar is a heteroaryl group 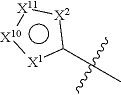
or an aryl or heteroaryl group 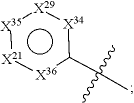
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²; 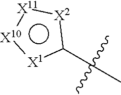
comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴; 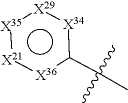
comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;
Y is N or CR²;
Z is N or CR;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A; and R⁴ and R⁵ taken together with the carbons connecting them form a four-membered to seven-membered ring B,
wherein ring A is 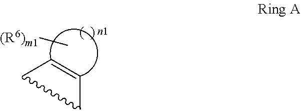
and ring B is 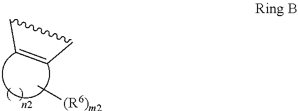
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is independently selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;
R¹⁵ is C₁-C₆ alkyl, C₁-C₆ haloalkyl, C₆-C₁₀ aryl, or 5- to 10-membered heteroaryl; and
each of R¹⁷ and R¹⁸ at each occurrence is independently selected from hydrogen and C₁-C₆ alkyl.

3. A compound of Formula I 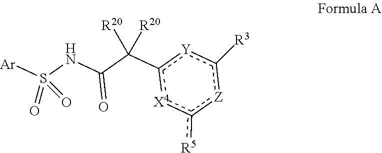
or a pharmaceutically acceptable salt thereof, wherein:
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²; 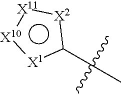
comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 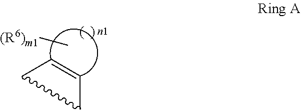
and ring B is 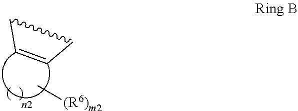
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, C₁-C₆ alkyl, oxo, C₁-C₆ alkoxy, NR¹¹R¹², ═NR¹³ COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and
each of R¹⁷ and R¹⁸ at each occurrence is independently selected from hydrogen and C₁-C₆ alkyl.

4. A compound of Formula IIa 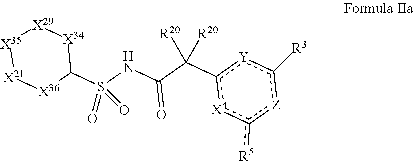
or a pharmaceutically acceptable salt thereof, wherein:
X³⁵ is N or CR³⁵;
X²¹ is N or CR²¹;
X³⁶ is N or CR³⁶;
X²⁹ is N or CR²⁹;
X³⁴ is N or CR³⁴; 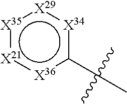
comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;
Y is N or CR²;
Z is N or CR;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 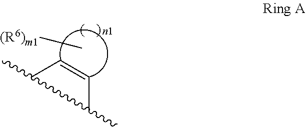
and ring B is 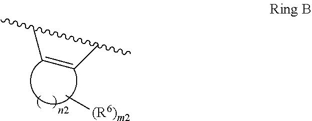
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ alkyl;
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R8, S(O₂)C₁-C₆ alkyl, S(O₂)NR¹⁷R8, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and
each of R¹⁷ and R⁸ at each occurrence is independently selected from hydrogen and C₁-C₆ alkyl.

5. A compound of Formula A 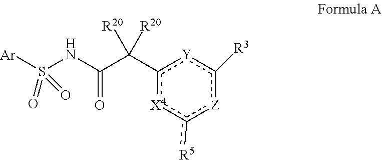
or a pharmaceutically acceptable salt thereof, wherein:
Ar is a heteroaryl group 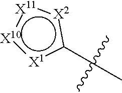
or an aryl or heteroaryl group 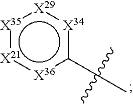
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR² 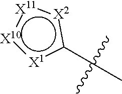
comprises at least one of CR⁴¹, CR¹⁰, CR¹, and CR⁴²;
X³⁵ is N or CR³⁵;
X²¹ is N or CR^{2′};
X³⁶ is N or CR³⁶;
X²⁹ is N or CR²⁹;
X³⁴ is N or CR³⁴; 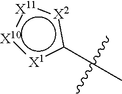
comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 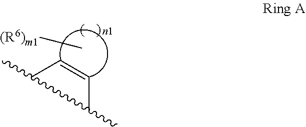
and ring B is 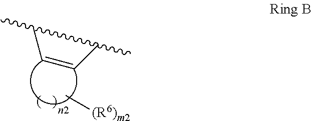
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³ COOC₁-C₆ alkyl, C₆-C₁₀ aryl, and CONR¹¹R¹²;
and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and
each of R¹⁷ and R¹⁸ at each occurrence is independently selected from hydrogen and C₁-C₆ alkyl.

6. A compound of Formula I 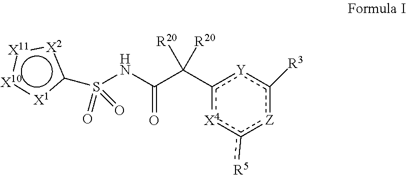
or a pharmaceutically acceptable salt thereof, wherein:
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR² 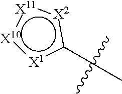
comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B, 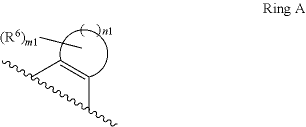
and ring B is 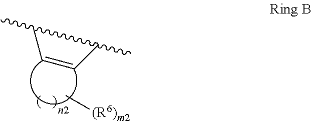
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹¹, oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³ COOC₁-C₆ alkyl, C₆-C₁₀ aryl, and CONR¹¹R¹²;
or any two of R⁴¹, R¹⁰, R¹, and R⁴² on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R8, S(O₂)C₁-C₆ alkyl, S(O₂)NR¹⁷R8, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl;
each of R⁷ and R⁸ at each occurrence is independently selected from hydrogen and C₁-C₆ alkyl.

7. A compound of Formula IIa 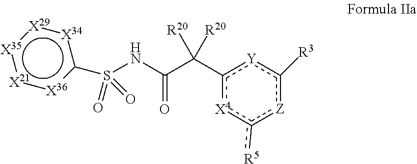
or a pharmaceutically acceptable salt thereof, wherein:
X³⁵ is N or CR³⁵;
X²¹ is N or CR²¹;
X³⁶ is N or CR³⁶;
X²⁹ is N or CR²⁹;
X³⁴ is N or CR³⁴; 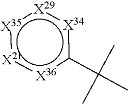
comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, Cl, F, CO₂C₁-C₆ alkyl, CO₂C₃-C₈ cycloalkyl, CONR¹¹R¹², C₁-C₆ alkyl optionally substituted with hydroxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen, and that R² and R⁴ are not both hydroxymethyl;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 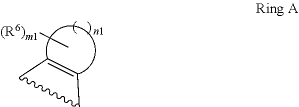
and ring B is 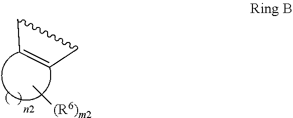
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,
wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO(5- to 10-membered heteroaryl), NHCO(3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or any two of R³⁴, R²⁹, R³⁵, R²¹ and R³⁶ on adjacent atoms, taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, CO₂R¹⁵ and CONR¹⁷R8;
R¹⁵ is C₁-C₆ alkyl; and
each of R⁷ and R¹⁸ at each occurrence is independently selected from hydrogen and C₁-C₆ alkyl.

8. A compound of Formula A 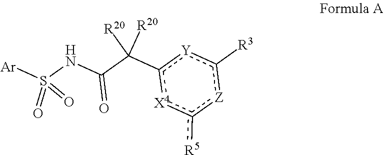
or a pharmaceutically acceptable salt thereof, wherein:
Ar is a heteroaryl group 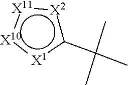
or an aryl or heteroaryl group 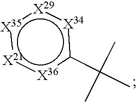
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²; 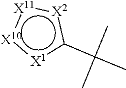
comprises at least one of CR⁴¹, CR¹⁰, CR¹, and CR⁴²;
X³⁵ is N or CR³⁵;
X²¹ is N or CR²¹;
X³⁶ is N or CR³⁶;
X²⁹ is N or CR²⁹;
X³⁴ is N or CR³⁴; 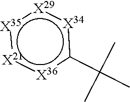
comprises at least two of CR³⁵, CR²¹, CR³⁶, CR²⁹, and CR³⁴;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 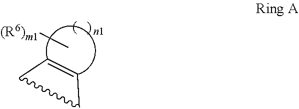
and ring B is 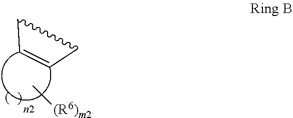
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
two of R⁴¹, R¹⁰, R¹, and R⁴² are present on adjacent atoms, and taken together with the atoms connecting them form at least one monocyclic or bicyclic 3-to-12-membered nonaromatic carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
two of R³⁴, R²⁹, R³⁵, R²¹ and R³⁶ are present on adjacent atoms, and taken together with the atoms connecting them, form at least one monocyclic or bicyclic 3-to-12-membered non-aromatic carbocyclic ring or at least one monocyclic or bicyclic 5- to 12-membered (e.g., non-aromatic) heterocyclic ring containing 1-3 heteroatoms independently selected from O, N, NH, NR¹³ and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R³⁴, R²⁹, R³⁵, R²¹ and R³⁶ that are not taken together with the atoms connecting them to form at least one ring, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl; and
each of R¹⁷ and R¹⁸ at each occurrence is independently selected from hydrogen and C₁-C₆ alkyl.

9. The compound of claim 8, wherein when R¹ and R¹⁰ are taken together with the atoms connecting them to form a 3-to-8-membered carbocyclic or heterocyclic ring containing 1 or 2 heteroatoms independently selected from O, N, and S;
then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR¹⁷R8, S(O₂)C₁-C₆ alkyl, S(O₂)NR¹⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl;
when two adjacent X²⁹, X³⁴, X²¹, and X³⁶ are other than N, and two of R³⁴, R²⁹, R³⁵, R² and R³⁶ that are on adjacent ring carbon atoms taken together with the atoms connecting them form a 6-membered aromatic ring, a five-to-eight-membered carbocyclic non-aromatic ring, a five- or six-membered heteroaromatic ring or a five-to-eight-membered heterocyclic non-aromatic ring, then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³ COOC₁-C₆ alkyl, C₆-C₁₀ aryl, and CONR¹¹R¹²; and
wherein R¹² is selected from C₁-C₆ haloalkyl, (C═NR¹⁵)NR¹⁷R¹⁸, S(O₂)C₁-C₆ alkyl, S(O₂)NR¹⁷R8, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; and
when two adjacent X²⁹, X³⁴, X²¹, and X³⁶ are other than N, and two of R³⁴, R²⁹, R³⁵, R²¹ and R³⁶ that are on adjacent ring carbon atoms taken together with the adjacent ring carbons form a 3-5 membered or 7-12 membered aromatic carbocyclic ring (e.g., 9-12 membered), a 3-4 membered or 9-12 membered non-aromatic carbocyclic ring, a 7-12-membered aromatic heterocyclic ring, or a 9-12-membered nonaromatic heterocyclic ring, then the carbocyclic or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
wherein each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to.

10. A compound of Formula A 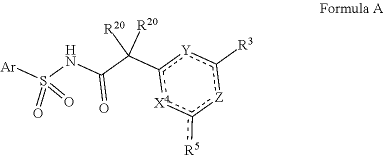
or a pharmaceutically acceptable salt thereof, wherein:
Ar is a heteroaryl group 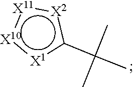
X¹ is O, S, N, CR⁴¹ or NR⁴¹;
X¹⁰ is O, S, N, CR¹⁰ or NR¹⁰;
X¹¹ is O, S, N, CR¹ or NR¹;
X² is O, S, N, CR⁴² or NR⁴²; 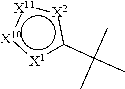
comprises at least one of CR⁴¹, CR¹⁰, CR¹, and CR⁴²;
X⁴ is CR⁴, N or NR²⁴;
each R²⁰ is the same or different and is independently selected from hydrogen and C₁-C₆ alkyl;
Y is N or CR²;
Z is N or CR⁸;
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl;
R² is hydrogen, C₁-C₆ alkoxy, halo, C₁-C₆ haloalkyl, C₃-C₇ cycloalkyl or C₁-C₆ alkyl optionally substituted with hydroxy;
R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
R⁴ is hydrogen, C₁-C₆ alkoxy, halo, C₁-C₆ haloalkyl, C₃-C₇ cycloalkyl or C₁-C₆ alkyl optionally substituted with hydroxy;
R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;
or R²⁴ is C₁-C₆ alkyl or C₃-C₈ cycloalkyl and R⁵ is ═O;
provided that at least one of R², R³, R⁴ and R⁵ is not hydrogen;
or R² and R³ taken together with the carbons connecting them form a four-membered to seven-membered ring A,
or R⁴ and R⁵ taken together with the carbons connecting them form a four-membered to seven-membered ring B,
or R² and R³ taken together with the carbons connecting them form a four-membered to seven-membered ring A and R⁴ and R⁵ taken together with the carbons connecting them form a four-membered to seven-membered ring B,
wherein ring A is 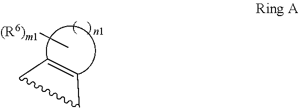
and ring B is 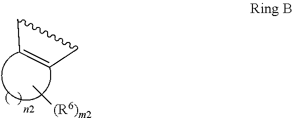
wherein
ring A is a carbocyclic ring or a heterocyclic ring containing 1 or 2 heteroatoms independently selected from O, N, and S;
n1 is from 2 to 5;
m1 is from 1 to 10;
wherein ring B is a carbocyclic ring or a heterocyclic ring containing 1 or 2 heteroatoms independently selected from O, N, and S;
n2 is from 2 to 5;
m2 is from 1 to 10;
wherein each R⁶ in each ring is the same or different and is selected from H, F, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;
two of R⁴¹, R¹⁰, R¹, and R⁴² are present on adjacent atoms, and taken together with the atoms connecting them form at least one monocyclic or bicyclic 3-to-12-membered nonaromatic carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to carbon, is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
each of the R¹, R¹⁰, R⁴¹ and R⁴² that are not taken together with the atoms connecting them to form at least one ring, when bonded to nitrogen, is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ alkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³ COOC₁-C₆ alkyl, C₆-C₁₀ aryl, and CONR¹¹R¹²;
R¹³ is C₁-C₆ alkyl;
each of R¹¹ and R¹² at each occurrence is independently selected from hydrogen, C₁-C₆ alkyl, C₁-C₆ haloalkyl, (C═NR¹⁵)NR¹⁷R¹⁸, S(O₂)C₁-C₆ alkyl, S(O₂)NR¹⁷R¹⁸, COR¹⁵, CO₂R¹⁵ and CON R¹⁷R¹⁸; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹¹ and R¹² taken together with the nitrogen they are attached to form a 3- to 7-membered ring optionally containing one or more heteroatoms in addition to the nitrogen they are attached to;
R¹⁵ is C₁-C₆ alkyl, C₁-C₆ haloalkyl, C₆-C₁₀ aryl, or 5- to 10-membered heteroaryl;
each of R¹⁷ and R¹⁸ at each occurrence is independently selected from hydrogen and C₁-C₆ alkyl.

11. The compound of claim 10, wherein when R¹ and R¹⁰ are taken together with the atoms connecting them to form a 3-to-8-membered carbocyclic or heterocyclic ring containing 1 or 2 heteroatoms independently selected from O, N, and S;
then the carbocyclic or heterocyclic ring is substituted with one or more substituents each independently selected from halo, C₁-C₆ alkyl, C₂-C₆ alkenyl, C₂-C₆ alkynyl, OC₃-C₁₀ cycloalkyl, CN, NR¹¹R¹², CONR¹¹R¹², OS(O₂)C₆-C₁₀ aryl, S(O₂)C₆-C₁₀ aryl, C₆-C₁₀ aryl, 5- to 10-membered heteroaryl, C₃-C₁₀ cycloalkyl, and 3- to 10-membered heterocycloalkyl;
wherein the C₁-C₆ alkyl, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹²; and
wherein R¹² is selected from C₁-C₆ haloalkyl, (C═NR¹⁵)NR⁷R8, S(O₂)C₁-C₆ alkyl, S(O₂)NR¹⁷R¹⁸, or C₁-C₆ alkyl substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl.

12. The compound of any one of claims 1, 2, 3, 5, 6, and 8-11, wherein the moiety

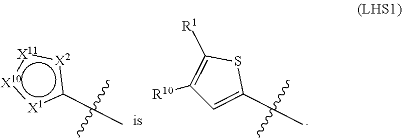

13. The compound of any one of claims 1, 2, 3, 5, 6, and 8-11, wherein the moiety

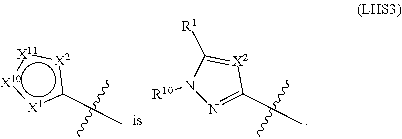

14. The compound of any one of claims 1, 2, 3, 5, 6, and 8-11, wherein LHS3 is

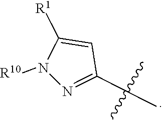

15. The compound of any one of claims 1, 2, 3, 5, 6, and 8-11, wherein the moiety

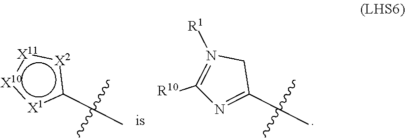

16. The compound of any one of claims 1, 2, 3, 5, 6, and 8-11, wherein the moiety

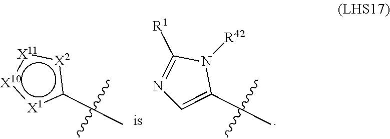

17. The compound of any one of claims 1, 2, 3, 5, 6, and 8-11, wherein the moiety

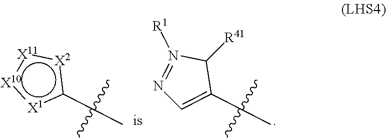

18. The compound of any one of claims 1, 2, 3, 5, 6, and 8-11, wherein the moiety

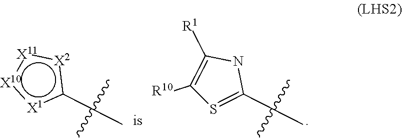

19. The compound of any one of claims 1, 2, 3, 5, 6, and 8-11, wherein the moiety

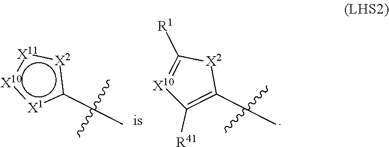

20. The compound of claim 19, wherein X¹⁰ is N; and X² is S.

21. The compound of claims 19-20, wherein LHS7 is

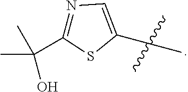 [SMILES: *c1cnc(C(C)(C)O)s1]

22. The compound of claim 19, wherein LHS7 is

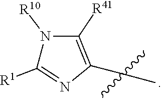

23. The compound of any one of claims 1, 2, 3, 5, 6, and 8-11 wherein the moiety

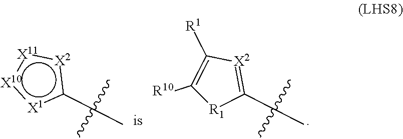

24. The compound of claim 23, wherein X¹ is S; and X² is CH.

25. The compound of claim 1, 2, 4, 5 or 7, wherein the moiety

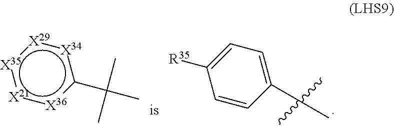

26. The compound of claim 1, 2, 4, 5 or 7, wherein the moiety

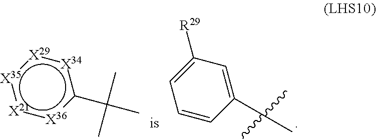

27. The compound of claim any one of claims 1, 2, 3, 5, 6, and 8-11, wherein the moiety

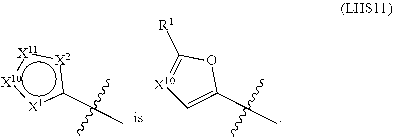

28. The compound of claim 1, 2, 4, 5, or 7, wherein the moiety

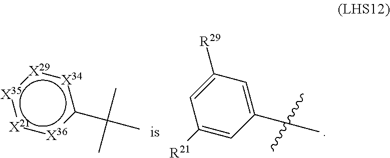

29. The compound of claim 1, 2, 4, 5, or 7, wherein the moiety is

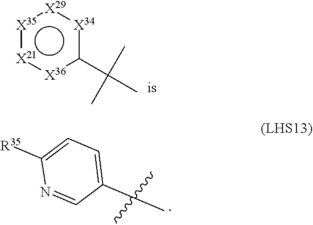

30. The compound of claim 1, 2, 4, 5, or 7-9, wherein the moiety

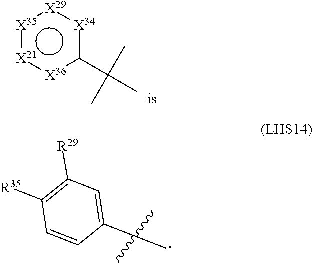

31. The compound of claim 1, 2, 4, 5, or 7, wherein the moiety 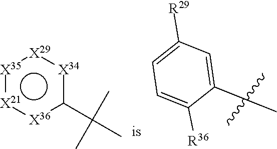
(LHS17).

32. The compound of claim 31, wherein LHS17 is

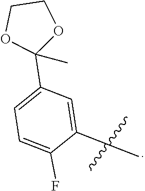 [SMILES: *c1cc(C2(C)OCCO2)ccc1F]

33. The compound of claim 1, 2, 4, 5, or 7, wherein the moiety

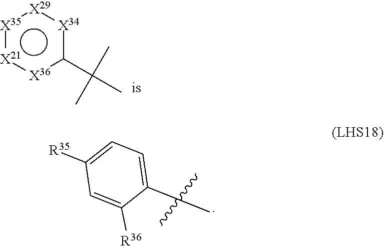

34. The compound of any one of the preceding claims, wherein the moiety

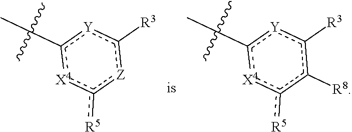

35. The compound of claim 34, wherein the moiety 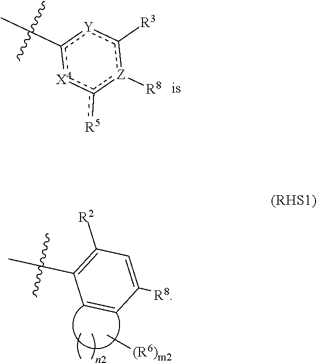
(RHS1).

36. The compound of claim 34, wherein the moiety

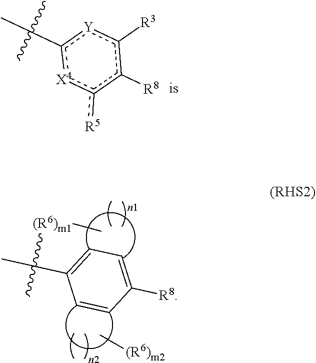

37. The compound of claim 34, wherein the moiety

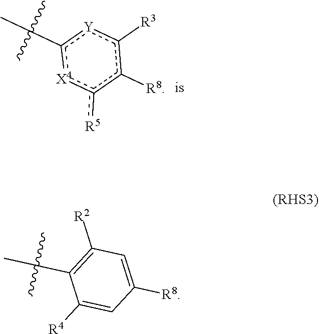

38. The compound of claim 34, wherein the moiety

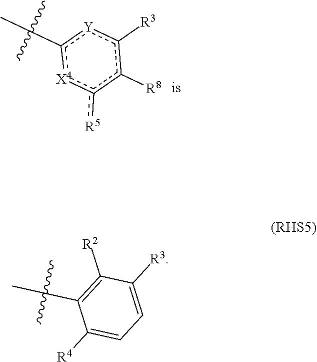

39. The compound of claim 38, wherein RHS5 is

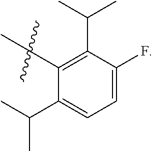 [SMILES: *c1c(C(C)C)ccc(F)c1C(C)C]

40. The compound of claim 34, wherein the moiety

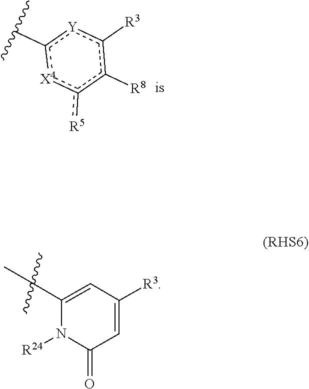

41. The compound of claim 34, wherein the moiety

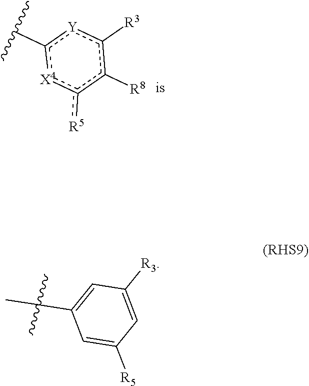

42. The compound of claim 34, wherein the moiety

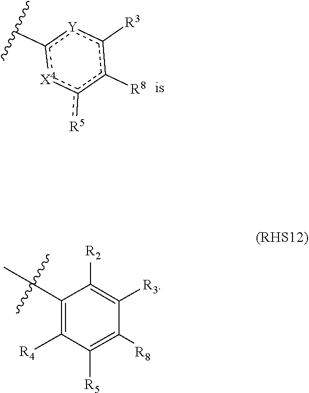

43. The compound of any one of claims 1-3, 5, 6, and 8-11, wherein X¹⁰ is CR¹⁰.

44. The compound of any one of claims 1-3, 5, 6, 8-11, 12-15, 17-18, 22, 23 and 43, wherein R¹⁰ is 2-hydroxy-2-propyl.

45. The compound of any one of claims 1-3, 5, 6, 8-11, 12-15, 17-18, 22, 23 and 43, wherein R¹⁰ is 1-hydroxy-1-cyclopropyl.

46. The compound of any one of claims 1-3, 5, 6, 8-11, 12-15, 17-18, 22, 23 and 43, wherein R¹⁰ is dimethylaminomethyl.

47. The compound of any one of claims 1-3, 5, 6, 8-11, 12-15, 17-18, 22, 23 and 43, wherein R¹⁰ is S(O₂)CH₃.

48. The compound of any one of claims 1-3, 5, 6, and 8-11, wherein X¹¹ is CR¹.

49. The compound of any one of claims 1-3, 5, 6, 8-11, 12-16, 18-19, 22, and 48, wherein R¹ is 2-hydroxy-2-propyl.

50. The compound of any one of claims 1-3, 5, 6, 8-11, 12-16, 18-19, 22, and 48, wherein R¹ is 1-hydroxy-1-cyclopropyl.

51. The compound of any one of claims 1-3, 5, 6, 8-11, 12-16, 18-19, 22, and 48, wherein R¹ is dimethylaminomethyl.

52. The compound of any one of claims 1-3, 5, 6, 8-11, 12-16, 18-19, 22, and 48, wherein R1 is S(O₂)CH₃.

53. The compound of any one of claims 1-3, 5, 6, and 8-11, wherein X¹⁰ is NR¹⁰.

54. The compound of claim 53, wherein R¹⁰ is isopropyl.

55. The compound of claim 53, wherein R¹⁰ is methyl.

56. The compound of claim 53, wherein R¹⁰ is benzyl.

57. The compound of claim 53, wherein R¹⁰ is phenyl.

58. The compound of any one of claims 1-3, 5, 6, 17, and 22, wherein R⁴¹ and R¹⁰, taken together with the atoms connecting them form a monocyclic or bicyclic 3-to-12-membered carbocyclic ring or a monocyclic or bicyclic 5- to 12-membered heterocyclic ring containing 1-3 heteroatoms independently selected from O, N, NH, NR¹³ and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

59. The compound of any one of claims 1-3, 5, 6, 12-15, 18, 22, and 23, wherein R¹⁰ and R¹, taken together with the atoms connecting them form a monocyclic or bicyclic 3-to-12-membered carbocyclic ring or a monocyclic or bicyclic 5- to 12-membered heterocyclic ring containing 1-3 heteroatoms independently selected from O, N, NH, NR¹³ and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

60. The compound of any one of claims 1-3, 5, 6, and 16, wherein R¹ and R⁴², taken together with the atoms connecting them form a monocyclic or bicyclic 3-to-12-membered carbocyclic ring or a monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

61. The compound of any one of claims 1, 2, 4, 5, and 7, wherein X³⁵ is CR³⁵.

62. The compound of any one of claims 1, 2, 4, 5, 7, 25, 29-30, 33, and 61 wherein R³⁵ is 2-hydroxy-2-propyl.

63. The compound of any one of claims 1, 2, 4, 5, 7, 25, 29-30, 33, and 61, wherein R³⁵ is 1-hydroxy-1-cyclopropyl.

64. The compound of any one of claims 1, 2, 4, 5, 7, 25, 29-30, 33, and 61, wherein R³⁵ is dimethylaminomethyl.

65. The compound of any one of claims 1, 2, 4, 5, 7, 25, 29-30, 33, and 61, wherein R³⁵ is S(O₂)CH₃.

66. The compound of any one of claims 1, 2, 4, 5, 7, 28, and 61, wherein X²¹ is CR²¹.

67. The compound of any one of claims 1, 2, 4, 5, 7, 28, 61, and 66, wherein R²¹ is 2-hydroxy-2-propyl.

68. The compound of any one of claims 1, 2, 4, 5, 7, 28, 61, and 66, wherein R²¹ is 1-hydroxy-1-cyclopropyl.

69. The compound of any one of claims 1, 2, 4, 5, 7, 28, 61, and 66, wherein R²¹ is dimethylaminomethyl.

70. The compound of any one of claims 1, 2, 4, 5, 7, 28, 61, and 66, wherein R²¹ is S(O₂)CH₃.

71. The compound of any one of claims 1, 2, 4, 5, 7, 28, 61, and 66, wherein R²¹ is halo.

72. The compound of any one of claims 1, 2, 4, 5, 7, 28, 61, and 66, wherein R²¹ is CH₃.

73. The compound of any one of claims 1, 2, 4, 5, 7, 26, 28, 30, 31, and 61, wherein R²⁹ is 2-hydroxy-2-propyl.

74. The compound of any one of claims 1, 2, 4, 5, 7, 26, 28, 30, 31, and 61, wherein R²⁹ is 1-hydroxy-1-cyclopropyl.

75. The compound of any one of claims 1, 2, 4, 5, 7, 26, 28, 30, 31, and 61, wherein R²⁹ is dimethylaminomethyl.

76. The compound of any one of claims 1, 2, 4, 5, 7, 26, 28, 30, 31, and 61, wherein R²⁹ is S(O₂)CH₃.

77. The compound of any one of claims 1, 2, 4, 5, 7, 26, 28, 30, 31, and 61, wherein R²⁹ is halo.

78. The compound of any one of claims 1, 2, 4, 5, 7, 26, 28, 30, 31, and 61, wherein R²⁹ is CH₃.

79. The compound of any one of claims 1, 2, 4, 5, 7, 31, and 33, wherein X³⁶ is CR³⁶.

80. The compound of any one of claims 1, 2, 4, 5, 7, 31, 33, and 79, wherein R³⁶ is halo.

81. The compound of any one of claims 1, 2, 4, 5, 7, 31, 33, and 79, wherein R³⁶ is CH₃.

82. The compound of any one of claims 1, 2, 4, 5, 7, and 61, wherein R³⁴ is halo.

83. The compound of any one of claims 1, 2, 4, 5, 7, and 61, wherein R³⁴ is CH₃.

84. The compound of any one of claims 1-83, wherein Y is CR², X⁴ is CR⁴, and Z is CR⁸.

85. The compound of claim 84, wherein R² is C₁-C₆ alkyl (e.g., isopropyl), halo (e.g., chloro), or C₃-C₇ cycloalkyl (e.g., cyclopropyl); R³ is hydrogen, halo (e.g., fluoro), or C₁-C₆ alkyl (e.g., isopropyl or methyl); R⁸ is hydrogen, halo (e.g., chloro or fluoro), CN, or C₁-C₆ haloalkyl (e.g., difluoromethyl); R⁵ is hydrogen or halo (e.g., fluoro); and R⁴ is halo (e.g., chloro), C₁-C₆ alkyl optionally substituted with hydroxy (e.g., isopropyl), or C₃-C₇ cycloalkyl (e.g., cyclopropyl);
or R² and R³ taken together with the carbons connecting them form a five-membered ring A,
or R⁴ and R⁵ taken together with the carbons connecting them form a five-membered ring B,
or R² and R³ taken together with the carbons connecting them form a five-membered ring A and R⁴ and R⁵ taken together with the carbons connecting them form a five-membered ring B,
wherein ring A is 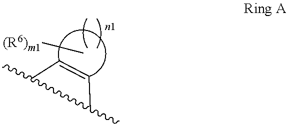
and ring B is 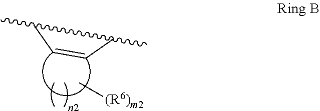
wherein
ring A is a carbocyclic ring;
n1 is 3;
m1 is 6;
wherein ring B is a carbocyclic ring;
n2 is 3;
m2 is 6;
wherein each R⁶ in each ring is the same or different and is selected from H or C₁-C₆ alkyl (e.g., methyl).

86. The compound of any one of claims 1-33 and 35-83, wherein Y is CR², X⁴ is CR⁴, and Z is N.

87. The compound of claim 86, wherein R² is C₁-C₆ alkyl (e.g., isopropyl) or halo (e.g., chloro); R³ is hydrogen or C₁-C₆ alkyl (e.g., isopropyl); R⁵ is hydrogen or halo (e.g., fluoro); R⁴ is C₁-C₆ alkyl (e.g., isopropyl);
or R² and R³ taken together with the carbons connecting them form a five-membered ring A,
or R⁴ and R⁵ taken together with the carbons connecting them form a five-membered ring B,
or R² and R³ taken together with the carbons connecting them form a five-membered ring A and R⁴ and R⁵ taken together with the carbons connecting them form a five-membered ring B,
wherein ring A is 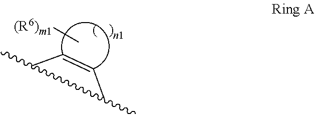
and ring B is 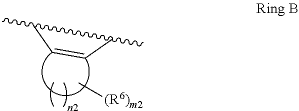
wherein
ring A is a carbocyclic ring;
n1 is 3;
m1 is 6;
wherein ring B is a carbocyclic ring;
n2 is 3;
m2 is 6;
wherein each R⁶ in each ring is the same or different and is selected from H or C₁-C₆ alkyl (e.g., methyl).

88. The compound of any one of the preceding claims, wherein each R²⁰ is hydrogen.

89. The compound of any one of claims 1-2, 3, 5, and 6, wherein
Ar is a heteroaryl group 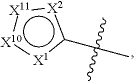
wherein
X¹ is O, S, N or CH;
X¹⁰ is N, CR¹⁰ or NR¹⁰;
X¹¹ is N, CR¹ or NR¹;
X² is O, S, N or CH;
each of R¹ and R¹⁰ when bonded to carbon is independently selected from H, C₁-C₆ alkyl, C₆-C₁₀ aryl, S(O₂)C₁-C₆ alkyl and C₃-C₇ cycloalkyl, wherein the C₁-C₆ alkyl and C₃-C₇ cycloalkyl is optionally substituted with one or more substituents each independently selected from hydroxy, oxo, C₁-C₆ alkoxy, and NR¹¹R¹²;
and each of R¹, R¹⁰ when bonded to nitrogen is independently selected from H, C₁-C₆ alkyl, C₆-C₁₀ aryl, and C₃-C₇ cycloalkyl, wherein the C₁-C₆ alkyl and C₃-C₇ cycloalkyl is optionally substituted with one or more substituents each independently selected from hydroxy and C₁-C₆ alkoxy;
R⁸ is selected from H, CN, Cl, F, C₁-C₆ alkyl, C₁-C₆ alkoxy, C₁-C₆ haloalkoxy, and C₁-C₆ haloalkyl;
R² is hydrogen, C₁-C₆ haloalkyl, C₃-C₇ cycloalkyl or C₁-C₆ alkyl;
R³ is hydrogen or halo;
R⁴ is hydrogen, C₁-C₆ haloalkyl, C₃-C₇ cycloalkyl or C₁-C₆ alkyl;
R⁵ is hydrogen or halo.

90. The compound of any one of claims 1-2, 3, 5, 6, and 8-11, wherein the compound of formula I is a compound of formula Ia 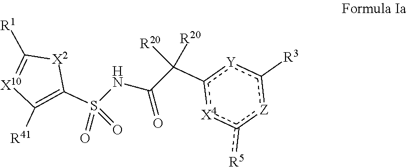
wherein
X¹⁰ is N or CR¹⁰;
and
X² is O, S, or NR⁴².

91. The compound of claim 90, wherein X¹⁰ is N; and X² is O.

92. The compound of claim 90, wherein X¹⁰ is N; and X² is S.

93. The compound of claim 90, wherein X¹⁰ is CR¹⁰; and X² is O.

94. The compound of claim 90, wherein X¹⁰ is CR¹⁰; and X² is S.

95. The compound of any one of claims 1, 2, 3, 5, 6, and 8-11, wherein the compound of formula I is a compound of formula Ib: 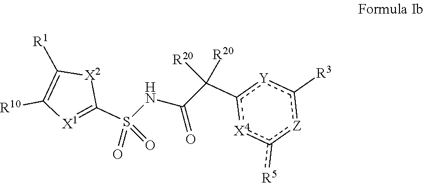
wherein
X¹ is O, S, or NR⁴¹;
and
X² is N or CR⁴².

96. The compound of claim 95, wherein X¹ is O; and X² is N.

97. The compound of claim 95, wherein X¹ is S; and X² is N.

98. The compound of claim 95, wherein X¹ is O; and X² is CR⁴².

99. The compound of claim 95, wherein X¹ is S; and X² is CR⁴².

100. The compound of any one of claims 90-99, wherein R¹ is 2-hydroxy-2-propyl.

101. The compound of any one of claims 90 and 93-99, wherein R¹⁰ is 2-hydroxy-2-propyl.

102. The compound of any one of claims 90-99, wherein R¹ is 1-hydroxy-1-cyclopropyl.

103. The compound of any one of claims 90 and 93-99, wherein R¹⁰ is 1-hydroxy-1-cyclopropyl.

104. The compound of any one of claims 90-95, wherein R⁴¹ is 2-hydroxy-2-propyl.

105. The compound of any one of claims 90, 95 and 98-99, wherein R⁴² is 2-hydroxy-2-propyl.

106. The compound of any one of claims 90-95, wherein R⁴¹ is 1-hydroxy-1-cyclopropyl.

107. The compound of any one of claims 90, 95 and 98-99, wherein R⁴² is 1-hydroxy-1-cyclopropyl.

108. The compound of any one of claims 90-99, wherein R¹ is dimethylaminomethyl.

109. The compound of any one of claims 90-99, wherein R¹ is S(O₂)CH₃.

110. The compound of any one of claims 90 and 93-99, wherein R¹⁰ is dimethylaminomethyl.

111. The compound of any one of claims 90 and 93-99, wherein R¹⁰ is S(O₂)CH₃.

112. The compound of any one of claims 90-95, wherein R⁴¹ is dimethylaminomethyl.

113. The compound of any one of claims 90-95, wherein R⁴¹ is S(O₂)CH₃.

114. The compound of any one of claims 90, 95 and 98-99, wherein R⁴² is dimethylaminomethyl.

115. The compound of any one of claims 90, 95 and 98-99, wherein R⁴² is S(O₂)CH₃.

116. The compound of any one of claims 1, 2, 4, 5, and 7, wherein
Ar is an aryl or heteroaryl group 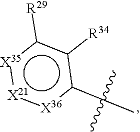
X³⁵ is CR³⁵
X²¹ is N or CR²¹;
X³⁶ is CR³⁶;
each of R³⁴, R²⁹, R³⁵, R²¹ and R³⁶ is independently selected from H, C₁-C₆ alkyl, halo, C₃-C₇ cycloalkyl, 3- to 7-membered nonaromatic monocyclic heterocycloalkyl, C₆-C₁₀ aryl, and S(O₂)C₁-C₆ alkyl;
wherein the C₁-C₆ alkyl, 3- to 7-membered nonaromatic monocyclic heterocycloalkyl, and C₃-C₇ cycloalkyl is optionally substituted with one or more substituents each independently selected from hydroxyl, C₁-C₆ alkyl, oxo, NR¹¹R¹², and 3- to 7-membered heterocycloalkyl, R⁸ is selected from H, CN, Cl, F, C₁-C₆ alkyl, C₁-C₆ alkoxy, C₁-C₆ haloalkoxy, and C₁-C₆ haloalkyl;
R² is hydrogen, C₁-C₆ haloalkyl, C₃-C₇ cycloalkyl or C₁-C₆ alkyl;
R³ is hydrogen or halo;
R⁴ is hydrogen, C₁-C₆ haloalkyl, C₃-C₇ cycloalkyl or C₁-C₆ alkyl;
R⁵ is hydrogen or halo.

117. The compound of claim 116, wherein R³⁵ is 2-hydroxy-2-propyl.

118. The compound of claim 116, wherein R³⁵ is 1-hydroxy-1-cyclopropyl.

119. The compound of claim 116, wherein R³⁵ is dimethylaminomethyl.

120. The compound of claim 116, wherein R³⁵ is S(O₂)CH₃.

121. The compound of claim 116, wherein R³⁵ is methyl.

122. The compound of claim 116, wherein R³⁵ halo.

123. The compound of claim 116, wherein R²¹ is 2-hydroxy-2-propyl.

124. The compound of claim 116, wherein R²¹ is 1-hydroxy-1-cyclopropyl.

125. The compound of claim 116, wherein R²¹ is dimethylaminomethyl.

126. The compound of claim 116, wherein R²¹ is S(O₂)CH₃.

127. The compound of claim 116, wherein R²¹ is methyl.

128. The compound of claim 116, wherein R²¹ halo.

129. The compound of claim 116, wherein R²⁹ is 2-hydroxy-2-propyl.

130. The compound of claim 116, wherein R²⁹ is 1-hydroxy-1-cyclopropyl.

131. The compound of claim 116, wherein R²⁹ is dimethylaminomethyl.

132. The compound of claim 116, wherein R²⁹ is S(O₂)CH₃.

133. The compound of claim 116, wherein R²⁹ is methyl.

134. The compound of claim 116, wherein R²⁹ halo.

135. The compound of claim 116, wherein R³⁶ is methyl.

136. The compound of claim 116, wherein R³⁶ halo.

137. The compound of claim 116, wherein R³⁴ is methyl.

138. The compound of claim 116, wherein R³⁴ halo.

139. The compound of claim 1-3, 5, 6, 8, and 10, wherein the ring 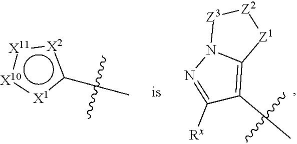
wherein:
R^{x} is selected from the group consisting of H and C₁-C₆ alkyl (e.g., methyl);
Z¹ is selected from the group consisting of O, NH, NCH₃, and —CH₂— optionally substituted with 1-2 R⁵⁰;
Z² is selected from the group consisting of NH, NCH₃, and —CH₂— optionally substituted with 1-2 R⁵⁰;
Z³ is selected from the group consisting of —CH₂— optionally substituted with 1-2 R⁵⁰, —CH₂CH₂— optionally substituted with 1-2 R⁵⁰, and -CH₂CH₂CH₂— optionally substituted with 1-2 R⁵⁰;
R⁵⁰ is selected from the group consisting of hydroxy, halo (e.g., fluoro), oxo, C₁-C₆ alkyl (e.g., methyl or ethyl) optionally substituted with one R⁵¹, C₁-C₆ alkoxy (e.g., methoxy, ethoxy, or isopropoxy) optionally substituted with one R⁵¹, NR¹¹R¹², 3- to 10-membered heterocycloalkyl (e.g., azetidinyl or pyrrolidinyl) optionally substituted with one R⁵¹, or at least one pair of R⁵⁰ on the same atom, taken together with the atom connecting them, independently forms a monocyclic C₃-C₄ carbocyclic ring or a monocyclic 3- to 4-membered heterocyclic ring containing 1 O atom;
R⁵¹ is selected from the group consisting of halo (e.g., fluoro), NR¹¹R¹², C₂-C₆ alkynyl (e.g., ethynyl), and C₁-C₆ alkoxy (e.g., methoxy);
R¹¹ and R¹² at each occurrence is independently selected from hydrogen, C₁-C₆ alkyl (e.g., methyl or ethyl), COR¹⁵, and CO₂R¹⁵; and
R¹⁵ is selected from the group consisting of: C₁-C₆ alkyl (e.g., methyl or t-butyl) and C₁-C₆ haloalkyl (e.g., trifluoromethyl).

140. The compound of any one of claims 1-3, 5-6, 8, and 10, wherein the ring 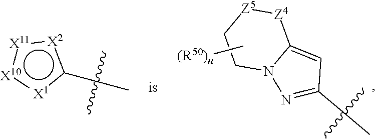
wherein
Z⁴ is selected from the group consisting of: —CH₂— optionally substituted with 1-2 R⁵⁰, O, —C(O)—, N—CH₃, and NH;
Z⁵ is selected from the group consisting of: O, —C(O)—, NH, N—CH₃, —CH₂— optionally substituted with 1-2 R⁵⁰, and —CH₂CH₂— optionally substituted with 1-2 R⁵⁰;
u is 0, 1, 2, 3, or 4;
R⁵⁰ is selected from the group consisting of: hydroxy, halo (e.g., fluoro), C₁-C₆ alkyl (e.g., methyl) optionally substituted with one R⁵¹, C₁-C₆ alkoxy (e.g., methoxy, isopropoxy, or t-butoxy) optionally substituted with one R⁵¹, NR¹¹R¹², oxo, 3- to 10-membered heterocycloalkyl (e.g., azetidinyl or pyrrolidinyl) optionally substituted with one R⁵¹;
R⁵¹ is selected from the group consisting of NR¹¹R¹², C₂-C₆ alkynyl (e.g., ethynyl), and C₁-C₆ alkoxy (e.g., methoxy);
R¹¹ and R¹² at each occurrence is independently selected from hydrogen, C₁-C₆ alkyl (e.g., methyl or ethyl), COR¹⁵, and CO₂R¹⁵; and
R¹⁵ is selected from the group consisting of: C₁-C₆ alkyl (e.g., t-butyl).

141. The compound of any one of claims 1-33 and 43-140, wherein the ring 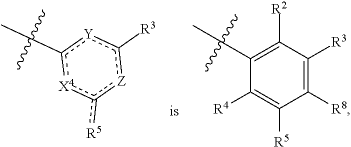
wherein:
R² is halo (e.g., chloro), C₃-C₇ cycloalkyl (e.g., cyclopropyl), or C₁-C₆ alkyl (e.g., isopropyl);
R³ is hydrogen, halo (e.g., fluoro), or C₁-C₆ alkyl (e.g., isopropyl);
R⁸ is selected from H, CN, halo (e.g., chloro or fluoro), and C₁-C₆ haloalkyl (e.g., difluoromethyl);
R⁵ is hydrogen, C₁-C₆ alkyl (e.g., isopropyl), C₃-C₇ cycloalkyl (e.g., cyclopropyl), or halo (e.g., fluoro); and
R⁴ is halo (e.g., chloro), C₃-C₇ cycloalkyl (e.g., cyclopropyl), or C₁-C₆ alkyl (e.g., isopropyl).

142. The compound any one of claims 1-33 and 43-140, wherein the ring 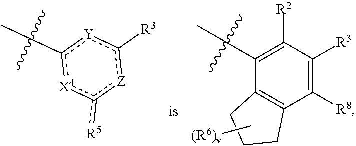
wherein:
R² is halo (e.g., chloro) or C₁-C₆ alkyl (e.g., isopropyl);
R³ is hydrogen;
or R² and R³ taken together with the carbons connecting them form a 5-membered carbocyclic ring;
R⁸ is selected from H, CN, and halo (e.g., chloro or fluoro);
v is 0 or 1; and
R⁶ is C₁-C₆ alkyl (e.g., methyl).

143. A compound selected from the group consisting of the compounds in Table 1A and pharmaceutically acceptable salts thereof.

144. A compound selected from the group consisting of the compounds below:

| Com- | |
|---|---|
| pound | Structure |
| 127 | 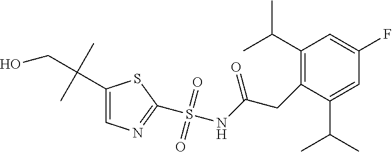 [SMILES: CC(C)c1cc(F)cc(C(C)C)c1CC(=O)NS(=O)(=O)c1ncc(C(C)(C)CO)s1] |
| 128 | 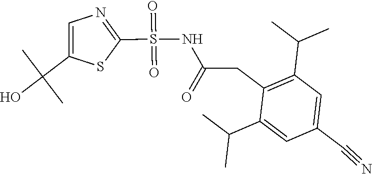 [SMILES: CC(C)c1cc(C#N)cc(C(C)C)c1CC(=O)NS(=O)(=O)c1ncc(C(C)(C)O)s1] |
| 129 | 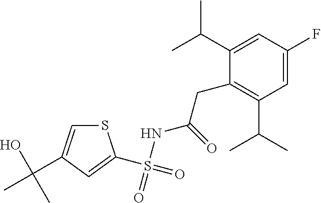 [SMILES: CC(C)c1cc(F)cc(C(C)C)c1CC(=O)NS(=O)(=O)c1cc(C(C)(C)O)cs1] |
| 130 | 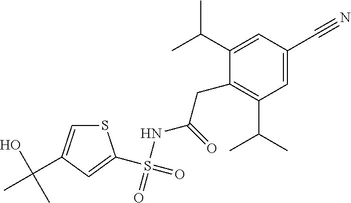 [SMILES: CC(C)c1cc(C#N)cc(C(C)C)c1CC(=O)NS(=O)(=O)c1cc(C(C)(C)O)cs1] |
| 131 | 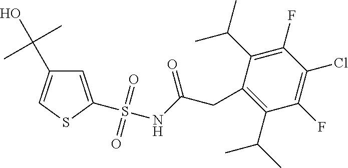 [SMILES: CC(C)c1c(F)c(Cl)c(F)c(C(C)C)c1CC(=O)NS(=O)(=O)c1cc(C(C)(C)O)cs1] |
| 132 | 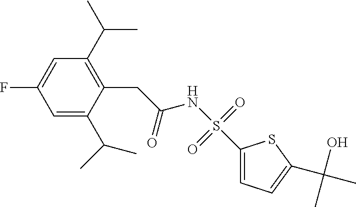 [SMILES: CC(C)c1cc(F)cc(C(C)C)c1CC(=O)NS(=O)(=O)c1ccc(C(C)(C)O)s1] |
| 133 | 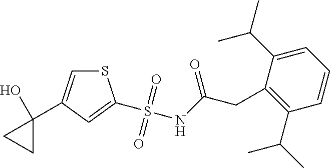 [SMILES: CC(C)c1cccc(C(C)C)c1CC(=O)NS(=O)(=O)c1cc(C2(O)CC2)cs1] |
| 134 | 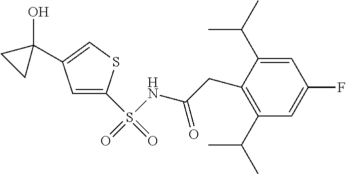 [SMILES: CC(C)c1cc(F)cc(C(C)C)c1CC(=O)NS(=O)(=O)c1cc(C2(O)CC2)cs1] |
| 135 | 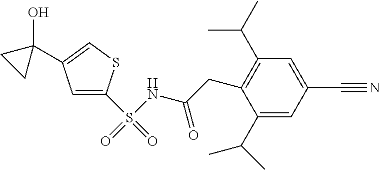 [SMILES: CC(C)c1cc(C#N)cc(C(C)C)c1CC(=O)NS(=O)(=O)c1cc(C2(O)CC2)cs1] |
| 136 | 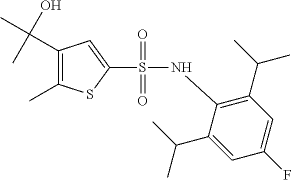 [SMILES: Cc1sc(S(=O)(=O)Nc2c(C(C)C)cc(F)cc2C(C)C)cc1C(C)(C)O] |
| 137 | 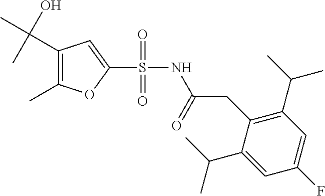 [SMILES: Cc1oc(S(=O)(=O)NC(=O)Cc2c(C(C)C)cc(F)cc2C(C)C)cc1C(C)(C)O] |
| 138 | 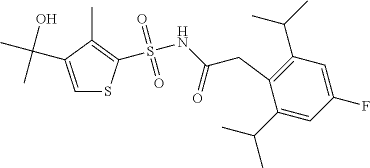 [SMILES: Cc1c(C(C)(C)O)csc1S(=O)(=O)NC(=O)Cc1c(C(C)C)cc(F)cc1C(C)C] |
| 139 | 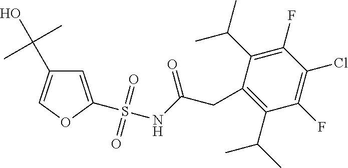 [SMILES: CC(C)c1c(F)c(Cl)c(F)c(C(C)C)c1CC(=O)NS(=O)(=O)c1cc(C(C)(C)O)co1] |
| 140 | 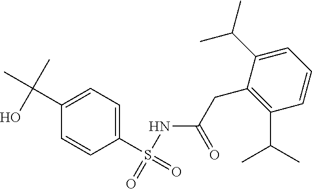 [SMILES: CC(C)c1cccc(C(C)C)c1CC(=O)NS(=O)(=O)c1ccc(C(C)(C)O)cc1] |
| 141 | 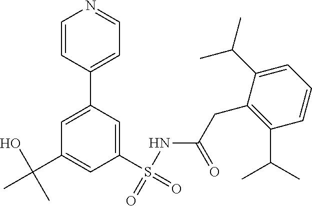 [SMILES: CC(C)c1cccc(C(C)C)c1CC(=O)NS(=O)(=O)c1cc(-c2ccncc2)cc(C(C)(C)O)c1] |
| 142 | 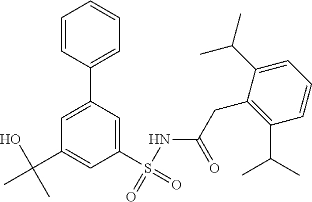 [SMILES: CC(C)c1cccc(C(C)C)c1CC(=O)NS(=O)(=O)c1cc(-c2ccccc2)cc(C(C)(C)O)c1] |
| 143 | 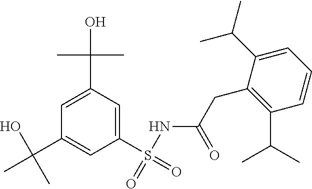 [SMILES: CC(C)c1cccc(C(C)C)c1CC(=O)NS(=O)(=O)c1cc(C(C)(C)O)cc(C(C)(C)O)c1] |
| 144 | 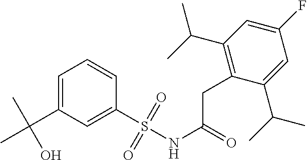 [SMILES: CC(C)c1cc(F)cc(C(C)C)c1CC(=O)NS(=O)(=O)c1cccc(C(C)(C)O)c1] |
| 145 | 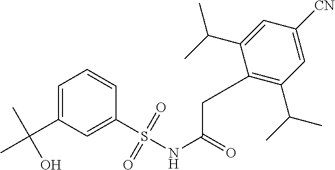 [SMILES: CC(C)c1cc(C#N)cc(C(C)C)c1CC(=O)NS(=O)(=O)c1cccc(C(C)(C)O)c1] |
| 146 | 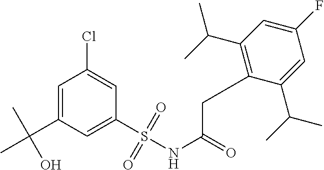 [SMILES: CC(C)c1cc(F)cc(C(C)C)c1CC(=O)NS(=O)(=O)c1cc(Cl)cc(C(C)(C)O)c1] |
| 147 | 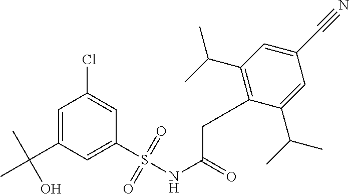 [SMILES: CC(C)c1cc(C#N)cc(C(C)C)c1CC(=O)NS(=O)(=O)c1cc(Cl)cc(C(C)(C)O)c1] |
| 148 | 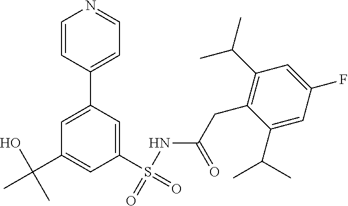 [SMILES: CC(C)c1cc(F)cc(C(C)C)c1CC(=O)NS(=O)(=O)c1cc(-c2ccncc2)cc(C(C)(C)O)c1] |
| 149 | 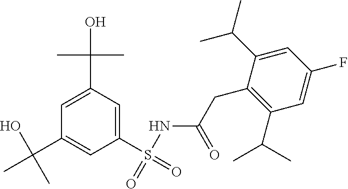 [SMILES: CC(C)c1cc(F)cc(C(C)C)c1CC(=O)NS(=O)(=O)c1cc(C(C)(C)O)cc(C(C)(C)O)c1] |
| 150 | 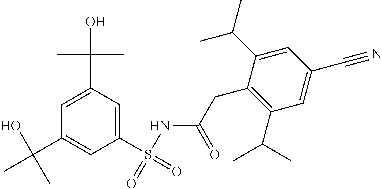 [SMILES: CC(C)c1cc(C#N)cc(C(C)C)c1CC(=O)NS(=O)(=O)c1cc(C(C)(C)O)cc(C(C)(C)O)c1] |
| 151 | 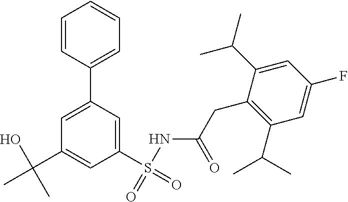 [SMILES: CC(C)c1cc(F)cc(C(C)C)c1CC(=O)NS(=O)(=O)c1cc(-c2ccccc2)cc(C(C)(C)O)c1] |
| 152 | 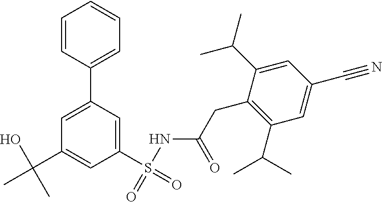 [SMILES: CC(C)c1cc(C#N)cc(C(C)C)c1CC(=O)NS(=O)(=O)c1cc(-c2ccccc2)cc(C(C)(C)O)c1] |
| 153 | 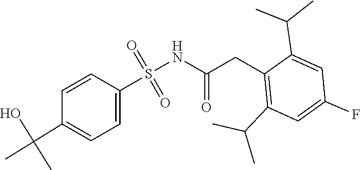 [SMILES: CC(C)c1cc(F)cc(C(C)C)c1CC(=O)NS(=O)(=O)c1ccc(C(C)(C)O)cc1] |
| 154 | 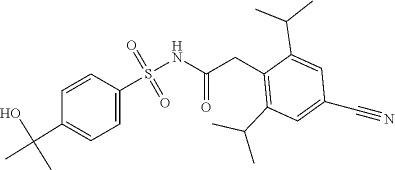 [SMILES: CC(C)c1cc(C#N)cc(C(C)C)c1CC(=O)NS(=O)(=O)c1ccc(C(C)(C)O)cc1] |
| 155 | 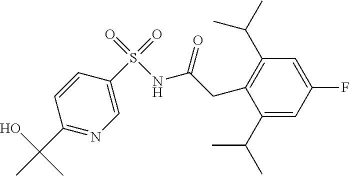 [SMILES: CC(C)c1cc(F)cc(C(C)C)c1CC(=O)NS(=O)(=O)c1ccc(C(C)(C)O)nc1] |
| 156 | 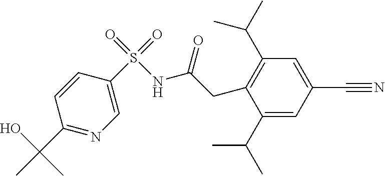 [SMILES: CC(C)c1cc(C#N)cc(C(C)C)c1CC(=O)NS(=O)(=O)c1ccc(C(C)(C)O)nc1] |
| 157 | 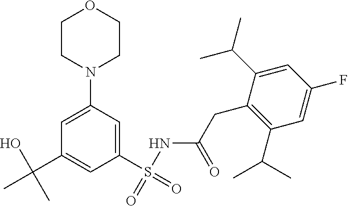 [SMILES: CC(C)c1cc(F)cc(C(C)C)c1CC(=O)NS(=O)(=O)c1cc(N2CCOCC2)cc(C(C)(C)O)c1] |
| 158 | 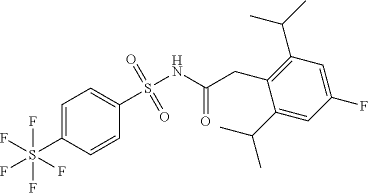 [SMILES: CC(C)c1cc(F)cc(C(C)C)c1CC(=O)NS(=O)(=O)c1ccc(S(F)(F)(F)(F)F)cc1] |
| 159 | 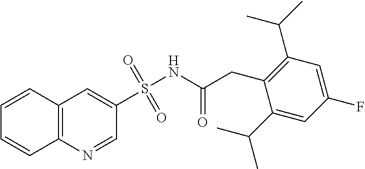 [SMILES: CC(C)c1cc(F)cc(C(C)C)c1CC(=O)NS(=O)(=O)c1cnc2ccccc2c1] |
| 160 | 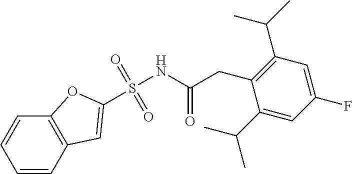 [SMILES: CC(C)c1cc(F)cc(C(C)C)c1CC(=O)NS(=O)(=O)c1cc2ccccc2o1] |
| 161 | 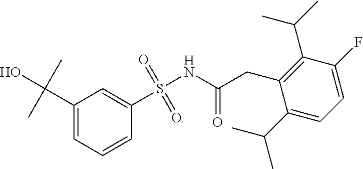 [SMILES: CC(C)c1ccc(F)c(C(C)C)c1CC(=O)NS(=O)(=O)c1cccc(C(C)(C)O)c1] |
| 162 | 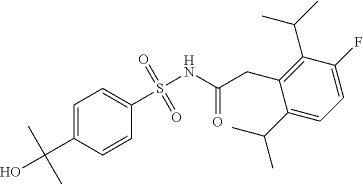 [SMILES: CC(C)c1ccc(F)c(C(C)C)c1CC(=O)NS(=O)(=O)c1ccc(C(C)(C)O)cc1] |
| 163 | 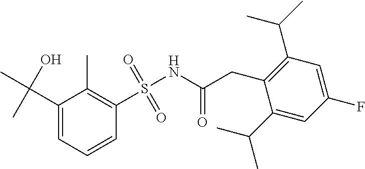 [SMILES: Cc1c(C(C)(C)O)cccc1S(=O)(=O)NC(=O)Cc1c(C(C)C)cc(F)cc1C(C)C] |
| 164 | 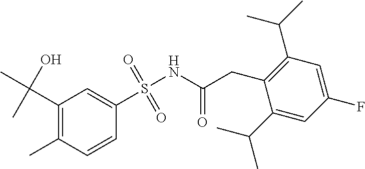 [SMILES: Cc1ccc(S(=O)(=O)NC(=O)Cc2c(C(C)C)cc(F)cc2C(C)C)cc1C(C)(C)O] |
| 165 | 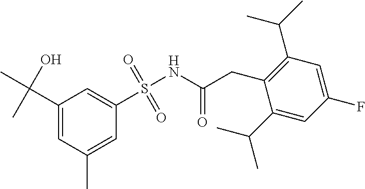 [SMILES: Cc1cc(C(C)(C)O)cc(S(=O)(=O)NC(=O)Cc2c(C(C)C)cc(F)cc2C(C)C)c1] |
| 166 | 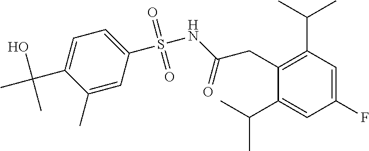 [SMILES: Cc1cc(S(=O)(=O)NC(=O)Cc2c(C(C)C)cc(F)cc2C(C)C)ccc1C(C)(C)O] |
| 167 | 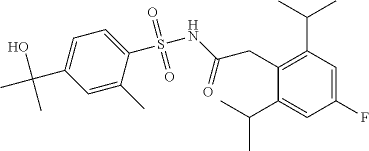 [SMILES: Cc1cc(C(C)(C)O)ccc1S(=O)(=O)NC(=O)Cc1c(C(C)C)cc(F)cc1C(C)C] |
| 168 | 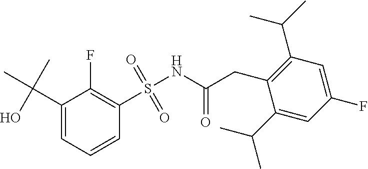 [SMILES: CC(C)c1cc(F)cc(C(C)C)c1CC(=O)NS(=O)(=O)c1cccc(C(C)(C)O)c1F] |
| 169 | 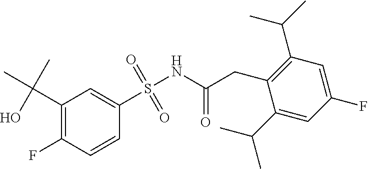 [SMILES: CC(C)c1cc(F)cc(C(C)C)c1CC(=O)NS(=O)(=O)c1ccc(F)c(C(C)(C)O)c1] |
| 170 | 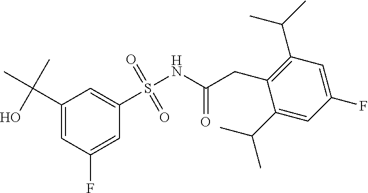 [SMILES: CC(C)c1cc(F)cc(C(C)C)c1CC(=O)NS(=O)(=O)c1cc(F)cc(C(C)(C)O)c1] |
| 171 | 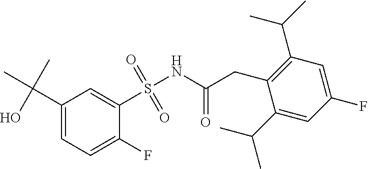 [SMILES: CC(C)c1cc(F)cc(C(C)C)c1CC(=O)NS(=O)(=O)c1cc(C(C)(C)O)ccc1F] |
| 172 | 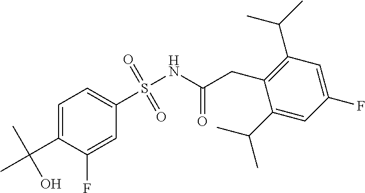 [SMILES: CC(C)c1cc(F)cc(C(C)C)c1CC(=O)NS(=O)(=O)c1ccc(C(C)(C)O)c(F)c1] |
| 173 | 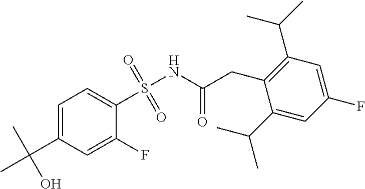 [SMILES: CC(C)c1cc(F)cc(C(C)C)c1CC(=O)NS(=O)(=O)c1ccc(C(C)(C)O)cc1F] |
| 174 | 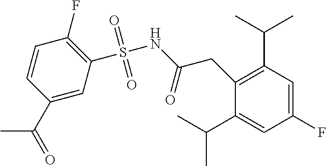 [SMILES: CC(=O)c1ccc(F)c(S(=O)(=O)NC(=O)Cc2c(C(C)C)cc(F)cc2C(C)C)c1] |
| 175 | 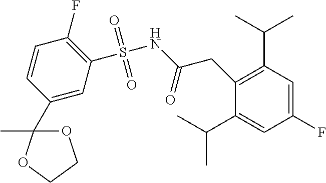 [SMILES: CC(C)c1cc(F)cc(C(C)C)c1CC(=O)NS(=O)(=O)c1cc(C2(C)OCCO2)ccc1F] |
| 176 | 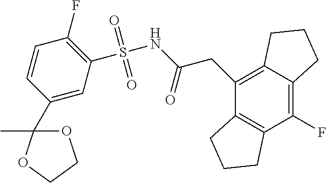 [SMILES: CC1(c2ccc(F)c(S(=O)(=O)NC(=O)Cc3c4c(c(F)c5c3CCC5)CCC4)c2)OCCO1] |
| 177 | 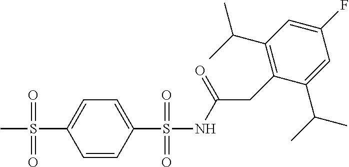 [SMILES: CC(C)c1cc(F)cc(C(C)C)c1CC(=O)NS(=O)(=O)c1ccc(S(C)(=O)=O)cc1] |
| 178 | 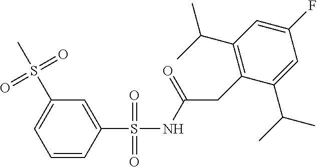 [SMILES: CC(C)c1cc(F)cc(C(C)C)c1CC(=O)NS(=O)(=O)c1cccc(S(C)(=O)=O)c1] |
| 179 | 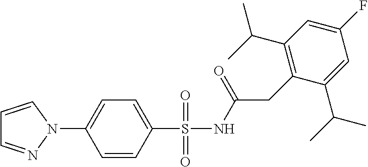 [SMILES: CC(C)c1cc(F)cc(C(C)C)c1CC(=O)NS(=O)(=O)c1ccc(-n2cccn2)cc1] |
| 180 | 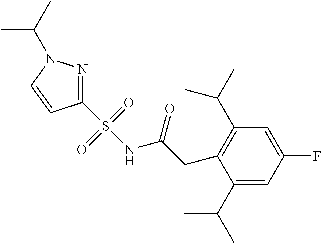 [SMILES: CC(C)c1cc(F)cc(C(C)C)c1CC(=O)NS(=O)(=O)c1ccn(C(C)C)n1] |
| 181 | 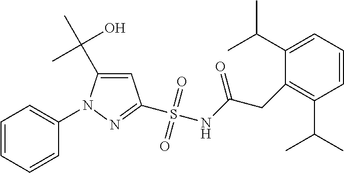 [SMILES: CC(C)c1cccc(C(C)C)c1CC(=O)NS(=O)(=O)c1cc(C(C)(C)O)n(-c2ccccc2)n1] |
| 182 | 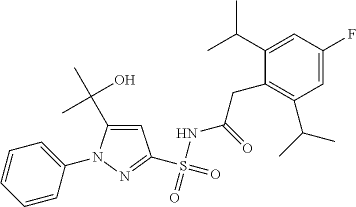 [SMILES: CC(C)c1cc(F)cc(C(C)C)c1CC(=O)NS(=O)(=O)c1cc(C(C)(C)O)n(-c2ccccc2)n1] |
| 183 | 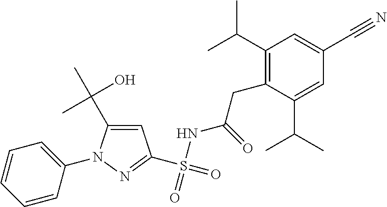 [SMILES: CC(C)c1cc(C#N)cc(C(C)C)c1CC(=O)NS(=O)(=O)c1cc(C(C)(C)O)n(-c2ccccc2)n1] |
| 184 | 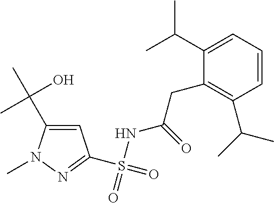 [SMILES: CC(C)c1cccc(C(C)C)c1CC(=O)NS(=O)(=O)c1cc(C(C)(C)O)n(C)n1] |
| 185 | 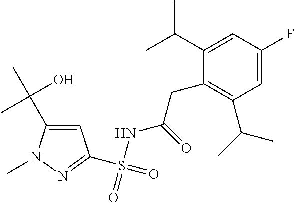 [SMILES: CC(C)c1cc(F)cc(C(C)C)c1CC(=O)NS(=O)(=O)c1cc(C(C)(C)O)n(C)n1] |
| 186 | 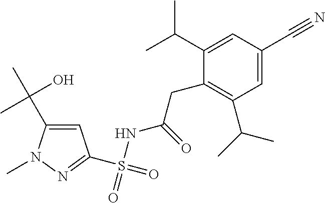 [SMILES: CC(C)c1cc(C#N)cc(C(C)C)c1CC(=O)NS(=O)(=O)c1cc(C(C)(C)O)n(C)n1] |
| 187 | 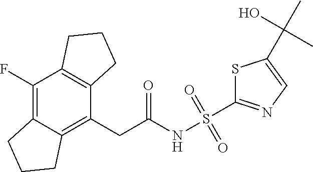 [SMILES: CC(C)(O)c1cnc(S(=O)(=O)NC(=O)Cc2c3c(c(F)c4c2CCC4)CCC3)s1] |
| 188 | 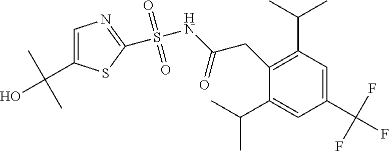 [SMILES: CC(C)c1cc(C(F)(F)F)cc(C(C)C)c1CC(=O)NS(=O)(=O)c1ncc(C(C)(C)O)s1] |
| 189 | 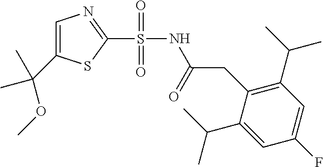 [SMILES: COC(C)(C)c1cnc(S(=O)(=O)NC(=O)Cc2c(C(C)C)cc(F)cc2C(C)C)s1] |
| 190 | 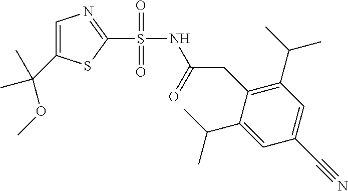 [SMILES: COC(C)(C)c1cnc(S(=O)(=O)NC(=O)Cc2c(C(C)C)cc(C#N)cc2C(C)C)s1] |
| 191 | 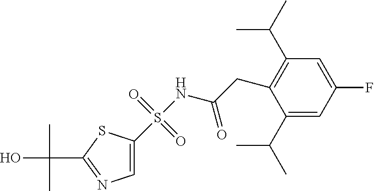 [SMILES: CC(C)c1cc(F)cc(C(C)C)c1CC(=O)NS(=O)(=O)c1cnc(C(C)(C)O)s1] |
| 192 | 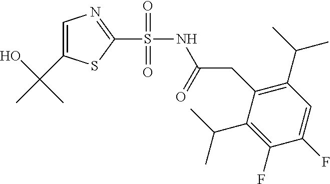 [SMILES: CC(C)c1cc(F)c(F)c(C(C)C)c1CC(=O)NS(=O)(=O)c1ncc(C(C)(C)O)s1] |
| 193 | 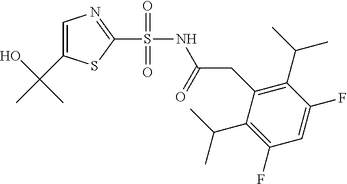 [SMILES: CC(C)c1c(F)cc(F)c(C(C)C)c1CC(=O)NS(=O)(=O)c1ncc(C(C)(C)O)s1] |
| 194 | 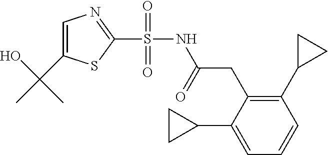 [SMILES: CC(C)(O)c1cnc(S(=O)(=O)NC(=O)Cc2c(C3CC3)cccc2C2CC2)s1] |
| 195 | 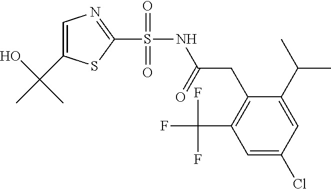 [SMILES: CC(C)c1cc(Cl)cc(C(F)(F)F)c1CC(=O)NS(=O)(=O)c1ncc(C(C)(C)O)s1] |
| 196 | 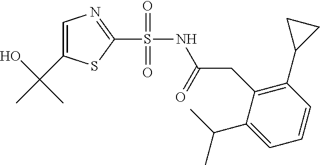 [SMILES: CC(C)c1cccc(C2CC2)c1CC(=O)NS(=O)(=O)c1ncc(C(C)(C)O)s1] |
| 197 | 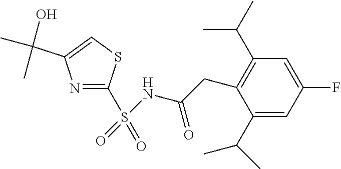 [SMILES: CC(C)c1cc(F)cc(C(C)C)c1CC(=O)NS(=O)(=O)c1nc(C(C)(C)O)cs1] |
| 198 | 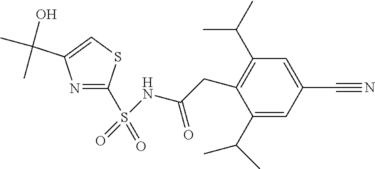 [SMILES: CC(C)c1cc(C#N)cc(C(C)C)c1CC(=O)NS(=O)(=O)c1nc(C(C)(C)O)cs1] |
| 199 | 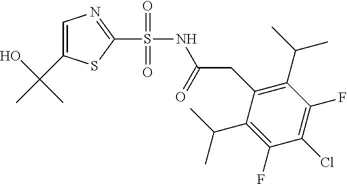 [SMILES: CC(C)c1c(F)c(Cl)c(F)c(C(C)C)c1CC(=O)NS(=O)(=O)c1ncc(C(C)(C)O)s1] |
| 200 | 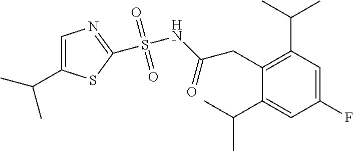 [SMILES: CC(C)c1cnc(S(=O)(=O)NC(=O)Cc2c(C(C)C)cc(F)cc2C(C)C)s1] |
| 201 | 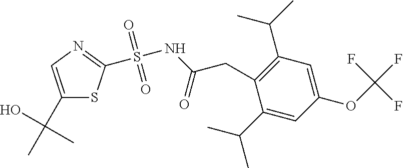 [SMILES: CC(C)c1cc(OC(F)(F)F)cc(C(C)C)c1CC(=O)NS(=O)(=O)c1ncc(C(C)(C)O)s1] |
| 202 | 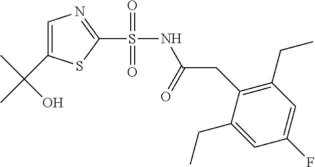 [SMILES: CCc1cc(F)cc(CC)c1CC(=O)NS(=O)(=O)c1ncc(C(C)(C)O)s1] |
| 203 | 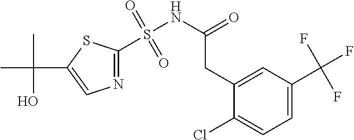 [SMILES: CC(C)(O)c1cnc(S(=O)(=O)NC(=O)Cc2cc(C(F)(F)F)ccc2Cl)s1] |
| 204 | 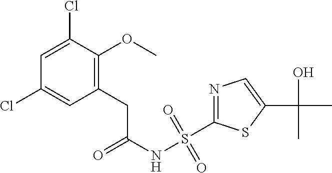 [SMILES: COc1c(Cl)cc(Cl)cc1CC(=O)NS(=O)(=O)c1ncc(C(C)(C)O)s1] |
| 205 | 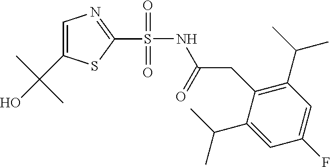 [SMILES: CC(C)c1cc(F)cc(C(C)C)c1CC(=O)NS(=O)(=O)c1ncc(C(C)(C)O)s1] |
| 206 | 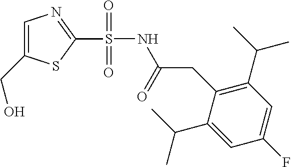 [SMILES: CC(C)c1cc(F)cc(C(C)C)c1CC(=O)NS(=O)(=O)c1ncc(CO)s1] |
| 207 | 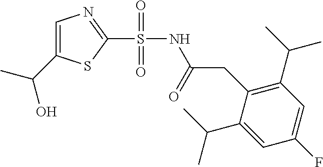 [SMILES: CC(C)c1cc(F)cc(C(C)C)c1CC(=O)NS(=O)(=O)c1ncc(C(C)O)s1] |
| 208 | 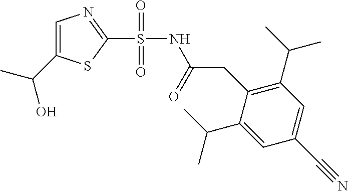 [SMILES: CC(C)c1cc(C#N)cc(C(C)C)c1CC(=O)NS(=O)(=O)c1ncc(C(C)O)s1] |
| 209 | 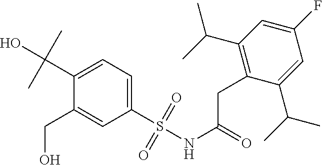 [SMILES: CC(C)c1cc(F)cc(C(C)C)c1CC(=O)NS(=O)(=O)c1ccc(C(C)(C)O)c(CO)c1] |
| 210 | 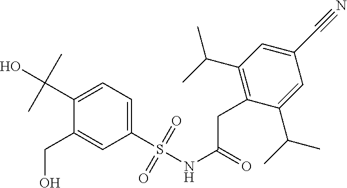 [SMILES: CC(C)c1cc(C#N)cc(C(C)C)c1CC(=O)NS(=O)(=O)c1ccc(C(C)(C)O)c(CO)c1] |
| 211 | 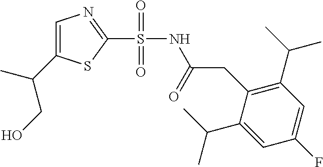 [SMILES: CC(C)c1cc(F)cc(C(C)C)c1CC(=O)NS(=O)(=O)c1ncc(C(C)CO)s1] |
| 212 | 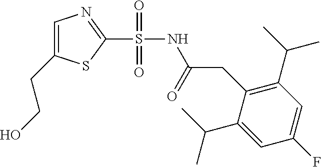 [SMILES: CC(C)c1cc(F)cc(C(C)C)c1CC(=O)NS(=O)(=O)c1ncc(CCO)s1] |
| 213 | 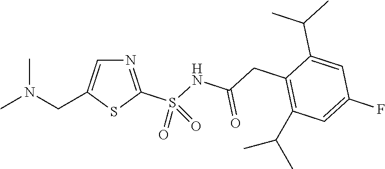 [SMILES: CC(C)c1cc(F)cc(C(C)C)c1CC(=O)NS(=O)(=O)c1ncc(CN(C)C)s1] |
| 214 | 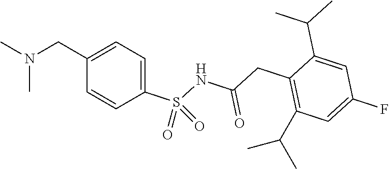 [SMILES: CC(C)c1cc(F)cc(C(C)C)c1CC(=O)NS(=O)(=O)c1ccc(CN(C)C)cc1] |
| 215 | 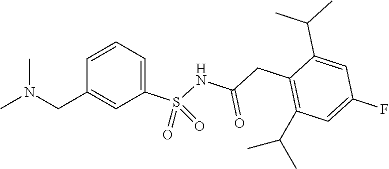 [SMILES: CC(C)c1cc(F)cc(C(C)C)c1CC(=O)NS(=O)(=O)c1cccc(CN(C)C)c1] |
| 216 | 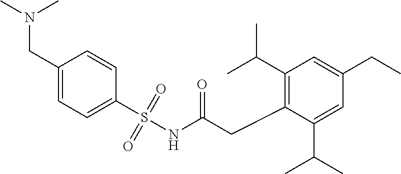 [SMILES: CCc1cc(C(C)C)c(CC(=O)NS(=O)(=O)c2ccc(CN(C)C)cc2)c(C(C)C)c1] |
| 217 | 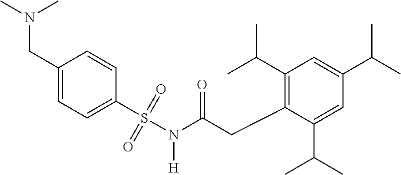 [SMILES: CC(C)c1cc(C(C)C)c(CC(=O)NS(=O)(=O)c2ccc(CN(C)C)cc2)c(C(C)C)c1] |
| 218 | 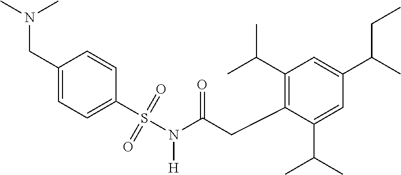 [SMILES: CCC(C)c1cc(C(C)C)c(CC(=O)NS(=O)(=O)c2ccc(CN(C)C)cc2)c(C(C)C)c1] |
| 219 | 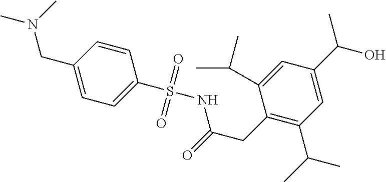 [SMILES: CC(C)c1cc(C(C)O)cc(C(C)C)c1CC(=O)NS(=O)(=O)c1ccc(CN(C)C)cc1] |

and pharmaceutically acceptable salts thereof.

145. A compound selected from the group consisting of the compounds in Table 1C, and pharmaceutically acceptable salts thereof.

146. A pharmaceutical composition comprising a compound or salt as claimed in any one of claims 1-145 and one or more pharmaceutically acceptable excipients.

147. A method for modulating NRLP3 activity, the method comprising contacting NRLP3 with an effective amount of a compound as claimed in any one of claims 1-145 or a pharmaceutical composition as claimed in claim 146.

148. The method of claim 147, wherein the modulating comprises antagonizing NRLP3.

149. The method of any one of claims 147-148, which is carried out in vitro.

150. The method of claim any one of claims 147-149, wherein the method comprises contacting a sample comprising one or more cells comprising NRLP3 with the compound.

151. The method of any one of claims 147, 148 and 150, which is carried out in vivo.

152. The method of claim 151, wherein the method comprises administering the compound to a subject having a disease in which NRLP3 signaling contributes to the pathology and/or symptoms and/or progression of the disease.

153. The method of claim 152, wherein the subject is a human.

154. A method of treating a disease, disorder or condition that is a metabolic disorder, comprising administering to a subject in need of such treatment an effective amount of a compound as claimed in any one of claims 1-145 or a pharmaceutical composition as claimed in claim 146.

155. The method of claim 154, wherein the metabolic disorder is Type 2 diabetes, atherosclerosis, obesity or gout.

156. A method of treating a disease, disorder or condition that is a disease of the central nervous system, comprising administering to a subject in need of such treatment an effective amount of a compound as claimed in any one of claims 1-145 or a pharmaceutical composition as claimed in claim 146.

157. The method of claim 156, wherein the disease of the central nervous system is Alzheimer's disease, multiple sclerosis, Amyotrophic Lateral Sclerosis or Parkinson's disease.

158. A method of treating a disease, disorder or condition that is lung disease, comprising administering to a subject in need of such treatment an effective amount of a compound as claimed in any one of claims 1-145 or a pharmaceutical composition as claimed in claim 146.

159. The method of claim 158, wherein the lung disease is asthma, COPD or pulmonary idiopathic fibrosis.

160. A method of treating a disease, disorder or condition that is liver disease, comprising administering to a subject in need of such treatment an effective amount of a compound as claimed in any one of claims 1-145 or a pharmaceutical composition as claimed in claim 146.

161. The method of claim 160, wherein the liver disease is NASH syndrome, viral hepatitis or cirrhosis.

162. A method of treating a disease, disorder or condition that is pancreatic disease, comprising administering to a subject in need of such treatment an effective amount of a compound as claimed in any one of claims 1-145 or a pharmaceutical composition as claimed in claim 146.

163. The method of claim 162, wherein the pancreatic disease is acute pancreatitis or chronic pancreatitis.

164. A method of treating a disease, disorder or condition that is kidney disease, comprising administering to a subject in need of such treatment an effective amount of a compound as claimed in any one of claims 1-145 or a pharmaceutical composition as claimed in claim 146.

165. The method of claim 164, wherein the kidney disease is acute kidney injury or chronic kidney injury.

166. A method of treating a disease, disorder or condition that is intestinal disease, comprising administering to a subject in need of such treatment an effective amount of a compound as claimed in any one of claims 1-145 or a pharmaceutical composition as claimed in claim 146.

167. The method of claim 166, wherein the intestinal disease is Crohn's disease or Ulcerative Colitis.

168. A method of treating a disease, disorder or condition that is skin disease, comprising administering to a subject in need of such treatment an effective amount of a compound as claimed in any one of claims 1-145 or a pharmaceutical composition as claimed in claim 146.

169. The method of claim 168, wherein the skin disease is psoriasis.

170. A method of treating a disease, disorder or condition that is musculoskeletal disease, comprising administering to a subject in need of such treatment an effective amount of a compound as claimed in any one of claims 1-145 or a pharmaceutical composition as claimed in claim 146.

171. The method of claim 170, wherein the musculoskeletal disease is scleroderma.

172. A method of treating a disease, disorder or condition that is a vessel disorder, comprising administering to a subject in need of such treatment an effective amount of a compound as claimed in any one of claims 1-145 or a pharmaceutical composition as claimed in claim 146.

173. The method of claim 172, wherein the vessel disorder is giant cell arteritis.

174. A method of treating a disease, disorder or condition that is a disorder of the bones, comprising administering to a subject in need of such treatment an effective amount of a compound as claimed in any one of claims 1-145 or a pharmaceutical composition as claimed in claim 146.

175. The method of claim 174, wherein the disorder of the bones is osteoarthritis, osteoporosis or osteopetrosis disorders.

176. A method of treating a disease, disorder or condition that is eye disease, comprising administering to a subject in need of such treatment an effective amount of a compound as claimed in any one of claims 1-145 or a pharmaceutical composition as claimed in claim 146.

177. The method of claim 176, wherein the eye disease is glaucoma or macular degeneration.

178. A method of treating a disease, disorder or condition that is a disease caused by viral infection, comprising administering to a subject in need of such treatment an effective amount of a compound as claimed in any one of claims 1-145 or a pharmaceutical composition as claimed in claim 146.

179. The method of claim 178, wherein the diseases caused by viral infection is HIV or AIDS.

180. A method of treating a disease, disorder or condition that is an autoimmune disease, comprising administering to a subject in need of such treatment an effective amount of a compound as claimed in any one of claims 1-145 or a pharmaceutical composition as claimed in claim 146.

181. The method of claim 180, wherein the autoimmune disease is Rheumatoid Arthritis, Systemic Lupus Erythematosus, Autoimmune Thyroiditis, Addison's disease, or pernicious anemia.

182. The method of claim 180, wherein the disease is cancer or aging.

183. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AMIL) chronic myeloid leukemia (CML); gastric cancer; and lung cancer metastasis, comprising administering to a subject in need of such treatment an effective amount of a compound as claimed in any one of claims 1-145 or a pharmaceutical composition as claimed in claim 146.

184. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis, comprising administering to a subject in need of such treatment an effective amount of a compound as claimed in any one of claims 1-145 or a pharmaceutical composition as claimed in claim 146.

185. The method of any one of claims 183-184, wherein the cancer is MDS.

186. The method of any one of claims 183-184, wherein the cancer is non-small lung cancer.

187. The method of any one of claims 183-184, wherein the cancer is acute lymphoblastic leukemia.

188. The method of any one of claims 183-184, wherein the cancer is LCH.

189. The method of any one of claims 183-184, wherein the cancer is multiple myeloma.

190. The method of any one of claims 183-184, wherein the cancer is promyelocytic leukemia.

191. The method of claim 183, wherein the cancer is acute myeloid leukemia (AMIL).

192. The method of claim 183, wherein the cancer is chronic myeloid leukemia (CML).

193. The method of any one of claims 183-184, wherein the cancer is gastric cancer.

194. The method of any one of claims 183-184, wherein the cancer is lung cancer metastasis.

195. The method of any one of claims 147-194, further comprising administering a therapeutically effective amount of an anti-TNFα agent to the subject.

196. The method of claim 195, wherein the NLRP3 antagonist is administered to the subject prior to administration of the anti-TNFα agent to the subject.

197. The method of claim 195, wherein the anti-TNFα agent is administered to the subject prior to the administration of the NLRP3 antagonist to the subject.

198. The method of claim 195, wherein the NLRP3 antagonist and the anti-TNFα agent are administered to the subject at substantially the same time.

199. The method of claim 195, wherein the NLRP3 antagonist and the anti-TNFα agent are formulated together in a single dosage form.

200. The compound of claim 1, wherein:
Ar is a heteroaryl group 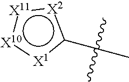
X¹ is N, CR⁴¹ or NR⁴¹;
X¹⁰ is N, CR¹⁰ or NR¹⁰;
X¹¹ is N, CR¹ or NR¹;
X² is N, CR⁴² or NR⁴²;
wherein at least two of R⁴¹, R¹⁰, R¹, and R⁴² are present and on adjacent atoms, such that taken together with the atoms connecting them form at least one monocyclic or bicyclic 3-to-12-membered carbocyclic ring or at least one monocyclic or bicyclic 5- to 12-membered heterocyclic ring containing 1-3 heteroatoms independently selected from O, N, NH, NR¹³, and S, wherein the carbocyclic ring or heterocyclic ring is optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

201. The compound of claim 200, wherein: 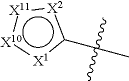
is a pyrazole.

202. The compound of claim 201, wherein:
the at least two of R⁴¹, R¹⁰, R¹, and R⁴² present on adjacent atoms and taken together with the atoms connecting them, form a monocyclic 5- to 12-membered heterocyclic ring containing 1-3 heteroatoms selected from 0 and N, optionally substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³ COOC₁-C₆ alkyl, C₆-C₁₀ aryl, and CONR¹¹R1.

203. The compound of any one of claims 200 to 202, wherein
Ar is selected from 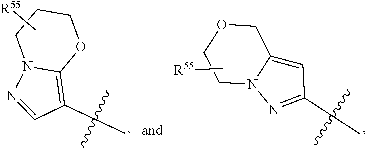
optionally substituted with one or more substituents R⁵⁵ wherein each R⁵ is independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR¹¹R¹², ═NR¹³, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR¹¹R¹², wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR¹¹R¹², ═NR¹³, COOC₁-C₆ alkyl, C₆-C₁₀ aryl, and CONR¹¹R¹².

204. The compound of any one of claims 200 to 203, wherein 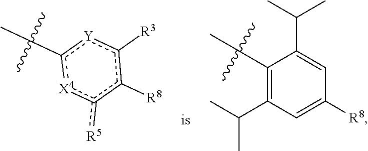
wherein the moiety
R⁸ is selected from H, CN, halo, CO₂C₁-C₆ alkyl, CO₂C₃-C₈ cycloalkyl, CONR¹¹R¹², C₁-C₆ alkyl optionally substituted with hydroxy, C₁-C₆ alkoxy, C₁-C₆ haloalkoxy, and C₁-C₆ haloalkyl.

205. The compound of claim 204, wherein R⁸ is CN.
